(19) United States
(12) Patent Application Publication
(10) Pub. No.: US 2026/0207563 A1
(43) Pub. Date: Jul. 23, 2026

(54) UREA COMPOUNDS AS ACTIVATORS OF POTASSIUM CHANNELS KV7.2/7.3 USEFUL IN THE TREATMENT OF CNS AND PNS DISORDERS

(71) Applicant: ANGELINI PHARMA S.P.A., Roma (IT)

(72) Inventors: Barbara GAROFALO, Verona (IT); Rosella OMBRATO, Roma (IT); Federica PRATI, Ciampino (RM) (IT); Thomas David PALLIN, Harlow (GB); Jamie David KNIGHT, Harlow (GB); Pascal SAVY, Harlow (GB)

(73) Assignee: ANGELINI PHARMA S.P.A., Roma (IT)

(21) Appl. No.: 19/135,729

(22) PCT Filed: Dec. 4, 2023
(86) PCT No.: PCT/EP2023/084100

§ 371 Date: Jun. 4, 2025

(30) Foreign Application Priority Data
Dec. 5, 2022 (IT) 102022000024963

(51) Int. Cl. A61K 31/435; A61K 31/437; A61K 31/4436; A61K 31/4523; A61K 31/453; A61K 31/454; A61K 31/4545; A61K 31/472; A61K 31/50; A61K 31/505; A61K 31/506; A61K 31/5377

(52) U.S. Cl. CPC A61K 31/435; A61K 31/437; A61K 31/4436; A61K 31/4523; A61K 31/453; A61K 31/454; A61K 31/4545; A61K 31/472; A61K 31/50; A61K 31/505; A61K 31/506; A61K 31/5377

(57) ABSTRACT

Compounds of formula (I), defined in the specification, capable of promoting the opening of Kv7.2/Kv7.3 potassium channels are useful for the treatment of disorders of the central nervous system (CNS), such as epilepsy and neurodegenerative disorders, and of the peripheral nervous system (PNS), such as chronic and neuropathic pain.

### FIELD OF THE INVENTION

The present invention relates to compounds capable of promoting the opening of Kv7.2/7.3 potassium channels and their use as a drug, particularly in the treatment of disorders of the central nervous system (CNS), e.g., epilepsy and neurodegenerative disorders, and of the peripheral nervous system (PNS), e.g., chronic and neuropathic pain.

### STATE OF THE ART

Voltage-gated potassium (Kv) channels conduct potassium (K+) ions across cell membranes in response to changes in membrane potential and can therefore regulate cellular excitability by modulating (increasing or decreasing) the cell's electrical activity.

Functional Kv channels exist as multimeric structures formed by the association of four alpha and four beta subunits. Alpha subunits comprise six transmembrane domains, a pore-forming loop, and a voltage sensor and are arranged symmetrically around a central pore. Beta or auxiliary subunits interact with alpha subunits and can modify the properties of the channel complex to include, but not limited to, alterations in electrophysiological or biophysical properties of the channel, levels, or expression patterns.

Nine families of Kv channel alpha subunits have been identified, referred to as Kv1-Kv9. The Kv7 channel family consists of at least five members including Kv7.1, Kv7.2, Kv7.3, Kv7.4, and Kv7.5. Alternatively, the members of this family are referred to by the gene names KCNQ1, KCNQ2, KCNQ3, KCNQ4, and KCNQ5, respectively (Dalby-Brown, et al., Current Topics in Medicinal Chemistry, 2006, 6(10), 999-1023).

As mentioned above, neuronal Kv7 potassium channels play a role in the control of neuronal excitation. Kv7 channels, particularly the Kv7.2/7.3 heterotetramers, underlie the M-current (Wang et al Science. 1998, Dec. 4; 282(5395):1890-1893).

The M current has a characteristic time and voltage dependence that results in the stabilization of the membrane potential in response to multiple excitatory stimuli. Thus, M current is involved in the control of neuronal excitability (Delmas & Brown, Nature, 2005, 6, 850-862).

The M current is a noninactivating potassium current found in many neuronal cell types. In each cell type, it is dominant in the control of membrane excitability being the only sustained current in the range of action potential initiation (Marrion, Annual Review Physiology, 1997, 59, 483-504).

The five members of this family of ion channels differ in their expression patterns. Expression of Kv7.1 is restricted to the heart, peripheral epithelium, and smooth muscle, whereas expression of Kv7.2, Kv7.3, Kv7.4, and Kv7.5 appears to be dominant in the nervous system that includes the hippocampus, cortex, ventral tegmental area, and dorsal root ganglion neurons. Kv7.4 is a subtype selectively expressed in the auditory pathway including hair cells of the inner ear. In addition to neurons, Kv7.4 and Kv7.5 are also expressed in various smooth muscle cells (Greene & Hoshi, Cellular and Molecular Life Sciences, 2017, 74(3), 495-508).

The KCNQ2 and KCNQ3 genes appear to be mutated in an inherited form of epilepsy known as benign familial neonatal seizures (Rogawski, Trends in Neuroscience 2000, 23, 393-398). Proteins encoded by the KCNQ2 and KCNQ3 genes are localized in pyramidal neurons of the human cortex and hippocampus, regions of the brain associated with seizure generation and propagation (Cooper et al., Proceedings National Academy of Science USA, 2000, 97(9), 4914-4919).

In addition, mRNAs for Kv7.2, Kv7.3, and Kv7.5 are expressed in astrocytes and glial cells. Thus, Kv7.2, Kv7.3, and Kv7.5 channels may help modulate synaptic activity in the CNS and contribute to the neuroprotective effects of KCNQ channel activators (Noda, et al., Society for Neuroscience Abstracts 2003, 53.9), which would be relevant to the treatment of neurodegenerative disorders such as, but not limited to, Alzheimer's disease, Parkinson's disease, and Huntington's chorea.

mRNAs for Kv7.2 and Kv7.3 are found in regions of the brain associated with anxiety and emotional behaviors such as depression and bipolar disorder, for example the hippocampus, ventral tegmental area, and amygdala (Saganich et al., Journal of Neuroscience, 2001, 21(13), 4609-4624; Friedman et al., Nat. Commun., 2016, 7, 11671).

Kv7.2/7.3 channels have also been reported to be upregulated in models of neuropathic pain (Wickenden, et al., Society for Neuroscience Abstracts, 2002, 454.7), and modulators of potassium channels have been hypothesized to be active in both neuropathic pain and epilepsy (Schroder et al., Neuropharmacology 2001, 40(7), 888-898). In addition to a role in neuropathic pain, mRNA expression for Kv7.2-5 in the trigeminal and dorsal root ganglia and in the trigeminal caudal nucleus implies that activators of these channels may also influence sensory processing of migraine pain (Goldstein et al., Society for Neuroscience Abstracts 2003, 53.8).

Retigabine and flupirtine are known Kv7.2/7.3 potassium channel activating compounds that have been used in the treatment of epilepsy, migraine, neuropathic pain, acute pain, and tinnitus. Retigabine has been withdrawn from the market because of its adverse side effects, particularly urinary retention and changes in retinal and skin pigmentation. Flupirtine should be used by individuals who do not respond to other analgesic treatments and for no longer than two weeks because of its hepatic toxicity.

### SUMMARY OF THE INVENTION

The Applicant has addressed the problem of providing novel therapies for the treatment of disorders of the central nervous system (CNS), e.g., epilepsy and neurodegenerative disorders, and the peripheral nervous system (PNS), e.g., chronic and neuropathic pain.

The Applicant focused its attention on potassium channel activating compounds Kv7.2/7.3, initiating research work that could provide alternative compounds to retigabine and flupirtine.

After extensive experimentation, the Applicant identified a number of novel compounds capable of acting as drugs, particularly for the treatment of disorders that are modulated by Kv7.2/7.3 potassium channels.

In particular, as demonstrated in the examples in the following experimental portion, the Applicant has identified a number of compounds capable of acting as activators of Kv7.2/7.3 potassium channels.

Based on the information known to the man skilled in the art, Applicant believes that these compounds may be effective in the treatment of a variety of disorders of the central nervous system (CNS), e.g., epilepsy and neurodegenerative disorders, and the peripheral nervous system (PNS), e.g., chronic, and neuropathic pain.

Thus, in a first aspect, the present invention relates to a Kv7.2/7.3 potassium channel activator compound, or a pharmaceutically acceptable salt thereof, for use in treating disorders that are modulated by KV7.2/KV7.3 potassium channels, preferably selected from the group consisting of central nervous system (CNS) and peripheral nervous system (PNS) disorders, said compound having the following general formula (I)

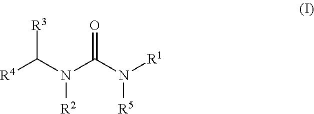

R1 is represented by -L1-A1, wherein
   L1 is a bond or a linear or branched C1-C6 alkyl chain, optionally substituted by one or more halogen atom or hydroxyl group, and optionally comprising an oxy (—O—) group within or at any end of the alkyl chain, and
   A1 is a hydrogen atom, or
      (i) an aromatic or aliphatic ring having three to six members, optionally containing one or more heteroatoms selected from the group consisting of N, O and S, unsubstituted or substituted with one or more substituents selected from the group consisting of halogen, hydroxyl, methoxy, CN, CF₃, C1-C3 alkyl chain optionally substituted by one or more halogen atom, and AR, wherein AR is an aromatic or aliphatic ring, optionally containing one or more heteroatoms selected from the group consisting of N, O and S, unsubstituted or substituted with one or more substituents selected from the group consisting of halogen, hydroxyl, methoxy, CN, CF₃, and C1-C3 alkyl chain optionally substituted by one or more halogen atom, or
      (ii) a fused or bridged bicyclic ring having five to twelve members, optionally containing one or more heteroatoms selected from the group consisting of N, O and S, unsubstituted or substituted with one or more substituents selected from the group consisting of halogen, hydroxyl, methoxy, CN, CF₃, O-Gr, and C1-C3 alkyl optionally substituted by one or more halogen atom, wherein Gr is an aliphatic ring having three to six members, optionally containing one or more heteroatoms selected from the group consisting of N, O and S, unsubstituted or substituted with one or more substituents selected from the group consisting of halogen, hydroxyl, methoxy, CN, CF₃, C1-C3 alkyl chain, optionally substituted by one or more halogen atom;
R2 is hydrogen, or a C1-C3 alkyl chain;
R3 is hydrogen or a C1-C3 alkyl chain optionally substituted by hydroxyl group, or an aliphatic ring, optionally containing one or more heteroatoms selected from the group consisting of N, O and S, unsubstituted or substituted with one or more substituents selected from the group consisting of halogen, hydroxyl, methoxy, CN, and CF₃;
R4 is represented by -L2-A2, wherein
   L2 is a bond or a C1-C3 alkyl chain, and
   A2 is an aromatic ring having 6 members and comprising one or two nitrogen atoms, optionally condensed with an aliphatic ring having 5 or 6 carbon atoms, said aromatic ring being substituted by one or more substituent selected from
      (i) a halogen atom,
      (ii) a C1-C3 alkyl chain, optionally substituted with one or more halogen atoms,
      (iii) an aliphatic ring having 4 to 6 members comprising one or more heteroatoms selected from O and N, optionally substituted with one or more halogen atom or C1-C3 alkyl chain optionally substituted with one or more halogen or hydroxyl, and
      (iv) a group represented by the following formula —S6-L3-A3, wherein S6 is an oxygen or nitrogen atom, L3 is a bond or a C1-C4 alkyl chain, and A3 is
         (a) an aliphatic or aromatic ring having 3 to 6 members, optionally comprising one or more heteroatoms selected from O and N, and optionally substituted with a halogen atom or a C1-C3 alkyl chain, optionally substituted with one or more halogen, hydroxyl, or CN, or
         (b) a C1-C3 alkyl chain, optionally substituted with one or more halogen atoms.
R5 is hydrogen, or a C1-C3 alkyl chain;
or R4 together with R2 or R3, and/or R5 together with R1, and/or R3 together with R2, form a five to six member aliphatic ring optionally substituted by an alkyl chain having 1-3 carbon atoms, a halogen atom, or a hydroxyl group, said aliphatic ring optionally comprising an oxygen atom and being optionally condensed with or substituted by a benzene or pyridine ring, said benzene or pyridine ring being optionally substituted by a halogen, or an alkyl or alkoxy chain having 1-3 carbon atoms, said chain being optionally substituted by one or more halogen atom;
provided that when R2, R3 and R5 are hydrogen atoms and R4 is

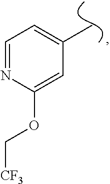

then R1 is different from

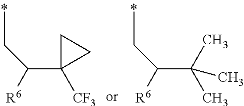

where R6 is H or OH, and
provided that when L1 is a bond, A1 is not a substituted or unsubstituted cyclobutane ring.

In a second aspect, the present invention relates to a Kv7.2/7.3 potassium channel activator compound, or a pharmaceutically acceptable salt thereof, selected from the following Table A and Table B.

In a third aspect, the present invention relates to a pharmaceutical composition comprising (i) a Kv7.2/7.3 potassium channel activating compound, or a pharmaceutically acceptable salt thereof, according to the second aspect of the present invention, and (ii) at least one pharmaceutically acceptable excipient.

Advantageously, the pharmaceutical composition according to the third aspect of the present invention can be used in the treatment of disorders that are modulated by Kv7.2/7.3 potassium channels, preferably in the treatment of central nervous system (CNS) and peripheral nervous system (PNS) disorders.

In a fourth aspect, the present invention relates to a method for treating disorders that are modulated by Kv7.2/7.3 potassium channels in a subject in need thereof comprising administering a therapeutically effective amount of a Kv7.2/7.3 potassium channel activating compound, or a pharmaceutically acceptable salt thereof, according to the first or second aspect of the present invention.

For the purposes of this description and the claims that follow, the phrase “pharmaceutically acceptable” is intended to define, without any particular limitation, any material suitable for the preparation of a pharmaceutical composition to be administered to a living being.

For the purposes of this description and the claims that follow, the phrase “therapeutically effective amount” means an amount of compound sufficient to alleviate, arrest, partially arrest, remove or delay the clinical manifestations of a certain disease and its complications in a therapeutic treatment comprising the administration of said compound.

For the purposes of this description and the claims that follow, the term “treatment” or “treating” means the management and care of a patient for the purpose of alleviating, arresting, partially arresting, removing or delaying the progress of the clinical manifestation of disease. The patient to be treated is preferably a mammal, particularly a human being.

For the purposes of the present description and the following claims, the expression “for example” and the terms “preferably”, “advantageously”, “particularly”, and the like are used to better illustrate the invention without adding any limitation to the scope of the invention, unless otherwise indicated.

For the purposes of the present description and the claims that follow, the phrase “KV7.2/KV7.3 potassium channel activator” refers to a compound that causes a shift in voltage dependence for channel opening to more negative potentials, meaning that the KV7.2/KV7.3 potassium channels open to more negative potentials in the presence of said compound, facilitating the transmission of ions through them.

### DETAILED DESCRIPTION OF THE INVENTION

A first aspect of the present invention relates to a Kv7.2/7.3 potassium channel activator compound, or a pharmaceutically acceptable salt thereof, for use in treating disorders that are modulated by KV7.2/KV7.3 potassium channels, preferably selected from the group consisting of central nervous system (CNS) and peripheral nervous system (PNS) disorders, said compound having the general formula (I) described above.

In an embodiment of the present invention, L1 is a bond or a C1-C2 alkyl chain.

In another embodiment, L1 is a linear or branched C3-C6 alkyl chain, optionally substituted by one or more halogen atom or hydroxyl group, and optionally comprising an oxy (—O—) group within or at any end of the alkyl chain, such as, for example, —CH₂—CH₂—CH₂—, —CH₂—CH₂—CH₂—CH₂—, —CHOH—C(CH₃)₂—CH₂—, —CH₂—CHOH—C(CH₃)₂—CH₂—, —CH₂—CH₂—CCF₃OH—CH₂—, —CH₂—CH₂—CCF₃OH—CH₂—, —CH₂—CH₂—O—CCF₃ CH₃—CH₂—, —CH₂—CCH₃OH—CH₂—.

In an embodiment of the present invention, A1 is hydrogen, or
(i) an aromatic or aliphatic ring selected from the group consisting of phenyl, cyclopropyl, cyclobutyl, cyclopentyl, and cyclohexyl, optionally substituted with one or more substituents selected from the group consisting of halogen, hydroxyl, methoxy, CN, CF₃, C1-C3 alkyl chain, optionally substituted by one or more halogen atom, or
(ii) a bicyclic ring selected from the group consisting of bicycle[1,1,1]pentane, indane (2,3-dihydro-1H-indene), 2,3-dihydro-1H-indole, 6,7-dihydro-5H-cyclopenta[c]pyridine, chromane (3,4-dihydro-2H-1-benzopyran), coumaran (2,3-dihydro-1-benzofuran), tetralin (1,2,3,4-tetrahydronaphthalene), thiochroman (3,4-dihydro-2H-1-benzothiopyran), 5,6-dihydro-4H-cyclopenta[b]thiophene, 6,7-dihydro-5H-cyclopenta[b]pyridine, 5,6,7,8-tetrahydroisoquinoline, 5,6,7,8-tetrahydroquinoxaline, 2,3,4,5-tetrahydro-1-benzoxepine, and 6,7,8,9-tetrahydro-5H-benzo[7]annulene, optionally substituted with one or more substituents selected from the group consisting of halogen, hydroxyl, methoxy, CN, CF₃, C1-C3 alkyl chain, optionally substituted by one or more halogen atom.

In a preferred embodiment of the present invention, L2 is a bond or a C1-C2 alkyl chain, more preferably a bond.

In a preferred embodiment of the present invention, A2 is pyridine, pyridazine, pyrimidine, or pyrazine, more preferably pyridine or pyrimidine, optionally condensed with an aliphatic ring having 5 or 6 carbon atoms.

In an embodiment of the present invention A2 is substituted by a halogen atom preferably selected from the group consisting of chlorine and fluorine.

In an embodiment of the present invention A2 is substituted by a C1-C3 alkyl chain, preferably selected from the group consisting of methyl, ethyl, propyl, and isopropyl, optionally substituted with one or more halogen atoms, preferably selected from the group consisting of chlorine and fluorine.

In an embodiment of the present invention A2 is substituted by an aliphatic ring having 4 to 6 members comprising one nitrogen atom, one oxygen atom, or both, optionally substituted with one or more halogen atom or C1-C3 alkyl chain optionally substituted with one or more halogen or hydroxyl, wherein the halogen atom is preferably selected from the group consisting of chlorine and fluorine. In a preferred embodiment, the aliphatic ring having 4 to 6 member is azetidine, pyrrolidine, piperidine, oxetane, tetrahydrofuran, tetrahydropyran oxazetidine, oxazolidine, or morpholine, more preferably azetidine, pyrrolidine, piperidine, or morpholine.

In an embodiment of the present invention A2 is substituted by a group represented by the formula —S6-L3-A3.

S6 is preferably an oxygen atom.

L3 is preferably a bond or a C1-C3 alkyl chain, more preferably selected from the group consisting of methyl, ethyl, propyl, and isopropyl, optionally substituted with one or more halogen atoms, preferably selected from the group consisting of chlorine and fluorine.

A3 is preferably an aliphatic ring having 3 to 6 members or an aromatic ring having 5 or 6 member, optionally comprising one or more heteroatoms selected from O and N, and optionally substituted with a halogen atom or a C1-C3 alkyl chain, optionally substituted with one or more halogen, hydroxyl, CN. In a more preferred embodiment, the aliphatic ring is cyclopropane, cyclobutane, azetidine, oxetane, tetrahydropyran. In a more preferred embodiment, the aromatic ring is phenyl, pyrrole, furan, oxazole, pyrimidine, or pyridine.

Alternatively, A3 is preferably a C1-C2 alkyl chain, optionally substituted with one or more halogen atoms, preferably selected from the group consisting of chlorine and fluorine. More preferably, A3 is a CF₃ or C2F₅ group.

In an embodiment of the present invention R4 together with R2 or R3 and/or R3 together with R2 forms a five to six member aliphatic ring, condensed with or substituted by a benzene or pyridine ring said benzene or pyridine ring being substituted by a halogen, or an alkyl or alkoxy chain having 1-3 carbon atoms, said chain being optionally substituted by one or more halogen atom.

In a further embodiment of the present invention R4 together with R2 or R3 and/or R3 together with R2 forms a five member aliphatic ring, condensed with or substituted by a pyridine ring said pyridine ring being substituted by an alkoxy chain having 1-3 carbon atoms, said chain being optionally substituted by one or more halogen atom, preferably one or more fluorine atom.

In a specific embodiment of the present invention R4 or R3 together with R2 forms the following rings A or B, wherein the asterisked nitrogen atom is from the ureido structure of formula (I):

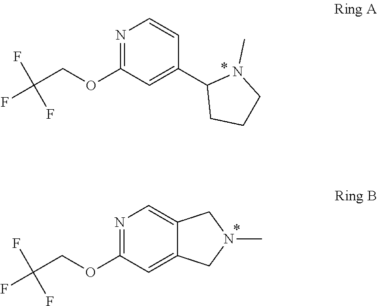 [SMILES: CN1CCCC1c1ccnc(OCC(F)(F)F)c1 ; CN1Cc2cnc(OCC(F)(F)F)cc2C1]

In a specific embodiment of the present invention R4 together with R3 form s the following ring C, wherein the asterisked bond is linked to the nitrogen atom of the ureido structure of formula (I):

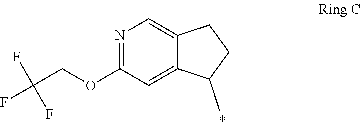 [SMILES: CC1CCc2cnc(OCC(F)(F)F)cc21]

In an embodiment of the present invention R5 together with R1 forms a five to six member aliphatic ring optionally substituted by an alkyl chain having 1-3 carbon atoms, said aliphatic ring optionally comprising an oxygen atom and being optionally condensed with a benzene ring, said benzene ring being optionally substituted by a halogen, or an alkyl or alkoxy chain having 1-3 carbon atoms, said chain being optionally substituted by one or more halogen atom.

In a further embodiment of the present invention R5 together with R1 forms a five to six member aliphatic ring optionally substituted by a methyl group, said aliphatic ring optionally comprising an oxygen atom and being optionally condensed with a benzene ring, said benzene ring being optionally substituted by a halogen.

In a specific embodiment of the present invention R5 together with R1 forms the following rings D and E, wherein the asterisked nitrogen atom is from the ureido structure of formula (I):

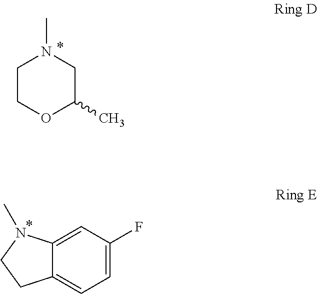

Advantageously, the second aspect of the present invention relates to a KV7.2/KV7.3 potassium channel activator compound, or a pharmaceutically acceptable salt thereof, selected from the group consisting of the compounds of the following Table A and Table B:

TABLE A

| No. | Structure |
|---|---|
| 1 | 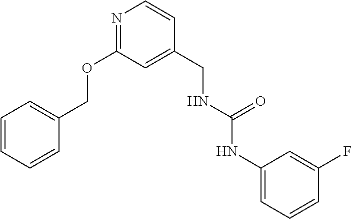 [SMILES: O=C(NCc1ccnc(OCc2ccccc2)c1)Nc1cccc(F)c1] |
| 2 | 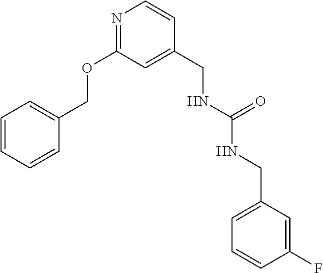 [SMILES: O=C(NCc1cccc(F)c1)NCc1ccnc(OCc2ccccc2)c1] |
| 3 | 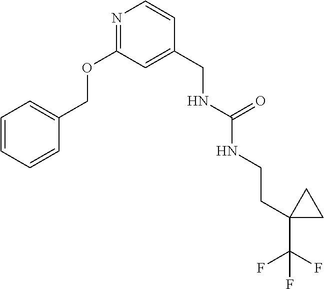 [SMILES: O=C(NCCC1(C(F)(F)F)CC1)NCc1ccnc(OCc2ccccc2)c1] |
| 4 | 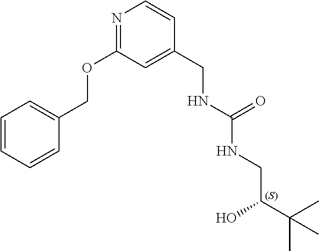 |
| 5 | 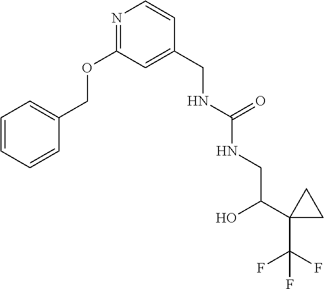 [SMILES: O=C(NCc1ccnc(OCc2ccccc2)c1)NCC(O)C1(C(F)(F)F)CC1] |
| 6 | 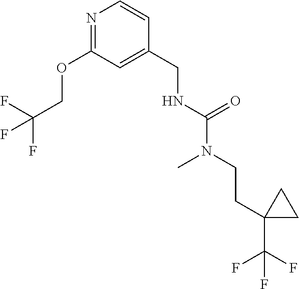 [SMILES: CN(CCC1(C(F)(F)F)CC1)C(=O)NCc1ccnc(OCC(F)(F)F)c1] |
| 7 | 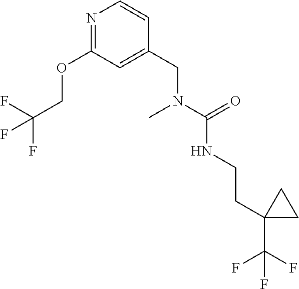 [SMILES: CN(Cc1ccnc(OCC(F)(F)F)c1)C(=O)NCCC1(C(F)(F)F)CC1] |
| 8 | 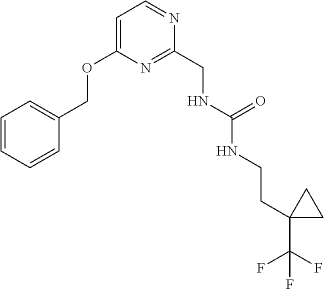 [SMILES: O=C(NCCC1(C(F)(F)F)CC1)NCc1nccc(OCc2ccccc2)n1] |
| 9 | 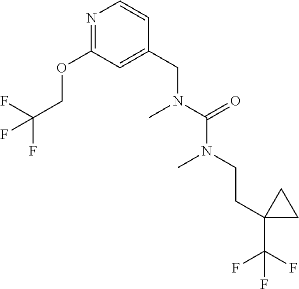 [SMILES: CN(CCC1(C(F)(F)F)CC1)C(=O)N(C)Cc1ccnc(OCC(F)(F)F)c1] |
| 10 | 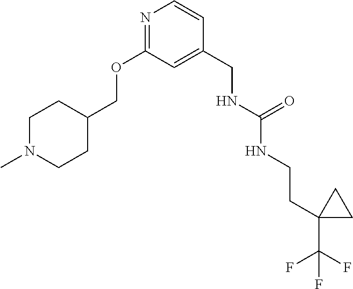 [SMILES: CN1CCC(COc2cc(CNC(=O)NCCC3(C(F)(F)F)CC3)ccn2)CC1] |
| 11 | 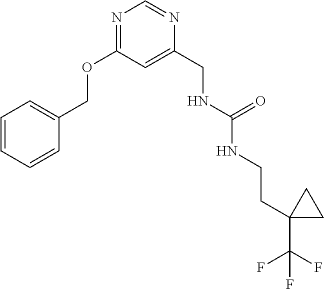 [SMILES: O=C(NCCC1(C(F)(F)F)CC1)NCc1cc(OCc2ccccc2)ncn1] |
| 12 | 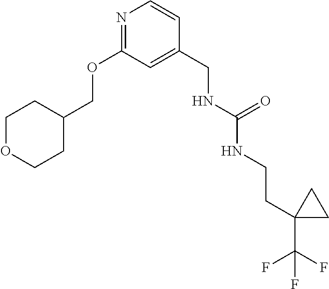 [SMILES: O=C(NCCC1(C(F)(F)F)CC1)NCc1ccnc(OCC2CCOCC2)c1] |
| 13 | 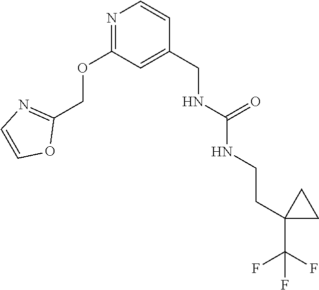 [SMILES: O=C(NCCC1(C(F)(F)F)CC1)NCc1ccnc(OCc2ncco2)c1] |
| 14 | 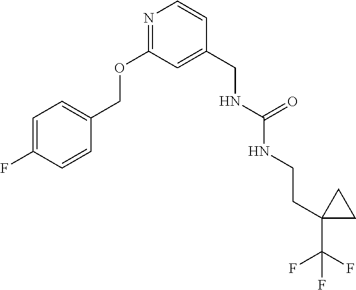 [SMILES: O=C(NCCC1(C(F)(F)F)CC1)NCc1ccnc(OCc2ccc(F)cc2)c1] |
| 15 | 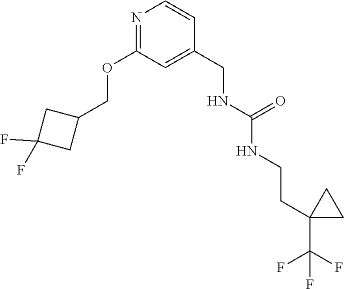 [SMILES: O=C(NCCC1(C(F)(F)F)CC1)NCc1ccnc(OCC2CC(F)(F)C2)c1] |
| 16 | 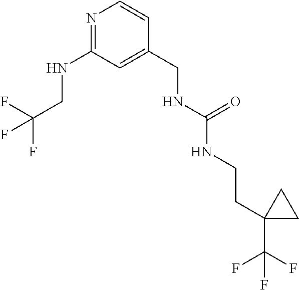 [SMILES: O=C(NCCC1(C(F)(F)F)CC1)NCc1ccnc(NCC(F)(F)F)c1] |
| 17 | 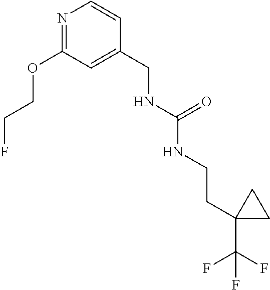 [SMILES: O=C(NCCC1(C(F)(F)F)CC1)NCc1ccnc(OCCF)c1] |
| 18 | 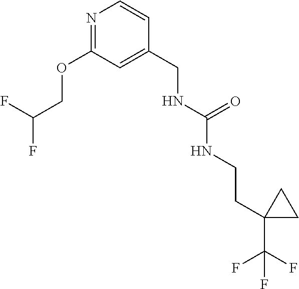 [SMILES: O=C(NCCC1(C(F)(F)F)CC1)NCc1ccnc(OCC(F)F)c1] |
| 19 | 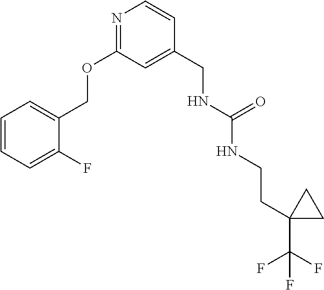 [SMILES: O=C(NCCC1(C(F)(F)F)CC1)NCc1ccnc(OCc2ccccc2F)c1] |
| 20 | 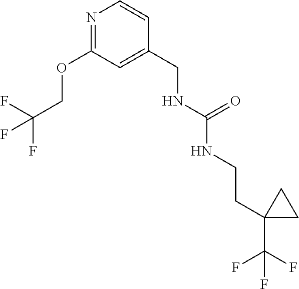 [SMILES: O=C(NCCC1(C(F)(F)F)CC1)NCc1ccnc(OCC(F)(F)F)c1] |
| 21 | 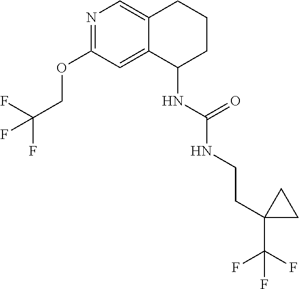 [SMILES: O=C(NCCC1(C(F)(F)F)CC1)NC1CCCc2cnc(OCC(F)(F)F)cc21] |
| 22 | 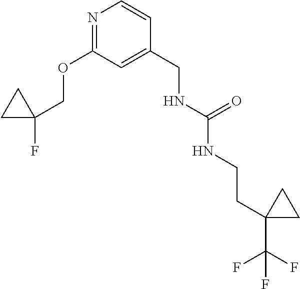 [SMILES: O=C(NCCC1(C(F)(F)F)CC1)NCc1ccnc(OCC2(F)CC2)c1] |
| 23 | 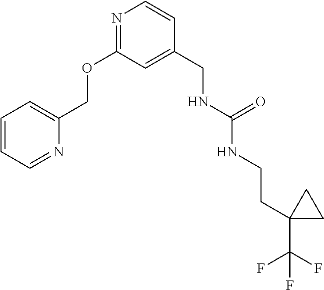 [SMILES: O=C(NCCC1(C(F)(F)F)CC1)NCc1ccnc(OCc2ccccn2)c1] |
| 24 | 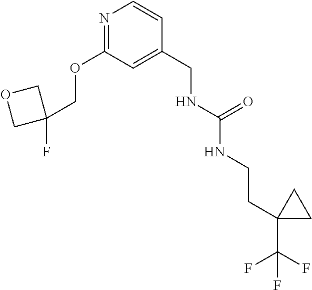 [SMILES: O=C(NCCC1(C(F)(F)F)CC1)NCc1ccnc(OCC2(F)COC2)c1] |
| 25 | 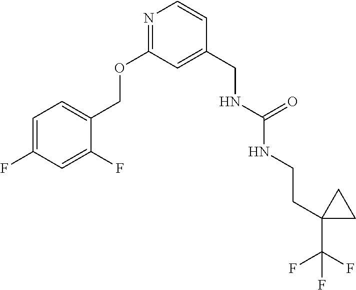 [SMILES: O=C(NCCC1(C(F)(F)F)CC1)NCc1ccnc(OCc2ccc(F)cc2F)c1] |
| 26 | 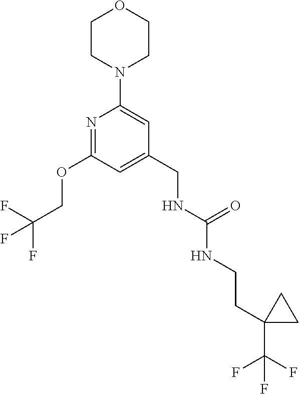 [SMILES: O=C(NCCC1(C(F)(F)F)CC1)NCc1cc(OCC(F)(F)F)nc(N2CCOCC2)c1] |
| 27 | 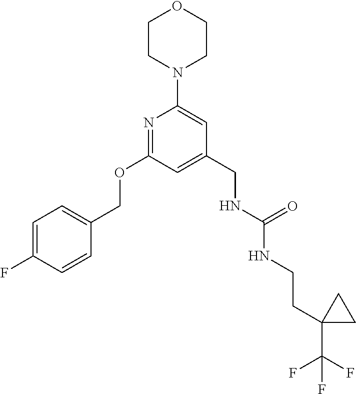 [SMILES: O=C(NCCC1(C(F)(F)F)CC1)NCc1cc(OCc2ccc(F)cc2)nc(N2CCOCC2)c1] |
| 28 | 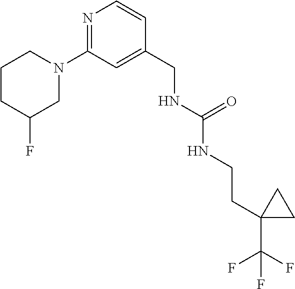 [SMILES: O=C(NCCC1(C(F)(F)F)CC1)NCc1ccnc(N2CCCC(F)C2)c1] |
| 29 | 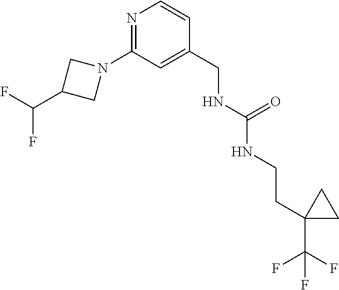 [SMILES: O=C(NCCC1(C(F)(F)F)CC1)NCc1ccnc(N2CC(C(F)F)C2)c1] |
| 30 | 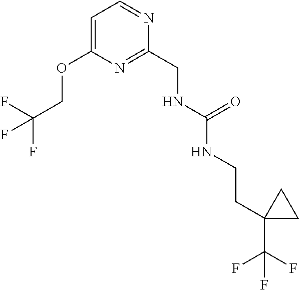 [SMILES: O=C(NCCC1(C(F)(F)F)CC1)NCc1nccc(OCC(F)(F)F)n1] |
| 31 | 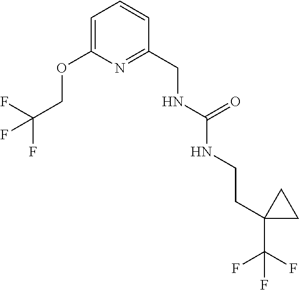 [SMILES: O=C(NCCC1(C(F)(F)F)CC1)NCc1cccc(OCC(F)(F)F)n1] |
| 32 | 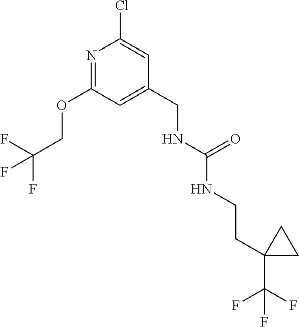 [SMILES: O=C(NCCC1(C(F)(F)F)CC1)NCc1cc(Cl)nc(OCC(F)(F)F)c1] |
| 33 | 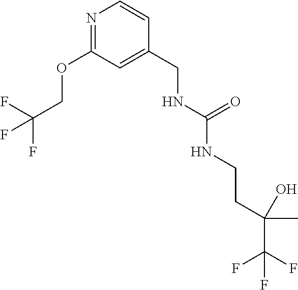 [SMILES: CC(O)(CCNC(=O)NCc1ccnc(OCC(F)(F)F)c1)C(F)(F)F] |
| 34 | 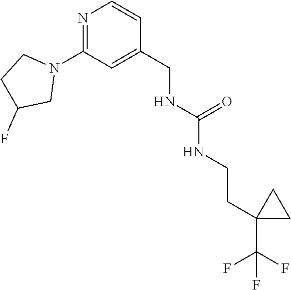 [SMILES: O=C(NCCC1(C(F)(F)F)CC1)NCc1ccnc(N2CCC(F)C2)c1] |
| 35 | 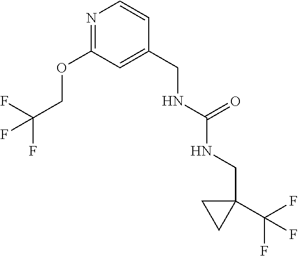 [SMILES: O=C(NCc1ccnc(OCC(F)(F)F)c1)NCC1(C(F)(F)F)CC1] |
| 36 | 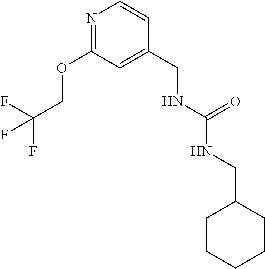 [SMILES: O=C(NCc1ccnc(OCC(F)(F)F)c1)NCC1CCCCC1] |
| 37 | 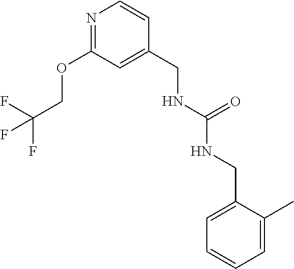 [SMILES: Cc1ccccc1CNC(=O)NCc1ccnc(OCC(F)(F)F)c1] |
| 38 | 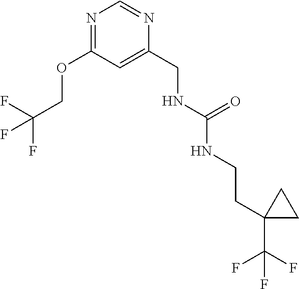 [SMILES: O=C(NCCC1(C(F)(F)F)CC1)NCc1cc(OCC(F)(F)F)ncn1] |
| 39 | 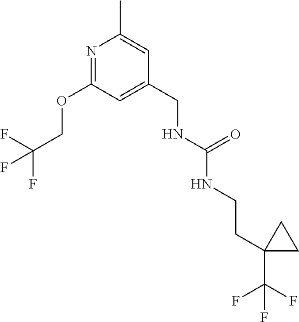 [SMILES: Cc1cc(CNC(=O)NCCC2(C(F)(F)F)CC2)cc(OCC(F)(F)F)n1] |
| 40 | 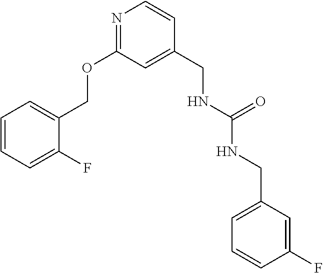 [SMILES: O=C(NCc1cccc(F)c1)NCc1ccnc(OCc2ccccc2F)c1] |
| 41 | 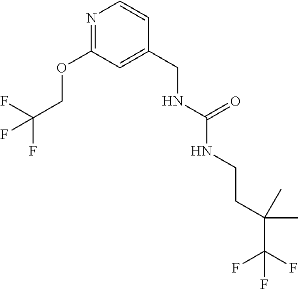 [SMILES: CC(C)(CCNC(=O)NCc1ccnc(OCC(F)(F)F)c1)C(F)(F)F] |
| 42 | 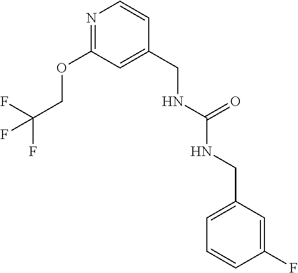 [SMILES: O=C(NCc1cccc(F)c1)NCc1ccnc(OCC(F)(F)F)c1] |
| 43 | 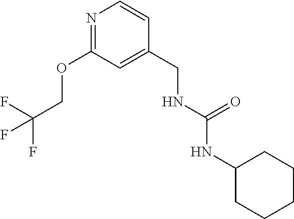 [SMILES: O=C(NCc1ccnc(OCC(F)(F)F)c1)NC1CCCCC1] |
| 44 | 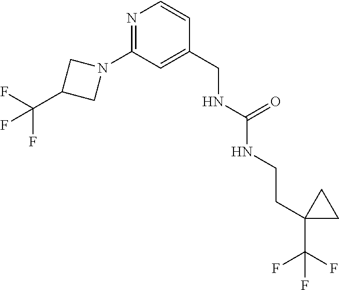 [SMILES: O=C(NCCC1(C(F)(F)F)CC1)NCc1ccnc(N2CC(C(F)(F)F)C2)c1] |
| 45 | 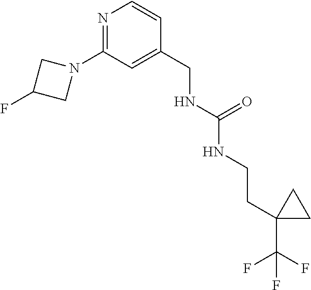 [SMILES: O=C(NCCC1(C(F)(F)F)CC1)NCc1ccnc(N2CC(F)C2)c1] |
| 46 | 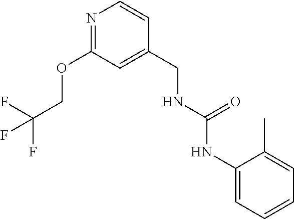 [SMILES: Cc1ccccc1NC(=O)NCc1ccnc(OCC(F)(F)F)c1] |
| 47 | 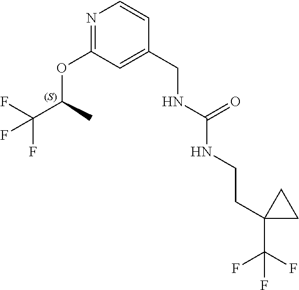 |
| 48 | 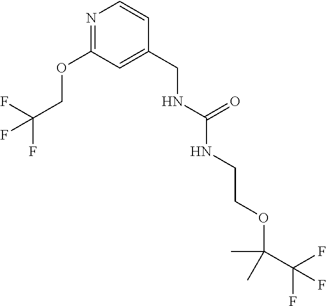 [SMILES: CC(C)(OCCNC(=O)NCc1ccnc(OCC(F)(F)F)c1)C(F)(F)F] |
| 49 | 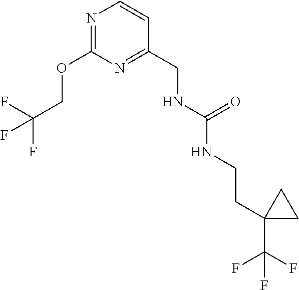 [SMILES: O=C(NCCC1(C(F)(F)F)CC1)NCc1ccnc(OCC(F)(F)F)n1] |
| 50 | 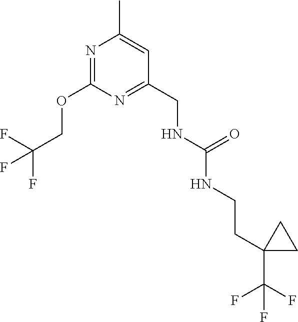 [SMILES: Cc1cc(CNC(=O)NCCC2(C(F)(F)F)CC2)nc(OCC(F)(F)F)n1] |
| 51 | 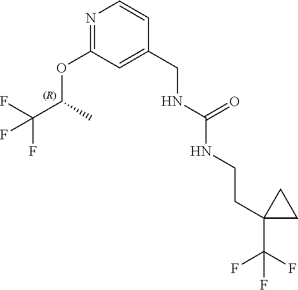 |
| 52 | 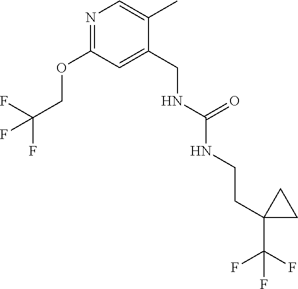 [SMILES: Cc1cnc(OCC(F)(F)F)cc1CNC(=O)NCCC1(C(F)(F)F)CC1] |
| 53 | 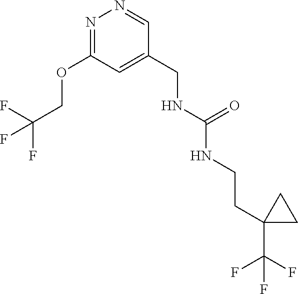 [SMILES: O=C(NCCC1(C(F)(F)F)CC1)NCc1cnnc(OCC(F)(F)F)c1] |
| 54 | 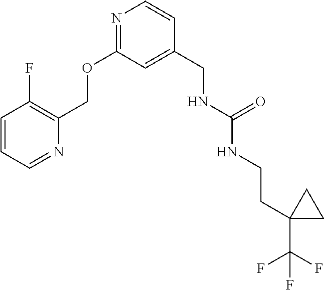 [SMILES: O=C(NCCC1(C(F)(F)F)CC1)NCc1ccnc(OCc2ncccc2F)c1] |
| 55 | 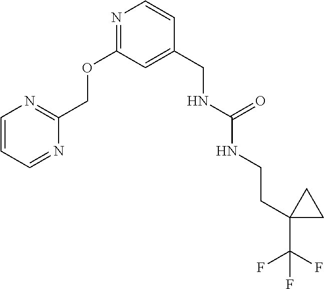 [SMILES: O=C(NCCC1(C(F)(F)F)CC1)NCc1ccnc(OCc2ncccn2)c1] |
| 56 | 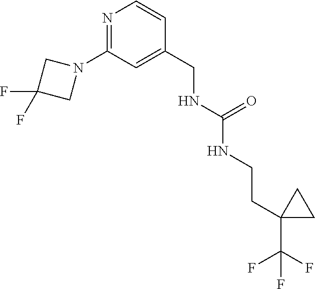 [SMILES: O=C(NCCC1(C(F)(F)F)CC1)NCc1ccnc(N2CC(F)(F)C2)c1] |
| 57 | 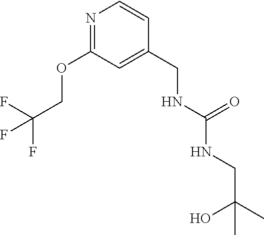 [SMILES: CC(C)(O)CNC(=O)NCc1ccnc(OCC(F)(F)F)c1] |
| 58 | 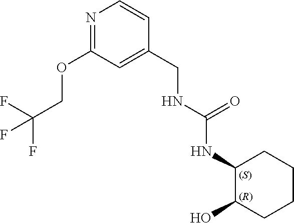 |
| 59 | 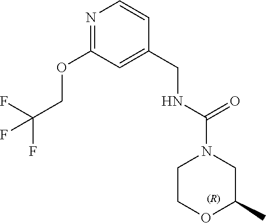 |
| 60 | 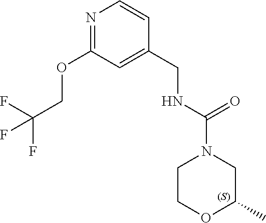 |
| 61 | 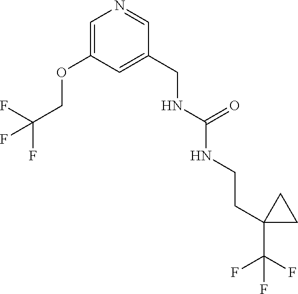 [SMILES: O=C(NCCC1(C(F)(F)F)CC1)NCc1cncc(OCC(F)(F)F)c1] |
| 62 | 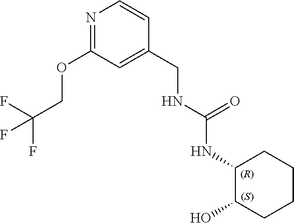 |
| 63 | 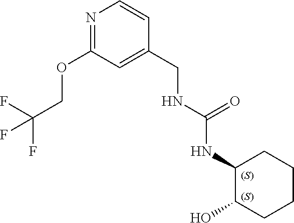 |
| 64 | 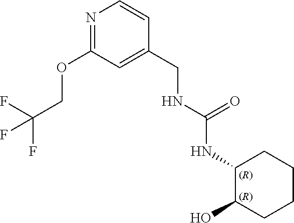 |
| 65 | 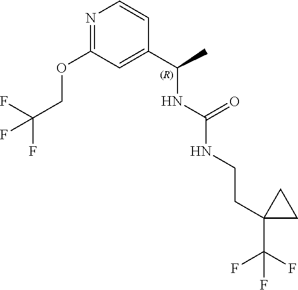 |
| 66 | 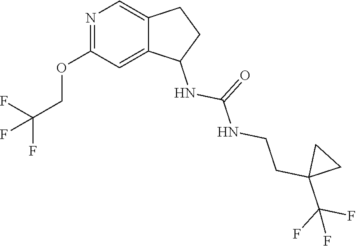 [SMILES: O=C(NCCC1(C(F)(F)F)CC1)NC1CCc2cnc(OCC(F)(F)F)cc21] |
| 67 | 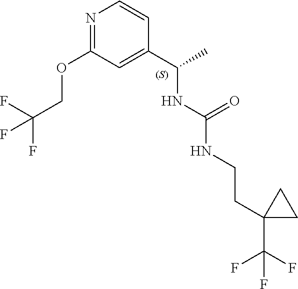 |
| 68 | 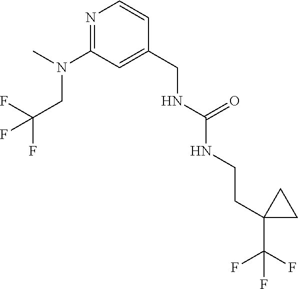 [SMILES: CN(CC(F)(F)F)c1cc(CNC(=O)NCCC2(C(F)(F)F)CC2)ccn1] |
| 69 | 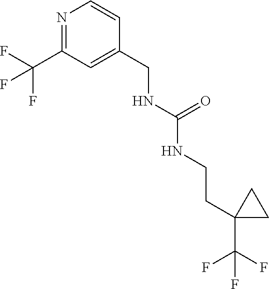 [SMILES: O=C(NCCC1(C(F)(F)F)CC1)NCc1ccnc(C(F)(F)F)c1] |
| 70 | 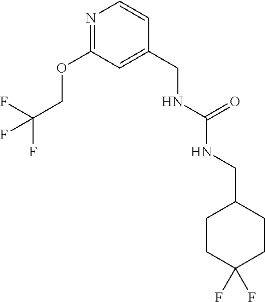 [SMILES: O=C(NCc1ccnc(OCC(F)(F)F)c1)NCC1CCC(F)(F)CC1] |
| 71 | 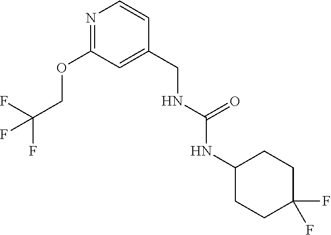 [SMILES: O=C(NCc1ccnc(OCC(F)(F)F)c1)NC1CCC(F)(F)CC1] |
| 72 | 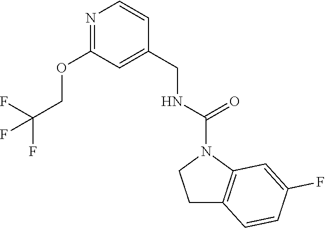 [SMILES: O=C(NCc1ccnc(OCC(F)(F)F)c1)N1CCc2ccc(F)cc21] |
| 73 | 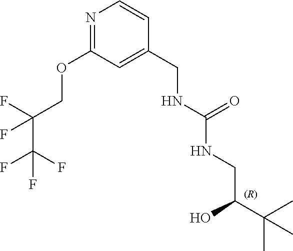 |
| 74 | 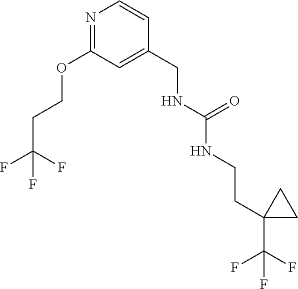 [SMILES: O=C(NCCC1(C(F)(F)F)CC1)NCc1ccnc(OCCC(F)(F)F)c1] |
| 75 | 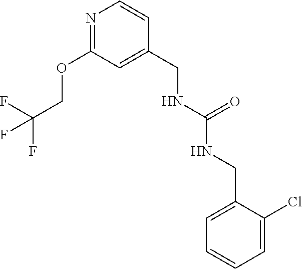 [SMILES: O=C(NCc1ccnc(OCC(F)(F)F)c1)NCc1ccccc1Cl] |
| 76 | 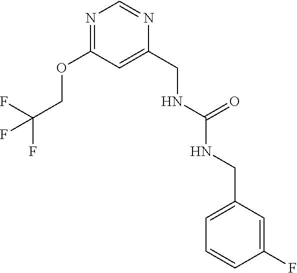 [SMILES: O=C(NCc1cccc(F)c1)NCc1cc(OCC(F)(F)F)ncn1] |
| 77 | 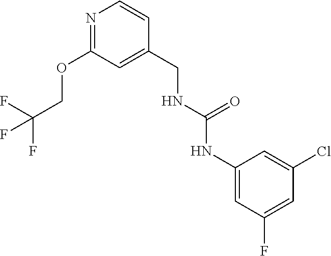 [SMILES: O=C(NCc1ccnc(OCC(F)(F)F)c1)Nc1cc(F)cc(Cl)c1] |
| 78 | 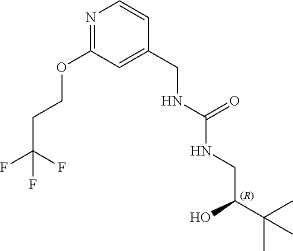 |
| 79 | 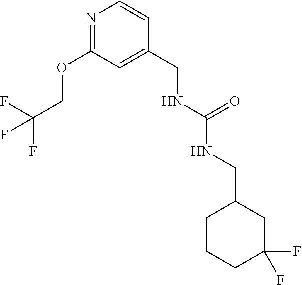 [SMILES: O=C(NCc1ccnc(OCC(F)(F)F)c1)NCC1CCCC(F)(F)C1] |
| 80 | 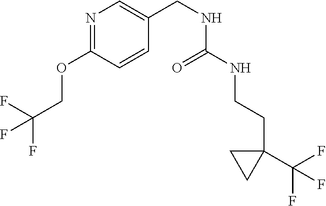 [SMILES: O=C(NCCC1(C(F)(F)F)CC1)NCc1ccc(OCC(F)(F)F)nc1] |
| 81 | 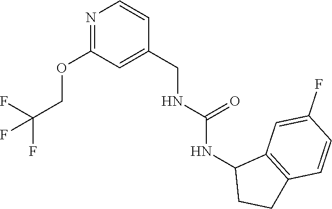 [SMILES: O=C(NCc1ccnc(OCC(F)(F)F)c1)NC1CCc2ccc(F)cc21] |
| 82 | 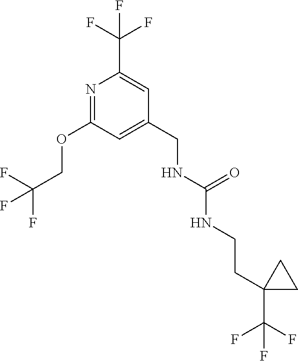 [SMILES: O=C(NCCC1(C(F)(F)F)CC1)NCc1cc(OCC(F)(F)F)nc(C(F)(F)F)c1] |
| 83 | 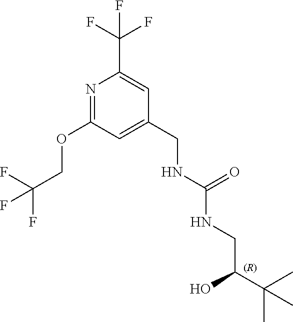 |
| 84 | 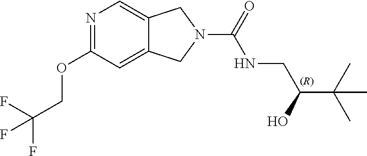 |
| 85 | 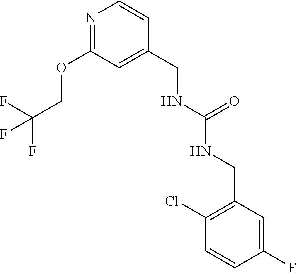 [SMILES: O=C(NCc1ccnc(OCC(F)(F)F)c1)NCc1cc(F)ccc1Cl] |
| 86 | 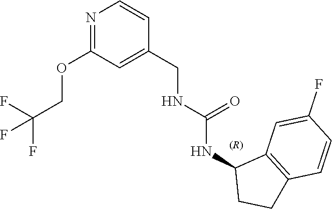 |
| 87 | 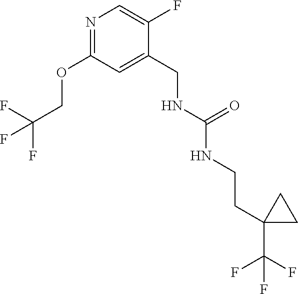 [SMILES: O=C(NCCC1(C(F)(F)F)CC1)NCc1cc(OCC(F)(F)F)ncc1F] |
| 88 | 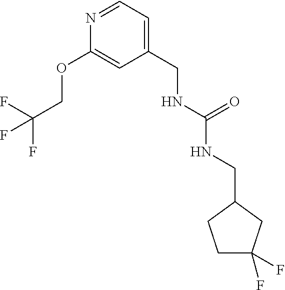 [SMILES: O=C(NCc1ccnc(OCC(F)(F)F)c1)NCC1CCC(F)(F)C1] |
| 89 | 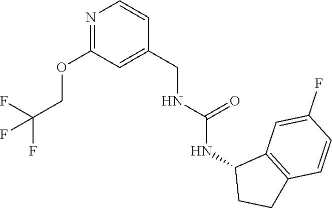 [SMILES: O=C(NCc1ccnc(OCC(F)(F)F)c1)N[C@H]1CCc2ccc(F)cc21] |
| 90 | 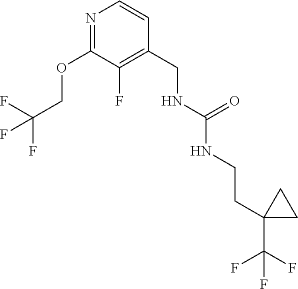 [SMILES: O=C(NCCC1(C(F)(F)F)CC1)NCc1ccnc(OCC(F)(F)F)c1F] |
| 91 | 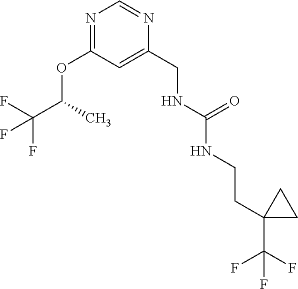 [SMILES: C[C@@H](Oc1cc(CNC(=O)NCCC2(C(F)(F)F)CC2)ncn1)C(F)(F)F] |
| 92 | 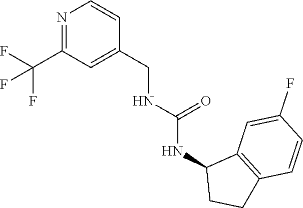 [SMILES: O=C(NCc1ccnc(C(F)(F)F)c1)N[C@@H]1CCc2ccc(F)cc21] |
| 93 | 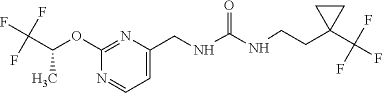 [SMILES: C[C@@H](Oc1nccc(CNC(=O)NCCC2(C(F)(F)F)CC2)n1)C(F)(F)F] |
| 94 | 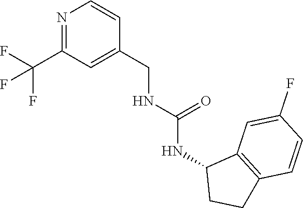 [SMILES: O=C(NCc1ccnc(C(F)(F)F)c1)N[C@H]1CCc2ccc(F)cc21] |
| 95 | 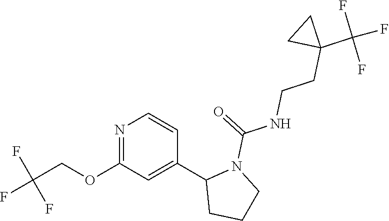 [SMILES: O=C(NCCC1(C(F)(F)F)CC1)N1CCCC1c1ccnc(OCC(F)(F)F)c1] |
| 96 | 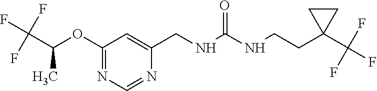 [SMILES: C[C@H](Oc1cc(CNC(=O)NCCC2(C(F)(F)F)CC2)ncn1)C(F)(F)F] |
| 97 | 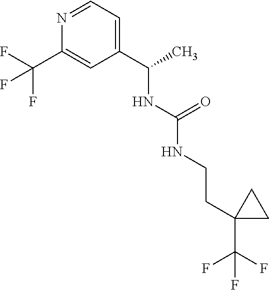 [SMILES: C[C@H](NC(=O)NCCC1(C(F)(F)F)CC1)c1ccnc(C(F)(F)F)c1] |
| 98 | 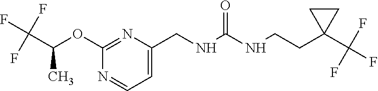 [SMILES: C[C@H](Oc1nccc(CNC(=O)NCCC2(C(F)(F)F)CC2)n1)C(F)(F)F] |
| 99 | 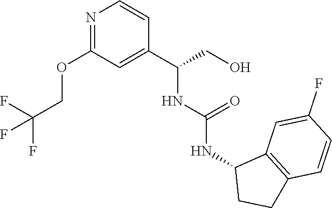 [SMILES: O=C(N[C@@H](CO)c1ccnc(OCC(F)(F)F)c1)N[C@H]1CCc2ccc(F)cc21] |
| 100 | 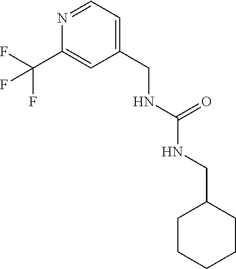 [SMILES: O=C(NCc1ccnc(C(F)(F)F)c1)NCC1CCCCC1] |
| 101 | 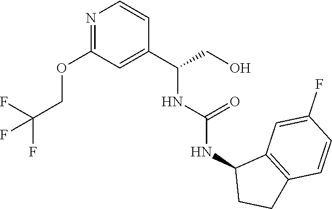 [SMILES: O=C(N[C@@H](CO)c1ccnc(OCC(F)(F)F)c1)N[C@@H]1CCc2ccc(F)cc21] |
| 102 | 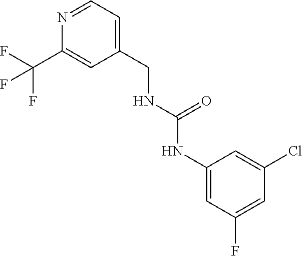 [SMILES: O=C(NCc1ccnc(C(F)(F)F)c1)Nc1cc(F)cc(Cl)c1] |
| 103 | 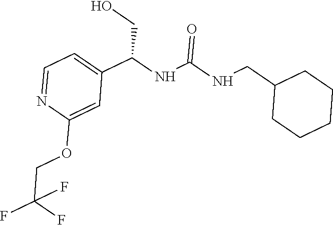 [SMILES: O=C(NCC1CCCCC1)N[C@@H](CO)c1ccnc(OCC(F)(F)F)c1] |
| 104 | 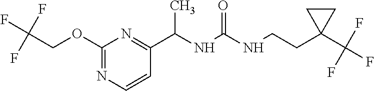 [SMILES: CC(NC(=O)NCCC1(C(F)(F)F)CC1)c1ccnc(OCC(F)(F)F)n1] |
| 105 | 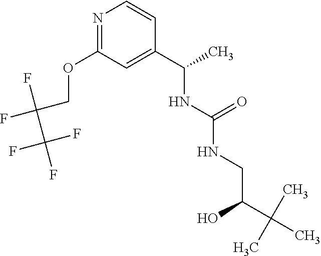 [SMILES: C[C@H](NC(=O)NC[C@H](O)C(C)(C)C)c1ccnc(OCC(F)(F)C(F)(F)F)c1] |
| 106 | 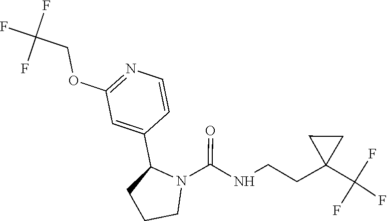 [SMILES: O=C(NCCC1(C(F)(F)F)CC1)N1CCC[C@H]1c1ccnc(OCC(F)(F)F)c1] |
| 107 | 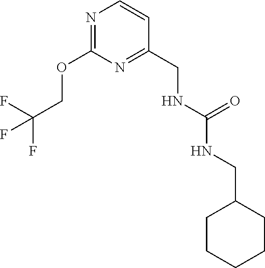 [SMILES: O=C(NCc1ccnc(OCC(F)(F)F)n1)NCC1CCCCC1] |
| 108 | 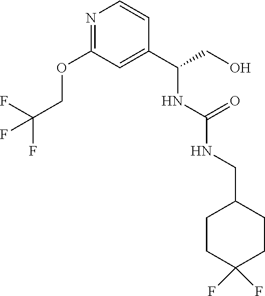 [SMILES: O=C(NCC1CCC(F)(F)CC1)N[C@@H](CO)c1ccnc(OCC(F)(F)F)c1] |
| 109 | 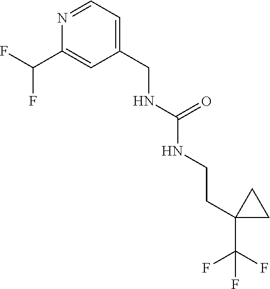 [SMILES: O=C(NCCC1(C(F)(F)F)CC1)NCc1ccnc(C(F)F)c1] |
| 110 | 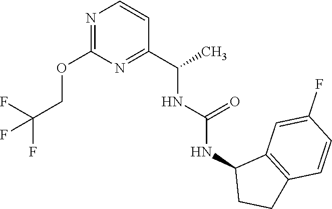 [SMILES: C[C@H](NC(=O)N[C@@H]1CCc2ccc(F)cc21)c1ccnc(OCC(F)(F)F)n1] |
| 111 | 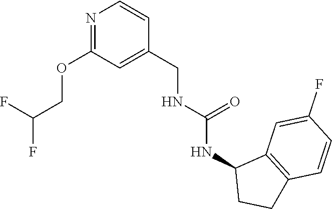 [SMILES: O=C(NCc1ccnc(OCC(F)F)c1)N[C@@H]1CCc2ccc(F)cc21] |
| 112 | 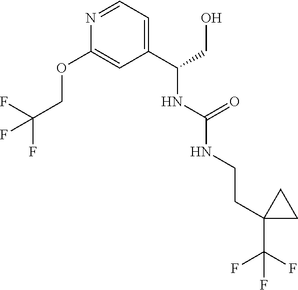 [SMILES: O=C(NCCC1(C(F)(F)F)CC1)N[C@@H](CO)c1ccnc(OCC(F)(F)F)c1] |
| 113 | 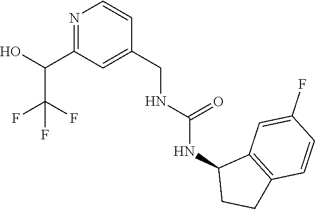 [SMILES: O=C(NCc1ccnc(C(O)C(F)(F)F)c1)N[C@@H]1CCc2ccc(F)cc21] |
| 114 | 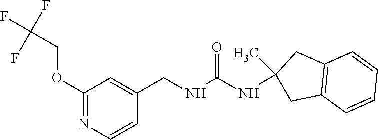 [SMILES: CC1(NC(=O)NCc2ccnc(OCC(F)(F)F)c2)Cc2ccccc2C1] |
| 115 | 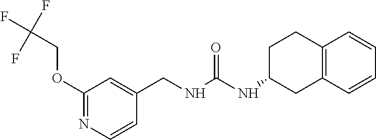 [SMILES: O=C(NCc1ccnc(OCC(F)(F)F)c1)N[C@@H]1CCc2ccccc2C1] |
| 116 | 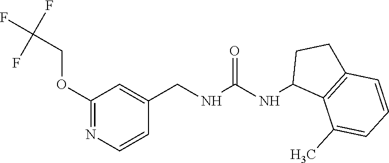 [SMILES: Cc1cccc2c1C(NC(=O)NCc1ccnc(OCC(F)(F)F)c1)CC2] |
| 117 | 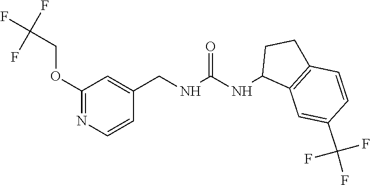 [SMILES: O=C(NCc1ccnc(OCC(F)(F)F)c1)NC1CCc2ccc(C(F)(F)F)cc21] |
| 118 | 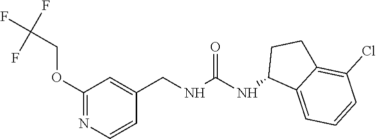 [SMILES: O=C(NCc1ccnc(OCC(F)(F)F)c1)N[C@@H]1CCc2c(Cl)cccc21] |
| 119 | 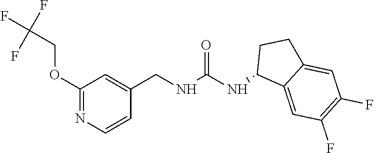 [SMILES: O=C(NCc1ccnc(OCC(F)(F)F)c1)N[C@@H]1CCc2cc(F)c(F)cc21] |
| 120 | 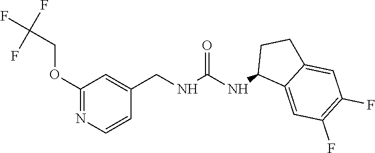 [SMILES: O=C(NCc1ccnc(OCC(F)(F)F)c1)N[C@H]1CCc2cc(F)c(F)cc21] |
| 121 | 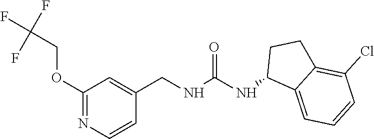 [SMILES: O=C(NCc1ccnc(OCC(F)(F)F)c1)N[C@@H]1CCc2c(Cl)cccc21] |
| 122 | 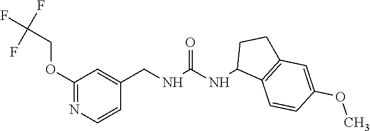 [SMILES: COc1ccc2c(c1)CCC2NC(=O)NCc1ccnc(OCC(F)(F)F)c1] |
| 123 | 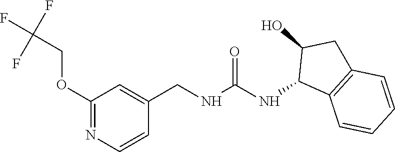 [SMILES: O=C(NCc1ccnc(OCC(F)(F)F)c1)N[C@H]1c2ccccc2C[C@@H]1O] |
| 124 | 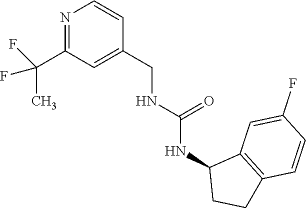 [SMILES: CC(F)(F)c1cc(CNC(=O)N[C@@H]2CCc3ccc(F)cc32)ccn1] |
| 125 | 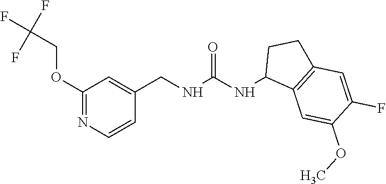 [SMILES: COc1cc2c(cc1F)CCC2NC(=O)NCc1ccnc(OCC(F)(F)F)c1] |
| 126 | 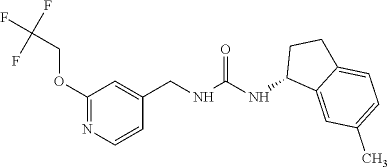 [SMILES: Cc1ccc2c(c1)[C@H](NC(=O)NCc1ccnc(OCC(F)(F)F)c1)CC2] |
| 127 | 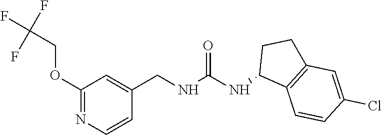 [SMILES: O=C(NCc1ccnc(OCC(F)(F)F)c1)N[C@@H]1CCc2cc(Cl)ccc21] |
| 128 | 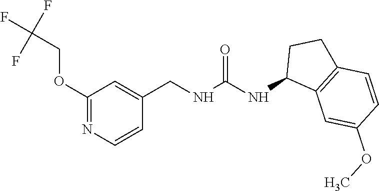 [SMILES: COc1ccc2c(c1)[C@@H](NC(=O)NCc1ccnc(OCC(F)(F)F)c1)CC2] |
| 129 | 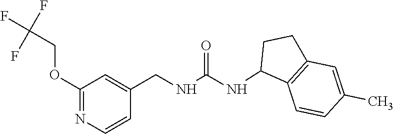 [SMILES: Cc1ccc2c(c1)CCC2NC(=O)NCc1ccnc(OCC(F)(F)F)c1] |
| 130 | 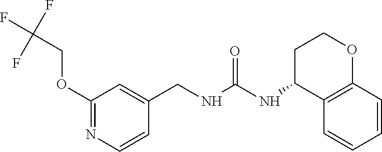 [SMILES: O=C(NCc1ccnc(OCC(F)(F)F)c1)N[C@@H]1CCOc2ccccc21] |
| 131 | 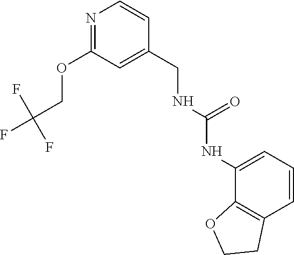 [SMILES: O=C(NCc1ccnc(OCC(F)(F)F)c1)Nc1cccc2c1OCC2] |
| 132 | 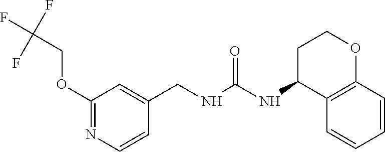 [SMILES: O=C(NCc1ccnc(OCC(F)(F)F)c1)N[C@H]1CCOc2ccccc21] |
| 133 | 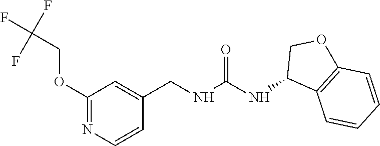 [SMILES: O=C(NCc1ccnc(OCC(F)(F)F)c1)N[C@@H]1COc2ccccc21] |
| 134 | 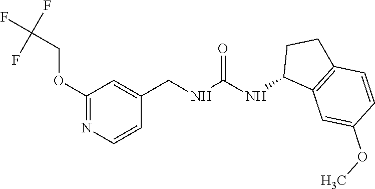 [SMILES: COc1ccc2c(c1)[C@H](NC(=O)NCc1ccnc(OCC(F)(F)F)c1)CC2] |
| 135 | 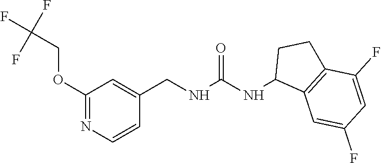 [SMILES: O=C(NCc1ccnc(OCC(F)(F)F)c1)NC1CCc2c(F)cc(F)cc21] |
| 136 | 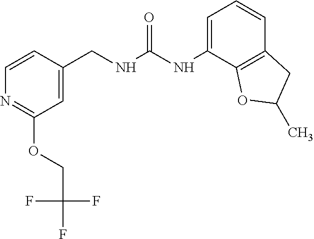 [SMILES: CC1Cc2cccc(NC(=O)NCc3ccnc(OCC(F)(F)F)c3)c2O1] |
| 137 | 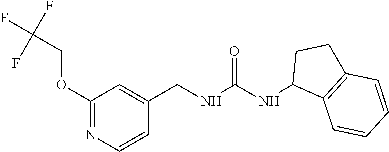 [SMILES: O=C(NCc1ccnc(OCC(F)(F)F)c1)NC1CCc2ccccc21] |
| 138 | 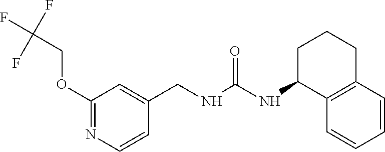 [SMILES: O=C(NCc1ccnc(OCC(F)(F)F)c1)N[C@H]1CCCc2ccccc21] |
| 139 | 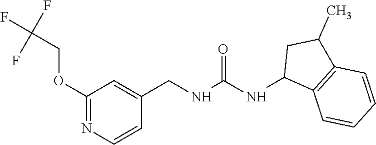 [SMILES: CC1CC(NC(=O)NCc2ccnc(OCC(F)(F)F)c2)c2ccccc21] |
| 140 | 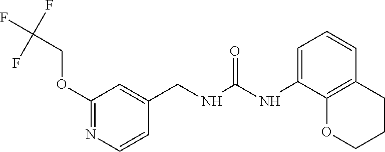 [SMILES: O=C(NCc1ccnc(OCC(F)(F)F)c1)Nc1cccc2c1OCCC2] |
| 141 | 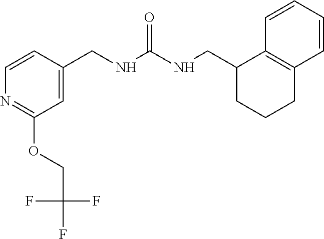 [SMILES: O=C(NCc1ccnc(OCC(F)(F)F)c1)NCC1CCCc2ccccc21] |
| 142 | 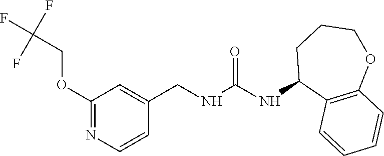 [SMILES: O=C(NCc1ccnc(OCC(F)(F)F)c1)N[C@H]1CCCOc2ccccc21] |
| 143 | 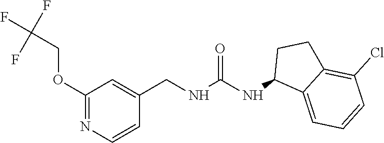 [SMILES: O=C(NCc1ccnc(OCC(F)(F)F)c1)N[C@H]1CCc2c(Cl)cccc21] |
| 144 | 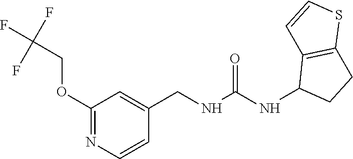 [SMILES: O=C(NCc1ccnc(OCC(F)(F)F)c1)NC1CCc2sccc21] |
| 145 | 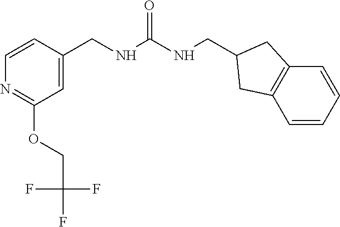 [SMILES: O=C(NCc1ccnc(OCC(F)(F)F)c1)NCC1Cc2ccccc2C1] |
| 146 | 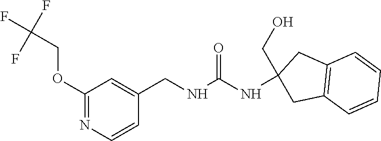 [SMILES: O=C(NCc1ccnc(OCC(F)(F)F)c1)NC1(CO)Cc2ccccc2C1] |
| 147 | 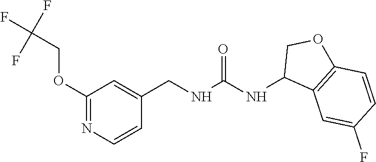 [SMILES: O=C(NCc1ccnc(OCC(F)(F)F)c1)NC1COc2ccc(F)cc21] |
| 148 | 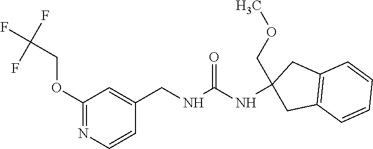 [SMILES: COCC1(NC(=O)NCc2ccnc(OCC(F)(F)F)c2)Cc2ccccc2C1] |
| 149 | 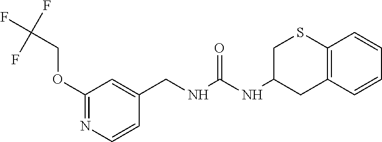 [SMILES: O=C(NCc1ccnc(OCC(F)(F)F)c1)NC1CSc2ccccc2C1] |
| 150 | 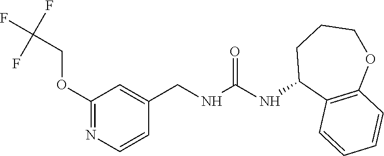 [SMILES: O=C(NCc1ccnc(OCC(F)(F)F)c1)N[C@@H]1CCCOc2ccccc21] |
| 151 | 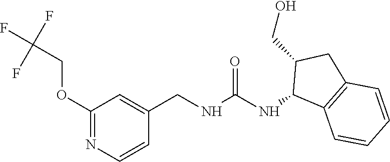 [SMILES: O=C(NCc1ccnc(OCC(F)(F)F)c1)N[C@H]1c2ccccc2C[C@H]1CO] |
| 152 | 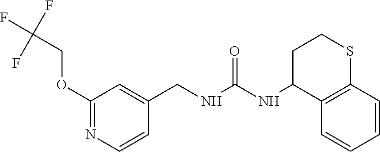 [SMILES: O=C(NCc1ccnc(OCC(F)(F)F)c1)NC1CCSc2ccccc21] |
| 153 | 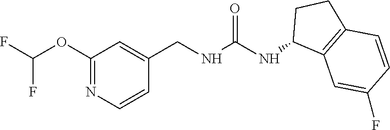 [SMILES: O=C(NCc1ccnc(OC(F)F)c1)N[C@@H]1CCc2ccc(F)cc21] |
| 154 | 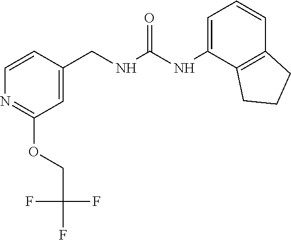 [SMILES: O=C(NCc1ccnc(OCC(F)(F)F)c1)Nc1cccc2c1CCC2] |
| 155 | 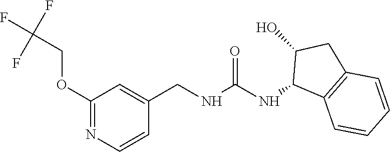 [SMILES: O=C(NCc1ccnc(OCC(F)(F)F)c1)N[C@H]1c2ccccc2C[C@H]1O] |
| 156 | 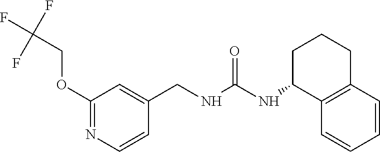 [SMILES: O=C(NCc1ccnc(OCC(F)(F)F)c1)N[C@@H]1CCCc2ccccc21] |
| 157 | 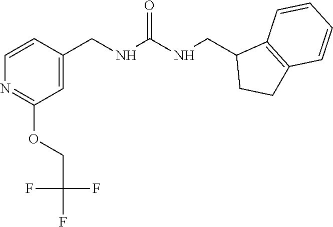 [SMILES: O=C(NCc1ccnc(OCC(F)(F)F)c1)NCC1CCc2ccccc21] |
| 158 | 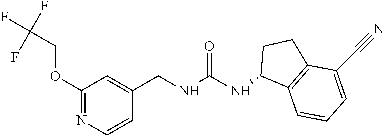 [SMILES: N#Cc1cccc2c1CC[C@H]2NC(=O)NCc1ccnc(OCC(F)(F)F)c1] |
| 159 | 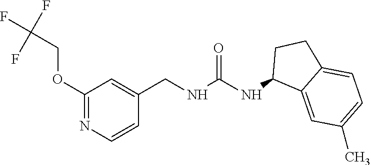 [SMILES: Cc1ccc2c(c1)[C@@H](NC(=O)NCc1ccnc(OCC(F)(F)F)c1)CC2] |
| 160 | 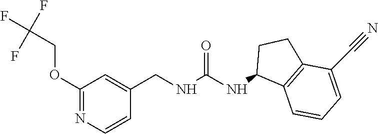 [SMILES: N#Cc1cccc2c1CC[C@@H]2NC(=O)NCc1ccnc(OCC(F)(F)F)c1] |
| 161 | 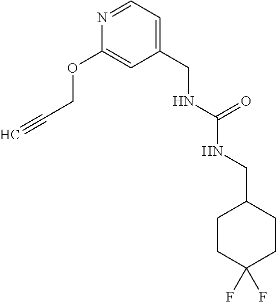 [SMILES: C#CCOc1cc(CNC(=O)NCC2CCC(F)(F)CC2)ccn1] |
| 162 | 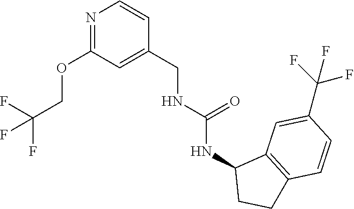 [SMILES: O=C(NCc1ccnc(OCC(F)(F)F)c1)N[C@@H]1CCc2ccc(C(F)(F)F)cc21] |
| 163 | 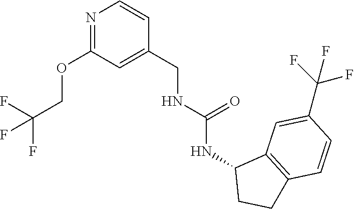 [SMILES: O=C(NCc1ccnc(OCC(F)(F)F)c1)N[C@H]1CCc2ccc(C(F)(F)F)cc21] |
| 164 | 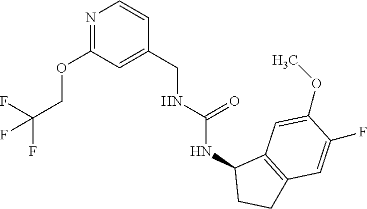 [SMILES: COc1cc2c(cc1F)CC[C@H]2NC(=O)NCc1ccnc(OCC(F)(F)F)c1] |
| 165 | 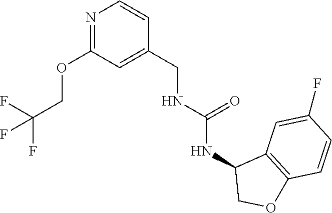 [SMILES: O=C(NCc1ccnc(OCC(F)(F)F)c1)N[C@@H]1COc2ccc(F)cc21] |
| 166 | 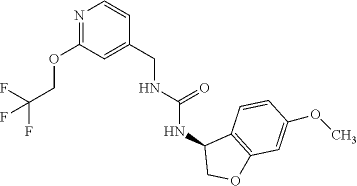 [SMILES: COc1ccc2c(c1)OC[C@H]2NC(=O)NCc1ccnc(OCC(F)(F)F)c1] |

TABLE B

| No. | Structure |
|---|---|
| 167 | 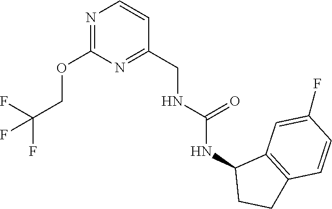 [SMILES: O=C(NCc1ccnc(OCC(F)(F)F)n1)N[C@@H]1CCc2ccc(F)cc21] |
| 168 | 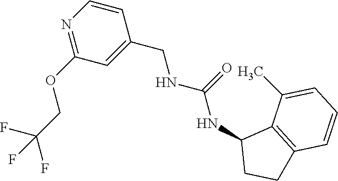 [SMILES: Cc1cccc2c1[C@H](NC(=O)NCc1ccnc(OCC(F)(F)F)c1)CC2] |
| 169 | 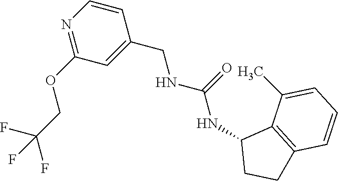 [SMILES: Cc1cccc2c1[C@@H](NC(=O)NCc1ccnc(OCC(F)(F)F)c1)CC2] |
| 170 | 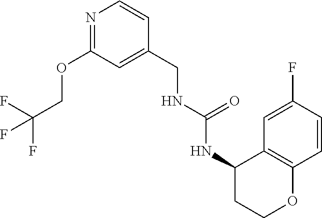 [SMILES: O=C(NCc1ccnc(OCC(F)(F)F)c1)N[C@@H]1CCOc2ccc(F)cc21] |
| 171 | 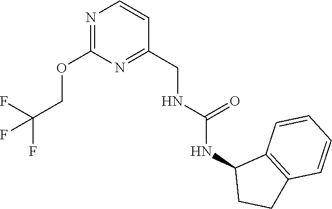 [SMILES: O=C(NCc1ccnc(OCC(F)(F)F)n1)N[C@@H]1CCc2ccccc21] |
| 172 | 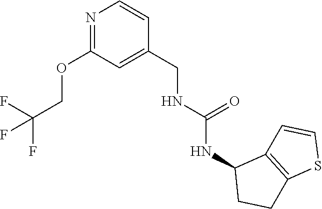 [SMILES: O=C(NCc1ccnc(OCC(F)(F)F)c1)N[C@@H]1CCc2sccc21] |
| 173 | 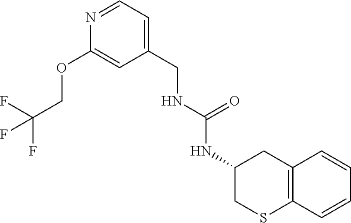 [SMILES: O=C(NCc1ccnc(OCC(F)(F)F)c1)N[C@H]1CSc2ccccc2C1] |
| 174 | 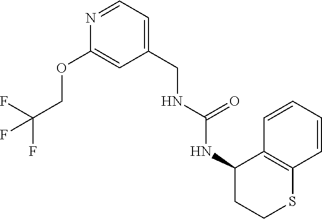 [SMILES: O=C(NCc1ccnc(OCC(F)(F)F)c1)N[C@@H]1CCSc2ccccc21] |
| 175 | 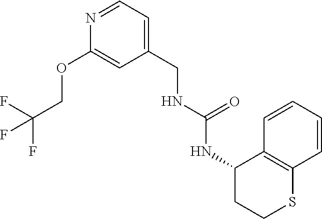 [SMILES: O=C(NCc1ccnc(OCC(F)(F)F)c1)N[C@H]1CCSc2ccccc21] |
| 176 | 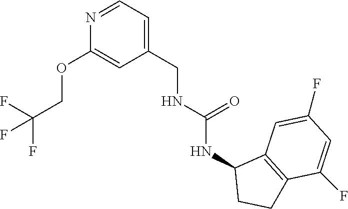 [SMILES: O=C(NCc1ccnc(OCC(F)(F)F)c1)N[C@@H]1CCc2c(F)cc(F)cc21] |
| 177 | 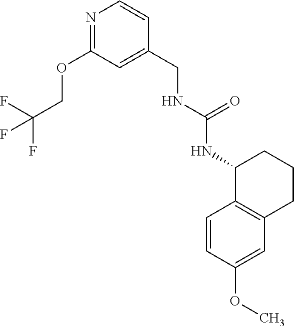 [SMILES: COc1ccc2c(c1)CCC[C@H]2NC(=O)NCc1ccnc(OCC(F)(F)F)c1] |
| 178 | 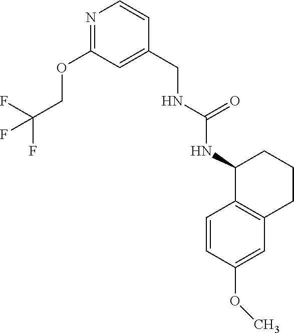 [SMILES: COc1ccc2c(c1)CCC[C@@H]2NC(=O)NCc1ccnc(OCC(F)(F)F)c1] |
| 179 | 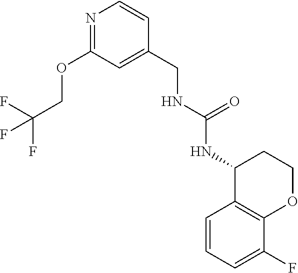 [SMILES: O=C(NCc1ccnc(OCC(F)(F)F)c1)N[C@@H]1CCOc2c(F)cccc21] |
| 180 | 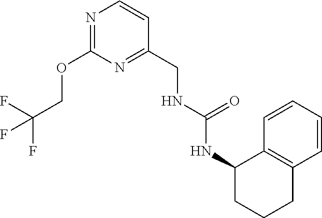 [SMILES: O=C(NCc1ccnc(OCC(F)(F)F)n1)N[C@@H]1CCCc2ccccc21] |
| 181 | 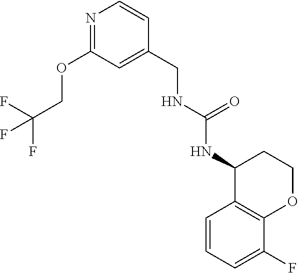 [SMILES: O=C(NCc1ccnc(OCC(F)(F)F)c1)N[C@H]1CCOc2c(F)cccc21] |
| 182 | 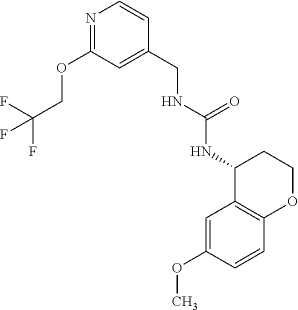 [SMILES: COc1ccc2c(c1)[C@H](NC(=O)NCc1ccnc(OCC(F)(F)F)c1)CCO2] |
| 183 | 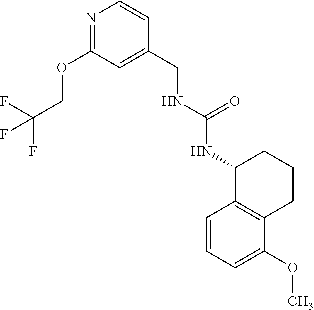 [SMILES: COc1cccc2c1CCC[C@H]2NC(=O)NCc1ccnc(OCC(F)(F)F)c1] |
| 184 | 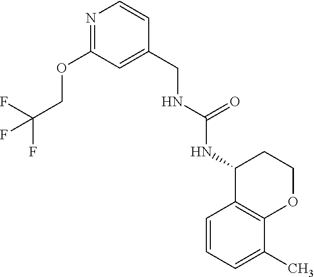 [SMILES: Cc1cccc2c1OCC[C@H]2NC(=O)NCc1ccnc(OCC(F)(F)F)c1] |
| 185 | 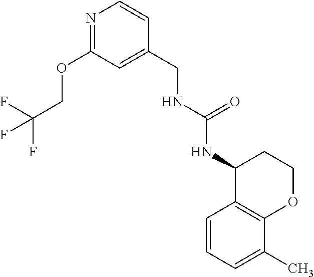 [SMILES: Cc1cccc2c1OCC[C@@H]2NC(=O)NCc1ccnc(OCC(F)(F)F)c1] |
| 186 | 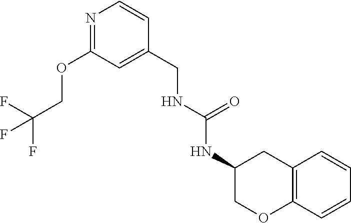 [SMILES: O=C(NCc1ccnc(OCC(F)(F)F)c1)N[C@@H]1COc2ccccc2C1] |
| 187 | 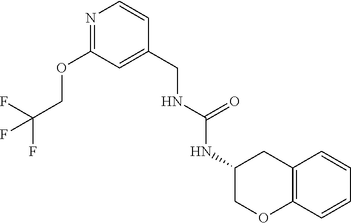 [SMILES: O=C(NCc1ccnc(OCC(F)(F)F)c1)N[C@H]1COc2ccccc2C1] |
| 188 | 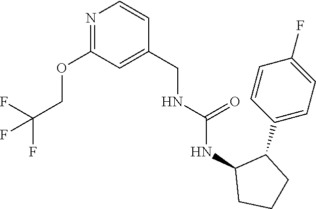 [SMILES: O=C(NCc1ccnc(OCC(F)(F)F)c1)N[C@@H]1CCC[C@H]1c1ccc(F)cc1] |
| 189 | 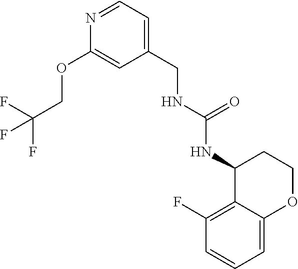 [SMILES: O=C(NCc1ccnc(OCC(F)(F)F)c1)N[C@H]1CCOc2cccc(F)c21] |
| 190 | 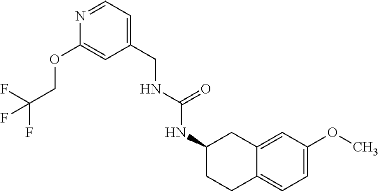 [SMILES: COc1ccc2c(c1)C[C@H](NC(=O)NCc1ccnc(OCC(F)(F)F)c1)CC2] |
| 191 | 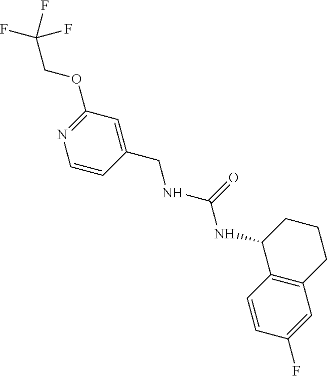 [SMILES: O=C(NCc1ccnc(OCC(F)(F)F)c1)N[C@@H]1CCCc2cc(F)ccc21] |
| 192 | 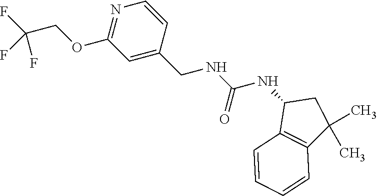 [SMILES: CC1(C)C[C@@H](NC(=O)NCc2ccnc(OCC(F)(F)F)c2)c2ccccc21] |
| 193 | 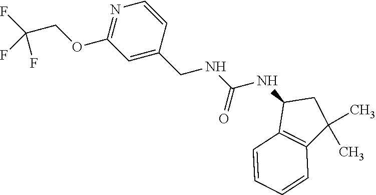 [SMILES: CC1(C)C[C@H](NC(=O)NCc2ccnc(OCC(F)(F)F)c2)c2ccccc21] |
| 194 | 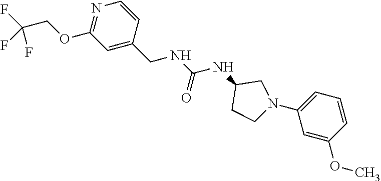 [SMILES: COc1cccc(N2CC[C@@H](NC(=O)NCc3ccnc(OCC(F)(F)F)c3)C2)c1] |
| 195 | 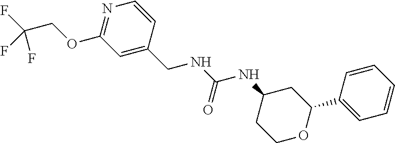 [SMILES: O=C(NCc1ccnc(OCC(F)(F)F)c1)N[C@@H]1CCO[C@@H](c2ccccc2)C1] |
| 196 | 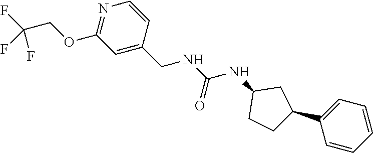 [SMILES: O=C(NCc1ccnc(OCC(F)(F)F)c1)N[C@@H]1CC[C@H](c2ccccc2)C1] |
| 197 | 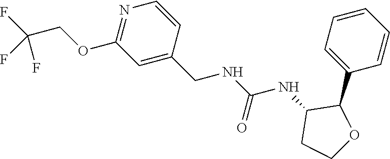 [SMILES: O=C(NCc1ccnc(OCC(F)(F)F)c1)N[C@H]1CCO[C@@H]1c1ccccc1] |
| 198 | 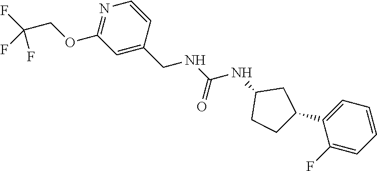 [SMILES: O=C(NCc1ccnc(OCC(F)(F)F)c1)N[C@H]1CC[C@@H](c2ccccc2F)C1] |
| 199 | 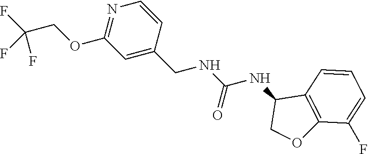 [SMILES: O=C(NCc1ccnc(OCC(F)(F)F)c1)N[C@@H]1COc2c(F)cccc21] |
| 200 | 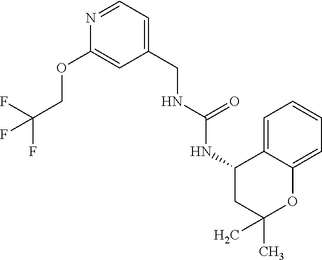 [SMILES: CC1(C)C[C@H](NC(=O)NCc2ccnc(OCC(F)(F)F)c2)c2ccccc2O1] |
| 201 | 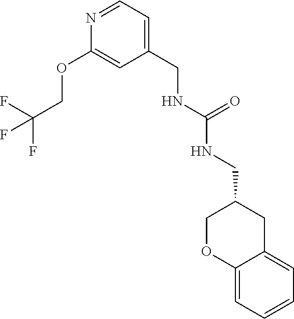 [SMILES: O=C(NCc1ccnc(OCC(F)(F)F)c1)NC[C@H]1COc2ccccc2C1] |
| 202 | 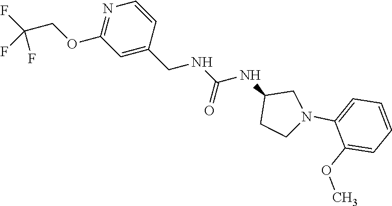 [SMILES: COc1ccccc1N1CC[C@@H](NC(=O)NCc2ccnc(OCC(F)(F)F)c2)C1] |
| 203 | 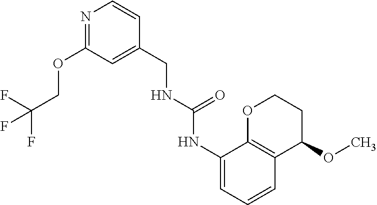 [SMILES: CO[C@@H]1CCOc2c(NC(=O)NCc3ccnc(OCC(F)(F)F)c3)cccc21] |
| 204 | 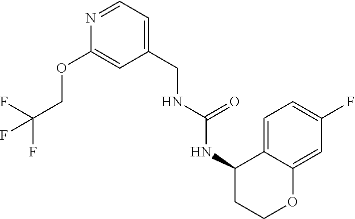 [SMILES: O=C(NCc1ccnc(OCC(F)(F)F)c1)N[C@@H]1CCOc2cc(F)ccc21] |
| 205 | 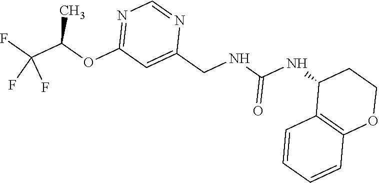 [SMILES: C[C@@H](Oc1cc(CNC(=O)N[C@@H]2CCOc3ccccc32)ncn1)C(F)(F)F] |
| 206 | 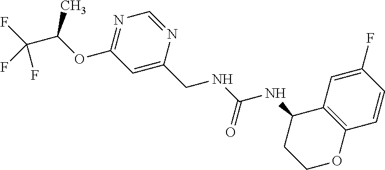 [SMILES: C[C@@H](Oc1cc(CNC(=O)N[C@@H]2CCOc3ccc(F)cc32)ncn1)C(F)(F)F] |
| 207 | 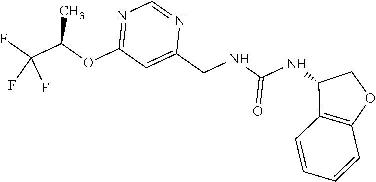 [SMILES: C[C@@H](Oc1cc(CNC(=O)N[C@@H]2COc3ccccc32)ncn1)C(F)(F)F] |
| 208 | 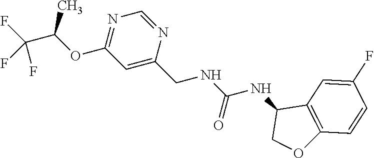 [SMILES: C[C@@H](Oc1cc(CNC(=O)N[C@@H]2COc3ccc(F)cc32)ncn1)C(F)(F)F] |
| 209 | 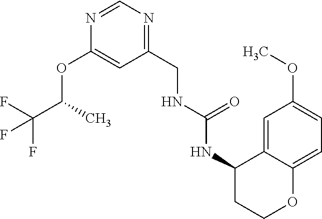 [SMILES: COc1ccc2c(c1)[C@H](NC(=O)NCc1cc(O[C@H](C)C(F)(F)F)ncn1)CCO2] |
| 210 | 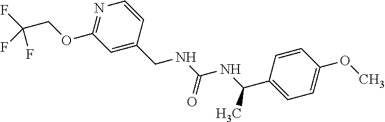 [SMILES: COc1ccc([C@@H](C)NC(=O)NCc2ccnc(OCC(F)(F)F)c2)cc1] |
| 211 | 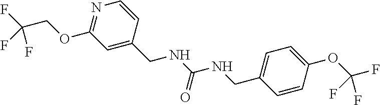 [SMILES: O=C(NCc1ccc(OC(F)(F)F)cc1)NCc1ccnc(OCC(F)(F)F)c1] |
| 212 | 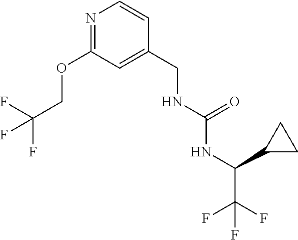 [SMILES: O=C(NCc1ccnc(OCC(F)(F)F)c1)N[C@@H](C1CC1)C(F)(F)F] |
| 213 | 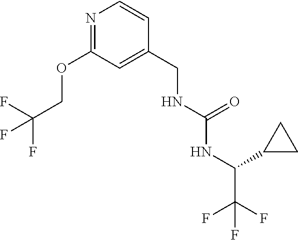 [SMILES: O=C(NCc1ccnc(OCC(F)(F)F)c1)N[C@H](C1CC1)C(F)(F)F] |
| 214 | 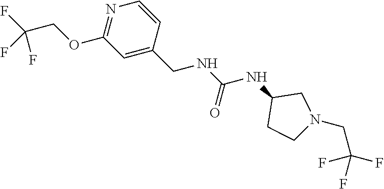 [SMILES: O=C(NCc1ccnc(OCC(F)(F)F)c1)N[C@@H]1CCN(CC(F)(F)F)C1] |
| 215 | 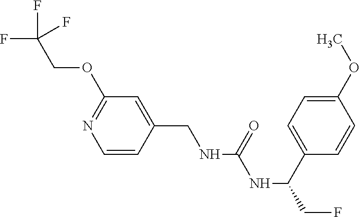 [SMILES: COc1ccc([C@H](CF)NC(=O)NCc2ccnc(OCC(F)(F)F)c2)cc1] |
| 216 | 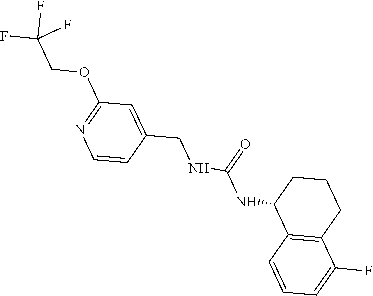 [SMILES: O=C(NCc1ccnc(OCC(F)(F)F)c1)N[C@@H]1CCCc2c(F)cccc21] |
| 217 | 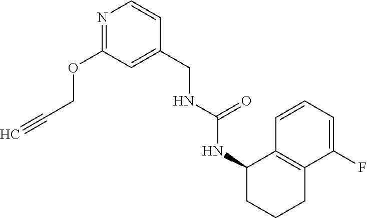 [SMILES: C#CCOc1cc(CNC(=O)N[C@@H]2CCCc3c(F)cccc32)ccn1] |
| 218 | 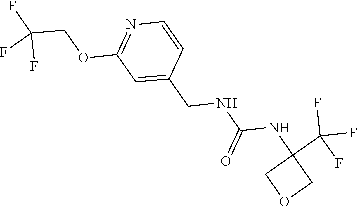 [SMILES: O=C(NCc1ccnc(OCC(F)(F)F)c1)NC1(C(F)(F)F)COC1] |
| 219 | 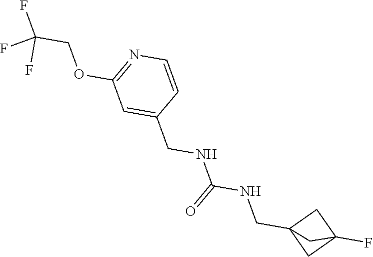 [SMILES: O=C(NCc1ccnc(OCC(F)(F)F)c1)NCC12CC(F)(C1)C2] |
| 220 | 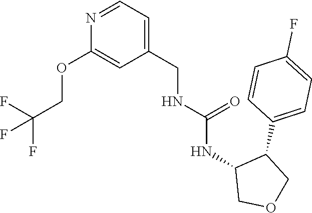 [SMILES: O=C(NCc1ccnc(OCC(F)(F)F)c1)N[C@H]1COC[C@H]1c1ccc(F)cc1] |
| 221 | 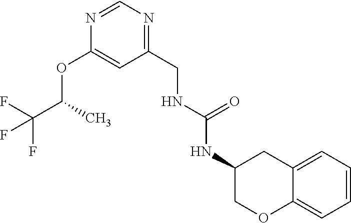 [SMILES: C[C@@H](Oc1cc(CNC(=O)N[C@@H]2COc3ccccc3C2)ncn1)C(F)(F)F] |
| 222 | 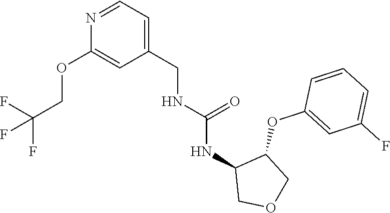 [SMILES: O=C(NCc1ccnc(OCC(F)(F)F)c1)N[C@@H]1COC[C@H]1Oc1cccc(F)c1] |
| 223 | 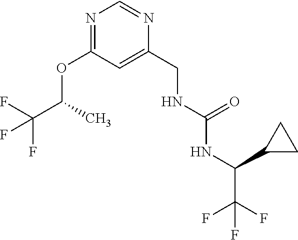 [SMILES: C[C@@H](Oc1cc(CNC(=O)N[C@@H](C2CC2)C(F)(F)F)ncn1)C(F)(F)F] |
| 224 | 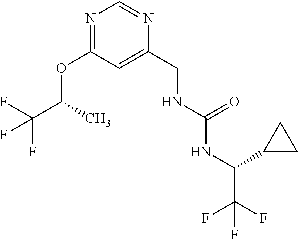 [SMILES: C[C@@H](Oc1cc(CNC(=O)N[C@H](C2CC2)C(F)(F)F)ncn1)C(F)(F)F] |
| 225 | 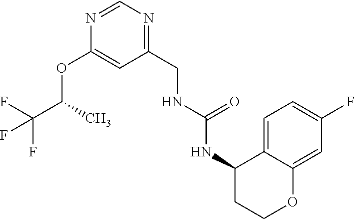 [SMILES: C[C@@H](Oc1cc(CNC(=O)N[C@@H]2CCOc3cc(F)ccc32)ncn1)C(F)(F)F] |
| 226 | 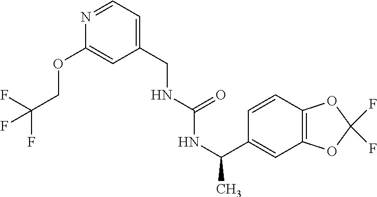 [SMILES: C[C@@H](NC(=O)NCc1ccnc(OCC(F)(F)F)c1)c1ccc2c(c1)OC(F)(F)O2] |
| 227 | 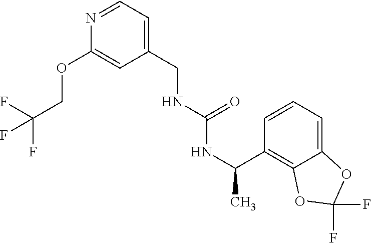 [SMILES: C[C@@H](NC(=O)NCc1ccnc(OCC(F)(F)F)c1)c1cccc2c1OC(F)(F)O2] |
| 228 | 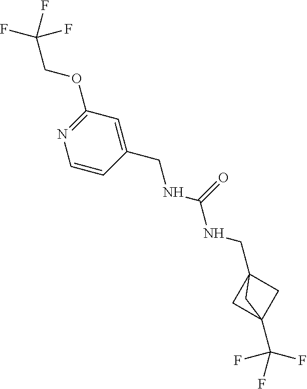 [SMILES: O=C(NCc1ccnc(OCC(F)(F)F)c1)NCC12CC(C(F)(F)F)(C1)C2] |
| 229 | 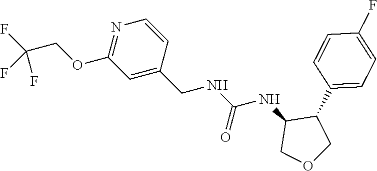 [SMILES: O=C(NCc1ccnc(OCC(F)(F)F)c1)N[C@@H]1COC[C@H]1c1ccc(F)cc1] |
| 230 | 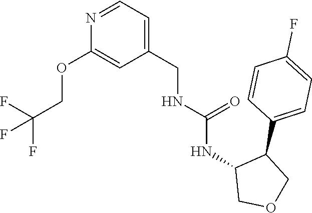 [SMILES: O=C(NCc1ccnc(OCC(F)(F)F)c1)N[C@H]1COC[C@@H]1c1ccc(F)cc1] |
| 231 | 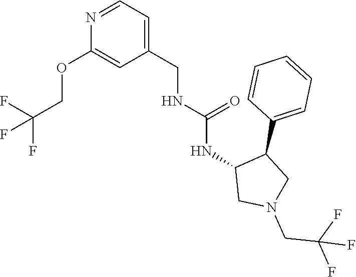 [SMILES: O=C(NCc1ccnc(OCC(F)(F)F)c1)N[C@H]1CN(CC(F)(F)F)C[C@@H]1c1ccccc1] |
| 232 | 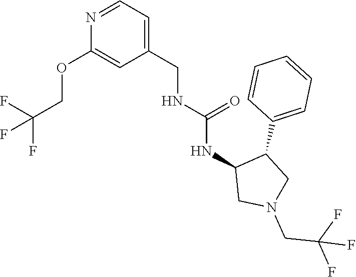 [SMILES: O=C(NCc1ccnc(OCC(F)(F)F)c1)N[C@@H]1CN(CC(F)(F)F)C[C@H]1c1ccccc1] |
| 233 | 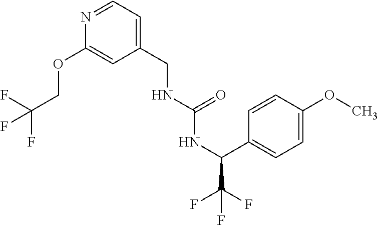 [SMILES: COc1ccc([C@H](NC(=O)NCc2ccnc(OCC(F)(F)F)c2)C(F)(F)F)cc1] |
| 234 | 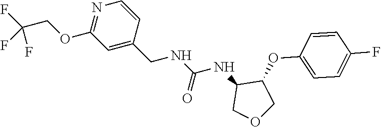 [SMILES: O=C(NCc1ccnc(OCC(F)(F)F)c1)N[C@@H]1COC[C@H]1Oc1ccc(F)cc1] |
| 235 | 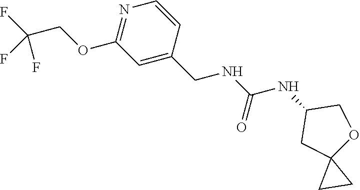 [SMILES: O=C(NCc1ccnc(OCC(F)(F)F)c1)N[C@@H]1COC2(CC2)C1] |
| 236 | 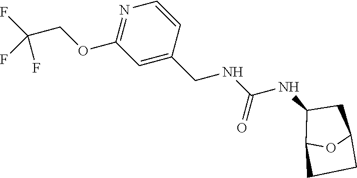 [SMILES: O=C(NCc1ccnc(OCC(F)(F)F)c1)N[C@H]1C[C@H]2CC[C@@H]1O2] |
| 237 | 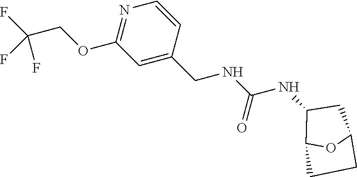 [SMILES: O=C(NCc1ccnc(OCC(F)(F)F)c1)N[C@@H]1C[C@@H]2CC[C@H]1O2] |
| 238 | 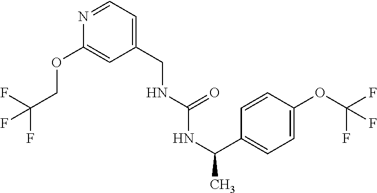 [SMILES: C[C@@H](NC(=O)NCc1ccnc(OCC(F)(F)F)c1)c1ccc(OC(F)(F)F)cc1] |
| 239 | 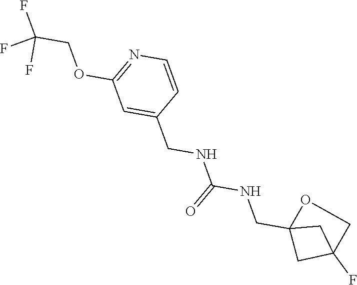 [SMILES: O=C(NCc1ccnc(OCC(F)(F)F)c1)NCC12CC(F)(CO1)C2] |
| 240 | 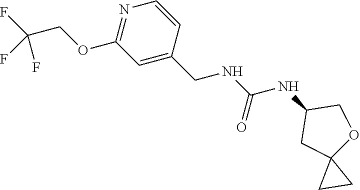 [SMILES: O=C(NCc1ccnc(OCC(F)(F)F)c1)N[C@H]1COC2(CC2)C1] |
| 241 | 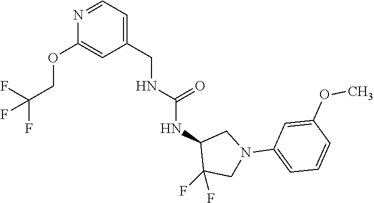 [SMILES: COc1cccc(N2C[C@H](NC(=O)NCc3ccnc(OCC(F)(F)F)c3)C(F)(F)C2)c1] |
| 242 | 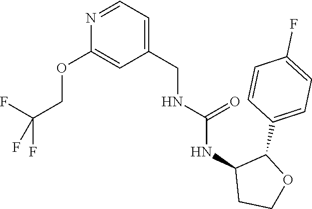 [SMILES: O=C(NCc1ccnc(OCC(F)(F)F)c1)N[C@@H]1CCO[C@H]1c1ccc(F)cc1] |
| 243 | 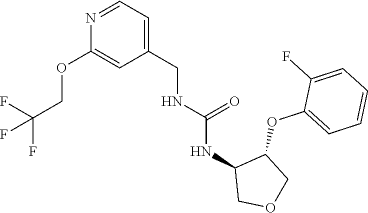 [SMILES: O=C(NCc1ccnc(OCC(F)(F)F)c1)N[C@@H]1COC[C@H]1Oc1ccccc1F] |
| 244 | 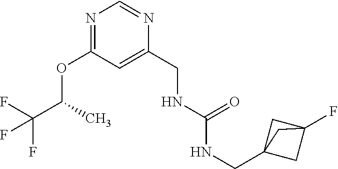 [SMILES: C[C@@H](Oc1cc(CNC(=O)NCC23CC(F)(C2)C3)ncn1)C(F)(F)F] |
| 245 | 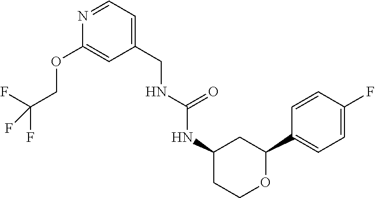 [SMILES: O=C(NCc1ccnc(OCC(F)(F)F)c1)N[C@@H]1CCO[C@H](c2ccc(F)cc2)C1] |
| 246 | 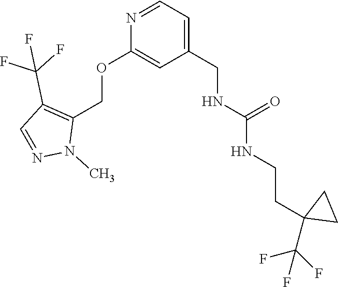 [SMILES: Cn1ncc(C(F)(F)F)c1COc1cc(CNC(=O)NCCC2(C(F)(F)F)CC2)ccn1] |
| 247 | 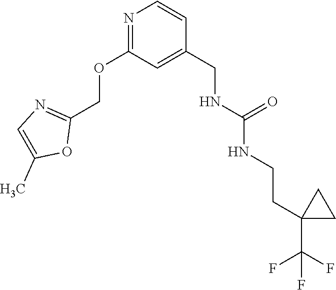 [SMILES: Cc1cnc(COc2cc(CNC(=O)NCCC3(C(F)(F)F)CC3)ccn2)o1] |
| 248 | 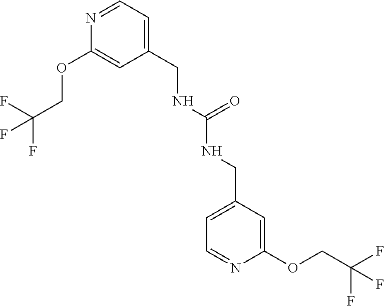 [SMILES: O=C(NCc1ccnc(OCC(F)(F)F)c1)NCc1ccnc(OCC(F)(F)F)c1] |
| 249 | 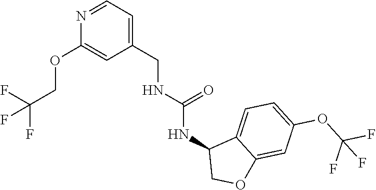 [SMILES: O=C(NCc1ccnc(OCC(F)(F)F)c1)N[C@@H]1COc2cc(OC(F)(F)F)ccc21] |
| 250 | 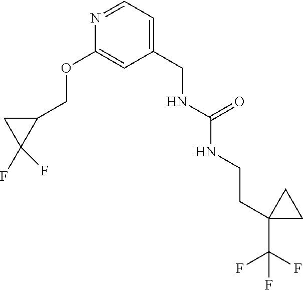 [SMILES: O=C(NCCC1(C(F)(F)F)CC1)NCc1ccnc(OCC2CC2(F)F)c1] |

Some compounds of the present invention may exist in tautomeric forms, and the invention includes all tautomeric forms of such compounds unless otherwise noted.

Unless otherwise stated, the structures depicted herein are also intended to include all stereochemical forms of the structure, i.e., the R and S configurations for each asymmetric center. Individual stereochemical isomers as well as enantiomeric and diastereomeric mixtures of the compounds according to the present invention are within the scope of the invention. The present invention includes any diastereomer or enantiomer substantially free of other isomers (>90%, and preferably >95%, free of other stereoisomers on a molar basis), as well as a mixture of such isomers.

Particular optical isomers can be obtained by resolution of racemic mixtures according to conventional processes, for example, by formation of diastereomeric salts, by treatment with an optically active acid or base and subsequent separation of the mixture of diastereomers by crystallization of the corresponding salt followed finally by liberation of the optically active bases from such salts. Examples of appropriate acids include tartaric, diacetyltartaric, dibenzoyltartaric, ditoluoyltartaric, and camphorosulfonic acids.

A different process for the separation of optical isomers involves the use of a chiral chromatographic column optimally chosen to maximize the separation of enantiomers. Still another method involves the synthesis of covalent diastereomers by reacting the compounds of the invention with an optically pure acid in an activated form or an optically pure isocyanate. The synthesized diastereomers can be separated by conventional means such as chromatography, distillation, crystallization or sublimation, and then hydrolyzed to provide the enantiomerically pure compound. The optically active compounds of the invention can be obtained using active starting materials. These isomers may be in the form of a free acid, a free base, an ester, or a salt.

Compounds of the present invention may exist in radiolabeled form, i.e., said compounds may contain one or more atoms containing an atomic mass or mass number different from the atomic mass or mass number normally found in nature. Radioisotopes of hydrogen, carbon, phosphorus, fluorine, and chlorine include ³H, ¹⁴C, ³²P, ³⁵S, ¹⁸F and ³⁶Cl, respectively. Compounds of the present invention that contain these radioisotopes and/or other radioisotopes of other atoms are within the scope of the present invention. The triziated radioisotopes, i.e., ³H, and carbon-14, i.e., ¹⁴C, are particularly preferred because of their ease of preparation and detectability.

The radiolabeled compounds of this invention can generally be prepared by methods well known to those skilled in the art. Conveniently, such radiolabeled compounds can be prepared by performing the procedures described herein by substituting a non-radiolabeled reagent for a readily available non-radiolabeled reagent.

Compounds according to the present invention are preferably used as salts with pharmaceutically acceptable organic and inorganic acids or bases.

Preferably, the pharmaceutically acceptable organic acids are chosen from the group consisting of oxalic, maleic, methanesulfonic, paratoluenesulfonic, succinic, citric, malic, tartaric and lactic acids.

Preferably, pharmaceutically acceptable organic bases are selected from the group consisting of tromethamine, lysine, arginine, glycine, alanine and ethanolamine.

Preferably, the pharmaceutically acceptable inorganic acids are chosen from the group consisting of hydrochloric, hydrobromic, phosphoric and sulfuric acids.

Preferably, the pharmaceutically acceptable inorganic bases are chosen from the group consisting of hydroxide or carbonate of alkaline or alkaline-earth metals, such as sodium, potassium and calcium.

The compounds of the present invention, or pharmaceutically acceptable salts thereof, can be prepared by a variety of procedures known to a man skilled in the art, some of which are described in the preparations illustrated in the examples of the experimental part. Intermediates and final compounds may be recovered by conventional methods well known in the art, such as, for example, extraction, evaporation, precipitation, chromatography, filtration, trituration, and crystallization. Reagents and starting materials are known and readily available to the man skilled in the art.

Advantageously, the compounds of the present invention are used as a drug, particularly in the treatment of disorders that are modulated by Kv7.2/7.3 potassium channels, preferably in the treatment of central nervous system (CNS) and peripheral nervous system (PNS) disorders.

Central nervous system (CNS) disorders that are preferably treated with the compounds of the present invention are, for example, epilepsy, epileptic syndromes, epileptic symptoms, epilepsy resistant or refractory to treatment, seizures, bipolar disorder, bipolar depression, schizophrenia, psychosis, mania, stress-related disorders, acute stress reactions, major depressive disorder, anxiety, panic attacks, social phobia, sleep disorders, attention deficit hyperactivity disorder, post-traumatic stress disorder, obsessive-compulsive disorder, impulsivity disorders, personality disorders, Huntington's disease, Alzheimer's disease, Parkinson's disease, multiple sclerosis, amyotrophic lateral sclerosis, tinnitus, and so on.

Advantageously, the central nervous system (CNS) disorders that are preferably treated with the compounds of the present invention are epilepsy, epileptic syndromes, epileptic symptoms, epilepsy resistant or refractory to treatment, seizures, bipolar disorder, bipolar depression, schizophrenia, and amyotrophic lateral sclerosis.

Peripheral nervous system (PNS) disorders that are preferably treated with the compounds of the present invention are, for example, migraine, chronic pain, acute pain, neuropathic pain, visceral pain, inflammatory pain, muscle pain, and so forth.

Advantageously, the peripheral nervous system (PNS) disorders that are preferably treated with the compounds of the present invention are neuropathic pain, chronic pain, visceral pain, and inflammatory pain.

Typically, the compounds of the present invention are administered in the form of a pharmaceutical composition comprising a pharmaceutically acceptable excipient.

Thus, one aspect of the present invention relates to a pharmaceutical composition comprising (i) a Kv7.2/7.3 potassium channel activating compound, or a pharmaceutically acceptable salt thereof, according to the first or second aspect of the present invention, and (ii) at least one pharmaceutically acceptable excipient.

Preferably, the pharmaceutical composition according to the present invention is for systemic use.

The pharmaceutical composition according to the present invention can be administered orally, parenterally, inhaled (spray, powder or aerosol), rectally, nasally, buccally, vaginally or via an implanted device.

The term parenteral as used herein includes subcutaneous, intracutaneous, intravenous, intramuscular, intraarticular, intrasynovial, intrasternal, intrathecal, intralesional and intracranial injection or infusion techniques.

More preferably, the pharmaceutical composition according to the present invention is formulated for oral or parenteral administration.

Preferably, the pharmaceutical composition according to the present invention is prepared in suitable dosage forms comprising an effective amount of at least one compound according to the first or second aspect of the present invention, a salt thereof with a pharmaceutically acceptable organic or inorganic acid or base, and at least one pharmaceutically acceptable excipient.

Examples of suitable dosage forms include tablets, capsules, coated tablets, granules and solutions and syrups for oral administration; suppositories for rectal or vaginal administration; and solutions, suspensions, dispersions or emulsions for administration by injection or infusion.

Preferred dosage forms include tablets, coated tablets, capsules and solutions for oral administration, and aqueous to non-aqueous sterile solutions for administration by injection or infusion.

The amount of compound according to the first or second aspect of the present invention, or a pharmacologically acceptable salt thereof, present in the pharmaceutical composition of the present invention may vary over a wide range depending on known factors, for example, the type of disease, the severity of the disease, the body weight of the patient, the dosage form, the route of administration chosen, the number of administrations per day, and the efficacy of the compound itself. However, a person skilled in the art can determine the optimal amount easily and routinely.

Typically, the amount of compound according to the first or second aspect of the present invention or a pharmacologically acceptable salt thereof in the pharmaceutical composition of the present invention will be such as to provide a level of administration from 0.0001 to 100 mg/kg/day. Preferably, the level of administration is from 0.001 to 50 mg/kg/day, and even more preferably from 0.01 to 10 mg/kg/day.

As known to the man skilled in the art, lower or higher doses than those mentioned above may be required. The specific dosage and treatment regimens for any particular patient will depend on a variety of factors, including the activity of the specific compound employed, age, body weight, general health status, gender, diet, time of administration, rate of excretion, combination of drugs, severity and course of disease and the patient's disposition to the disease and the judgment of the treating physician.

The dosage forms of the pharmaceutical composition of the present invention can be prepared according to techniques well known to a man skilled in the pharmaceutical art, including mixing, granulation, compression, dissolution, sterilization, and the like.

Advantageously, such dosage forms are formulated to provide controlled release of the active ingredient over time. In particular, depending on the type of therapy, the required release time may be very short, normal or long.

Preferably, the pharmaceutical composition of the present invention is contained in a single dosage form, to be administered once a day, or several times (two, three or four) a day.

The pharmaceutically acceptable excipient may be selected from the group consisting of thickeners, glidants, binders, disintegrants, fillers, diluents, preservatives, stabilizers, surfactants, buffers, fluidizers, lubricants, humectants, absorbents, salts to regulate osmotic pressure, emulsifiers, flavorings, colorants, sweeteners, and the like.

Particularly preferred excipients include water, ethanol, propylene glycol, glycerol, polyethylene glycols, polyoxamers, mono-, di- and tri-glycerides, coconut oil, palm oil, sodium carbonate, magnesium carbonate, magnesium stearate, stearic acid, talc, sugars, lactose, mannitol, sorbitol, polysorbate, povidone, pectin, dextrin, starch (especially corn starch), sodium starch glycolate, croscarmellose sodium, sucrose, cyclodextrin, gelatin, microcrystalline cellulose, methylcellulose, ethylcellulose, sodium carboxymethylcellulose, povidone, glyceryl monostearate, hypromellose, cocoa butter, titanium dioxide (E171), red iron oxide and yellow iron oxide (E172), and the like.

### EXPERIMENTAL PART

The following examples are intended to further illustrate the present invention, but do not limit it.

### Example 1

### Analytical Methods

Analytical data is included within the procedures below, in the illustrations of the general procedures, or in the tables of examples. Unless otherwise stated, all ¹H NMR data were collected on a Bruker Avance 400 MHz equipped with 5 mm QNP probe or Bruker Avance III 400 MHz, 5 mm BBFO probe or Fourier 300 MHz, 5 mm dual probe instruments and chemical shifts are quoted in parts per million (ppm). LC/MS was performed on Acquity UPLC H-Class (quaternary pump/PDA detector) coupled to QDa Mass Spectrometer or Acquity UPLC (binary pump/PDA detector) coupled to ZQ Mass Spectrometer or Acquity UPLC with Waters DAD coupled to SQD2 Mass Spectrometer. LC/MS data is referenced to LC/MS conditions using the method number provided in Table 1.

TABLE 1
LC/MS analysis methods

| Method | Conditions |
|---|---|
| A | LC/MS analysis condition: Column: Acquity UPLC HSS C18 1.8 μm, 100 × |
| | 2.1 mm plus guard cartridge, maintained at temperature. Mobile phase: |
| | MeCN (0.1% formic acid) in water (0.1% formic acid). Gradient from 5% to |
| | 95% within 6.8 min; Flow rate: 0.4 ml/min; Wavelength: 210-400 nm DAD. |
| | Waters DAD + Waters SQD2, single quadrapole UPLC-MS. |
| B | LC/MS analysis condition: Column: Acquity UPLC BEH C18 1.7 μm, 100 × |
| | 2.1 mm, maintained at 40° C. Mobile phase: MeCN (0.1% ammonia) in water |
| | (0.1% ammonia). Gradient from 5% to 95% within 6.8 min; Flow rate: 0.4 |
| | ml/min; Wavelength: 200-500 nm DAD. Acquity UPLC with PDA detector and |
| | ZQ Mass Spectrometer. |
| C | LC/MS analysis condition: Column: Acquity UPLC BEH C18 1.7 μm, 100 × |
| | 2.1 mm, maintained at 40° C. Mobile phase: MeCN (0.1% formic acid) in water |
| | (0.1% formic acid). Gradient from 5% to 80% within 2.25 min; Flow rate: 1.5 |
| | ml/min; Wavelength: 210-400 nm DAD. Acquity UPLC with PDA detector and |
| | QDa Mass Spectrometer. |
| D | LC/MS analysis condition: Column: Acquity UPLC BEH C18 1.7 μm, 50 × 2.1 |
| | mm, maintained at 40° C. Mobile phase: MeCN (0.03% ammonia) in water |
| | (0.03% ammonia). Gradient from 8% to 97% within 1.9 min; Flow rate: 0.8 |
| | ml/min; Wavelength: 190-400 nm DAD. Acquity UPLC H-Class with PDA |
| | detector and QDa Mass Spectrometer. |
| E | LC/MS analysis condition: Column: Acquity UPLC BEH Shield RP18 1.7 μm, |
| | 100 × 2.1 mm, maintained at temperature. Mobile phase: MeCN in water (10 |
| | mM ammonium hydrogen carbonate). Gradient from 5% to 95% within 6.8 |
| | min; Flow rate: 0.4 ml/min; Wavelength: 210-400 nm DAD. Acquity UPLC with |
| | Waters DAD and Waters SQD2, single quadrapole UPLC-MS. |
| F | LC/MS analysis condition: Column: Acquity UPLC BEH C18 1.7 μm, 100 × |
| | 2.1 mm, maintained at 40° C. Mobile phase: MeCN (0.1% formic acid) in water |
| | (0.1% formic acid). Gradient from 5% to 95% within 6.8 min; Flow rate: 0.4 |
| | ml/min; Wavelength: 200-500 nm DAD. Acquity UPLC with PDA detector and |
| | ZQ Mass Spectrometer. |
| G | LC/MS analysis condition: Column: Acquity UPLC BEH C18 1.7 μm, 50 × 2.1 |
| | mm, maintained at 40° C. Mobile phase: MeCN (0.03% ammonia) in water |
| | (0.03% ammonia). Gradient from 3% to 50% within 2.0 min; Flow rate: 0.8 |
| | ml/min; Wavelength: 190-400 nm DAD. Acquity UPLC H-Class with PDA |
| | detector and QDa Mass Spectrometer. |
| H | LC/MS analysis condition: Column: Acquity UPLC BEH C18 1.7 μm, 100 × |
| | 2.1 mm, maintained at 40° C. Mobile phase: MeCN (0.1% formic acid) in water |
| | (0.1% formic acid). Gradient from 5% to 95% within 2.5 min; Flow rate: 1.5 |
| | ml/min; Wavelength: 210-400 nm DAD. Acquity UPLC with PDA detector and |
| | QDa Mass Spectrometer. |

### Purification Methods

For the general procedures, intermediate and final compounds may be purified by any technique or combination of techniques known to one skilled in the art. Some examples that are not limiting include flash chromatography performed on the COMBIFLASH® Companion purification system or the Biotage SP1 purification system, products were purified using an Isolute® SPE Si II cartridge, (‘Isolute SPE Si cartridge’ refers to a pre-packed polypropylene column containing unbonded activated silica with irregular particles with average size of 50 μm and nominal 60 Å porosity), and a solvent or combination of solvents (heptane, EtOAc, DCM, MeOH, MeCN, water, etc.) that elutes the desired compounds; RP-HPLC purification performed on Waters Mass Directed FractionLynx systems (2767 autosampler, System Fluidics Organiser, 2998 Photodiode array, 2545 pump, 3×515 pump, QDa mass spectrometer), Gilson system (GX281 autosampler, 322 pump, 155 UV/VIS detector), Interchim PuriFlash 4125 coupled to a UV DAD (see Table 2 for some non-limiting conditions); SFC purification performed on a Waters Thar Prep100 system (P200 CO2 pump, 2545 modifier pump, 2998 UV/VIS detector, 2767 liquid handler with Stacked Injection Module) or Waters Thar Investigator semi preparative system (Waters Fluid Delivery Module, 2998 UV/VIS detector, Waters Fraction Collection Module) (see Table 2 for some non-limiting conditions); recrystallization from an appropriate solvent (MeOH, EtOH, i-PrOH, EtOAc, toluene, etc.) or combination of solvents (EtOAc/heptane, EtOAc/MeOH, etc.); precipitation from a combination of solvents (DMF/water, DMSO/DCM, EtOAc/heptane, etc.); trituration with an appropriate solvent (EtOAc, DCM, MeCN, MeOH, EtOH, i-PrOH, n-PrOH, etc.); extractions by dissolving a compound in a liquid and washing with an appropriately immiscible liquid (DCM/water, EtOAc/water, DM/saturated NaHCO₃, EtOAc/saturated NaHCO₃, DCM/10% aqueous H, EtOAc/10% aqueous HCl, etc.); and/or distillation (simple, fractional, Kugelrohr, etc.). Descriptions of these techniques can be found in the following references: Gordon, A. J. and Ford, R. A. “The Chemist's Companion”, 1972; Palleros, D. R. “Experimental Organic Chemistry”, 2000; Still, W. C., Kahn and M. Mitra, A. J. Org. Chem. 1978, 43(14), 2923-2925; Yan, B. “Analysis and Purification Methods in Combinatorial Chemistry” 2003; Harwood, L. M., Moody, C. J. and Percy, J. M. “Experimental Organic Chemistry: Standard and Microscale, 2^{nd} Edition”, 1999.

TABLE 2
RP-HPLC and SFC purification methods

| Method | Conditions |
|---|---|
| 1 | RP-HPLC purification condition: Column Waters Xbridge Phenyl 10 μm, |
| | 19 × 150 mm. Mobile phase: MeOH in water (10 mM NH₄CO₃); Flow rate: 20 |
| | ml/min; Wavelength: 210-400 nm DAD. Sample injected in DMSO (+optional |
| | formic acid and water), 28 min non-linear gradient from 40% to 100% MeOH, |
| | centered around a specific focused gradient. |
| 2 | RP-HPLC purification condition: Column Waters Sunfire C18 10 μm, 19 × |
| | 150 mm. Mobile phase: MeCN in water (0.1% NH₄CO₃); Flow rate: 20 |
| | ml/min; Wavelength: 210-400 nm DAD. Sample injected in DMSO (+optional |
| | formic acid and water), 28 min non-linear gradient from 20% to 80% MeCN, |
| | centered around a specific focused gradient. |
| 3 | RP-HPLC purification condition: Column Waters Sunfire C18 10 μm, 19 × |
| | 150 mm. Mobile phase: MeCN in water (0.1% HCOOH); Flow rate: 20 |
| | ml/min; Wavelength: 210-400 nm DAD. Sample injected in DMSO (+optional |
| | formic acid and water), 28 min non-linear gradient from 20% to 80% MeCN, |
| | centered around a specific focused gradient. |
| 4 | RP-HPLC purification condition: Column Waters Sunfire C18 10 μm, 19 × |
| | 150 mm. Mobile phase: MeCN in water (0.1% CF₃COOH); Flow rate: 20 |
| | ml/min; Wavelength: 210-400 nm DAD. Sample injected in DMSO (+optional |
| | formic acid and water), 28 min non-linear gradient from 35% to 45% MeCN, |
| | centered around a specific focused gradient. |
| 5 | SFC purification condition (chiral): Column LUX Cellulose-3 5 μm, 21.2 × 250 |
| | mm. Mobile phase: MeOH (0.1% DEA)/CO₂; Flow rate: 100 ml/min; |
| | Wavelength: 270 nm DAD. Sample injected in optional MeOH, MeCN or |
| | DCM alone or in combination, 6 min non-linear gradient from 5% to 95% |
| | MeOH, centered around a specific focused gradient. |
| 6 | RP-HPLC purification condition: Column Waters Luna Phenyl-Hexyl 10 μm, |
| | 21.2 × 150 mm. Mobile phase: MeOH in water (0.1% HCOOH); Flow rate: |
| | 20 ml/min; Wavelength: 210-400 nm DAD. Sample injected in DMSO |
| | (+optional formic acid and water), 28 min non-linear gradient from 20% to 80% |
| | MeOH, centered around a specific focused gradient. |
| 7 | Interchim RP-HPLC purification condition: Column PuriFlash C18HP 15 μm, |
| | 40 × 215 mm. Mobile phase: MeCN in water (0.1% HCOOH); Flow rate: 32 |
| | ml/min; Wavelength: 210-400 nm DAD. Sample injected in DMSO (+optional |
| | formic acid and water), 40 min non-linear gradient from 5% to 95% MeCN, |
| | centered around a specific focused gradient. |
| 8 | SFC purification condition (chiral): Column GREENSEP 2-EP 5 μm, 20 × 250 |
| | mm. Mobile phase: MeOH (0.1% NH₄OH)/CO₂; Flow rate: 100 ml/min; |
| | Wavelength: 270 nm DAD. Sample injected in optional MeOH, MeCN or |
| | DCM alone or in combination, 6 min non-linear gradient from 5% to 15% |
| | MeOH, centered around a specific focused gradient. |

### Preparations and Examples

All starting materials are commercially available from Sigma-Aldrich (including Fluka and Discovery CPR) or Acros unless otherwise noted after the chemical name. Reagent/reactant names given are as named on the commercial bottle or as generated by IUPAC conventions or ChemDraw 16.0. None of the specific conditions and reagents noted herein is to be construed as limiting the scope of the invention and are provided for illustrative purposes only.

### ABBREVIATIONS

° C. Degrees Celsius
CAS Chemical Abstracts Service
CDCl₃ Deuterated chloroform
CDI 1,1′-Carbonyldiimidazole
CF₃COOH Trifluoroacetic acid
DAD Diode array detector
DCM Dichloromethane
DEA Diethylamine
DMF N,N-Dimethylformamide
DMSO Dimethyl sulfoxide
DMSO-d₆ Deuterated dimethyl sulfoxide
EtOAc Ethyl acetate
EtOH Ethanol
h Hour(s)
HBTU N,N,N′,N′-Tetramethyl-O-(1H-benzotriazol-1-yl)uronium hexafluorophosphate
HCl Hydrochloride
HCOOH Formic acid
i-PrOH Isopropyl alcohol
IMS Industrial methylated spirits
LC/MS Liquid Chromatography/Mass Spectrometry
m/z Mass-to-charge ratio
MeCN Acetonitrile
MeOD Deuterated methanol
MeOH Methanol
MgSO₄ Magnesium sulphate
MHz Megahertz
Min Minute(s)
MS Mass Spectrometer
Na₂SO₄ Sodium sulphate
NaHCO₃ Sodium hydrogen carbonate
NH₄CO₃ Ammonium hydrogen carbonate
NH₄OH Ammonium hydroxide
NMR Nuclear Magnetic Resonance
PDA Photodiode array
RP-HPLC Reverse Phase-High Performance Liquid Chromatography
Rₜ Retention time
RT Room temperature
SFC Supercritical Fluid Chromatography
tBuBrettPhos Pd G3 [(2-Di-tert-butylphosphino-3,6-dimethoxy-2′,4′,6′-triisopropyl-1,1′-biphenyl)-2-(2′-amino-1,1′-biphenyl)]palladium(II) methanesulfonate
THF Tetrahydrofuran
UPLC Ultra Performance Liquid Chromatography
UV/VIS Ultraviolet/Visible

The synthesis of compounds 1-88 can be accomplished as described below. The synthesis of compounds 89-250 can be accomplished with similar methods by modification of the starting reagents.

### Compound 1

### 1-((2-(Benzyloxy)pyridin-4-yl)methyl)-3-(3-fluorophenyl)urea

A reaction vessel was charged with (2-(benzyloxy)pyridin-4-yl)methanamine (CAS: 869293-84-5, 75 mg, 0.35 mmol), CDI (60 mg, 0.37 mmol) and solvated in DCM (2.5 mL). N,N-diisopropylethylamine (0.12 mL, 0.70 mmol) was added and the reaction was stirred at RT under a nitrogen atmosphere for 30 min. 3-Fluoroaniline (CAS: 372-19-0, 0.040 mL, 0.42 mmol) was added and the reaction was stirred at RT under a nitrogen atmosphere for 21.5 h. The reaction was next heated to 45° C. and stirred at 45° C. for 24 h. The reaction mixture was next partitioned between DCM and distilled water. The organic layer was separated. The combined organic layers were dried (Na₂SO₄) and concentrated in vacuo. The title compound was purified by reverse phase HPLC (Table 2, Method 1) to afford an off-white solid (57 mg, 46%).

¹H NMR (400 MHz, DMSO-d₆) δ 8.96 (s, 1H), 8.11 (dd, J=0.48 Hz, 5.24 Hz, 1H), 7.47-7.25 (m, 7H), 7.08-7.09 (m, 1H), 6.94-6.93 (m, 1H), 6.82 (m, 1H), 6.76 (m, 2H), 5.36 (s, 2H), 4.31 (d, J=6.16 Hz, 2H).

LC/MS (Table 1, Method A) Rt=5.07 min; MS m/z: 352 [M+H]⁺.

### Compound 2

### 1-((2-(Benzyloxy)pyridin-4-yl)methyl)-3-(3-fluorobenzyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(benzyloxy)pyridin-4-yl)methanamine (CAS: 869293-84-5) and 3-fluorobenzylamine (CAS: 100-82-3). The title compound was purified by reverse phase HPLC (Table 2, Method 2) followed by reverse phase HPLC (Table 2, Method 1) to afford an off-white solid (60 mg, 47%).

¹H NMR (400 MHz, DMSO-d₆) δ 8.08 (dd, J=0.52 Hz, 5.20 Hz, 1H), 7.47-7.30 (m, 6H), 7.13-7.01 (m, 3H) 6.88 (dd, J=1.32 Hz, 5.20 Hz, 1H) 6.71 (s, 1H), 6.69-6.61 (m, 2H), 5.35 (s, 2H), 4.28-4.21 (m, 4H).

LC/MS (Table 1, Method A) Rₜ=4.79 min; MS m/z: 366 [M+H]⁺.

### Compound 3

### 1-((2-(Benzyloxy)pyridin-4-yl)methyl)-3-(2-(1-(trifluoromethyl)cyclopropyl) ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(benzyloxy)pyridin-4-yl)methanamine (CAS: 869293-84-5) and 2-(1-(trifluoromethyl)cyclopropyl)ethan-1-amine hydrochloride (CAS: 1454690-80-2). The title compound was purified by reverse phase HPLC (Table 2, Method 3) to afford an off-white solid (62 mg, 45%).

¹H NMR (400 MHz, DMSO-d₆) δ 8.08 (d, J=5.6 Hz, 1H), 7.46-7.32 (m, 5H), 6.87 (dd, J=1.4, 5.3 Hz, 1H), 6.69 (s, 1H), 6.52 (t, J=6.1 Hz, 1H), 6.08 (t, J=5.8 Hz, 1H), 5.35-5.34 (m, 2H), 4.19 (d, J=6.1 Hz, 2H), 3.12 (dd, J=6.1, 15.2 Hz, 2H), 1.71-1.66 (m, 2H), 0.92-0.88 (m, 2H), 0.76 (s, 2H).

LC/MS (Table 1, Method A) Rₜ=5.21 min; MS m/z: 394 [M+H]⁺.

### Compound 4

### (S)-1-((2-(Benzyloxy)pyridin-4-yl)methyl)-3-(2-hydroxy-3,3-dimethylbutyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(benzyloxy)pyridin-4-yl)methanamine (CAS: 869293-84-5) and (S)-1-amino-3,3-dimethylbutan-2-ol hydrochloride (CAS: 2460615-95-4). The title compound was purified by reverse phase HPLC (Table 2, Method 3) to afford an off-white solid (53 mg, 42%).

¹H NMR (400 MHz, DMSO-d₆) δ 8.08 (d, J=5.3 Hz, 1H), 7.46-7.32 (m, 6H), 6.88 (dd, J=1.3, 5.3 Hz, 1H), 6.72-6.62 (m, 2H), 5.98 (dd, J=3.6, 7.3 Hz, 1H), 5.35 (s, 2H), 4.74 (d, J=5.6 Hz, 1H), 4.21 (d, J=6.1 Hz, 2H), 3.10-3.04 (m, 1H), 2.75-2.67 (m, 1H), 0.86-0.85 (m, 9H).

LC/MS (Table 1, Method A) Rₜ=4.59 min; MS m/z: 358 [M+H]⁺.

### Compound 5

### (±)-1-((2-(Benzyloxy)pyridin-4-yl)methyl)-3-(2-hydroxy-2-(1-(trifluoromethyl)cyclo propyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(benzyloxy)pyridin-4-yl)methanamine (CAS: 869293-84-5) and (±)-2-amino-1-(1-(trifluoromethyl)cyclopropyl)ethan-1-olhydrochloride (prepared according to WO2020163268 preparation 3). The title compound was purified by reverse phase HPLC (Table 2, Method 3) to afford an off-white oil (58 mg, 41%).

¹H NMR (400 MHz, DMSO-d₆) δ 8.09 (d, J=5.3 Hz, 1H), 7.46-7.32 (m, 5H), 6.88 (dd, J=1.1, 5.3 Hz, 1H), 6.71-6.58 (m, 2H), 6.14 (dd, J=4.4, 7.2 Hz, 1H), 5.38-5.34 (m, 3H), 4.21 (dd, J=2.9, 5.8 Hz, 2H), 3.60-3.30 (m, 2H, two protons partially obscured by solvent peak), 2.95-2.87 (m, 1H), 0.87-0.84 (m, 4H).

LC/MS (Table 1, Method A) Rₜ=4.74 min; MS m/z: 410 [M+H]⁺.

### Compound 6

### 1-Methyl-3-((2-(2,2,2-trifluoroethoxy)pyridin-4-yl)methyl)-1-(2-(1-(trifluoromethyl) cyclopropyl)ethyl)urea

### (i) tert-Butyl (2-(1-(trifluoromethyl)cyclopropyl)ethyl)carbamate

A reaction vessel was charged with 2-(1-(trifluoromethyl)cyclopropyl)ethan-1-amine hydrochloride (CAS: 1454690-80-2, 50 mg, 0.264 mmol) and solvated in THF (0.80 mL) and distilled water (0.80 mL). Potassium carbonate (182 mg, 1.32 mmol) and di-tert-butyl dicarbonate (0.12 mL, 0.527 mmol) were added and the reaction was stirred at RT for 2 h. The reaction mixture was next partitioned between EtOAc and distilled water. The organic layer was separated. The combined organic layers were washed with saturated brine, dried (Na₂SO₄) and concentrated in vacuo. The residue was purified by flash column chromatography (cyclohexane to EtOAc, gradient elution) to afford the title compound (63 mg, 94%).

¹H NMR (300 MHz, CDCl₃) δ 4.66 (s, 1H), 3.25 (q, J=7.1 Hz, 2H), 1.75 (t, J=7.6 Hz, 2H), 1.44 (s, 9H), 1.00-0.94 (m, 2H), 0.63 (s, 2H).

### (ii) tert-Butyl methyl(2-(1-(trifluoromethyl)cyclopropyl)ethyl)carbamate

To a solution tert-butyl (2-(1-(trifluoromethyl)cyclopropyl)ethyl)carbamate ((Compound 6, step (i), 63 mg, 0.249 mmol)) in anhydrous THF (1.0 mL) at 0° C. under a nitrogen atmosphere was added sodium hydride (60%, 10 mg, 0.249 mmol). The reaction stirred at 0° C. for 30 min. Iodomethane (0.017 mL, 0.274 mmol) was added and the reaction stirred at 0° C. for 1 h. The reaction was allowed to warm to RT and was stirred at RT for 2 h. The reaction was next partitioned between EtOAc and distilled water. The organic layer was separated. The combined organic layers were washed with saturated brine, dried (Na₂SO₄) and concentrated in vacuo to afford the title compound (50 mg, 75%).

¹H NMR (300 MHz, CDCl₃) δ 3.36-3.28 (m, 2H), 2.85 (s, 3H), 1.80-1.69 (m, 2H), 1.45 (s, 9H), 0.97 (s, 2H), 0.69-0.56 (m, 2H).

### (iii) N-Methyl-2-(1-(trifluoromethyl)cyclopropyl)ethan-1-amine hydrochloride

To a solution of tert-butyl methyl(2-(1-(trifluoromethyl)cyclopropyl)ethyl)carbamate ((Compound 6, Step (ii), 282 mg, 1.06 mmol)) in DCM (4.0 mL) at 0° C. was added 4M HCl in 1,4-dioxane (4.0 mL, 15.8 mmol). The reaction was allowed to warm to RT and the reaction stirred at RT for 2 h. The reaction mixture was concentrated in vacuo to afford the title compound (210 mg, 98%).

¹H NMR (300 MHz, MeOD) δ 3.19-3.12 (m, 2H), 2.71 (s, 3H), 1.96-1.90 (m, 2H), 1.09-1.03 (m, 2H), 0.83-0.76 (m, 2H).

### (iv) 1-Methyl-3-((2-(2,2,2-trifluoroethoxy)pyridin-4-yl)methyl)-1-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine dihydrochloride (CAS: 2460508-43-2) and N-Methyl-2-(1-(trifluoromethyl)cyclopropyl)ethan-1-amine hydrochloride ((Compound 6, step (iii)). The title compound was purified by flash column chromatography (DCM to EtOAc, gradient elution) to afford an off-white solid (31 mg, 27%).

¹H NMR (400 MHz, DMSO-d₆) δ 8.09 (dd, J=0.6, 5.3 Hz, 1H), 6.97 (dd, J=1.3, 5.3 Hz, 1H), 6.93 (t, J=5.8 Hz, 1H), 6.76 (dd, J=0.6, 1.3 Hz, 1H), 4.97 (q, J=9.2 Hz, 2H), 4.22 (d, J=5.9 Hz, 2H), 3.31-3.28 (m, 2H), 2.83 (s, 3H), 1.74-1.68 (m, 2H), 0.92-0.87 (m, 2H), 0.79-0.74 (m, 2H).

LC/MS (Table 1, Method B) Rₜ=4.71 min; MS m/z: 400 [M+H]⁺.

### Compound 7

### 1-Methyl-1-((2-(2,2,2-trifluoroethoxy)pyridin-4-yl)methyl)-3-(2-(1-(trifluoromethyl) cyclopropyl)ethyl)urea

### (i) tert-Butyl ((2-(2,2,2-trifluoroethoxy)pyridin-4-yl)methyl)carbamate

The title compound was prepared using an analogous reaction protocol to that described for Compound 6: 1-Methyl-3-((2-(2,2,2-trifluoroethoxy)pyridin-4-yl)methyl)-1-(2-(1-(trifluoromethyl)cyclopropyl)ethyl)urea, step (i), from the appropriate commercial starting material, (2-(2,2,2-trifluoroethoxy)pyridin-4-yl)methanamine dihydrochloride (CAS: 2460508-43-2). The residue was purified by flash column chromatography (cyclohexane to EtOAc, gradient elution) to afford the title compound (184 mg, 87%).

LC/MS (Table 1, Method C) Rₜ=1.72 min; MS m/z: 307 [M+H]⁺.

### (ii) tert-Butyl methyl((2-(2,2,2-trifluoroethoxy)pyridin-4-yl)methyl)carbamate

The title compound was prepared using an analogous reaction protocol to that described for Compound 6: 1-Methyl-3-((2-(2,2,2-trifluoroethoxy)pyridin-4-yl)methyl)-1-(2-(1-(trifluoromethyl)cyclopropyl)ethyl)urea, step (ii), from the appropriate starting material, tert-butyl ((2-(2,2,2-trifluoroethoxy)pyridin-4-yl)methyl)carbamate (Compound 7, step (i)). The compound was purified by flash column chromatography (cyclohexane to EtOAc, gradient elution) to afford the title compound (358 mg, 89%).

LC/MS (Table 1, Method C) Rₜ=1.89 min; MS m/z: 321 [M+H]⁺.

### (iii) N-Methyl-1-(2-(2,2,2-trifluoroethoxy)pyridin-4-yl)methanamine dihydrochloride

The title compound was prepared using an analogous reaction protocol to that described for Compound 6: 1-Methyl-3-((2-(2,2,2-trifluoroethoxy)pyridin-4-yl)methyl)-1-(2-(1-(trifluoromethyl)cyclopropyl)ethyl)urea, step (iii), from the appropriate starting material, tert-butyl methyl((2-(2,2,2-trifluoroethoxy)pyridin-4-yl)methyl)carbamate (Compound 7, step (ii)). This afforded the title compound (305 mg, 93%).

LC/MS (Table 1, Method D) Rₜ=1.19 min; MS m/z: 221 [M+H]⁺.

### (iv) 1-Methyl-1-((2-(2,2,2-trifluoroethoxy)pyridin-4-yl)methyl)-3-(2-(1-(trifluoromethyl) cyclopropyl) ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, N-methyl-1-(2-(2,2,2-trifluoroethoxy)pyridin-4-yl)methanamine dihydrochloride (Compound 7, step (iii)) and 2-(1-(trifluoromethyl)cyclopropyl)ethan-1-amine hydrochloride (CAS: 1454690-80-2). The title compound was purified by reverse phase HPLC (Table 2, Method 1, non-linear gradient from 20% to 80% MeOH) to afford an off-white solid (17 mg, 15%).

¹H NMR (400 MHz, DMSO-d₆) δ 8.14 (d, J=5.2 Hz, 1H), 6.92 (dd, J=1.3, 5.3 Hz, 1H), 6.70 (s, 1H), 6.55 (t, J=5.7 Hz, 1H), 4.99 (q, J=9.2 Hz, 2H), 4.44 (s, 2H), 3.20-3.13 (m, 2H), 2.80 (s, 3H), 1.76-1.70 (m, 2H), 0.93-0.89 (m, 2H), 0.76-0.73 (m, 2H).

LC/MS (Table 1, Method E) Rₜ=4.83 min; MS m/z: 400 [M+H]⁺.

### Compound 8

### 1-((4-(Benzyloxy)pyrimidin-2-yl)methyl)-3-(2-(1-(trifluoromethyl)cyclopropyl) ethyl)urea

### (i) tert-Butyl (tert-butoxycarbonyl)((4-chloropyrimidin-2-yl)methyl)carbamate

To a suspension of di-tert-butyl-iminodicarboxylate (1.13 g, 5.18 mmol) and sodium iodide (1.48 g, 9.89 mmol) in THF (10.0 mL) at 0° C. under a nitrogen atmosphere was added sodium hydride (60%, 217 mg, 5.42 mmol). The reaction was stirred at 0° C. for 30 min. This was followed by the dropwise addition of 4-chloro-2-(chloromethyl)pyrimidine (CAS: 3842-28-2, 768 mg, 4.71 mmol) in THF (10.0 mL). The reaction was allowed to warm to RT and stirred at RT for 16 h. The reaction mixture was next partitioned with EtOAc and distilled water. The organic layer was separated. The combined organic layers were washed with saturated brine, dried (Na₂SO₄) and concentrated in vacuo. The residue was purified by flash column chromatography (cyclohexane to EtOAc, gradient elution) to afford the title compound (177 mg, 74%).

LC/MS (Table 1, Method C) Rₜ=1.85 min; MS m/z: 366 [M+H]⁺.

### (ii) tert-Butyl ((4-(benzyloxy)pyrimidin-2-yl)methyl)carbamate

To a suspension of sodium hydride (60%, 25 mg, 0.618 mmol) in THF (1.0 mL) at 0° C. under a nitrogen atmosphere was added benzyl alcohol (CAS: 100-51-6, 0.064 mL, 0.618 mmol) and the reaction stirred at 0° C. for 30 min. This was followed by the dropwise addition of tert-butyl (tert-butoxycarbonyl)((4-chloropyrimidin-2-yl)methyl)carbamate (Example 8, step (i), 177 mg, 0.515 mmol) in THF (1.0 mL) and the reaction stirred at 0° C. for 15 min. The reaction was allowed to warm to RT and next partitioned between distilled water and EtOAc. The organic layer was separated. The combined organic layer was washed with saturated brine, dried (Na₂SO₄) and concentrated in vacuo. The residue was purified by flash column chromatography (cyclohexane to EtOAc, gradient elution) to afford the title compound (76 mg, 47%).

LC/MS (Table 1, Method C) Rₜ=1.70 min; MS m/z: 316 [M+H]⁺.

### (iii) (4-(Benzyloxy)pyrimidin-2-yl)methanamine hydrochloride

The title compound was prepared using an analogous reaction protocol to that described for Compound 6: 1-Methyl-3-((2-(2,2,2-trifluoroethoxy)pyridin-4-yl)methyl)-1-(2-(1-(trifluoromethyl)cyclopropyl)ethyl)urea, step (iii), from the appropriate starting material, tert-butyl ((4-(benzyloxy)pyrimidin-2-yl)methyl)carbamate (Compound 8, step (ii)). This afforded the title compound (61 mg, quantitative).

¹H NMR (400 MHz, MeOD) δ 8.54 (d, J=6.0 Hz, 1H), 7.48-7.44 (m, 2H), 7.41-7.32 (m, 3H), 6.93 (d, J=6.0 Hz, 1H), 5.52 (s, 2H), 4.31 (s, 2H).

### (iv) 1-((4-(Benzyloxy)pyrimidin-2-yl)methyl)-3-(2-(1-(trifluoromethyl)cyclopropyl)ethyl) urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (4-(Benzyloxy)pyrimidin-2-yl)methanamine hydrochloride (Compound 8, step (iii)) and 2-(1-(trifluoromethyl)cyclopropyl)ethan-1-amine hydrochloride (CAS: 1454690-80-2). The title compound was purified by flash column chromatography (DCM to EtOAc, gradient elution) to afford an off-white solid (25 mg, 45%).

¹H NMR (400 MHz, DMSO-d₆) δ 8.47 (d, J=5.8 Hz, 1H), 7.50-7.46 (m, 2H), 7.42-7.32 (m, 3H), 6.83 (d, J=5.8 Hz, 1H), 6.38 (t, J=5.9 Hz, 1H), 6.30 (t, J=5.6 Hz, 1H), 5.43 (s, 2H), 4.31 (d, J=5.8 Hz, 2H), 3.16-3.08 (m, 2H), 1.69-1.65 (m, 2H), 0.90-0.86 (m, 2H), 0.76-0.71 (m, 2H).

LC/MS (Table 1, Method F) Rₜ=4.30 min; MS m/z: 395 [M+H]⁺.

### Compound 9

### 1,3-Dimethyl-1-((2-(2,2,2-trifluoroethoxy)pyridin-4-yl)methyl)-3-(2-(1-(trifluoromethyl)cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, N-Methyl-1-(2-(2,2,2-trifluoroethoxy)pyridin-4-yl)methanamine dihydrochloride ((Compound 7, Step (iii)) and N-Methyl-2-(1-(trifluoromethyl)cyclopropyl)ethan-1-amine hydrochloride ((Compound 6, Step (iii)). The title compound was purified by flash column chromatography (DCM to EtOAc, gradient elution) to afford an off-white solid (47 mg, 80%).

¹H NMR (400 MHz, DMSO-d₆) δ 8.14 (dd, J=0.6, 5.2 Hz, 1H), 6.99 (dd, J=1.4, 5.2 Hz, 1H), 6.81 (dd, J=0.7, 1.3 Hz, 1H), 4.98 (q, J=9.1 Hz, 2H), 4.29 (s, 2H), 3.23-3.18 (m, 2H), 2.76 (s, 3H), 2.70 (s, 3H), 1.81-1.75 (m, 2H), 0.92-0.88 (m, 2H), 0.77-0.71 (m, 2H).

LC/MS (Table 1, Method F) Rₜ=5.25 min; MS m/z: 414 [M+H]⁺.

### Compound 10

### 1-((2-((1-Methylpiperidin-4-yl)methoxy)pyridin-4-yl)methyl)-3-(2-(1-(trifluoromethyl) cyclopropyl)ethyl)urea

### (i) 2-((1-Methylpiperidin-4-yl)methoxy)isonicotinonitrile

To a suspension of sodium hydride (60%, 95 mg, 2.38 mmol) in THF (15.0 mL) at 0° C. under a nitrogen atmosphere was added 4-(hydroxymethyl)-1-methylpiperidine (CAS: 20691-89-8, 0.29 mL, 2.17 mmol). This was followed by the dropwise addition of 2-chloro-4-pyridinecarbonitrile (CAS: 33252-30-1, 300 mg, 2.17 mmol) in THF (5.0 mL). The reaction was allowed to warm to RT and stirred at RT for 72 h. The reaction was next heated to 60° C. and stirred at 60° C. for 2 h. The reaction mixture was allowed to cool to RT, quenched by the addition of distilled water and next partitioned with EtOAc. The organic layer was separated. The combined organic layer was washed with saturated brine, dried (Na₂SO₄) and concentrated in vacuo. The residue was purified by flash column chromatography (DCM to MeOH, gradient elution) to afford the title compound (201 mg, 40%).

LC/MS (Table 1, Method D) Rt=1.24 min; MS m/z: 232 [M+H]⁺.

### (ii) (2-((1-Methylpiperidin-4-yl)methoxy)pyridin-4-yl)methanamine

To a solution of 2M lithium aluminium hydride (0.61 mL, 1.21 mmol) in THF (8.0 mL) at 0° C. under a nitrogen atmosphere was added 2-((1-methylpiperidin-4-yl)methoxy)isonicotinonitrile (280 mg, 1.21 mmol) in THF (4.0 mL) via dropwise addition. The reaction stirred at 0° C. for 2 h. The reaction mixture was quenched by the addition of distilled water, 2M sodium hydroxide and additional distilled water. The reaction was stirred for 10 min and allowed to warm to RT. MgSO₄ was added. The reaction was filtered and the filtrate was concentrated in vacuo. The residue was purified by flash column chromatography (DCM to DCM:MeOH [2M NH₃]90:10, gradient elution) to afford the title compound (100 mg, 35%).

LC/MS (Table 1, Method D) Rt=0.93 min; MS m/z: 236 [M+H]⁺.

### (iii) 1-((2-((1-Methylpiperidin-4-yl)methoxy)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-((1-Methylpiperidin-4-yl)methoxy)pyridin-4-yl)methanamine (Compound 10, step (ii)) and 2-(1-(trifluoromethyl)cyclopropyl)ethan-1-amine hydrochloride (CAS: 1454690-80-2). The title compound was purified by reverse phase HPLC (Table 2, Method 1, non-linear gradient from 20% to 80% MeOH) to afford an off-white solid (34 mg, 39%).

¹H NMR (400 MHz, DMSO-d₆) δ 8.04 (d, J=5.3 Hz, 1H), 6.82 (dd, J=1.4, 5.3 Hz, 1H), 6.61 (s, 1H), 6.50 (t, J=6.1 Hz, 1H), 6.08 (t, J=5.8 Hz, 1H), 4.17 (d, J=6.1 Hz, 2H), 4.09 (d, J=6.1 Hz, 2H), 3.16-3.09 (m, 2H), 2.77 (d, J=11.3 Hz, 2H), 2.15 (s, 3H), 1.88-1.80 (m, 2H), 1.73-1.65 (m, 5H), 1.33-1.22 (m, 2H), 0.92-0.88 (m, 2H), 0.77-0.74 (m, 2H).

LC/MS (Table 1, Method A) Rₜ=2.70 min; MS m/z: 415 [M+H]⁺.

### Compound 11

### 1-((6-(Benzyloxy)pyrimidin-4-yl)methyl)-3-(2-(1-(trifluoromethyl)cyclopropyl)ethyl) urea

### (i) 6-(Benzyloxy)pyrimidine-4-carbonitrile

The title compound was prepared using an analogous reaction protocol to that described for Compound 10: 1-((2-((1-Methylpiperidin-4-yl)methoxy)pyridin-4-yl)methyl)-3-(2-(1-(trifluoromethyl)cyclopropyl)ethyl)urea, step (i), from the appropriate commercial starting materials, 6-chloropyrimidine-4-carbonitrile (CAS: 939986-65-9) and benzyl alcohol (CAS: 100-51-6). The compound was purified by flash column chromatography (cyclohexane to EtOAc, gradient elution) to afford the title compound (200 mg, 21%).

LC/MS (Table 1, Method C) Rₜ=1.66 min; MS m/z: 212 [M+H]⁺.

### (ii) (6-(Benzyloxy)pyrimidin-4-yl)methanamine

A reaction vessel was charged with 6-(Benzyloxy)pyrimidine-4-carbonitrile (145 mg, 0.686 mmol) and solvated in EtOAc (4.3 mL). Acetic acid (0.74 mL) and 10% palladium on carbon (37 mg, 0.0343 mmol) were added under a nitrogen atmosphere. The reaction was next evacuated and placed under a hydrogen atmosphere. The reaction stirred at RT for 45 min under a hydrogen atmosphere. The reaction mixture was filtered through celite, under a nitrogen atmosphere and washed with EtOAc. The filtrate was concentrated in vacuo. The residue was purified by SCX-2 column chromatography (DCM to DCM:MeOH [2M NH₃]90:10, gradient elution) to afford the title compound (105 mg, 71%).

LC/MS (Table 1, Method C) Rₜ=1.60 min; MS m/z: 216 [M+H]⁺.

### (iii) 1-((6-(Benzyloxy)pyrimidin-4-yl)methyl)-3-(2-(1-(trifluoromethyl)cyclopropyl)ethyl) urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (6-(Benzyloxy)pyrimidin-4-yl)methanamine (Compound 11, step (ii)) and 2-(1-(trifluoromethyl)cyclopropyl)ethan-1-amine hydrochloride (CAS: 1454690-80-2). The title compound was purified by reverse phase HPLC (Table 2, Method 1) followed by reverse phase HPLC (Table 2, Method 3) to afford an off-white solid (150 mg, 82%).

¹H NMR (400 MHz, DMSO-d₆) δ 8.72 (d, J=1.0 Hz, 1H), 7.48-7.35 (m, 5H), 6.74 (d, J=1.0 Hz, 1H), 6.56 (t, J=6.0 Hz, 1H), 6.20 (t, J=5.8 Hz, 1H), 5.43 (s, 2H), 4.22 (d, J=5.9 Hz, 2H), 3.16-3.09 (m, 2H), 1.68 (t, J=7.8 Hz, 2H), 0.92-0.87 (m, 2H), 0.76 (s, 2H).

LC/MS (Table 1, Method A) Rₜ=4.93 min; MS m/z: 395 [M+H]⁺.

### Compound 12

### 1-((2-((Tetrahydro-2H-pyran-4-yl)methoxy)pyridin-4-yl)methyl)-3-(2-(1-(trifluoromethyl)cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-((tetrahydro-2H-pyran-4-yl)methoxy)pyridin-4-yl)methanamine and 2-(1-(trifluoromethyl)cyclopropyl)ethan-1-amine hydrochloride (CAS: 1454690-80-2). (2-((Tetrahydro-2H-pyran-4-yl)methoxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl) cyclopropyl)ethyl)urea, steps (i-ii), from the appropriate commercial starting materials. The title compound was purified by reverse phase HPLC (Table 2, Method 1, non-linear gradient from 20% to 80% MeOH) to afford an off-white solid (45 mg, 47%).

¹H NMR (400 MHz, DMSO-d₆) δ 8.05 (d, J=5.3 Hz, 1H), 6.83 (dd, J=1.3, 5.2 Hz, 1H), 6.62 (s, 1H), 6.51 (t, J=6.1 Hz, 1H), 6.08 (t, J=5.8 Hz, 1H), 4.14 (dd, J=6.3, 27.7 Hz, 4H), 3.87 (dd, J=2.6, 11.3 Hz, 2H), 3.36-3.30 (m, 2H, two protons partially obscured by solvent peak), 3.16-3.08 (m, 2H), 2.06-1.94 (m, 1H), 1.72-1.61 (m, 4H), 1.37-1.25 (m, 2H), 0.92-0.88 (m, 2H), 0.77-0.73 (m, 2H).

LC/MS (Table 1, Method A) Rₜ=3.84 min; MS m/z: 402 [M+H]⁺.

### Compound 13

### 1-((2-(Oxazol-2-ylmethoxy)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(oxazol-2-ylmethoxy)pyridin-4-yl)methanamine and 2-(1-(trifluoromethyl)cyclopropyl)ethan-1-amine hydrochloride (CAS: 1454690-80-2). (2-(Oxazol-2-ylmethoxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl) cyclopropyl)ethyl)urea, steps (i-ii), from the appropriate commercial starting materials. The title compound was purified by reverse phase HPLC (Table 2, Method 3) to afford an off-white solid (16 mg, 21%).

¹H NMR (400 MHz, DMSO-d₆) δ 8.13-8.06 (m, 2H), 7.24 (d, J=0.9 Hz, 1H), 6.91 (dd, J=1.4, 5.3 Hz, 1H), 6.71 (s, 1H), 6.56-6.51 (m, 1H), 6.10 (t, J=5.8 Hz, 1H), 5.44-5.44 (m, 2H), 4.21-4.18 (m, 2H), 3.12 (dd, J=6.1, 15.2 Hz, 2H), 1.71-1.66 (m, 2H), 0.92-0.88 (m, 2H), 0.77-0.73 (m, 2H).

LC/MS (Table 1, Method A) Rₜ=3.86 min; MS m/z: 385 [M+H]⁺.

### Compound 14

### 1-((2-((4-Fluorobenzyl)oxy)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-((4-fluorobenzyl)oxy)pyridin-4-yl)methanamine and 2-(1-(trifluoromethyl)cyclopropyl)ethan-1-amine hydrochloride (CAS: 1454690-80-2). (2-((4-Fluorobenzyl)oxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl)cyclopropyl)ethyl)urea, steps (i-ii), from the appropriate commercial starting materials. The title compound was purified by reverse phase HPLC (Table 2, Method 3) to afford an off-white solid (40 mg, 49%).

¹H NMR (400 MHz, DMSO-d₆) δ 8.08 (d, J=5.5 Hz, 1H), 7.52-7.47 (m, 2H), 7.23-7.18 (m, 2H), 6.87 (dd, J=1.4, 5.3 Hz, 1H), 6.68 (s, 1H), 6.51 (t, J=6.1 Hz, 1H), 6.08 (t, J=5.8 Hz, 1H), 5.33-5.32 (m, 2H), 4.18 (d, J=6.1 Hz, 2H), 3.16-3.08 (m, 2H), 1.71-1.65 (m, 2H), 0.92-0.88 (m, 2H), 0.75 (s, 2H).

LC/MS (Table 1, Method A) Rₜ=4.88 min; MS m/z: 412 [M+H]⁺.

### Compound 15

### 1-((2-((3,3-Difluorocyclobutyl)methoxy)pyridin-4-yl)methyl)-3-(2-(1-(trifluoromethyl)cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, 2-((3,3-difluorocyclobutyl)methoxy)pyridin-4-yl)methanamine (CAS: 2098043-37-7) and 2-(1-(trifluoromethyl)cyclopropyl)ethan-1-amine hydrochloride (CAS: 1454690-80-2). The title compound was purified by reverse phase HPLC (Table 2, Method 1) to afford an off-white solid (67 mg, 63%).

¹H NMR (400 MHz, DMSO-d₆) δ 8.06 (d, J=5.3 Hz, 1H), 6.85 (dd, J=1.4, 5.3 Hz, 1H), 6.64 (s, 1H), 6.51 (t, J=6.1 Hz, 1H), 6.08 (t, J=5.9 Hz, 1H), 4.31 (d, J=6.5 Hz, 2H), 4.18 (d, J=6.1 Hz, 2H), 3.12 (dd, J=6.0, 15.3 Hz, 2H), 2.77-2.65 (m, 3H), 2.49-2.38 (m, 2H), 1.71-1.66 (m, 2H), 0.92-0.88 (m, 2H), 0.77-0.74 (m, 2H).

LC/MS (Table 1, Method A) Rₜ=4.61 min; MS m/z: 408 [M+H]⁺.

### Compound 16

### 1-((2-((2,2,2-Trifluoroethyl)amino)pyridin-4-yl)methyl)-3-(2-(1-(trifluoromethyl) cyclopropyl)ethyl)urea

### (i) 2-((2,2,2-Trifluoroethyl)amino)isonicotinonitrile

A reaction vessel was charged with 2,2,2-trifluoroethylamine (CAS: 753-90-2, 0.39 mL, 4.91 mmol), 4-cyano-2-fluoropyridine (CAS: 3939-14-8, 600 mg, 4.91 mmol) and the reaction was next heated under microwave irradiation at 160° C. for 12 h. The reaction was allowed to cool to RT and next concentrated in vacuo. The residue was purified by flash column chromatography (DCM to DCM:MeOH [2M NH₃]90:10, gradient elution) to afford the title compound (473 mg, 48%).

LC/MS (Table 1, Method D) Rₜ=1.23 min; MS m/z: 202 [M+H]⁺.

### (ii) 4-(Aminomethyl)-N-(2,2,2-trifluoroethyl)pyridin-2-amine

The title compound was prepared using an analogous reaction protocol to that described for Compound 10: 1-((2-((1-Methylpiperidin-4-yl)methoxy)pyridin-4-yl)methyl)-3-(2-(1-(trifluoromethyl)cyclopropyl)ethyl)urea, step (ii), from the appropriate starting material 2-((2,2,2-trifluoroethyl)amino)isonicotinonitrile (Compound 16, step (i)). The compound was purified by flash column chromatography (DCM to DCM:MeOH [2M NH₃]90:10, gradient elution) to afford the title compound (107 mg, 22%).

LC/MS (Table 1, Method D) Rₜ=0.87 min; MS m/z: 206 [M+H]⁺.

### (iii) 1-((2-((2,2,2-Trifluoroethyl)amino)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, 4-(aminomethyl)-N-(2,2,2-trifluoroethyl)pyridin-2-amine (Compound 16, step (ii)) and 2-(1-(trifluoromethyl)cyclopropyl)ethan-1-amine hydrochloride (CAS: 1454690-80-2). The title compound was purified by reverse phase HPLC (Table 2, Method 1) to afford an off-white solid (44 mg, 45%).

¹H NMR (400 MHz, DMSO-d₆) δ 7.93 (d, J=5.1 Hz, 1H), 7.12 (t, J=6.7 Hz, 1H), 6.49-6.41 (m, 3H), 6.03 (t, J=5.8 Hz, 1H), 4.18-4.06 (m, 4H), 3.13 (dd, J=6.1, 15.5 Hz, 2H), 1.71-1.66 (m, 2H), 0.93-0.89 (m, 2H), 0.76 (t, J=6.0 Hz, 2H).

LC/MS (Table 1, Method A) Rₜ=3.03 min; MS m/z: 385 [M+H]⁺.

### Compound 17

### 1-((2-(2-Fluoroethoxy)pyridin-4-yl)methyl)-3-(2-(1-(trifluoromethyl)cyclopropyl) ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-(2-fluoroethoxy)pyridin-4-yl)methanamine and 2-(1-(trifluoromethyl)cyclopropyl)ethan-1-amine hydrochloride (CAS: 1454690-80-2). (2-(2-Fluoroethoxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl)cyclopropyl)ethyl)urea, steps (i-ii), from the appropriate commercial starting materials. The title compound was purified by flash column chromatography (DCM to EtOAc, gradient elution) to afford an off-white solid (97 mg, 76%).

¹H NMR (400 MHz, DMSO-d₆) δ 8.05 (dd, J=0.6, 5.3 Hz, 1H), 6.86 (dd, J=1.4, 5.3 Hz, 1H), 6.66 (dd, J=0.7, 1.4 Hz, 1H), 6.52 (t, J=5.9 Hz, 1H), 6.09 (t, J=5.6 Hz, 1H), 4.80-4.65 (m, 2H), 4.53-4.43 (m, 2H), 4.17 (d, J=6.3 Hz, 2H), 3.15-3.08 (m, 2H), 1.70-1.65 (m, 2H), 0.91-0.87 (m, 2H), 0.77-0.71 (m, 2H).

LC/MS (Table 1, Method F) Rₜ=3.78 min; MS m/z: 350 [M+H]⁺.

### Compound 18

### 1-((2-(2,2-Difluoroethoxy)pyridin-4-yl)methyl)-3-(2-(1-(trifluoromethyl)cyclopropyl) ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2-difluoroethoxy)pyridin-4-yl)methanamine (CAS: 1432680-35-7) and 2-(1-(trifluoromethyl)cyclopropyl)ethan-1-amine hydrochloride (CAS: 1454690-80-2). The title compound was purified by flash column chromatography (DCM to EtOAc, gradient elution) to afford an off-white solid (112 mg, 87%).

¹H NMR (400 MHz, DMSO-d₆) δ 8.07 (dd, J=0.5, 5.3 Hz, 1H), 6.91 (dd, J=1.3, 5.2 Hz, 1H), 6.70 (dd, J=0.7, 1.3 Hz, 1H), 6.54 (t, J=6.0 Hz, 1H), 6.36 (tt, J=3.6, 54.8 Hz, 1H), 6.10 (t, J=5.9 Hz, 1H), 4.54 (dt, J=3.7, 15.1 Hz, 2H), 4.18 (d, J=6.6 Hz, 2H), 3.15-3.07 (m, 2H), 1.70-1.64 (m, 2H), 0.92-0.87 (m, 2H), 0.77-0.71 (m, 2H).

LC/MS (Table 1, Method F) Rₜ=4.16 min; MS m/z: 368 [M+H]⁺.

### Compound 19

### 1-((2-((2-Fluorobenzyl)oxy)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-((2-fluorobenzyl)oxy)pyridin-4-yl)methanamine and 2-(1-(trifluoromethyl)cyclopropyl)ethan-1-amine hydrochloride (CAS: 1454690-80-2). (2-((2-Fluorobenzyl)oxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl)cyclopropyl)ethyl)urea, steps (i-ii), from the appropriate commercial starting materials. The title compound was purified by reverse phase HPLC (Table 2, Method 1) to afford an off-white solid (71 mg, 67%).

¹H NMR (400 MHz, DMSO-d₆) δ 8.10-8.08 (m, 1H), 7.56-7.51 (m, 1H), 7.45-7.38 (m, 1H), 7.27-7.20 (m, 2H), 6.88 (dd, J=1.4, 5.3 Hz, 1H), 6.69 (s, 1H), 6.51 (t, J=6.1 Hz, 1H), 6.08 (t, J=5.8 Hz, 1H), 5.39 (s, 2H), 4.20-4.17 (m, 2H), 3.12 (dd, J=6.1, 15.2 Hz, 2H), 1.71-1.65 (m, 2H), 0.92-0.87 (m, 2H), 0.77-0.74 (m, 2H).

LC/MS (Table 1, Method A) Rₜ=4.93 min; MS m/z: 412 [M+H]⁺.

### Compound 20

### 1-((2-(2,2-Difluoropropoxy)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-(2,2-difluoropropoxy)pyridin-4-yl)methanamine and 2-(1-(trifluoromethyl)cyclopropyl)ethan-1-amine hydrochloride (CAS: 1454690-80-2). (2-(2,2-Difluoropropoxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl)cyclopropyl)ethyl)urea, steps (i-ii), from the appropriate commercial starting materials. The title compound was purified by reverse phase HPLC (Table 2, Method 3) followed by reverse phase HPLC (Table 2, Method 4) to afford an off-white solid (60 mg, 44%).

¹H NMR (400 MHz, DMSO-d₆) δ 8.08 (dd, J=0.6, 5.2 Hz, 1H), 6.92 (dd, J=1.3, 5.3 Hz, 1H), 6.73 (dd, J=0.7, 1.3 Hz, 1H), 6.53 (t, J=6.0 Hz, 1H), 6.10 (t, J=5.4 Hz, 1H), 4.55 (t, J=13.2 Hz, 2H), 4.20 (d, J=5.9 Hz, 2H), 3.16-3.09 (m, 2H), 1.73 (t, J=19.2 Hz, 3H), 1.72-1.66 (m, 2H), 0.92-0.89 (m, 2H), 0.78-0.73 (m, 2H).

LC/MS (Table 1, Method A) Rₜ=4.57 min; MS m/z: 382 [M+H]⁺.

### Compound 21

### 1-(3-(2,2,2-Trifluoroethoxy)-5,6,7,8-tetrahydroisoquinolin-5-yl)-3-(2-(1-(trifluoromethyl)cyclopropyl)ethyl)urea

### (i) (E)-3-Chloro-7,8-dihydroisoquinolin-5(6H)-one oxime

To a solution of 3-chloro-5,6,7,8-tetrahydroisoquinoline (CAS: 875249-27-7, 900 mg, 5.37 mmol) in THF (3.0 mL) under a nitrogen atmosphere was added 1M potassium tert-butoxide in THF (11.0 mL, 10.7 mmol). The reaction was stirred at RT for 18 h. The reaction was allowed to cool to 0° C. and tert-butyl nitrite (2.1 mL, 17.2 mmol) was added. The reaction was allowed to warm to RT and the reaction stirred at RT for 5.5 h. The reaction mixture was quenched when poured onto a saturated aqueous brine solution and next partitioned with EtOAc. The organic layer was separated. The combined organic layers were dried (Na₂SO₄) and concentrated in vacuo. The residue was triturated with DCM to afford the title compound (980 mg, 90%).

LC/MS (Table 1, Method D) Rₜ=1.09 min; MS m/z: 197 [M+H]⁺.

### (ii) 3-Chloro-5,6,7,8-tetrahydroisoquinolin-5-amine

A reaction vessel was charged with (E)-3-Chloro-7,8-dihydroisoquinolin-5(6H)-one oxime ((Compound 21, step (i), 200 mg, 1.02 mmol)) and solvated in acetic acid (8.0 mL). Zinc (333 mg, 5.09 mmol) was added and the reaction was stirred at RT for 1 h. Additional zinc (333 mg, 5.09 mmol) was added and the reaction was stirred at RT for 45 min (1.45 h in total). The reaction mixture was filtered through celite and washed with EtOH. The filtrate was concentrated in vacuo and next partitioned between EtOAc and aqueous saturated sodium hydrogen carbonate. The organic layer was separated. The combined organic layers were dried (Na₂SO₄) and concentrated in vacuo to afford the title compound (124 mg, 64%).

LC/MS (Table 1, Method D) Rₜ=0.92 min; MS m/z: 183 [M+H]⁺.

### (iii) 1-(3-Chloro-5,6,7,8-tetrahydroisoquinolin-5-yl)-3-(2-(1-(trifluoromethyl)cyclopropyl) ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, 3-Chloro-5,6,7,8-tetrahydroisoquinolin-5-amine (Compound 21, step ii)) and 2-(1-(trifluoromethyl)cyclopropyl)ethan-1-amine hydrochloride (CAS: 1454690-80-2). The compound was purified by flash column chromatography (cyclohexane to EtOAc, gradient elution) to afford the title compound (155 mg, 60%).

LC/MS (Table 1, Method D) Rₜ=1.35 min; MS m/z: 362 [M+H]⁺.

### (iv) 1-(3-(2,2,2-Trifluoroethoxy)-5,6,7,8-tetrahydroisoquinolin-5-yl)-3-(2-(1-(trifluoromethyl) cyclopropyl)ethyl)urea

A reaction vessel was charged with 1-(3-chloro-5,6,7,8-tetrahydroisoquinolin-5-yl)-3-(2-(1-(trifluoromethyl)cyclopropyl)ethyl)urea ((Compound 21, step (iii), 53 mg, 0.133 mmol), tBuBrettPhos Pd G3 (11 mg, 0.0133 mmol), sodium tert-butoxide (64 mg, 0.667 mmol) and solvated in 2,2,2-trifluoroethanol (0.078 mL, 1.07 mmol) and 1,4-dioxane (0.50 mL) under a nitrogen atmosphere. The reaction was set to stir at RT and next heated to 60° C. The reaction was stirred at 60° C. for 3 h. The reaction was allowed to cool to RT. The reaction mixture was filtered through celite and washed with EtOAc. The filtrate was concentrated in vacuo. The residue was purified by flash column chromatography (cyclohexane to EtOAc, gradient elution) followed by reverse phase HPLC (Table 2, Method 3) to afford the title compound as an off-white solid (53 mg, 93%).

¹H NMR (400 MHz, DMSO-d₆) δ 7.96 (s, 1H), 6.72 (s, 1H), 6.45-6.41 (m, 1H), 5.91 (t, J=5.8 Hz, 1H), 5.03-4.88 (m, 2H), 4.76-4.68 (m, 1H), 3.17 (dd, J=6.3, 14.8 Hz, 2H), 2.71-2.65 (m, 2H), 1.98-1.82 (m, 2H), 1.75-1.69 (m, 3H), 1.62-1.52 (m, 1H), 0.94-0.89 (m, 2H), 0.78 (t, J=5.6 Hz, 2H).

LC/MS (Table 1, Method E) Rₜ=4.96 min; MS m/z: 426 [M+H]⁺.

### Compound 22

### 1-((2-((1-Fluorocyclopropyl)methoxy)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-((1-fluorocyclopropyl)methoxy)pyridin-4-yl)methanamine and 2-(1-(trifluoromethyl)cyclopropyl)ethan-1-amine hydrochloride (CAS: 1454690-80-2). (2-((1-fluorocyclopropyl)methoxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl) cyclopropyl)ethyl)urea, steps (i-ii), from the appropriate commercial starting materials. The title compound was purified by reverse phase HPLC (Table 2, Method 2) to afford an off-white solid (60 mg, 39%).

¹H NMR (400 MHz, DMSO-d₆) δ 8.05 (d, J=5.3 Hz, 1H), 6.87 (dd, J=1.3, 5.3 Hz, 1H), 6.70 (s, 1H), 6.52 (t, J=6.1 Hz, 1H), 6.09 (t, J=5.8 Hz, 1H), 4.59 (s, 1H), 4.53 (s, 1H), 4.21-4.17 (m, 2H), 3.13 (dd, J=6.1, 15.3 Hz, 2H), 1.72-1.67 (m, 2H), 1.22-1.06 (m, 2H), 0.93-0.84 (m, 4H), 0.76 (s, 2H).

LC/MS (Table 1, Method A) Rₜ=4.44 min; MS m/z: 376 [M+H]⁺.

### Compound 23

### 1-((2-(Pyridin-2-ylmethoxy)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-(pyridin-2-ylmethoxy)pyridin-4-yl)methanamine and 2-(1-(trifluoromethyl)cyclopropyl)ethan-1-amine hydrochloride (CAS: 1454690-80-2). (2-(Pyridin-2-ylmethoxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl)cyclopropyl)ethyl)urea, steps (i-ii), from the appropriate commercial starting materials. The title compound was purified by reverse phase HPLC (Table 2, Method 3) to afford an off-white solid (61 mg, 41%).

¹H NMR (400 MHz, DMSO-d₆) δ 8.57-8.55 (m, 1H), 8.07-8.05 (m, 1H), 7.83-7.78 (m, 1H), 7.44-7.41 (m, 1H), 7.35-7.31 (m, 1H), 6.88 (dd, J=1.3, 5.2 Hz, 1H), 6.76 (s, 1H), 6.56-6.51 (m, 1H), 6.10 (t, J=5.8 Hz, 1H), 5.43-5.42 (m, 2H), 4.22-4.19 (m, 2H), 3.13 (dd, J=6.1, 15.2 Hz, 2H), 1.72-1.67 (m, 2H), 0.92-0.88 (m, 2H), 0.78-0.75 (m, 2H).

LC/MS (Table 1, Method A) Rₜ=3.36 min; MS m/z: 395 [M+H]⁺.

### Compound 24

### 1-((2-((3-Fluorooxetan-3-yl)methoxy)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-((3-fluorooxetan-3-yl)methoxy)pyridin-4-yl)methanamine and 2-(1-(trifluoromethyl)cyclopropyl)ethan-1-amine hydrochloride (CAS: 1454690-80-2). (2-((3-Fluorooxetan-3-yl)methoxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl) cyclopropyl)ethyl)urea, steps (i-i), from the appropriate commercial starting materials. The title compound was purified by reverse phase HPLC (Table 2, Method 3) followed by reverse phase HPLC (Table 2, Method 2, non-linear gradient from 5% to 60% MeCN) to afford an off-white solid (15 mg, 13%).

¹H NMR (400 MHz, DMSO-d₆) δ 8.09 (d, J=5.3 Hz, 2H), 6.90 (dd, J=1.4, 5.3 Hz, 2H), 6.69 (s, 2H), 6.52 (t, J=6.1 Hz, 2H), 6.09 (t, J=5.8 Hz, 2H), 4.74-4.66 (m, 12H), 4.19 (d, J=6.0 Hz, 4H), 3.13 (dd, J=6.1, 15.1 Hz, 4H), 1.71-1.66 (m, 4H), 0.92-0.88 (m, 4H), 0.76 (s, 4H).

LC/MS (Table 1, Method A) Rₜ=3.94 min; MS m/z: 392 [M+H]⁺.

### Compound 25

### 1-((2-((2,4-Difluorobenzyl)oxy)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea from the appropriate I starting materials, (2-((2,4-difluorobenzyl)oxy)pyridin-4-yl)methanamine and 2-(1-(trifluoromethyl)cyclopropyl)ethan-1-amine hydrochloride (CAS: 1454690-80-2). (2-((2,4-difluorobenzyl)oxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl) cyclopropyl)ethyl)urea, steps (i-i), from the appropriate commercial starting materials. The title compound was purified by reverse phase HPLC (Table 2, Method 1) followed by SFC purification (Table 2, Method 5) to afford an off-white solid (39 mg, 37%).

¹H NMR (400 MHz, DMSO-d₆) δ 8.09 (d, J=5.3 Hz, 1H), 7.64-7.57 (m, 1H), 7.33-7.27 (m, 1H), 7.14-7.08 (m, 1H), 6.88 (dd, J=1.4, 5.3 Hz, 1H), 6.67 (s, 1H), 6.51 (t, J=6.1 Hz, 1H), 6.07 (t, J=5.8 Hz, 1H), 5.35 (s, 2H), 4.20-4.17 (m, 2H), 3.12 (dd, J=6.1, 15.2 Hz, 2H), 1.71-1.65 (m, 2H), 0.92-0.87 (m, 2H), 0.75 (s, 2H).

LC/MS (Table 1, Method A) Rₜ=4.92 min; MS m/z: 430 [M+H]⁺.

### Compound 26

### 1-((2-Morpholino-6-(2,2,2-trifluoroethoxy)pyridin-4-yl)methyl)-3-(2-(1-(trifluoromethyl)cyclopropyl)ethyl)urea

### (i) 2-Chloro-6-morpholinoisonicotinonitrile

A reaction vessel was charged with 2,6-dichloropyridine-4-carbonitrile (CAS: 32710-65-9, 977 mg, 5.65 mmol) and solvated in EtOH (50 mL). Triethylamine (0.79 mL, 5.65 mmol) and morpholine (0.49 mL, 5.65 mmol) were added under a nitrogen atmosphere. The reaction was set to stir at room temperature and next heated to 70° C. The reaction stirred at 70° C. for 5.5 h. The reaction mixture was allowed to cool to RT and concentrated in vacuo. The residue was next partitioned between EtOAc and distilled water. The organic layers were separated. The combined organic layer was washed with saturated brine, dried (MgSO₄) and concentrated in vacuo. The residue was purified by flash column chromatography (cyclohexane to THF, gradient elution) to afford the title compound (805 mg, 64%).

LC/MS (Table 1, Method A) Rₜ=1.58 min; MS m/z: 224 [M+H]⁺.

### (ii) 2-Morpholino-6-(2,2,2-trifluoroethoxy)isonicotinonitrile

A reaction vessel was charged with 2-chloro-6-morpholinoisonicotinonitrile (340 mg, 1.52 mmol), 2,2,2-trifluoroethanol (CAS: 75-89-8, 0.11 mL, 1.52 mmol), cesium carbonate (1.49 g, 4.56 mmol), Xantphos PdG4 (73 mg, 0.0760 mmol) and solvated in toluene (15.0 mL). The reaction was purged and placed under a nitrogen atmosphere. The reaction was set to stir at RT and next heated to 80° C. The reaction stirred at 80° C. for 72 h. The reaction mixture was allowed to cool to RT and concentrated in vacuo. The reaction was next partitioned between EtOAc and distilled water. The organic layer was separated.

The combined organic layers were washed with saturated brine, dried (Na₂SO₄) and concentrated in vacuo. The residue was purified by flash column chromatography (cyclohexane to EtOAc, gradient elution) to afford the title compound (410 mg, 87%).

LC/MS (Table 1, Method A) Rₜ=1.74 min; MS m/z: 288 [M+H]⁺.

### (iii) (2-Morpholino-6-(2,2,2-trifluoroethoxy)pyridin-4-yl)methanamine

The title compound was prepared using an analogous reaction protocol to that described for Compound 10: 1-((2-((1-Methylpiperidin-4-yl)methoxy)pyridin-4-yl)methyl)-3-(2-(1-(trifluoromethyl) cyclopropyl)ethyl)urea, step (ii), from the appropriate starting material, 2-morpholino-6-(2,2,2-trifluoroethoxy)isonicotinonitrile (Compound 26, step (ii)). This afforded the title compound (465 mg, quantitative).

LC/MS (Table 1, Method D) Rₜ=1.35 min; MS m/z: 292 [M−H]⁺.

### (iv) 1-((2-Morpholino-6-(2,2,2-trifluoroethoxy)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea from the appropriate starting materials, (2-Morpholino-6-(2,2,2-trifluoroethoxy)pyridin-4-yl)methanamine (Compound 26, step (iii)) and 2-(1-(trifluoromethyl)cyclopropyl)ethan-1-aminehydrochloride (CAS: 1454690-80-2). The title compound was purified by reverse phase HPLC (Table 2, Method 3) to afford an off-white solid (116 mg, 47%).

¹H NMR (400 MHz, DMSO-d₆) δ 6.45 (t, J=6.0 Hz, 1H), 6.31 (s, 1H), 6.08 (s, 1H), 6.05-6.01 (m, 1H), 4.92 (q, J=9.2 Hz, 2H), 4.11 (d, J=6.0 Hz, 2H), 3.72-3.69 (m, 4H), 3.44-3.39 (m, 4H), 3.16-3.09 (m, 2H), 1.71-1.66 (m, 2H), 0.92-0.88 (m, 2H), 0.78-0.72 (m, 2H).

LC/MS (Table 1, Method E) Rₜ=4.77 min; MS m/z: 471 [M+H]⁺.

### Compound 27

### 1-((2-((4-Fluorobenzyl)oxy)-6-morpholinopyridin-4-yl)methyl)-3-(2-(1-(trifluoromethyl)cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-((4-fluorobenzyl)oxy)-6-morpholinopyridin-4-yl)methanamine and 2-(1-(trifluoromethyl)cyclopropyl)ethan-1-amine hydrochloride (CAS: 1454690-80-2). (2-((4-Fluorobenzyl)oxy)-6-morpholinopyridin-4-yl)methanamine, was in turn prepared following an analogous reaction protocol to that described for Compound 26: 1-((2-Morpholino-6-(2,2,2-trifluoroethoxy)pyridin-4-yl)methyl)-3-(2-(1-(trifluoromethyl)cyclopropyl)ethyl)urea, steps (i-iii), from the appropriate commercial starting materials. The title compound was purified by reverse phase HPLC (Table 2, Method 2) to afford an off-white solid (27 mg, 29%).

¹H NMR (400 MHz, DMSO-d₆) δ 7.49-7.43 (m, 2H), 7.22-7.16 (m, 2H), 6.41 (t, J=6.2 Hz, 1H), 6.20 (s, 1H), 6.02-5.98 (m, 2H), 5.27 (s, 2H), 4.09 (d, J=5.7 Hz, 2H), 3.71-3.67 (m, 4H), 3.42-3.38 (m, 4H), 3.15-3.09 (m, 2H), 1.70-1.65 (m, 2H), 0.92-0.88 (m, 2H), 0.77-0.72 (m, 2H).

LC/MS (Table 1, Method A) Rₜ=5.21 min; MS m/z: 497 [M+H]⁺.

### Compound 28

### (±)-1-((2-(3-Fluoropiperidin-1-yl)pyridin-4-yl)methyl)-3-(2-(1-(trifluoromethyl) cyclopropyl)ethyl)urea

### (i) (±)-2-(3-Fluoropiperidin-1-yl)isonicotinonitrile

A reaction vessel was charged with 4-cyano-2-fluoropyridine (CAS: 3939-14-8, 675 mg, 5.53 mmol), (±)-3-fluoropiperidine hydrochloride (CAS: 116574-75-5, 842 mg, 6.03 mmol) and solvated in EtOH (10.0 mL) under a nitrogen atmosphere. Triethylamine (1.9 mL, 13.8 mmol) was added. The reaction was set to stir at RT and next heated to 70° C. The reaction was stirred at 70° C. for 18 h. The reaction mixture was allowed to cool to RT and was next partitioned between DCM and distilled water. The organic layer was separated. The combined organic layer was dried (MgSO₄) and concentrated in vacuo. The residue was purified by flash column chromatography (cyclohexane to EtOAc, gradient elution) to afford the title compound (730 mg, 64%).

LC/MS (Table 1, Method C) Rₜ=1.78 min; MS m/z: 206 [M+H]⁺.

### (ii) (±)-(2-(3-Fluoropiperidin-1-yl)pyridin-4-yl)methanamine

The title compound was prepared using an analogous reaction protocol to that described for Compound 10: 1-((2-((1-Methylpiperidin-4-yl)methoxy)pyridin-4-yl)methyl)-3-(2-(1-(trifluoromethyl), step (ii), from the appropriate starting material, (±)-2-(3-fluoropiperidin-1-yl)isonicotinonitrile (Compound 28, step (i)). The compound was purified by flash column chromatography (DCM to DCM:MeOH [2M NH₃]90:10, gradient elution) to afford the title compound (520 mg, 70%).

LC/MS (Table 1, Method D) Rₜ=1.30 min; MS m/z: 210 [M+H]⁺.

### (iii) (±)-1-((2-(3-Fluoropiperidin-1-yl)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (±)-(2-(3-Fluoropiperidin-1-yl)pyridin-4-yl)methanamine (Compound 28, step (ii)) and 2-(1-(trifluoromethyl)cyclopropyl)ethan-1-amine hydrochloride (CAS: 1454690-80-2). The title compound was purified by reverse phase HPLC (Table 2, Method 3, non-linear gradient from 5% to 60% MeCN) to afford a white solid (63 mg, 34%).

¹H NMR (400 MHz, DMSO-d₆) δ 8.01 (d, J=5.0 Hz, 1H), 6.68 (s, 1H), 6.50 (d, J=5.0 Hz, 1H), 6.44 (dd, J=6.1, 6.1 Hz, 1H), 6.03 (dd, J=5.8, 5.8 Hz, 1H), 4.83-4.64 (m, 1H), 4.12 (d, J=6.0 Hz, 2H), 3.75-3.55 (m, 3H), 3.42-3.39 (m, 1H), 3.13 (dd, J=6.2, 15.1 Hz, 2H), 2.01-1.73 (m, 3H), 1.71-1.66 (m, 2H), 1.58-1.49 (m, 1H), 0.92-0.88 (m, 2H), 0.76 (s, 2H).

LC/MS (Table 1, Method E) Rₜ=4.17 min; MS m/z: 389 [M+H]⁺.

### Compound 29

### 1-((2-(3-(Difluoromethyl)azetidin-1-yl)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-(3-(difluoromethyl)azetidin-1-yl)pyridin-4-yl)methanamine and 2-(1-(trifluoromethyl)cyclopropyl)ethan-1-amine hydrochloride (CAS: 1454690-80-2). (2-(3-(Difluoromethyl)azetidin-1-yl)pyridin-4-yl)methanamine was in turn prepared following an analogous reaction protocol to that described for Compound 28: (±)-1-((2-(3-Fluoropiperidin-1-yl)pyridin-4-yl)methyl)-3-(2-(1-(trifluoromethyl) cyclopropyl) ethyl)urea, steps (i-i), from the appropriate commercial starting materials. The title compound was purified by reverse phase HPLC (Table 2, Method 2, non-linear gradient from 5% to 60% MeCN) to afford a white solid (22 mg, 35%).

¹H NMR (400 MHz, DMSO-d₆) δ 7.99 (dd, J=0.6, 5.2 Hz, 1H), 6.56-6.02 (m, 5H), 4.12 (d, J=6.1 Hz, 2H), 4.02 (t, J=8.2 Hz, 2H), 3.87-3.82 (m, 2H), 3.30-3.17 (m, 1H), 3.12 (q, J=7.1 Hz, 2H), 1.69 (t, J=8.1 Hz, 2H), 0.92-0.88 (m, 2H), 0.76 (s, 2H).

LC/MS (Table 1, Method A) Rₜ=3.01 min; MS m/z: 393 [M+H]⁺.

### Compound 30

### 1-((4-(2,2,2-Trifluoroethoxy)pyrimidin-2-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (4-(2,2,2-trifluoroethoxy)pyrimidin-2-yl)methanamine, hydrochloride (CAS: 1196154-15-0) and 2-(1-(trifluoromethyl)cyclopropyl)ethan-1-amine hydrochloride (CAS: 1454690-80-2). The title compound was purified by reverse phase HPLC (Table 2, Method 2) to afford a white solid (56 mg, 40%).

¹H NMR (400 MHz, DMSO-d₆) δ 8.59 (d, J=5.8 Hz, 1H), 7.01 (d, J=5.6 Hz, 1H), 6.41 (dd, J=5.8, 5.8 Hz, 1H), 6.28 (dd, J=5.9, 5.9 Hz, 1H), 5.16-5.09 (m, 2H), 4.34 (d, J=5.8 Hz, 2H), 3.13 (dd, J=6.1, 15.4 Hz, 2H), 1.71-1.66 (m, 2H), 0.93-0.88 (m, 2H), 0.76 (s, 2H).

LCMS (Table 1, Method E) Rₜ=4.18 min MS, m/z: 387 [M+H]⁺.

### Compound 31

### 1-((6-(2,2,2-Trifluoroethoxy)pyridin-2-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (6-(2,2,2-trifluoroethoxy)pyridin-2-yl)methanamine (CAS: 1250054-65-9) and 2-(1-(trifluoromethyl)cyclopropyl)ethan-1-amine hydrochloride (CAS: 1454690-80-2). The title compound was purified by reverse phase HPLC (Table 2, Method 3) to afford an off-white solid (77 mg, 47%).

¹H NMR (400 MHz, DMSO-d₆) δ 7.79-7.75 (m, 1H), 6.97 (d, J=6.9 Hz, 1H), 6.83 (d, J=7.9 Hz, 1H), 6.48 (t, J=6.0 Hz, 1H), 6.15 (t, J=5.8 Hz, 1H), 5.01 (q, J=9.2 Hz, 2H), 4.23 (d, J=6.0 Hz, 2H), 3.17-3.10 (m, 2H), 1.69 (t, J=7.8 Hz, 2H), 0.93-0.88 (m, 2H), 0.77-0.74 (m, 2H).

LCMS (Table 1, Method A) Rₜ=4.87 min MS, m/z: 386 [M+H]⁺.

### Compound 32

### 1-((2-Chloro-6-(2,2,2-trifluoroethoxy)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-chloro-6-(2,2,2-trifluoroethoxy)pyridin-4-yl)methanamine and 2-(1-(trifluoromethyl)cyclopropyl)ethan-1-amine hydrochloride (CAS: 1454690-80-2). (2-Chloro-6-(2,2,2-trifluoroethoxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl) cyclopropyl)ethyl)urea, steps (i-ii), from the appropriate commercial starting materials. The title compound was purified by flash column chromatography (cyclohexane to EtOAc:IMS 3:1, gradient elution) followed by flash column chromatography (DCM to EtOAc, gradient elution) to afford a white solid (63 mg, 56%).

¹H NMR (400 MHz, DMSO-d₆) δ 7.07 (d, J=1.0 Hz, 1H), 6.79 (d, J=1.1 Hz, 1H), 6.60 (t, J=6.0 Hz, 1H), 6.17 (t, J=5.4 Hz, 1H), 4.96 (q, J=9.1 Hz, 2H), 4.20 (d, J=5.8 Hz, 2H), 3.14-3.08 (m, 2H), 1.71-1.65 (m, 2H), 0.91-0.87 (m, 2H), 0.76-0.71 (m, 2H).

LCMS (Table 1, Method F) Rₜ=4.92 min MS, m/z: 420 [M+H]⁺.

### Compound 33

### 1-(4,4,4-Trifluoro-3-hydroxy-3-methylbutyl)-3-((2-(2,2,2-trifluoroethoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine hydrochloride (CAS: 2044704-69-8) and 4-amino-1,1,1-trifluoro-2-methylbutan-2-ol (CAS: 911060-86-1). The title compound was purified by reverse phase HPLC (Table 2, Method 3) to afford a white solid (57 mg, 35%).

¹H NMR (400 MHz, DMSO-d₆) δ 8.11 (d, J=5.3 Hz, 1H), 6.98 (d, J=5.3 Hz, 1H), 6.78 (s, 1H), 6.55 (dd, J=6.1, 6.1 Hz, 1H), 6.11 (dd, J=5.6, 5.6 Hz, 1H), 5.90 (s, 1H), 4.99 (q, J=9.1 Hz, 2H), 4.22 (d, J=6.1 Hz, 2H), 3.26-3.12 (m, 2H), 1.79-1.66 (m, 2H), 1.27 (s, 3H).

LCMS (Table 1, Method E) Rₜ=3.95 min MS, m/z: 390 [M+H]⁺.

### Compound 34

### (±)-1-((2-(3-Fluoropyrrolidin-1-yl)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (±)-(2-(3-fluoropyrrolidin-1-yl)pyridin-4-yl)methanamine and 2-(1-(trifluoromethyl)cyclopropyl)ethan-1-amine hydrochloride (CAS: 1454690-80-2). (±)-(2-(3-fluoropyrrolidin-1-yl)pyridin-4-yl)methanamine was in turn prepared following an analogous reaction protocol to that described for Compound 28: (±)-1-((2-(3-Fluoropiperidin-1-yl)pyridin-4-yl)methyl)-3-(2-(1-(trifluoromethyl)cyclopropyl)ethyl)urea, steps (i-ii), from the appropriate commercial starting materials. The title compound was purified by reverse phase HPLC (Table 2, Method 2, non-linear gradient from 5% to 60% MeCN) to afford a white solid (52 mg, 28%).

¹H NMR (400 MHz, DMSO-d₆) δ 7.99 (d, J=5.5 Hz, 1H), 6.48-6.42 (m, 2H), 6.34 (s, 1H), 6.03 (t, J=5.8 Hz, 1H), 5.52-5.36 (m, 1H), 4.13 (d, J=6.1 Hz, 2H), 3.73-3.50 (m, 2H), 3.44-3.37 (m, 1H), 3.29 (s, 1H), 3.13 (dd, J=6.1, 15.1 Hz, 2H), 2.36-2.10 (m, 2H), 1.72-1.66 (m, 2H), 0.92-0.88 (m, 2H), 0.76 (dd, J=6.7, 6.7 Hz, 2H).

LC/MS (Table 1, Method A) Rₜ=2.90 min; MS m/z: 375 [M+H]⁺.

### Compound 35

### 1-((2-(2,2,2-Trifluoroethoxy)pyridin-4-yl)methyl)-3-((1-(trifluoromethyl) cyclopropyl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and (1-(trifluoromethyl)cyclopropyl)methanamine hydrochloride (CAS: 1783418-59-6). The title compound was purified by reverse phase HPLC (Table 2, Method 2) to afford an off-white solid (65 mg, 53%).

¹H NMR (400 MHz, DMSO-d₆) δ 8.12 (d, J=5.3 Hz, 1H), 6.97 (dd, J=1.4, 5.3 Hz, 1H), 6.78 (s, 1H), 6.57 (t, J=6.1 Hz, 1H), 6.33 (t, J=6.1 Hz, 1H), 4.99 (q, J=9.2 Hz, 2H), 4.24 (d, J=6.1 Hz, 2H), 3.40-3.31 (m, 2H), 0.90-0.79 (m, 4H).

LCMS (Table 1, Method A) Rₜ=4.57 min MS, m/z: 372 [M+H]⁺.

### Compound 36

### 1-(Cyclohexylmethyl)-3-((2-(2,2,2-trifluoroethoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and cyclohexylmethanamine (CAS: 3218-02-8). The title compound was purified by reverse phase HPLC (Table 2, Method 1) to afford an off-white solid (35 mg, 30%).

¹H NMR (400 MHz, DMSO-d₆) δ 8.11 (d, J=5.3 Hz, 1H), 6.98 (dd, J=1.3, 5.3 Hz, 1H), 6.77 (s, 1H), 6.38 (t, J=5.9 Hz, 1H), 6.10 (t, J=5.8 Hz, 1H), 4.99 (q, J=9.2 Hz, 2H), 4.22 (d, J=6.1 Hz, 2H), 2.87 (t, J=6.3 Hz, 2H), 1.69-1.61 (m, 5H), 1.39-1.31 (m, 1H), 1.25-1.06 (m, 3H), 0.92-0.82 (m, 2H).

LCMS (Table 1, Method E) Rₜ=4.72 min MS, m/z: 346 [M+H]⁺.

### Compound 37

### 1-(2-Methylbenzyl)-3-((2-(2,2,2-trifluoroethoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and 2-methylbenzylamine (CAS: 89-93-0). The title compound was purified by reverse phase HPLC (Table 2, Method 2) to afford an off-white solid (47 mg, 42%).

¹H NMR (400 MHz, DMSO-d₆) δ 8.12 (dd, J=0.5, 5.4 Hz, 1H), 7.24-7.20 (m, 1H), 7.18-7.14 (m, 3H), 7.00 (dd, J=1.3, 5.3 Hz, 1H), 6.81 (s, 1H), 6.55 (t, J=6.1 Hz, 1H), 6.49 (t, J=5.9 Hz, 1H), 5.00 (q, J=9.1 Hz, 2H), 4.26 (d, J=6.2 Hz, 2H), 4.23 (d, J=5.9 Hz, 2H), 2.28-2.27 (m, 3H).

LCMS (Table 1, Method A) Rₜ=4.64 min MS, m/z: 354 [M+H]⁺.

### Compound 38

### 1-((6-(2,2,2-Trifluoroethoxy)pyrimidin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea from the appropriate starting materials, (6-(2,2,2-trifluoroethoxy)pyrimidin-4-yl)methanamine and 2-(1-(trifluoromethyl)cyclopropyl)ethan-1-amine hydrochloride (CAS: 1454690-80-2). (6-(2,2,2-Trifluoroethoxy)pyrimidin-4-yl)methanamine was in turn prepared following an analogous reaction protocol to that described for Compound 11: 1-((6-(Benzyloxy)pyrimidin-4-yl)methyl)-3-(2-(1-(trifluoromethyl)cyclopropyl)ethyl)urea, step (i-ii), from the appropriate commercial starting materials. The title compound was purified by flash column chromatography (DCM to DCM:MeOH [2M NH₃]90:10, gradient elution) to afford a white solid (37 mg, 40%).

¹H NMR (400 MHz, DMSO-d₆) δ 8.76 (d, J=1.1 Hz, 1H), 6.84 (d, J=1.0 Hz, 1H), 6.58 (t, J=5.9 Hz, 1H), 6.23 (t, J=5.6 Hz, 1H), 5.09 (q, J=9.1 Hz, 2H), 4.24 (d, J=6.3 Hz, 2H), 3.15-3.09 (m, 2H), 1.67 (t, J=7.8 Hz, 2H), 0.91-0.87 (m, 2H), 0.77-0.71 (m, 2H).

LCMS (Table 1, Method B) Rₜ=4.16 min MS, m/z: 387 [M+H]⁺.

### Compound 39

### 1-((2-Methyl-6-(2,2,2-trifluoroethoxy)pyridin-4-yl)methyl)-3-(2-(1-(trifluoromethyl) cyclopropyl)ethyl)urea

### (i) 1-((2-Chloro-6-methylpyridin-4-yl)methyl)-3-(2-(1-(trifluoromethyl)cyclopropyl) ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-chloro-6-methyl-4-pyridyl)methanamine dihydrochloride (CAS: 1909336-63-5) and 2-(1-(trifluoromethyl)cyclopropyl)ethan-1-amine hydrochloride (CAS: 1454690-80-2). The compound was purified by flash column chromatography (DCM to EtOAc, gradient elution) to afford the title compound (103 mg, 70%).

LC/MS (Table 1, Method A) Rₜ=1.46 min; MS m/z: 336 [M+H]⁺.

### (ii) 1-((2-Methyl-6-(2,2,2-trifluoroethoxy)pyridin-4-yl)methyl)-3-(2-(1-(trifluoromethyl) cyclopropyl)ethyl)urea

The title compound was prepared using an analogous reaction protocol to that described for Compound 21: 1-(3-(2,2,2-Trifluoroethoxy)-5,6,7,8-tetrahydroisoquinolin-5-yl)-3-(2-(1-(trifluoromethyl)cyclopropyl)ethyl)urea, step (iv), from the appropriate commercial starting materials 1-((2-chloro-6-methylpyridin-4-yl)methyl)-3-(2-(1-(trifluoromethyl)cyclopropyl) ethyl)urea (Compound 39, step (i)) and 2,2,2-trifluoroethanol (CAS: 75-89-8). The title compound was purified by flash column chromatography (DCM to EtOAc, gradient elution) to afford an off-white solid (84 mg, 67%).

¹H NMR (400 MHz, DMSO-d₆) δ 6.80 (s, 1H), 6.56 (s, 1H), 6.52 (t, J=6.1 Hz, 1H), 6.10 (t, J=5.8 Hz, 1H), 4.95 (q, J=9.2 Hz, 2H), 4.15 (d, J=6.1 Hz, 2H), 3.14-3.08 (m, 2H), 2.37 (s, 3H), 1.70-1.64 (m, 2H), 0.91-0.86 (m, 2H), 0.75-0.73 (m, 2H).

LC/MS (Table 1, Method F) Rₜ=4.79 min; MS m/z: 400 [M+H]⁺.

### Compound 40

### 1-(3-Fluorobenzyl)-3-((2-((2-fluorobenzyl)oxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-((2-fluorobenzyl)oxy)pyridin-4-yl)methanamine and 3-fluorobenzylamine (CAS: 100-82-3). (2-((2-Fluorobenzyl)oxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl)cyclopropyl)ethyl)urea, steps (i-ii), from the appropriate commercial starting materials. The title compound was purified by flash column chromatography (DCM to EtOAc, gradient elution) to afford a white solid (15 mg, 37%).

¹H NMR (400 MHz, DMSO-d₆) δ 8.08 (d, J=5.6 Hz, 1H), 7.52 (dt, J=1.8, 7.5 Hz, 1H), 7.44-7.38 (m, 1H), 7.36-7.30 (m, 1H), 7.27-7.19 (m, 2H), 7.10-7.00 (m, 3H), 6.88 (dd, J=1.3, 5.3 Hz, 1H), 6.70-6.63 (m, 3H), 5.37 (s, 2H), 4.25-4.20 (m, 4H).

LCMS (Table 1, Method F) Rₜ=4.46 min MS, m/z: 384 [M+H]⁺.

### Compound 41

### 1-(4,4,4-Trifluoro-3,3-dimethylbutyl)-3-((2-(2,2,2-trifluoroethoxy)pyridin-4-yl) methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 56129-93-6) and 4,4,4-trifluoro-3,3-dimethylbutan-1-amine hydrochloride (CAS: 1454690-74-4). The title compound was purified by reverse phase HPLC (Table 2, Method 2) to afford an off-white solid (89 mg, 69%).

¹H NMR (400 MHz, DMSO-d₆) δ 8.12 (d, J=5.3 Hz, 1H), 6.98 (dd, J=1.4, 5.3 Hz, 1H), 6.78 (s, 1H), 6.53 (t, J=6.3 Hz, 1H), 6.13 (t, J=5.8 Hz, 1H), 4.99 (q, J=9.1 Hz, 2H), 4.22 (d, J=6.1 Hz, 2H), 3.14-3.07 (m, 2H), 1.62-1.57 (m, 2H), 1.11 (s, 6H).

LCMS (Table 1, Method A) Rₜ=4.83 min MS, m/z: 388 [M+H]⁺.

### Compound 42

### 1-(3-Fluorobenzyl)-3-((2-(2,2,2-trifluoroethoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 56129-93-6) and 3-fluorobenzylamine (CAS: 100-82-3). The title compound was triturated with diethyl ether to afford an off-white solid (42 mg, 37%).

¹H NMR (400 MHz, DMSO-d₆) δ 8.10 (d, J=5.2 Hz, 1H), 7.38-7.32 (m, 1H), 7.11-7.02 (m, 3H), 6.98 (dd, J=1.4, 4.9 Hz, 1H), 6.78 (s, 1H), 6.71 (td, J=6.1, 15.3 Hz, 2H), 4.98 (q, J=9.1 Hz, 2H), 4.24 (d, J=6.2 Hz, 4H).

LCMS (Table 1, Method B) Rₜ=4.28 min MS, m/z: 358 [M+H]⁺.

### Compound 43

### 1-Cyclohexyl-3-((2-(2,2,2-trifluoroethoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 56129-93-6) and cyclohexylamine (CAS: 108-91-8). The title compound was triturated with diethyl ether to afford an off-white solid (75 mg, 71%).

¹H NMR (400 MHz, DMSO-d₆) δ 8.10 (d, J=5.2 Hz, 1H), 6.96 (dd, J=1.1, 5.2 Hz, 1H), 6.76 (s, 1H), 6.32 (t, J=6.0 Hz, 1H), 5.99 (d, J=8.0 Hz, 1H), 4.98 (q, J=9.2 Hz, 2H), 4.20 (d, J=6.1 Hz, 2H), 1.77-1.73 (m, 2H), 1.67-1.62 (m, 2H), 1.52 (dd, J=3.8, 8.4 Hz, 1H), 1.31-1.05 (m, 6H).

LCMS (Table 1, Method F) Rₜ=4.38 min MS, m/z: 332 [M+H]⁺.

### Compound 44

### 1-((2-(3-(Trifluoromethyl)azetidin-1-yl)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-(3-(trifluoromethyl)azetidin-1-yl)pyridin-4-yl)methanamine and 2-(1-(trifluoromethyl)cyclopropyl)ethan-1-amine hydrochloride (CAS: 1454690-80-2). (2-(3-(Trifluoromethyl)azetidin-1-yl)pyridin-4-yl)methanamine was in turn prepared following an analogous reaction protocol to that described for Compound 28: (±)-1-((2-(3-Fluoropiperidin-1-yl)pyridin-4-yl)methyl)-3-(2-(1-(trifluoromethyl)cyclo propyl)ethyl)urea, steps (i-ii), from the appropriate commercial starting materials. The title compound was purified by reverse phase HPLC (Table 2, Method 6) to afford a white solid (118 mg, 60%).

¹H NMR (400 MHz, DMSO-d₆) δ 8.02 (d, J=5.1 Hz, 1H), 6.60-6.57 (m, 1H), 6.46 (dd, J=6.1, 6.1 Hz, 1H), 6.30 (s, 1H), 6.05 (dd, J=5.8, 5.8 Hz, 1H), 4.18-4.12 (m, 4H), 3.91 (dd, J=5.3, 8.7 Hz, 2H), 3.78-3.68 (m, 1H), 3.12 (dd, J=6.2, 15.0 Hz, 2H), 1.71-1.66 (m, 2H), 0.92-0.88 (m, 2H), 0.76 (s, 2H).

LCMS (Table 1, Method A) Rₜ=3.18 min MS, m/z: 411 [M+H]⁺.

### Compound 45

### 1-((2-(3-Fluoroazetidin-1-yl)pyridin-4-yl)methyl)-3-(2-(1-(trifluoromethyl) cyclopropyl)ethyl)urea

### (i) 2-(3-Fluoroazetidin-1-yl)isonicotinonitrile

The title compound was prepared using an analogous reaction protocol to that described for Compound 28: (±)-1-((2-(3-Fluoropiperidin-1-yl)pyridin-4-yl)methyl)-3-(2-(1-(trifluoromethyl)cyclopropyl)ethyl)urea, step (i), from the appropriate commercial starting materials, 4-cyano-2-fluoropyridine (CAS: 3939-14-8) and 3-fluoroazetidine hydrochloride (CAS: 690257-76-2). The compound was purified by flash column chromatography (cyclohexane to EtOAc, gradient elution) to afford the title compound (387 mg, 68%).

LC/MS (Table 1, Method C) Rₜ=1.16 min; MS m/z: 178 [M+H]⁺.

### (ii) (2-(3-Fluoroazetidin-1-yl)pyridin-4-yl)methanamine

The title compound was prepared using an analogous reaction protocol to that described for Compound 11: 1-((6-(Benzyloxy)pyrimidin-4-yl)methyl)-3-(2-(1-(trifluoromethyl)cyclopropyl)ethyl)urea, step (ii) from the appropriate starting material 2-(3-fluoroazetidin-1-yl)isonicotinonitrile (Compound 45, step (i)). The compound was purified by flash column chromatography (cyclohexane to EtOAc:IMS 3:1, gradient elution) to afford the title compound (94 mg, 46%).

LC/MS (Table 1, Method G) Rₜ=1.01 min; MS m/z: 182 [M+H]⁺.

### (iii) 1-((2-(3-Fluoroazetidin-1-yl)pyridin-4-yl)methyl)-3-(2-(1-(trifluoromethyl)cyclopropyl) ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-(3-fluoroazetidin-1-yl)pyridin-4-yl)methanamine (Compound 45, step (ii)) and 2-(1-(trifluoromethyl)cyclopropyl)ethan-1-amine hydrochloride (CAS: 1454690-80-2). The title compound was purified by reverse phase HPLC (Table 2, Method 6) to afford an off-white solid (84 mg, 45%).

¹H NMR (400 MHz, DMSO-d₆) δ 8.01 (d, J=5.5 Hz, 1H), 6.57 (d, J=5.3 Hz, 1H), 6.45 (t, J=6.1 Hz, 1H), 6.29 (s, 1H), 6.04 (t, J=5.8 Hz, 1H), 5.60-5.40 (m, 1H), 4.30-4.19 (m, 2H), 4.13 (d, J=6.1 Hz, 2H), 4.01-3.91 (m, 2H), 3.12 (dd, J=6.2, 15.1 Hz, 2H), 1.71-1.66 (m, 2H), 0.93-0.88 (m, 2H), 0.75 (dd, J=6.8, 6.8 Hz, 2H).

LC/MS (Table 1, Method A) Rₜ=2.84 min; MS m/z: 361 [M+H]⁺.

### Compound 46

### 1-(o-Tolyl)-3-((2-(2,2,2-trifluoroethoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and o-toluidine (CAS: 95-53-4). The title compound was purified by flash column chromatography (DCM to EtOAc, gradient elution) to afford an off-white solid (51 mg, 45%).

¹H NMR (400 MHz, DMSO-d₆) δ 8.14 (d, J=5.2 Hz, 1H), 7.86 (s, 1H), 7.78 (d, J=7.6 Hz, 1H), 7.15-7.03 (m, 4H), 6.92-6.85 (m, 2H), 4.99 (q, J=9.1 Hz, 2H), 4.33 (d, J=6.0 Hz, 2H), 2.20 (s, 3H).

LC/MS (Table 1, Method F) Rₜ=4.39 min; MS m/z: 340 [M+H]⁺.

### Compound 47

### (S)-1-(2-(1-(Trifluoromethyl)cyclopropyl)ethyl)-3-((2-((1,1,1-trifluoropropan-2-yl)oxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (S)-(2-((1,1,1-trifluoropropan-2-yl)oxy)pyridin-4-yl)methanamine and 2-(1-(trifluoromethyl)cyclopropyl)ethan-1-amine hydrochloride (CAS: 1454690-80-2). (S)-(2-((1,1,1-Trifluoropropan-2-yl)oxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl)cyclopropyl)ethyl)urea, steps (i-ii), from the appropriate commercial starting materials. The title compound was purified by reverse phase HPLC (Table 2, Method 3, non-linear gradient from 40% to 100% MeCN) to afford an off-white solid (108 mg, 45%).

¹H NMR (400 MHz, DMSO-d₆) δ 8.11 (d, J=4.9 Hz, 1H), 6.96 (d, J=5.3 Hz, 1H), 6.73 (s, 1H), 6.54 (dd, J=6.1, 6.1 Hz, 1H), 6.11 (dd, J=5.8, 5.8 Hz, 1H), 5.93-5.85 (m, 1H), 4.21 (d, J=6.1 Hz, 2H), 3.13 (dd, J=6.1, 15.1 Hz, 2H), 1.72-1.66 (m, 2H), 1.44 (d, J=6.4 Hz, 3H), 0.92-0.88 (m, 2H), 0.75 (dd, J=6.7, 6.7 Hz, 2H).

LC/MS (Table 1, Method E) Rₜ=4.80 min; MS m/z: 400 [M+H]⁺.

### Compound 48

### 1-(2-((1,1,1-Trifluoro-2-methylpropan-2-yl)oxy)ethyl)-3-((2-(2,2,2-trifluoroethoxy) pyridin-4-yl)methyl)urea

### (i) tert-Butyl (2-((1,1,1-trifluoro-2-methylpropan-2-yl)oxy)ethyl)carbamate

To a solution of 3-boc-1,2,3-oxathiazolidine 2,2-dioxide (400 mg, 1.79 mmol) and 2-trifluoromethyl-2-propanol (0.27 mL, 2.51 mmol) in DMF (3.0 mL) at 0° C. under a nitrogen atmosphere was added sodium hydride (60%, 93 mg, 2.33 mmol). The reaction was allowed to warm to RT and next heated to 60° C. for 18 h. The reaction mixture was next partitioned between EtOAc and distilled water. The organic layer was separated. The combined organic layer was washed with saturated brine, dried (MgSO₄) and concentrated in vacuo to afford the title compound (327 mg, 86%).

¹H NMR (400 MHz, CDCl₃) δ 4.87-4.87 (m, 1H), 3.57 (t, J=5.0 Hz, 2H), 3.33-3.25 (m, 2H), 1.45 (s, 9H), 1.35 (s, 6H).

### (ii) 2-((1,1,1-Trifluoro-2-methylpropan-2-yl)oxy)ethan-1-amine hydrochloride

The title compound was prepared using an analogous reaction protocol to that described for Compound 6: 1-Methyl-3-((2-(2,2,2-trifluoroethoxy)pyridin-4-yl)methyl)-1-(2-(1-(trifluoromethyl)cyclopropyl)ethyl)urea, step (iii), from the appropriate starting tert-butyl (2-((1,1,1-trifluoro-2-methylpropan-2-yl)oxy)ethyl)carbamate ((Compound 48, step (i)). The compound was triturated with diethyl ether to afford the title compound (216 mg, 86%).

¹H NMR (400 MHz, DMSO-d₆) δ 8.08 (s, 2H), 3.70 (t, J=5.3 Hz, 2H), 2.95 (t, J=5.4 Hz, 2H), 1.36 (s, 6H).

### (iii) 1-(2-((1,1,1-Trifluoro-2-methylpropan-2-yl)oxy)ethyl)-3-((2-(2,2,2-trifluoroethoxy) pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and 2-((1,1,1-trifluoro-2-methylpropan-2-yl)oxy)ethan-1-amine hydrochloride (Compound 48, step (ii)). The title compound was purified by flash column chromatography (DCM to EtOAc, gradient elution) to afford an off-white solid (91 mg, 72%).

¹H NMR (400 MHz, DMSO-d₆) δ 8.09 (d, J=5.2 Hz, 1H), 6.96 (dd, J=0.9, 5.3 Hz, 1H), 6.76 (s, 1H), 6.63 (t, J=6.1 Hz, 1H), 6.11 (t, J=5.8 Hz, 1H), 4.97 (q, J=9.1 Hz, 2H), 4.22 (d, J=6.1 Hz, 2H), 3.47 (t, J=5.5 Hz, 2H), 3.18-3.12 (m, 2H), 1.31 (s, 6H).

LCMS (Table 1, Method F) Rₜ=4.47 min MS, m/z: 404 [M+H]⁺.

### Compound 49

### 1-((2-(2,2,2-Trifluoroethoxy)pyrimidin-4-yl)methyl)-3-(2-(1-(trifluoromethyl) cyclopropyl)ethyl)urea

### (i) (2-Chloropyrimidin-4-yl)methyl 4-methylbenzenesulfonate

To a solution of (2-chloropyrimidin-4-yl)methanol (CAS: 34953-87-2, 397 mg, 2.75 mmol), p-toluenesulfonyl chloride (628 mg, 3.30 mmol) in THF (15.0 mL) at 0° C. was added triethylamine (0.51 mL, 3.65 mmol). The reaction was stirred at 0° C. for 3 h. The reaction was allowed to warm to RT and stirred at RT for 16 h. The reaction mixture was next partitioned between EtOAc and distilled water. The organic layer was separated. The combined organic layer was washed with saturated brine, dried (Na₂SO₄) and concentrated in vacuo. The residue was purified by flash column chromatography (cyclohexane to EtOAc, gradient elution) to afford the title compound (570 mg, 69%). The product was advanced into step (ii) without characterisation.

### (ii) tert-Butyl (tert-butoxycarbonyl)((2-chloropyrimidin-4-yl)methyl)carbamate

The title compound was prepared using an analogous reaction protocol to that described for Compound 8: 1-((4-(Benzyloxy)pyrimidin-2-yl)methyl)-3-(2-(1-(trifluoromethyl) cyclopropyl)ethyl)urea, step (i), from the appropriate starting material, (2-chloropyrimidin-4-yl)methyl 4-methylbenzenesulfonate (Compound 49, step (i)). The compound was purified by flash column chromatography (cyclohexane to EtOAc, gradient elution) to afford the title compound (638 mg, quantitative).

LC/MS (Table 1, Method C) Rₜ=1.88 min; MS m/z: 366 [M+H]⁺.

### (iii) tert-Butyl ((2-(2,2,2-trifluoroethoxy)pyrimidin-4-yl)methyl)carbamate

The title compound was prepared using an analogous reaction protocol to that described for Compound 21: 1-(3-(2,2,2-Trifluoroethoxy)-5,6,7,8-tetrahydroisoquinolin-5-yl)-3-(2-(1-(trifluoromethyl) cyclopropyl)ethyl)urea, step (iv), from the appropriate starting materials, tert-butyl (tert-butoxycarbonyl)((2-chloropyrimidin-4-yl)methyl)carbamate (Compound 49, step (ii)) and 2,2,2-trifluoroethanol (CAS: 75-89-8). The compound was purified by flash column chromatography (cyclohexane to EtOAc, gradient elution) to afford the title compound (47 mg, 8%).

LC/MS (Table 1, Method C) Rₜ=1.61 min; MS m/z: 308 [M+H]⁺.

### (iv) (2-(2,2,2-Trifluoroethoxy)pyrimidin-4-yl)methanamine hydrochloride

The title compound was prepared using an analogous reaction protocol to that described for Compound 6: 1-Methyl-3-((2-(2,2,2-trifluoroethoxy)pyridin-4-yl)methyl)-1-(2-(1-(trifluoromethyl)cyclopropyl)ethyl)urea, step (iii), from the appropriate starting material, tert-butyl ((2-(2,2,2-trifluoroethoxy)pyrimidin-4-yl)methyl)carbamate (Compound 49, step (iii)). This afforded the title compound (38 mg, quantitative).

LC/MS (Table 1, Method A) Rₜ=0.92 min; MS m/z: 208 [M+H]⁺.

### (v) 1-((2-(2,2,2-Trifluoroethoxy)pyrimidin-4-yl)methyl)-3-(2-(1-(trifluoromethyl) cyclopropyl) ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyrimidin-4-yl)methanamine hydrochloride (Compound 49, step (iv)) and 2-(1-(trifluoromethyl)cyclopropyl)ethan-1-amine hydrochloride (CAS: 1454690-80-2). The title compound was purified by flash column chromatography (DCM to EtOAc, gradient elution) to afford an off-white solid (33 mg, 54%).

¹H NMR (400 MHz, DMSO-d₆) δ 8.58 (d, J=5.1 Hz, 1H), 7.09 (d, J=5.1 Hz, 1H), 6.60 (dd, J=6.0, 6.0 Hz, 1H), 6.26 (dd, J=5.8, 5.8 Hz, 1H), 5.03 (q, J=9.0 Hz, 2H), 4.24 (d, J=5.9 Hz, 2H), 3.11 (dd, J=6.1, 15.2 Hz, 2H), 1.70-1.65 (m, 2H), 0.91-0.86 (m, 2H), 0.74 (s, 2H).

LCMS (Table 1, Method F) Rₜ=4.12 min MS, m/z: 387 [M+H]⁺.

### Compound 50

### 1-((6-Methyl-2-(2,2,2-trifluoroethoxy)pyrimidin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (6-methyl-2-(2,2,2-trifluoroethoxy)pyrimidin-4-yl)methanamine and 2-(1-(trifluoromethyl)cyclopropyl)ethan-1-amine hydrochloride (CAS: 1454690-80-2). (6-Methyl-2-(2,2,2-trifluoroethoxy)pyrimidin-4-yl)methanamine was in turn prepared following an analogous reaction protocol to that described for Compound 49: 1-((2-(2,2,2-Trifluoroethoxy)pyrimidin-4-yl)methyl)-3-(2-(1-(trifluoromethyl)cyclopropyl)ethyl)urea, steps (i-iv), from the appropriate commercial starting materials. The title compound was purified by flash column chromatography (DCM to EtOAc, gradient elution) to afford an off-white solid (75 mg, 68%).

¹H NMR (400 MHz, DMSO-d₆) δ 6.96 (s, 1H), 6.55 (dd, J=6.0, 6.0 Hz, 1H), 6.23 (dd, J=5.8, 5.8 Hz, 1H), 5.01 (q, J=9.0 Hz, 2H), 4.20 (d, J=5.9 Hz, 2H), 3.11 (dd, J=6.1, 15.1 Hz, 2H), 2.41 (s, 3H), 1.70-1.65 (m, 2H), 0.91-0.86 (m, 2H), 0.74 (dd, J=6.7, 6.7 Hz, 2H).

LCMS (Table 1, Method F) Rₜ=4.31 min MS, m/z: 401 [M+H]⁺.

### Compound 51

### (R)-1-(2-(1-(Trifluoromethyl)cyclopropyl)ethyl)-3-((2-((1,1,1-trifluoropropan-2-yl)oxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (R)-(2-((1,1,1-trifluoropropan-2-yl)oxy)pyridin-4-yl)methanamine and 2-(1-(trifluoromethyl)cyclopropyl)ethan-1-amine hydrochloride (CAS: 1454690-80-2). (R)-(2-((1,1,1-Trifluoropropan-2-yl)oxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl)cyclopropyl)ethyl)urea, steps (i-ii), from the appropriate commercial starting materials. The title compound was purified by reverse phase HPLC (Table 2, Method 2) to afford an off-white solid (67 mg, 55%).

¹H NMR (400 MHz, DMSO-d₆) δ 8.11 (d, J=5.5 Hz, 1H), 6.96 (d, J=5.3 Hz, 1H), 6.72 (s, 1H), 6.54 (dd, J=6.1, 6.1 Hz, 1H), 6.11 (dd, J=5.8, 5.8 Hz, 1H), 5.93-5.85 (m, 1H), 4.21 (d, J=6.1 Hz, 2H), 3.13 (dd, J=6.1, 15.1 Hz, 2H), 1.72-1.66 (m, 2H), 1.44 (d, J=6.5 Hz, 3H), 0.92-0.88 (m, 2H), 0.78-0.74 (m, 2H).

LC/MS (Table 1, Method A) Rₜ=4.99 min; MS m/z: 400 [M+H]⁺.

### Compound 52

### 1-((5-Methyl-2-(2,2,2-trifluoroethoxy)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (5-methyl-2-(2,2,2-trifluoroethoxy)pyridin-4-yl)methanamine and 2-(1-(trifluoromethyl)cyclopropyl)ethan-1-amine hydrochloride (CAS: 1454690-80-2). (5-Methyl-2-(2,2,2-trifluoroethoxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl) cyclopropyl)ethyl)urea, steps (i-i), from the appropriate commercial starting materials. The title compound was purified by flash column chromatography (DCM to EtOAc, gradient elution) to afford an off-white solid (88 mg, 67%).

¹H NMR (400 MHz, DMSO-d₆) δ 7.91 (s, 1H), 6.68 (s, 1H), 6.49 (dd, J=6.0, 6.0 Hz, 1H), 6.12 (dd, J=5.8, 5.8 Hz, 1H), 4.94 (q, J=9.1 Hz, 2H), 4.16 (d, J=5.9 Hz, 2H), 3.13 (dd, J=6.2, 15.0 Hz, 2H), 2.16 (s, 3H), 1.71-1.66 (m, 2H), 0.92-0.87 (m, 2H), 0.75 (dd, J=6.7, 6.7 Hz, 2H).

LCMS (Table 1, Method F) Rₜ=4.75 min MS, m/z: 400 [M+H]⁺.

### Compound 53

### 1-((6-(2,2,2-Trifluoroethoxy)pyridazin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (6-(2,2,2-trifluoroethoxy)pyridazin-4-yl)methanamine and 2-(1-(trifluoromethyl)cyclopropyl)ethan-1-amine hydrochloride (CAS: 1454690-80-2). (6-(2,2,2-trifluoroethoxy)pyridazin-4-yl)methanamine was in turn prepared following an analogous reaction protocol to that described for Compound 11: 1-((6-(Benzyloxy)pyrimidin-4-yl)methyl)-3-(2-(1-(trifluoromethyl)cyclopropyl)ethyl)urea, steps (i-ii), from the appropriate commercial starting materials. The title compound was purified by flash column chromatography (DCM to EtOAc, gradient elution) to afford an off-white solid (59 mg, 52%).

¹H NMR (400 MHz, DMSO-d₆) δ 8.89 (d, J=1.7 Hz, 1H), 7.10 (d, J=1.0 Hz, 1H), 6.61 (dd, J=6.0, 6.0 Hz, 1H), 6.21 (dd, J=5.7, 5.7 Hz, 1H), 5.18 (q, J=9.0 Hz, 2H), 4.25 (d, J=5.9 Hz, 2H), 3.15-3.07 (m, 2H), 1.70-1.65 (m, 2H), 0.91-0.86 (m, 2H), 0.74 (s, 2H).

LCMS (Table 1, Method F) Rₜ=4.11 min MS, m/z: 387 [M+H]⁺.

### Compound 54

### 1-((2-((3-Fluoropyridin-2-yl)methoxy)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-((3-fluoropyridin-2-yl)methoxy)pyridin-4-yl)methanamine and 2-(1-(trifluoromethyl)cyclopropyl)ethan-1-amine hydrochloride (CAS: 1454690-80-2). (2-((3-Fluoropyridin-2-yl)methoxy)pyridin-4-yl)methanamine was in turn prepared following an analogous reaction protocol to that described for Compound 10: 1-((2-((1-Methylpiperidin-4-yl)methoxy)pyridin-4-yl)methyl)-3-(2-(1-(trifluoromethyl) cyclopropyl)ethyl)urea, steps (i-ii), from the appropriates starting materials. The title compound was purified by flash column chromatography (DCM to EtOAc, gradient elution) to afford an off-white solid (65 mg, 43%).

¹H NMR (400 MHz, DMSO-d₆) δ 8.42-8.39 (m, 1H), 8.04 (d, J=5.3 Hz, 1H), 7.78-7.73 (m, 1H), 7.51-7.46 (m, 1H), 6.86 (dd, J=1.3, 5.3 Hz, 1H), 6.66 (s, 1H), 6.50 (t, J=6.2 Hz, 1H), 6.06 (t, J=5.8 Hz, 1H), 5.46-5.45 (m, 2H), 4.17 (d, J=6.1 Hz, 2H), 3.14-3.07 (m, 2H), 1.69-1.64 (m, 2H), 0.90-0.85 (m, 2H), 0.75-0.71 (m, 2H).

LCMS (Table 1, Method F) Rₜ=4.05 min MS, m/z: 413 [M+H]⁺.

### Compound 55

### 1-((2-(Pyrimidin-2-ylmethoxy)pyridin-4-yl)methyl)-3-(2-(1-(trifluoromethyl) cyclopropyl)ethyl)urea

### (i) 2-(Pyrimidin-2-ylmethoxy)isonicotinonitrile

The title compound was prepared using an analogous reaction protocol to that described for Compound 10: 1-((2-((1-Methylpiperidin-4-yl)methoxy)pyridin-4-yl)methyl)-3-(2-(1-(trifluoromethyl)cyclopropyl)ethyl)urea, step (i), from the appropriate commercial starting materials, 4-cyano-2-fluoropyridine (CAS: 3939-14-8) and pyrimidin-2-ylmethanol (CAS: 42839-09-8). The compound was triturated with diethyl ether to afford the title compound (889 mg, 79%).

LCMS (Table 1, Method D) Rₜ=1.03 min MS, m/z: 213 [M+H]⁺.

### (ii) (2-(Pyrimidin-2-ylmethoxy)pyridin-4-yl)methanamine

Sodium borohydride (107 mg, 2.83 mmol) was added portionwise to a solution of 2-(pyrimidin-2-ylmethoxy)isonicotinonitrile (Compound 55, step (i), 200 mg, 0.942 mmol), nickel(II) chloride hexahydrate (269 mg, 1.13 mmol) in EtOH (10.0 mL) at 0° C. under a nitrogen atmosphere. The reaction was allowed to warm to RT and stirred at RT for 18 h. The reaction mixture was next partitioned between DCM and distilled water. The organic layer was separated. The combined organic layer was dried (Na₂SO₄) and concentrated in vacuo. The residue was purified by flash column chromatography (DCM to DCM:MeOH [2M NH₃]90:10, gradient elution) to afford the title compound (71 mg, 35%).

LCMS (Table 1, Method D) Rₜ=0.76 min MS, m/z: 217 [M+H]⁺.

### (iii) 1-((2-(Pyrimidin-2-ylmethoxy)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-(pyrimidin-2-ylmethoxy)pyridin-4-yl)methanamine (Compound 55, step (ii)) and 2-(1-(trifluoromethyl)cyclopropyl)ethan-1-amine hydrochloride (CAS: 1454690-80-2). The title compound was purified by flash column chromatography (EtOAc to MeOH, gradient elution) to afford an off-white solid (52 mg, 44%).

¹H NMR (400 MHz, DMSO-d₆) δ 8.76 (s, 1H), 8.75 (s, 1H), 7.93 (d, J=5.2 Hz, 1H), 7.40 (t, J=4.9 Hz, 1H), 6.81 (dd, J=1.3, 5.3 Hz, 1H), 6.73 (s, 1H), 6.53 (t, J=6.1 Hz, 1H), 6.09 (t, J=5.8 Hz, 1H), 5.50 (s, 2H), 4.19 (d, J=6.1 Hz, 2H), 3.16-3.08 (m, 2H), 1.71-1.65 (m, 2H), 0.91-0.86 (m, 2H), 0.77-0.72 (m, 2H).

LCMS (Table 1, Method F) Rₜ=3.42 min MS, m/z: 396 [M+H]⁺.

### Compound 56

### 1-((2-(3,3-Difluoroazetidin-1-yl)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-(3,3-difluoroazetidin-1-yl)pyridin-4-yl)methanamine and 2-(1-(trifluoromethyl)cyclopropyl)ethan-1-amine hydrochloride (CAS: 1454690-80-2). (2-(3,3-Difluoroazetidin-1-yl)pyridin-4-yl)methanamine was in turn prepared following an analogous reaction protocol to that described for Compound 45: 1-((2-(3-Fluoroazetidin-1-yl)pyridin-4-yl)methyl)-3-(2-(1-(trifluoromethyl)cyclopropyl)ethyl)urea, steps (i-ii). The title compound was purified by reverse phase HPLC (Table 2, Method 6) to afford an off-white solid (55 mg, 53%).

¹H NMR (400 MHz, DMSO-d₆) δ 8.06 (d, J=5.5 Hz, 1H), 6.67 (d, J=5.3 Hz, 1H), 6.48 (dd, J=6.1, 6.1 Hz, 1H), 6.42 (s, 1H), 6.06 (dd, J=5.8, 5.8 Hz, 1H), 4.35 (dd, J=12.5, 12.5 Hz, 4H), 4.15 (d, J=6.1 Hz, 2H), 3.13 (dd, J=6.1, 15.1 Hz, 2H), 1.71-1.66 (m, 2H), 0.93-0.88 (m, 2H), 0.75 (dd, J=6.8, 6.8 Hz, 2H).

LCMS (Table 1, Method E) Rₜ=3.99 min MS, m/z: 379 [M+H]⁺.

### Compound 57

### 1-(2-Hydroxy-2-methylpropyl)-3-((2-(2,2,2-trifluoroethoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and 1-amino-2-methyl-2-propanol (CAS: 2854-16-2). The reaction mixture was filtered and washed with DCM to afford the title compound as a white solid (68 mg, 67%).

¹H NMR (400 MHz, DMSO-d₆) δ 8.10 (d, J=5.3 Hz, 1H), 6.97 (d, J=5.3 Hz, 1H), 6.78 (s, 1H), 6.60 (t, J=6.1 Hz, 1H), 6.06 (t, J=5.7 Hz, 1H), 4.97 (q, J=9.1 Hz, 2H), 4.48 (s, 1H), 4.23 (d, J=6.1 Hz, 2H), 2.97 (d, J=5.9 Hz, 2H), 1.05 (s, 6H).

LCMS (Table 1, Method F) Rₜ=3.28 min MS, m/z: 322 [M+H]⁺.

### Compound 58

### 1-((1S,2R)-2-Hydroxycyclohexyl)-3-((2-(2,2,2-trifluoroethoxy)pyridin-4-yl)methyl) urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and (1R,2S)-cis-2-aminocyclohexanol hydrochloride (CAS: 190792-72-4). The title compound was purified by flash column chromatography (cyclohexane to EtOAc:IMS 3:1, gradient elution) followed by flash column chromatography (DCM to MeOH, gradient elution) to afford a white solid (60 mg, 54%).

¹H NMR (400 MHz, DMSO-d₆) δ 8.10 (dd, J=0.5, 5.3 Hz, 1H), 6.96 (dd, J=1.2, 5.3 Hz, 1H), 6.78-6.76 (m, 1H), 6.61 (t, J=6.1 Hz, 1H), 5.89 (d, J=8.4 Hz, 1H), 4.98 (q, J=9.1 Hz, 2H), 4.64 (d, J=4.0 Hz, 1H), 4.21 (d, J=6.1 Hz, 2H), 3.71-3.65 (m, 1H), 3.50-3.43 (m, 1H), 1.68-1.39 (m, 6H), 1.30-1.18 (m, 2H).

LC/MS (Table 1, Method F) Rₜ=3.65 min; MS m/z: 348 [M+H]⁺.

### Compound 59

### (R)-2-Methyl-N-((2-(2,2,2-trifluoroethoxy)pyridin-4-yl)methyl)morpholine-4-carboxamide

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and (R)-2-methylmorpholine hydrochloride (CAS: 168038-14-0). The title compound was purified by flash column chromatography (cyclohexane to EtOAc, gradient elution) to afford an off-white solid (75 mg, 69%).

¹H NMR (400 MHz, DMSO-d₆) δ 8.10 (dd, J=0.5, 5.3 Hz, 1H), 7.17 (t, J=5.9 Hz, 1H), 6.98 (dd, J=1.3, 5.3 Hz, 1H), 6.79-6.77 (m, 1H), 4.97 (q, J=9.1 Hz, 2H), 4.24 (d, J=5.6 Hz, 2H), 3.85-3.72 (m, 3H), 3.46-3.35 (m, 2H), 2.83-2.74 (m, 1H), 2.47-2.42 (m, 1H), 1.08 (d, J=6.1 Hz, 3H).

LC/MS (Table 1, Method F) Rₜ=3.71 min; MS m/z: 334 [M+H]⁺.

### Compound 60

### (S)-2-Methyl-N-((2-(2,2,2-trifluoroethoxy)pyridin-4-yl)methyl)morpholine-4-carboxamide

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and (S)-2-methylmorpholine hydrochloride (CAS: 1147108-99-3). The title compound was purified by flash column chromatography (cyclohexane to EtOAc:IMS 3:1, gradient elution) to afford an off-white solid (79 mg, 73%).

¹H NMR (400 MHz, DMSO-d₆) δ 8.10 (dd, J=0.5, 5.3 Hz, 1H), 7.17 (t, J=5.8 Hz, 1H), 6.99 (dd, J=1.3, 5.3 Hz, 1H), 6.79-6.77 (m, 1H), 4.98 (q, J=9.1 Hz, 2H), 4.25 (d, J=5.6 Hz, 2H), 3.85-3.72 (m, 3H), 3.45-3.35 (m, 2H), 2.83-2.74 (m, 1H), 2.47-2.43 (m, 1H), 1.08 (d, J=6.2 Hz, 3H).

LC/MS (Table 1, Method F) Rₜ=3.70 min; MS m/z: 334 [M+H]⁺.

### Compound 61

### 2-(6-Oxa-3-azabicyclo[3.1.1]heptan-3-yl)-N-((2-(2,2,2-trifluoroethoxy)pyridin-4-yl)methyl)acetamide

### (i) tert-Butyl ((5-(2,2,2-trifluoroethoxy)pyridin-3-yl)methyl)carbamate

To a solution of 5-(2,2,2-trifluoroethoxy)pyridine-3-carbonitrile (CAS: 1211581-84-8, 355 mg, 1.76 mmol) in anhydrous MeOH (10.0 mL) at 0° C. under a nitrogen atmosphere was added di-tert-butyl dicarbonate (767 mg, 3.51 mmol) and nickel (II) chloride hexahydrate (41.7 mg, 0.176 mmol). The reaction was set to stir at 0° C. and sodium borohydride (465 mg, 12.3 mmol) was added portionwise. The reaction was allowed to warm to RT and the reaction was stirred at RT for 1 h. The reaction mixture was quenched by the addition of diethylenetriamine (0.19 mL, 1.76 mmol) and the reaction stirred for 30 min at RT. The reaction was next partitioned between DCM and saturated aqueous sodium hydrogen carbonate. The organic layer was separated. The combined organic layers were dried (MgSO₄) and concentrated in vacuo. The residue was purified by flash column chromatography (cyclohexane to EtOAc, gradient elution) to afford the title compound (513 mg, 83%).

LC/MS (Table 1, Method C) Rₜ=1.49 min; MS m/z: 307 [M+H]⁺.

### (ii) (5-(2,2,2-trifluoroethoxy)pyridin-3-yl)methanamine dihydrochloride

The title compound was prepared using an analogous reaction protocol to that described for Compound 6: 1-Methyl-3-((2-(2,2,2-trifluoroethoxy)pyridin-4-yl)methyl)-1-(2-(1-(trifluoromethyl)cyclopropyl)ethyl)urea, step (iii), from the appropriate starting material tert-butyl ((5-(2,2,2-trifluoroethoxy)pyridin-3-yl)methyl)carbamate (Compound 61, step (i)). This afforded the title compound (181 mg, quantitative).

LC/MS (Table 1, Method C) Rₜ=0.25 min; MS m/z: 207 [M+H]⁺.

### (iii) 2-(6-Oxa-3-azabicyclo[3.1.1]heptan-3-yl)-N-((2-(2,2,2-trifluoroethoxy)pyridin-4-yl)methyl)acetamide

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (5-(2,2,2-trifluoroethoxy)pyridin-3-yl)methanamine dihydrochloride (Compound 61, step (ii)) and 2-(1-(trifluoromethyl)cyclopropyl)ethan-1-amine hydrochloride (CAS: 1454690-80-2). The title compound was purified by flash column chromatography (DCM to DCM:MeOH [2M NH₃]90:10, gradient elution) followed by flash column chromatography (DCM to MeOH, gradient elution) to afford a white solid (128 mg, 50%).

¹H NMR (400 MHz, DMSO-d₆) δ 8.27 (d, J=2.9 Hz, 1H), 8.17 (d, J=1.3 Hz, 1H), 7.35 (dd, J=1.9, 2.6 Hz, 1H), 6.49 (t, J=6.0 Hz, 1H), 6.04 (t, J=5.8 Hz, 1H), 4.85 (q, J=8.8 Hz, 2H), 4.21 (d, J=6.1 Hz, 2H), 3.11 (q, J=7.1 Hz, 2H), 1.67 (t, J=7.8 Hz, 2H), 0.90-0.86 (m, 2H), 0.76-0.70 (m, 2H).

LCMS (Table 1, Method F) Rₜ=3.87 min MS, m/z: 386 [M+H]⁺.

### Compound 62

### 1-((1R,2S)-2-Hydroxycyclohexyl)-3-((2-(2,2,2-trifluoroethoxy)pyridin-4-yl)methyl) urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and (1 S,2R)-2-aminocyclohexan-1-ol hydrochloride (CAS: 200352-28-9). The title compound was purified by flash column chromatography (cyclohexane to EtOAc:IMS 3:1, gradient elution) followed by flash column chromatography (DCM to MeOH, gradient elution) to afford a white solid (33 mg, 28%).

¹H NMR (400 MHz, DMSO-d₆) δ 8.10 (dd, J=0.6, 5.3 Hz, 1H), 6.97 (dd, J=1.3, 5.3 Hz, 1H), 6.78-6.76 (m, 1H), 6.61 (t, J=6.1 Hz, 1H), 5.89 (d, J=8.4 Hz, 1H), 4.97 (q, J=9.1 Hz, 2H), 4.65 (d, J=4.0 Hz, 1H), 4.21 (d, J=6.0 Hz, 2H), 3.71-3.66 (m, 1H), 3.51-3.44 (m, 1H), 1.68-1.39 (m, 6H), 1.30-1.19 (m, 2H).

LC/MS (Table 1, Method F) Rₜ=3.64 min; MS m/z: 348 [M+H]⁺.

### Compound 63

### 1-((1S,2S)-2-Hydroxycyclohexyl)-3-((2-(2,2,2-trifluoroethoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and (1 S,2S)-2-aminocyclohexan-1-ol (CAS: 74111-21-0). The reaction mixture was filtered and washed with DCM to afford the title compound as a white solid (62 mg, 57%).

¹H NMR (400 MHz, DMSO-d₆) δ 8.10 (dd, J=0.4, 5.2 Hz, 1H), 6.98 (dd, J=1.2, 5.3 Hz, 1H), 6.80-6.78 (m, 1H), 6.47 (t, J=6.1 Hz, 1H), 5.99 (d, J=6.5 Hz, 1H), 4.98 (q, J=9.1 Hz, 2H), 4.73 (d, J=4.6 Hz, 1H), 4.22 (d, J=6.1 Hz, 2H), 3.25-3.13 (m, 2H), 1.91-1.78 (m, 2H), 1.63-1.53 (m, 2H), 1.25-1.05 (m, 4H).

LC/MS (Table 1, Method F) Rₜ=3.79 min; MS m/z: 348 [M+H]⁺.

### Compound 64

### 1-((1R,2R)-2-Hydroxycyclohexyl)-3-((2-(2,2,2-trifluoroethoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and (1R,2R)-2-aminocyclohexan-1-ol (CAS: 931-16-8). The reaction mixture was filtered and washed with DCM to afford the title compound as a white solid (64 mg, 59%).

¹H NMR (400 MHz, DMSO-d₆) δ 8.10 (d, J=5.3 Hz, 1H), 6.98 (dd, J=1.3, 5.3 Hz, 1H), 6.79 (s, 1H), 6.47 (t, J=6.1 Hz, 1H), 5.99 (d, J=6.5 Hz, 1H), 4.97 (q, J=9.1 Hz, 2H), 4.73 (d, J=4.5 Hz, 1H), 4.22 (d, J=6.1 Hz, 2H), 3.24-3.13 (m, 2H), 1.91-1.78 (m, 2H), 1.63-1.53 (m, 2H), 1.25-1.05 (m, 4H).

LC/MS (Table 1, Method F) Rₜ=3.79 min; MS m/z: 348 [M+H]⁺.

### Compound 65

### (R)-1-(1-(2-(2,2,2-Trifluoroethoxy)pyridin-4-yl)ethyl)-3-(2-(1-(trifluoromethyl) cyclopropyl)ethyl)urea

### (i) N-Methoxy-N-methyl-2-(2,2,2-trifluoroethoxy)isonicotinamide

A reaction vessel was charged with 2-(2,2,2-trifluoroethoxy)pyridine-4-carboxylic acid (CAS: 262296-01-5, 2.05 g, 9.08 mmol), HBTU (4.13 g, 10.9 mmol), N,O-dimethylhydroxylamine hydrochloride (1.06 g, 10.9 mmol) and solvated in DCM (50.0 mL). N,N-Diisopropylethylamine (4.0 mL, 22.7 mmol) was added and the reaction was stirred at RT for 18 h. The reaction was next partitioned between EtOAc and distilled water. The organic layer was separated. The combined organic layers were washed with saturated brine, dried (Na₂SO₄) and concentrated in vacuo. The residue was purified by flash column chromatography (cyclohexane to EtOAc:IMS 3:1, gradient elution) to afford the title compound (2.26 g, 94%).

LC/MS (Table 1, Method C) Rₜ=1.52 min; MS m/z: 265 [M+H]⁺.

### (ii) 1-(2-(2,2,2-Trifluoroethoxy)pyridin-4-yl)ethan-1-one

To a solution of N-Methoxy-N-methyl-2-(2,2,2-trifluoroethoxy)isonicotinamide ((Compound 65, step (i), 1.75 g, 6.62 mmol)) in THF (40.0 mL) at 0° C. was added 3M methyl magnesium bromide solution (4.4 mL, 13.2 mmol). The reaction was allowed to warm to RT and the reaction stirred at RT for 1 h. The reaction mixture was quenched when poured onto an aqueous saturated sodium hydrogen carbonate solution and next partitioned with EtOAc. The organic layer was separated. The combined organic layers were washed with saturated brine, dried (MgSO4) and concentrated in vacuo to afford the title compound (1.29 g, 97%).

LC/MS (Table 1, Method C) Rₜ=1.65 min; MS m/z: 220 [M+H]⁺.

### (iii) (R,E)-2-Methyl-N-(1-(2-(2,2,2-trifluoroethoxy)pyridin-4-yl)ethylidene)propane-2-sulfinamide

A reaction vessel was charged with 1-(2-(2,2,2-Trifluoroethoxy)pyridin-4-yl)ethan-1-one ((Compound 65, step (ii), 370 mg, 1.64 mmol)), (R)-2-methyl-2-propanesulfinamide (218 mg, 1.80 mmol) and solvated in THF (10.0 mL). Titanium(IV) ethoxide (0.69 mL, 3.28 mmol) was added and the reaction was set to stir at RT and next heated to 70° C. The reaction stirred at 70° C. for 24 h. The reaction was allowed to cool to RT. The reaction mixture was next partitioned between EtOAc and saturated brine. The organic layer was separated. The combined organic layer was dried (MgSO₄) and concentrated in vacuo to afford the title compound (635 mg, quantitative).

LC/MS (Table 1, Method C) Rₜ=1.80 min; MS m/z: 323 [M+H]⁺.

### (iv) (R)-2-Methyl-N—((R)-1-(2-(2,2,2-trifluoroethoxy)pyridin-4-yl)ethyl)propane-2-sulfinamide

To a solution of (R,E)-2-Methyl-N-(1-(2-(2,2,2-trifluoroethoxy)pyridin-4-yl)ethylidene)propane-2-sulfinamide ((Compound 65, step (iii), 635 mg, 1.89 mmol)) in THF (10.0 mL) at −78° C. was added 1M diisobutylaluminum hydride (4.3 mL, 4.26 mmol) via dropwise addition. The reaction was stirred at −78° C. for 18 h. MeOH (5 mL) was added and the reaction was allowed to warm to RT. The reaction mixture was next partitioned between EtOAc and 2M aqueous sodium hydroxide solution. The organic layer was separated. The combined organic layer was washed with saturated brine, dried (MgSO₄) and concentrated in vacuo. The residue was purified by flash column chromatography (DCM to MeOH, gradient elution) to afford the title compound (111 mg, 18%).

LC/MS (Table 1, Method F) Rₜ=4.41 min; MS m/z: 325 [M+H]⁺.

### (v) (R)-1-(2-(2,2,2-Trifluoroethoxy)pyridin-4-yl)ethan-1-amine hydrochloride

The title compound was prepared using an analogous reaction protocol to that described for Compound 6: 1-Methyl-3-((2-(2,2,2-trifluoroethoxy)pyridin-4-yl)methyl)-1-(2-(1-(trifluoromethyl)cyclopropyl)ethyl)urea, step (iii), from the appropriate starting material (R)-2-Methyl-N—((R)-1-(2-(2,2,2-trifluoroethoxy)pyridin-4-yl)ethyl)propane-2-sulfinamide (Compound 65, step (iv)). This afforded the title compound (108 mg, quantitative).

¹H NMR (400 MHz, DMSO) δ 8.60-8.60 (m, 2H), 8.26 (d, J=5.3 Hz, 1H), 7.25 (d, J=5.4 Hz, 1H), 7.12 (s, 1H), 5.02 (q, J=9.1 Hz, 2H), 4.47-4.41 (m, 1H), 1.49 (d, J=6.8 Hz, 3H).

### (vi) (R)-1-(1-(2-(2,2,2-Trifluoroethoxy)pyridin-4-yl)ethyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (R)-1-(2-(2,2,2-Trifluoroethoxy)pyridin-4-yl)ethan-1-amine hydrochloride (Compound 65, step (v)) and 2-(1-(trifluoromethyl)cyclopropyl)ethan-1-amine hydrochloride (CAS: 1454690-80-2). The title compound was purified by reverse phase HPLC (Table 2, Method 7) to afford a white solid (50 mg, 64%).

¹H NMR (400 MHz, DMSO-d₆) δ 8.10 (d, J=5.3 Hz, 1H), 7.01 (dd, J=1.2, 5.3 Hz, 1H), 6.82 (s, 1H), 6.54 (d, J=7.9 Hz, 1H), 5.91 (dd, J=5.8, 5.8 Hz, 1H), 4.96 (q, J=9.1 Hz, 2H), 4.74-4.64 (m, 1H), 3.11-3.04 (m, 2H), 1.64 (dd, J=6.4, 9.0 Hz, 2H), 1.28 (d, J=7.1 Hz, 3H), 0.89-0.85 (m, 2H), 0.71 (dd, J=6.4, 6.4 Hz, 2H).

LC/MS (Table 1, Method F) Rₜ=4.66 min; MS m/z: 399 [M+H]⁺.

### Compound 66

### (±)-1-(3-(2,2,2-Trifluoroethoxy)-6,7-dihydro-5H-cyclopenta[c]pyridin-5-yl)-3-(2-(1-(trifluoromethyl)cyclopropyl)ethyl)urea

### (i) S-((6-Chloropyridin-3-yl)methyl)O-ethyl carbonodithioate

A reaction vessel was charged with 2-chloro-5-(chloromethyl)pyridine (CAS: 70258-18-3, 5.00 g, 30.9 mmol) and solvated in acetone (25.0 mL). Potassium ethoxymethanedithioate (5.44 g, 33.9 mmol) was added portionwise and the reaction was stirred at RT for 18 h. The reaction mixture was next partitioned between diethyl ether and distilled water. The organic layer was separated. The combined organic layer was dried (Na₂SO₄) and concentrated in vacuo to afford the title compound (7.27 g, 95%).

LC/MS (Table 1, Method D) Rₜ=1.49 min; MS m/z: 248 [M+H]⁺.

### (ii) (±)-S-(3-(6-Chloropyridin-3-yl)-1-(1,3-dioxoisoindolin-2-yl)propyl)O-ethyl carbonodithioate

A reaction vessel was charged with S-((6-chloropyridin-3-yl)methyl)O-ethyl carbonodithioate ((Compound 66, step (i), 7.27 g, 29.3 mmol)), N-vinylphthalimide (1.27 g, 7.33 mmol) and solvated in EtOAc (30.0 mL). Lauroyl peroxide (73 mg, 0.183 mmol) was added and the reaction was set to stir at RT. The reaction was next heated to reflux. Additional lauroyl peroxide (73 mg, 0.183 mmol) was added on each hour whilst the reaction was stirred at reflux for 13 h. The reaction mixture was allowed to cool to RT and next concentrated in vacuo. The residue was purified directly by flash column chromatography (pentane to diethyl ether, gradient elution) to afford the title compound (924 mg, 30%).

LC/MS (Table 1, Method D) Rₜ=1.62 min; MS m/z: 421 [M+H]⁺.

### (iii) (±)-2-(3-Chloro-6,7-dihydro-5H-cyclopenta[c]pyridin-5-yl)isoindoline-1,3-dione

A reaction vessel was charged with (±)-S-(3-(6-chloropyridin-3-yl)-1-(1,3-dioxoisoindolin-2-yl)propyl)O-ethyl carbonodithioate ((Compound 66, step (ii), 906 mg, 2.15 mmol)), trifluoroacetic acid (0.19 mL, 2.44 mmol) and solvated in 1,2-dichloroethane (20.0 mL). Lauroyl peroxide (172 mg, 0.430 mmol) was added and the reaction was set to stir at RT. The reaction was next heated to reflux. Additional lauroyl peroxide (172 mg, 0.430 mmol) was added on each hour whilst the reaction was stirred at reflux for 5 h. The reaction mixture was allowed to cool to RT and concentrated in vacuo. The residue was purified directly by flash column chromatography (pentane to diethyl ether, gradient elution) to afford the title compound (119 mg, 19%).

LC/MS (Table 1, Method D) Rₜ=1.40 min; MS m/z: 299 [M+H]⁺.

### (iv) (±)-2-(3-(2,2,2-Trifluoroethoxy)-6,7-dihydro-5H-cyclopenta[c]pyridin-5-yl)isoindoline-1,3-dione

The title compound was prepared using an analogous reaction protocol to that described for Compound 21: 1-(3-(2,2,2-Trifluoroethoxy)-5,6,7,8-tetrahydroisoquinolin-5-yl)-3-(2-(1-(trifluoromethyl)cyclopropyl)ethyl)urea, step (iv), from the appropriate starting materials, (±)-2-(3-chloro-6,7-dihydro-5H-cyclopenta[c]pyridin-5-yl)isoindoline-1,3-dione ((Compound 66, step (iii)) and 2,2,2-trifluoroethanol (CAS: 75-89-8). The compound was purified by flash column chromatography (pentane to diethyl ether, gradient elution) to afford the title compound (74 mg, 41%).

LC/MS (Table 1, Method D) Rₜ=1.64 min; MS m/z: 362 [M+H]⁺.

### (v) (±)-3-(2,2,2-Trifluoroethoxy)-6,7-dihydro-5H-cyclopenta[c]pyridin-5-amine

A reaction vessel was charged with (±)-2-(3-(2,2,2-trifluoroethoxy)-6,7-dihydro-5H-cyclopenta[c]pyridin-5-yl)isoindoline-1,3-dione ((Compound 66, step (iv), 74 mg, 0.204 mmol)) and solvated in MeOH (2.0 mL). 1M hydrazine in THF (0.41 mL, 0.409 mmol) was added and the reaction was heated to 60° C. for 18 h. The reaction mixture was allowed to cool to RT and concentrated in vacuo. The residue was purified directly by flash column chromatography (DCM to DCM:MeOH [2M NH₃]90:10, gradient elution) to afford the title compound (32 mg, 67%).

¹H NMR (400 MHz, CDCl₃) δ 7.94 (s, 1H), 6.81 (s, 1H), 4.78-4.71 (m, 2H), 4.32-4.27 (m, 1H), 2.94-2.87 (m, 1H), 2.79-2.70 (m, 1H), 2.57-2.49 (m, 1H), 1.78-1.67 (m, 1H), 1.51-1.58 (s, 2H).

### (vi) (±)-1-(3-(2,2,2-Trifluoroethoxy)-6,7-dihydro-5H-cyclopenta[c]pyridin-5-yl)-3-(2-(1-(trifluoromethyl)cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (±)-3-(2,2,2-trifluoroethoxy)-6,7-dihydro-5H-cyclopenta[c]pyridin-5-amine (Compound 66, step (v)) and 2-(1-(trifluoromethyl)cyclopropyl)ethan-1-amine hydrochloride (CAS: 1454690-80-2). The title compound was purified by flash column chromatography (DCM to EtOAc, gradient elution to afford an off-white solid (25 mg, 42%).

¹H NMR (400 MHz, DMSO-d₆) δ 8.02 (s, 1H), 6.68 (s, 1H), 6.43 (d, J=8.1 Hz, 1H), 5.89 (t, J=5.8 Hz, 1H), 5.07-4.89 (m, 3H), 3.18-3.11 (m, 2H), 2.89-2.81 (m, 1H), 2.77-2.66 (m, 1H), 2.44-2.31 (m, 1H), 1.77-1.67 (m, 3H), 0.92-0.88 (m, 2H), 0.78-0.75 (m, 2H).

LC/MS (Table 1, Method B) Rₜ=4.76 min; MS m/z: 412 [M+H]⁺.

### Compound 67

### (S)-1-(1-(2-(2,2,2-Trifluoroethoxy)pyridin-4-yl)ethyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (S)-1-(2-(2,2,2-trifluoroethoxy)pyridin-4-yl)ethan-1-amine hydrochloride and 2-(1-(trifluoromethyl)cyclopropyl)ethan-1-amine hydrochloride (CAS: 1454690-80-2). (S)-1-(2-(2,2,2-trifluoroethoxy)pyridin-4-yl)ethan-1-amine hydrochloride was in turn prepared following an analogous reaction protocol to that described for Compound 65: (R)-1-(1-(2-(2,2,2-Trifluoroethoxy)pyridin-4-yl)ethyl)-3-(2-(1-(trifluoromethyl)cyclopropyl)ethyl)urea, steps (i-v), from the appropriate commercial auxiliary (S)-2-methyl-2-propanesulfinamide (CAS: 343338-28-3). The title compound was purified by reverse phase HPLC (Table 2, Method 7) to afford a white solid (45 mg, 58%).

¹H NMR (400 MHz, DMSO-d₆) δ 8.10 (d, J=5.3 Hz, 1H), 7.01 (dd, J=1.0, 5.3 Hz, 1H), 6.82 (s, 1H), 6.54 (d, J=7.9 Hz, 1H), 5.90 (dd, J=5.8, 5.8 Hz, 1H), 4.96 (q, J=9.1 Hz, 2H), 4.73-4.65 (m, 1H), 3.11-3.04 (m, 2H), 1.64 (dd, J=6.4, 9.0 Hz, 2H), 1.28 (d, J=7.1 Hz, 3H), 0.89-0.85 (m, 2H), 0.72 (s, 2H).

LC/MS (Table 1, Method F) Rₜ=4.66 min; MS m/z: 400 [M+H]⁺.

### Compound 68

### 1-((2-(Methyl(2,2,2-trifluoroethyl)amino)pyridin-4-yl)methyl)-3-(2-(1-(trifluoromethyl)cyclopropyl)ethyl)urea

### (i) 2-(Methyl(2,2,2-trifluoroethyl)amino)isonicotinonitrile

The title compound was prepared using an analogous reaction protocol to that described for Compound 28: (±)-1-((2-(3-Fluoropiperidin-1-yl)pyridin-4-yl)methyl)-3-(2-(1-(trifluoromethyl)cyclopropyl)ethyl)urea, step (i), from the appropriate commercial starting materials, 4-cyano-2-fluoropyridine (CAS: 3939-14-8) and 2,2,2-trifluoro-N-methyl-ethanamine hydrochloride (CAS: 2730-52-1). The compound was purified by flash column chromatography (cyclohexane to EtOAc, gradient elution) to afford the title compound (187 mg, 38%).

LC/MS (Table 1, Method H) Rₜ=1.72 min; MS m/z: 216 [M+H]⁺.

### (ii) 4-(Aminomethyl)-N-methyl-N-(2,2,2-trifluoroethyl)pyridin-2-amine

The title compound was prepared using an analogous reaction protocol to that described for Compound 11: 1-((6-(Benzyloxy)pyrimidin-4-yl)methyl)-3-(2-(1-(trifluoromethyl)cyclopropyl)ethyl)urea, step (ii), from the appropriate starting material, 2-(methyl(2,2,2-trifluoroethyl)amino)isonicotinonitrile (Compound 68, step (i)). The compound was purified by flash column chromatography (DCM to DCM:MeOH [2M NH₃]90:10, gradient elution) to afford the title compound (100 mg, 52%).

LC/MS (Table 1, Method D) Rₜ=1.13 min; MS m/z: 220 [M+H]⁺.

### (iii) 1-((2-(Methyl(2,2,2-trifluoroethyl)amino)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, 4-(aminomethyl)-N-methyl-N-(2,2,2-trifluoroethyl)pyridin-2-amine (Compound 68, step (ii)) and 2-(1-(trifluoromethyl)cyclopropyl)ethan-1-amine hydrochloride (CAS: 1454690-80-2). The title compound was purified by flash column chromatography (cyclohexane to EtOAc, gradient elution) followed by flash column chromatography (DCM to EtOAc, gradient elution) to afford a white solid (43 mg, 45%).

¹H NMR (400 MHz, DMSO-d₆) δ 8.03 (d, J=5.1 Hz, 1H), 6.60-6.56 (m, 2H), 6.46 (t, J=6.1 Hz, 1H), 6.03 (t, J=5.8 Hz, 1H), 4.47 (q, J=9.6 Hz, 2H), 4.14 (d, J=6.1 Hz, 2H), 3.15-3.07 (m, 2H), 3.05 (s, 3H), 1.70-1.64 (m, 2H), 0.91-0.86 (m, 2H), 0.76-0.72 (m, 2H).

LC/MS (Table 1, Method F) Rₜ=3.22 min; MS m/z: 399 [M+H]⁺.

### Compound 69

### 1-(2-(1-(Trifluoromethyl)cyclopropyl)ethyl)-3-((2-(trifluoromethyl)pyridin-4-yl) methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(trifluoromethyl)pyridin-4-yl)methanamine (CAS: 916304-20-6) and 2-(1-(trifluoromethyl)cyclopropyl)ethan-1-amine hydrochloride (CAS: 1454690-80-2). The title compound was purified by reverse phase HPLC (Table 2, Method 3) to afford a white solid (79 mg, 72%).

¹H NMR (400 MHz, DMSO-d₆) δ 8.68 (d, J=5.0 Hz, 1H), 7.71 (s, 1H), 7.54 (d, J=5.0 Hz, 1H), 6.65 (dd, J=6.1, 6.1 Hz, 1H), 6.19 (dd, J=5.8, 5.8 Hz, 1H), 4.32 (d, J=6.1 Hz, 2H), 3.11 (dd, J=6.2, 15.1 Hz, 2H), 1.70-1.65 (m, 2H), 0.91-0.86 (m, 2H), 0.73 (dd, J=6.8, 6.8 Hz, 2H).

LC/MS (Table 1, Method F) Rₜ=4.19 min; MS m/z: 356 [M+H]⁺.

### Compound 70

### 1-((4,4-Difluorocyclohexyl)methyl)-3-((2-(2,2,2-trifluoroethoxy)pyridin-4-yl) methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and (4,4-difluorocyclohexyl)methanamine (CAS: 810659-05-3). The title compound was purified by reverse phase HPLC (Table 2, Method 7) to afford a white solid (38 mg, 36%).

¹H NMR (400 MHz, DMSO-d₆) δ 8.10 (d, J=5.3 Hz, 1H), 6.96 (d, J=5.3 Hz, 1H), 6.76 (s, 1H), 6.42 (dd, J=6.1, 6.1 Hz, 1H), 6.19 (dd, J=6.0, 6.0 Hz, 1H), 4.97 (q, J=9.1 Hz, 2H), 4.21 (d, J=6.2 Hz, 2H), 2.93 (dd, J=6.3, 6.3 Hz, 2H), 2.02-1.95 (m, 2H), 1.83-1.68 (m, 4H), 1.49 (dd, J=3.5, 10.4 Hz, 1H), 1.19-1.11 (m, 2H).

LC/MS (Table 1, Method F) Rₜ=4.32 min; MS m/z: 382 [M+H]⁺.

### Compound 71

### 1-(4,4-Difluorocyclohexyl)-3-((2-(2,2,2-trifluoroethoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and 4,4-difluorocyclohexan-1-amine hydrochloride (CAS: 675112-70-6). The title compound was purified by reverse phase HPLC (Table 2, Method 7) to afford a white solid (53 mg, 52%).

¹H NMR (400 MHz, DMSO-d₆) δ 8.10 (d, J=5.3 Hz, 1H), 6.97 (d, J=5.3 Hz, 1H), 6.77 (s, 1H), 6.33 (dd, J=6.1, 6.1 Hz, 1H), 6.19 (d, J=7.7 Hz, 1H), 4.98 (q, J=9.1 Hz, 2H), 4.23-4.20 (m, 2H), 3.59 (d, J=8.4 Hz, 1H), 2.01-1.77 (m, 6H), 1.49-1.37 (m, 2H).

LC/MS (Table 1, Method F) Rₜ=4.22 min; MS m/z: 368 [M+H]⁺.

### Compound 72

### 6-Fluoro-N-((2-(2,2,2-trifluoroethoxy)pyridin-4-yl)methyl)indoline-1-carboxamide

### (i) 6-Fluoroindoline-1-carbonyl chloride

To a solution of 6-fluoroindoline hydrochloride (250 mg, 1.44 mmol) and pyridine (0.23 mL, 2.88 mmol) in DCM (5.0 mL) at 0° C. was added triphosgene (214 mg, 0.720 mmol) via dropwise addition. The reaction was allowed to warm to RT and stirred at RT for 18 h. The reaction mixture was quenched by the addition of 1 M HCl aqueous solution and next partitioned with DCM. The organic layer was separated. The combined organic layers were washed with saturated brine, dried (Na₂SO₄) and concentrated in vacuo to afford the title compound (358 mg, quantitative).

LC/MS (Table 1, Method D) Rₜ=1.55 min; MS m/z: 200 [M+H]⁺.

### (ii) 6-Fluoro-N-((2-(2,2,2-trifluoroethoxy)pyridin-4-yl)methyl)indoline-1-carboxamide

To a solution of 6-fluoroindoline-1-carbonyl chloride (106 mg, 0.534 mmol) and N,N-diisopropylethylamine (0.21 mL, 1.21 mmol) in DCM (2.0 mL) at 0° C. under nitrogen was added a solution of (2-(2,2,2-trifluoroethoxy)pyridin-4-yl)methanamine (CAS: 561297-93-6, 100 mg, 0.485 mmol) in DCM (2.0 mL). The reaction was allowed to warm to RT and stirred at RT for 18 h. The reaction mixture was next partitioned between DCM and distilled water. The organic layer was separated. The combined organic layers were washed with saturated brine, dried (Na₂SO₄) and concentrated in vacuo. The residue was purified by flash column chromatography (DCM to EtOAc, gradient elution) to afford the title compound as an off-white solid (105 mg, 57%).

¹H NMR (400 MHz, DMSO-d₆) δ 8.12 (d, J=5.3 Hz, 1H), 7.57 (dd, J=2.6, 11.3 Hz, 1H), 7.42 (t, J=5.9 Hz, 1H), 7.14 (dd, J=6.0, 8.1 Hz, 1H), 7.06 (dd, J=1.3, 5.3 Hz, 1H), 6.87 (s, 1H), 6.63 (dq, J=8.2, 3.9 Hz, 1H), 4.98 (q, J=9.1 Hz, 2H), 4.34 (d, J=5.8 Hz, 2H), 4.04 (t, J=8.7 Hz, 2H), 3.12 (t, J=8.7 Hz, 2H).

LC/MS (Table 1, Method F) Rₜ=4.82 min; MS m/z: 370 [M+H]⁺.

### Compound 73

### (R)-1-(2-Hydroxy-3,3-dimethylbutyl)-3-((2-(2,2,3,3,3-pentafluoropropoxy)pyridin-4-yl)methyl)urea

### (i) 2-(2,2,3,3,3-Pentafluoropropoxy)isonicotinonitrile

The title compound was prepared using an analogous reaction protocol to that described for Compound 26: 1-((2-Morpholino-6-(2,2,2-trifluoroethoxy)pyridin-4-yl)methyl)-3-(2-(1-(trifluoromethyl)cyclopropyl)ethyl)urea, step (ii), from the appropriate commercial starting materials, 2-chloro-4-pyridinecarbonitrile (CAS: 33252-30-1) and 2,2,3,3,3-pentafluoro-1-propanol (CAS: 422-05-9). The compound was purified by flash column chromatography (cyclohexane to EtOAc, gradient elution) to afford the title compound (420 mg, 46%).

LC/MS (Table 1, Method D) Rₜ=1.58 min; MS m/z: 253 [M+H]⁺.

### (ii) (2-(2,2,3,3,3-Pentafluoropropoxy)pyridin-4-yl)methanamine

The title compound was prepared using an analogous reaction protocol to that described for Compound 11: 1-((6-(Benzyloxy)pyrimidin-4-yl)methyl)-3-(2-(1-(trifluoromethyl)cyclopropyl)ethyl)urea, step (ii), from the appropriate starting material 2-(2,2,3,3,3-Pentafluoropropoxy)isonicotinonitrile (Compound 73, step (i)). This afforded the title compound (350 mg, 65%). The product was advanced into step (iii) without characterisation.

### (iii) (R)-1-(2-Hydroxy-3,3-dimethylbutyl)-3-((2-(2,2,3,3,3-pentafluoropropoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-(2,2,3,3,3-Pentafluoropropoxy)pyridin-4-yl)methanamine (Compound 73, step (ii)) and (R)-1-amino-3,3-dimethylbutan-2-ol hydrochloride (CAS: 2089245-23-6). The title compound was purified by flash column chromatography (DCM to MeOH, gradient elution) to afford a white solid (97 mg, 69%).

¹H NMR (400 MHz, DMSO-d₆) δ 8.11 (d, J=5.3 Hz, 1H), 6.98 (dd, J=1.3, 5.3 Hz, 1H), 6.77 (s, 1H), 6.66 (t, J=6.1 Hz, 1H), 5.98 (dd, J=3.5, 7.3 Hz, 1H), 5.07 (ddd, J=13.8, 13.8, 1.0 Hz, 2H), 4.72 (d, J=5.6 Hz, 1H), 4.22 (dd, J=2.5, 6.0 Hz, 2H), 3.40-3.34 (m, 1H), 3.06 (dq, J=2.6, 5.0 Hz, 1H), 2.70 (ddd, J=3.7, 9.4, 13.2 Hz, 1H), 0.84 (s, 9H).

LC/MS (Table 1, Method F) Rₜ=4.40 min; MS m/z: 400 [M+H]⁺.

### Compound 74

### 1-(2-(1-(Trifluoromethyl)cyclopropyl)ethyl)-3-((2-(3,3,3-trifluoropropoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials, (2-(3,3,3-trifluoropropoxy)pyridin-4-yl)methanamine dihydrochloride and 2-(1-(trifluoromethyl)cyclopropyl)ethan-1-amine hydrochloride (CAS: 1454690-80-2). (2-(3,3,3-trifluoropropoxy)pyridin-4-yl)methanamine dihydrochloride was in turn prepared following an analogous reaction protocol to that described for Compound 61: 2-(6-Oxa-3-azabicyclo[3.1.1]heptan-3-yl)-N-((2-(2,2,2-trifluoroethoxy)pyridin-4-yl)methyl)acetamide, steps (i-iii), from the appropriate commercial starting materials. The title compound was purified by reverse phase HPLC (Table 2, Method 2, non-linear gradient from 5% to 60% MeCN) to afford an off-white solid (90 mg, 65%).

¹H NMR (400 MHz, DMSO-d₆) δ 8.08 (d, J=5.3 Hz, 1H), 6.88 (dd, J=1.4, 5.4 Hz, 1H), 6.64 (s, 1H), 6.52 (t, J=6.1 Hz, 1H), 6.09 (t, J=5.8 Hz, 1H), 4.47 (t, J=6.0 Hz, 2H), 4.18 (d, J=6.1 Hz, 2H), 3.12 (q, J=7.1 Hz, 2H), 2.84-2.72 (m, 2H), 1.68 (t, J=7.8 Hz, 2H), 0.92-0.88 (m, 2H), 0.76 (s, 2H).

LC/MS (Table 1, Method A) Rₜ=4.64 min; MS m/z: 400 [M+H]⁺.

### Compound 75

### 1-(2-Chlorobenzyl)-3-((2-(2,2,2-trifluoroethoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and 2-chlorobenzylamine (CAS: 89-97-4). The title compound was purified by reverse phase HPLC (Table 2, Method 6, non-linear gradient from 40% to 100% MeOH) followed by reverse phase HPLC (Table 2, Method 3) to afford an off-white solid (38 mg, 41%).

¹H NMR (400 MHz, DMSO-d₆) δ 8.12 (dd, J=0.5, 5.3 Hz, 1H), 7.44 (dd, J=1.3, 7.5 Hz, 1H), 7.38-7.27 (m, 3H), 7.00 (dd, J=1.3, 5.3 Hz, 1H), 6.81 (s, 1H), 6.72 (t, J=6.3 Hz, 1H), 6.67 (t, J=6.0 Hz, 1H), 5.00 (q, J=9.2 Hz, 2H), 4.32 (d, J=5.9 Hz, 2H), 4.26 (d, J=6.0 Hz, 2H).

LC/MS (Table 1, Method E) Rₜ=4.58 min; MS m/z: 374 [M+H]⁺.

### Compound 76

### 1-(3-Fluorobenzyl)-3-((6-(2,2,2-trifluoroethoxy)pyrimidin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (6-(2,2,2-trifluoroethoxy)pyrimidin-4-yl)methanamine dihydrochloride and 3-fluorobenzylamine (CAS: 100-82-3). (6-(2,2,2-trifluoroethoxy)pyrimidin-4-yl)methanamine dihydrochloride was in turn prepared following an analogous reaction protocol to that described for Compound 61: 2-(6-Oxa-3-azabicyclo[3.1.1]heptan-3-yl)-N-((2-(2,2,2-trifluoroethoxy)pyridin-4-yl)methyl)acetamide, steps (i-iii). The title compound was purified by reverse phase HPLC (Table 2, Method 2, non-linear gradient from 5% to 60% MeCN) followed by reverse phase HPLC (Table 2, Method 3) to afford an off-white solid (22 mg, 17%).

¹H NMR (400 MHz, DMSO-d₆) δ 8.79 (d, J=1.0 Hz, 1H), 7.39-7.33 (m, 1H), 7.13-7.02 (m, 3H), 6.87 (s, 1H), 6.81 (t, J=6.1 Hz, 1H), 6.70 (t, J=6.1 Hz, 1H), 5.10 (q, J=9.0 Hz, 2H), 4.30 (d, J=6.0 Hz, 2H), 4.26 (d, J=6.0 Hz, 2H).

LC/MS (Table 1, Method A) Rₜ=4.10 min; MS m/z: 359 [M+H]⁺.

### Compound 77

### 1-(3-Chloro-5-fluorophenyl)-3-((2-(2,2,2-trifluoroethoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and 3-chloro-5-fluoroaniline (CAS: 4863-91-6). The title compound was purified by reverse phase HPLC (Table 2, Method 7) to afford a white solid (17 mg, 16%).

¹H NMR (400 MHz, DMSO-d₆) δ 9.20 (s, 1H), 8.13 (d, J=5.3 Hz, 1H), 7.35 (dd, J=2.0, 2.0 Hz, 1H), 7.31-7.26 (m, 1H), 7.04-7.00 (m, 2H), 6.93-6.89 (m, 1H), 6.83 (s, 1H), 5.02-4.94 (m, 2H), 4.31 (d, J=5.9 Hz, 2H).

LC/MS (Table 1, Method B) Rₜ=4.98 min; MS m/z: 378 [M+H]⁺.

### Compound 78

### (R)-1-(2-Hydroxy-3,3-dimethylbutyl)-3-((2-(3,3,3-trifluoropropoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(3,3,3-trifluoropropoxy)pyridin-4-yl)methanamine dihydrochloride and (R)-1-amino-3,3-dimethylbutan-2-ol hydrochloride (CAS: 2089245-23-6). (2-(3,3,3-trifluoropropoxy)pyridin-4-yl)methanamine was in turn prepared following an analogous reaction protocol to that described for Compound 61: 2-(6-Oxa-3-azabicyclo[3.1.1]heptan-3-yl)-N-((2-(2,2,2-trifluoroethoxy)pyridin-4-yl)methyl)acetamide, steps (i-iii), from the appropriate commercial starting materials. The title compound was purified by reverse phase HPLC (Table 2, Method 3) to afford an off-white solid (45 mg, 70%).

¹H NMR (400 MHz, DMSO-d₆) δ 8.09 (d, J=5.3 Hz, 1H), 6.89 (dd, J=1.2, 5.3 Hz, 1H), 6.65 (s, 2H), 5.98 (q, J=3.6 Hz, 1H), 4.74 (d, J=5.6 Hz, 1H), 4.48 (t, J=6.0 Hz, 2H), 4.20 (dd, J=2.0, 5.9 Hz, 2H), 3.42-3.37 (m, 1H), 3.10-3.04 (m, 1H), 2.85-2.67 (m, 3H), 0.85 (s, 9H).

LC/MS (Table 1, Method F) Rₜ=4.03 min; MS m/z: 364 [M+H]⁺.

### Compound 79

### 1-((3,3-Difluorocyclohexyl)methyl)-3-((2-(2,2,2-trifluoroethoxy)pyridin-4-yl)methyl) urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and (3,3-difluorocyclohexyl)methanamine (CAS: 1379151-12-8). The title compound was purified by reverse phase HPLC (Table 2, Method 7) to afford a white solid (61 mg, 63%).

¹H NMR (400 MHz, DMSO-d₆) δ 8.09 (d, J=5.3 Hz, 1H), 6.96 (d, J=5.3 Hz, 1H), 6.76 (s, 1H), 6.42 (dd, J=6.1, 6.1 Hz, 1H), 6.21 (dd, J=6.0, 6.0 Hz, 1H), 4.97 (q, J=9.1 Hz, 2H), 4.21 (d, J=6.1 Hz, 2H), 3.01-2.90 (m, 2H), 2.01-1.93 (m, 2H), 1.79-1.58 (m, 4H), 1.50-1.32 (m, 2H), 1.01-0.88 (m, 1H).

LC/MS (Table 1, Method F) Rₜ=4.36 min; MS m/z: 382 [M+H]⁺.

### Compound 80

### 1-((6-(2,2,2-Trifluoroethoxy)pyridin-3-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (6-(2,2,2-trifluoroethoxy)pyridin-3-yl)methanamine (CAS: 771584-26-0) and 2-(1-(trifluoromethyl)cyclopropyl)ethan-1-amine hydrochloride (CAS: 1454690-80-2). The title compound was purified by reverse phase HPLC (Table 2, Method 8) to afford a white solid (18 mg, 18%).

¹H NMR (400 MHz, DMSO-d₆) δ 8.07 (d, J=2.0 Hz, 1H), 7.69 (dd, J=2.4, 8.5 Hz, 1H), 6.95 (d, J=8.5 Hz, 1H), 6.44 (dd, J=6.0, 6.0 Hz, 1H), 5.99 (dd, J=5.8, 5.8 Hz, 1H), 4.98 (q, J=9.2 Hz, 2H), 4.15 (d, J=6.0 Hz, 2H), 3.11 (dd, J=6.1, 15.2 Hz, 2H), 1.70-1.64 (m, 2H), 0.92-0.87 (m, 2H), 0.74 (dd, J=6.7, 6.7 Hz, 2H).

LC/MS (Table 1, Method A) Rₜ=4.61 min; MS m/z: 386 [M+H]⁺.

### Compound 81

### (±)-1-(6-Fluoro-2,3-dihydro-1H-inden-1-yl)-3-((2-(2,2,2-trifluoroethoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and (±)-6-fluoro-2,3-dihydro-1H-inden-1-amine hydrochloride (CAS: 1191908-44-7). The title compound was purified by flash column chromatography (DCM to EtOAc, gradient elution) to afford a white solid (51 mg, 54%).

¹H NMR (400 MHz, DMSO-d₆) δ 8.12 (dd, J=0.5, 5.3 Hz, 1H), 7.27-7.22 (m, 1H), 7.04-6.98 (m, 2H), 6.94 (dd, J=2.2, 9.0 Hz, 1H), 6.82 (s, 1H), 6.57 (d, J=8.3 Hz, 1H), 6.46 (t, J=6.1 Hz, 1H), 5.11 (q, J=7.9 Hz, 1H), 4.98 (q, J=9.2 Hz, 2H), 4.28 (d, J=6.2 Hz, 2H), 2.91-2.82 (m, 1H), 2.78-2.69 (m, 1H), 2.47-2.39 (m, 1H), 1.82-1.72 (m, 1H).

LC/MS (Table 1, Method F) Rₜ=4.57 min; MS m/z: 384 [M+H]⁺.

### Compound 82

### 1-((2-(2,2,2-Trifluoroethoxy)-6-(trifluoromethyl)pyridin-4-yl)methyl)-3-(2-(1-(trifluoromethyl)cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)-6-(trifluoromethyl)pyridin-4-yl)methanamine and 2-(1-(trifluoromethyl)cyclopropyl)ethan-1-amine hydrochloride (CAS: 1454690-80-2). (2-(2,2,2-trifluoroethoxy)-6-(trifluoromethyl) pyridin-4-yl)methanamine was in turn prepared following an analogous reaction protocol to that described for Compound 11: 1-((6-(Benzyloxy)pyrimidin-4-yl)methyl)-3-(2-(1-(trifluoromethyl)cyclopropyl)ethyl)urea, step (i-ii), from the appropriate commercial starting materials. The title compound was purified by flash column chromatography (DCM to MeOH, gradient elution) to afford an off-white solid (51 mg, 31%).

¹H NMR (400 MHz, DMSO-d₆) δ 7.48 (s, 1H), 7.09 (s, 1H), 6.63 (t, J=6.1 Hz, 1H), 6.19 (t, J=5.8 Hz, 1H), 5.02 (q, J=9.0 Hz, 2H), 4.29 (d, J=6.1 Hz, 2H), 3.11 (dd, J=6.2, 15.0 Hz, 2H), 1.70-1.65 (m, 2H), 0.90-0.86 (m, 2H), 0.75-0.71 (m, 2H).

LC/MS (Table 1, Method F) Rₜ=5.11 min; MS m/z: 454 [M+H]⁺.

### Compound 83

### (R)-1-(2-Hydroxy-3,3-dimethylbutyl)-3-((2-(2,2,2-trifluoroethoxy)-6-(trifluoromethyl) pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)-6-(trifluoromethyl)pyridin-4-yl)methanamine and (R)-1-amino-3,3-dimethylbutan-2-ol hydrochloride (CAS: 2089245-23-6). (2-(2,2,2-trifluoroethoxy)-6-(trifluoromethyl)pyridin-4-yl)methanamine was in turn prepared following an analogous reaction protocol to that described for Compound 11: 1-((6-(Benzyloxy)pyrimidin-4-yl)methyl)-3-(2-(1-(trifluoromethyl)cyclopropyl) ethyl) urea, step (i-ii), from the appropriate commercial starting materials. The title compound was purified by reverse phase HPLC (Table 2, Method 3, non-linear gradient from 40% to 100% MeCN) to afford an off-white solid (41 mg, 51%).

¹H NMR (400 MHz, DMSO-d₆) δ 7.50 (s, 1H), 7.12 (s, 1H), 6.76 (t, J=6.1 Hz, 1H), 6.10-6.05 (m, 1H), 5.04 (q, J=9.0 Hz, 2H), 4.73 (d, J=5.5 Hz, 1H), 4.35-4.31 (m, 2H), 3.10-3.05 (m, 1H), 2.77-2.68 (m, 2H), 0.85 (s, 9H).

LC/MS (Table 1, Method A) Rₜ=4.80 min; MS m/z: 418 [M+H]⁺.

### Compound 84

### (R)—N-(2-Hydroxy-3,3-dimethylbutyl)-6-(2,2,2-trifluoroethoxy)-1,3-dihydro-2H-pyrrolo[3,4-c]pyridine-2-carboxamide

### (i) tert-Butyl 6-(2,2,2-trifluoroethoxy)-1,3-dihydro-2H-pyrrolo[3,4-c]pyridine-2-carboxylate

The title compound was prepared using an analogous reaction protocol to that described for Compound 21: 1-(3-(2,2,2-Trifluoroethoxy)-5,6,7,8-tetrahydroisoquinolin-5-yl)-3-(2-(1-(trifluoromethyl)cyclopropyl)ethyl)urea, step (iv), from the appropriate commercial starting materials, tert-butyl 6-chloro-1,3-dihydropyrrolo[3,4-c]pyridine-2-carboxylate (CAS: 1700330-18-2) and 2,2,2-trifluoroethanol (CAS: 75-89-8). The compound was purified by flash column chromatography (cyclohexane to EtOAc, gradient elution) to afford the title compound (187 mg, 80%).

¹H NMR (400 MHz, CDCl₃) δ 8.03 (d, J=16.4 Hz, 1H), 6.76 (d, J=21.8 Hz, 1H), 4.76 (dd, J=7.5, 17.5 Hz, 2H), 4.65 (s, 2H), 4.62 (s, 2H), 1.52 (s, 9H).

### (ii) 6-(2,2,2-Trifluoroethoxy)-2,3-dihydro-1H-pyrrolo[3,4-c]pyridine

To a solution of tert-butyl 6-(2,2,2-trifluoroethoxy)-1,3-dihydro-2H-pyrrolo[3,4-c]pyridine-2-carboxylate ((Compound 84, step (i), 187 mg, 0.588 mmol)) in DCM (1.0 mL) at 0° C. was added trifluoroacetic acid (0.45 mL, 5.88 mmol). The reaction was allowed to warm to RT and stirred at RT for 18 h. The reaction mixture was concentrated in vacuo. The residue was purified by SCX-2 column chromatography (DCM to DCM:MeOH [2M NH₃]90:10, gradient elution) to afford the title compound (106 mg, 83%).

LC/MS (Table 1, Method D) Rₜ=1.00 min; MS m/z: 219 [M+H]⁺.

### (iii) 6-(2,2,2-Trifluoroethoxy)-1,3-dihydro-2H-pyrrolo[3,4-c]pyridine-2-carbonyl chloride

The title compound was prepared using an analogous reaction protocol to that described for Compound 72: 6-Fluoro-N-((2-(2,2,2-trifluoroethoxy)pyridin-4-yl)methyl)indoline-1-carboxamide, step (i), from the appropriate starting material, 6-(2,2,2-trifluoroethoxy)-2,3-dihydro-1H-pyrrolo[3,4-c]pyridine (Compound 84, step (ii)). This afforded the title compound (71 mg, 84%).

LC/MS (Table 1, Method D) Rₜ=1.68 min; MS m/z: 281 [M+H]⁺.

### (iv) (R)—N-(2-Hydroxy-3,3-dimethylbutyl)-6-(2,2,2-trifluoroethoxy)-1,3-dihydro-2H-pyrrolo[3,4-c]pyridine-2-carboxamide

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, 6-(2,2,2-Trifluoroethoxy)-1,3-dihydro-2H-pyrrolo[3,4-c]pyridine-2-carbonyl chloride (Compound 84, step (iii)) and (R)-1-amino-3,3-dimethylbutan-2-ol hydrochloride (CAS: 2089245-23-6). The title compound was purified by flash column chromatography (DCM to MeOH, gradient elution) to afford an off-white solid (31 mg, 33%).

¹H NMR (400 MHz, DMSO-d₆) δ 8.17 (s, 1H), 6.98 (s, 1H), 6.27 (t, J=5.5 Hz, 1H), 4.99 (q, J=9.1 Hz, 2H), 4.78 (d, J=4.4 Hz, 1H), 4.60-4.56 (m, 4H), 3.42-3.35 (m, 1H), 3.20-3.15 (m, 1H), 2.86-2.78 (m, 1H), 0.87 (s, 9H).

LC/MS (Table 1, Method F) Rₜ=4.07 min; MS m/z: 362 [M+H]⁺.

### Compound 85

### 1-(2-Chloro-5-fluorobenzyl)-3-((2-(2,2,2-trifluoroethoxy)pyridin-4-yl)methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and 2-chloro-5-fluorobenzylamine (CAS: 202522-23-4). The title compound was purified by flash column chromatography (DCM to EtOAc, gradient elution) followed by reverse phase HPLC (Table 2, Method 8) to afford an off-white solid (5.9 mg, 6%).

¹H NMR (400 MHz, DMSO-d₆) δ 8.12 (d, J=5.3 Hz, 1H), 7.49 (dd, J=5.1, 8.8 Hz, 1H), 7.19-7.09 (m, 2H), 7.00 (dd, J=1.3, 5.3 Hz, 1H), 6.81 (t, J=6.2 Hz, 2H), 6.76 (t, J=6.0 Hz, 1H), 4.99 (q, J=9.1 Hz, 2H), 4.28 (dd, J=6.1, 9.5 Hz, 4H).

LC/MS (Table 1, Method B) Rₜ=4.64 min; MS m/z: 392 [M+H]⁺.

### Compound 86

### (R)-1-(6-fluoro-2,3-dihydro-1H-inden-1-yl)-3-((2-(2,2,2-trifluoroethoxy)pyridin-4-yl) methyl)urea

The title compound was prepared using an analogous reaction protocol to that described for Compound 1: 1-((2-(Benzyloxy)pyridin-4-yl)methyl)-3-(3-fluorophenyl)urea, from the appropriate commercial starting materials, (2-(2,2,2-trifluoroethoxy)pyridin-4-yl)methanamine (CAS: 561297-93-6) and (R)-6-fluoro-2,3-dihydro-1H-inden-1-amine (CAS: 731859-02-2). The title compound was purified by flash column chromatography (DCM to MeOH, gradient elution) followed by reverse phase HPLC (Table 2, Method 7, non-linear gradient 30% to 95% MeCN) to afford a white solid (49 mg, 52%).

¹H NMR (400 MHz, DMSO-d₆) δ 8.12 (d, J=5.3 Hz, 1H), 7.24 (dd, J=5.3, 8.2 Hz, 1H), 7.04-6.98 (m, 2H), 6.94 (dd, J=2.2, 9.0 Hz, 1H), 6.82 (s, 1H), 6.58 (d, J=8.3 Hz, 1H), 6.47 (t, J=6.0 Hz, 1H), 5.11 (q, J=8.2 Hz, 1H), 4.98 (q, J=9.1 Hz, 2H), 4.28 (d, J=6.1 Hz, 2H), 2.86 (ddd, J=3.0, 8.7, 15.5 Hz, 1H), 2.78-2.68 (m, 1H), 2.47-2.38 (m, 1H), 1.82-1.72 (m, 1H).

LC/MS (Table 1, Method F) Rₜ=4.61 min; MS m/z: 384 [M+H]⁺.

### Compound 87

### 1-((5-Fluoro-2-(2,2,2-trifluoroethoxy)pyridin-4-yl)methyl)-3-(2-(1-(trifluoromethyl) cyclopropyl)ethyl)urea

### (i) tert-Butyl ((2-chloro-5-fluoropyridin-4-yl)methyl)carbamate

The title compound was prepared using an analogous reaction protocol to that described for Compound 61: 2-(6-Oxa-3-azabicyclo[3.1.1]heptan-3-yl)-N-((2-(2,2,2-trifluoroethoxy)pyridin-4-yl)methyl)acetamide, step (i), from the appropriate commercial starting material, 2-chloro-5-fluoro-pyridine-4-carbonitrile (CAS: 1057319-20-6). The compound was purified by flash column chromatography (cyclohexane to EtOAc, gradient elution) to afford the title compound (266 mg, 23%).

LC/MS (Table 1, Method D) Rₜ=1.57 min; MS m/z: 261 [M+H]⁺.

### (ii) tert-Butyl ((5-fluoro-2-(2,2,2-trifluoroethoxy)pyridin-4-yl)methyl)carbamate

The title compound was prepared using an analogous reaction protocol to that described for Compound 21: 1-(3-(2,2,2-Trifluoroethoxy)-5,6,7,8-tetrahydroisoquinolin-5-yl)-3-(2-(1-(trifluoromethyl)cyclopropyl)ethyl)urea, step (iv), from the appropriate starting materials, tert-butyl ((2-chloro-5-fluoropyridin-4-yl)methyl)carbamate (Compound 87, step (i)) and 2,2,2-trifluoroethanol (CAS: 75-89-8). The compound was purified by flash column chromatography (cyclohexane to EtOAc, gradient elution) to afford the title compound (76 mg, 76%).

LC/MS (Table 1, Method D) Rₜ=1.78 min; MS m/z: 325 [M+H]⁺.

### (iii) (5-Fluoro-2-(2,2,2-trifluoroethoxy)pyridin-4-yl)methanamine hydrochloride

The title compound was prepared using an analogous reaction protocol to that described for Compound 6: 1-Methyl-3-((2-(2,2,2-trifluoroethoxy)pyridin-4-yl)methyl)-1-(2-(1-(trifluoromethyl)cyclopropyl)ethyl)urea, step (iii), from the appropriate starting material, tert-butyl ((5-fluoro-2-(2,2,2-trifluoroethoxy)pyridin-4-yl)methyl)carbamate (Compound 87, step (ii)). The compound was triturated with diethyl ether to afford the title compound (52 mg, 85%).

LC/MS (Table 1, Method D) Rₜ=1.12 min; MS m/z: 225 [M+H]⁺.

### (iv) 1-((5-Fluoro-2-(2,2,2-trifluoroethoxy)pyridin-4-yl)methyl)-3-(2-(1-(trifluoromethyl) cyclopropyl)ethyl)urea

The title compound was prepared using an analogous reaction protocol to that described for Compound 1: 1-((2-(Benzyloxy)pyridin-4-yl)methyl)-3-(3-fluorophenyl)urea, from the appropriate starting materials (5-fluoro-2-(2,2,2-trifluoroethoxy)pyridin-4-yl)methanamine hydrochloride (Compound 87, step (iii)) and 2-(1-(trifluoromethyl)cyclopropyl)ethan-1-amine hydrochloride (CAS: 1454690-80-2). The title compound was purified by flash column chromatography (DCM to MeOH, gradient elution) to afford a white solid (27 mg, 37%).

¹H NMR (400 MHz, DMSO-d₆) δ 8.11 (d, J=1.4 Hz, 1H), 6.78 (d, J=5.0 Hz, 1H), 6.56 (t, J=6.0 Hz, 1H), 6.19 (t, J=5.7 Hz, 1H), 4.94 (q, J=9.1 Hz, 2H), 4.25 (d, J=6.1 Hz, 2H), 3.15-3.07 (m, 2H), 1.70-1.65 (m, 2H), 0.91-0.86 (m, 2H), 0.76-0.71 (m, 2H).

LC/MS (Table 1, Method B) Rₜ=4.71 min; MS m/z: 404 [M+H]⁺.

### Compound 88

### 1-((3,3-Difluorocyclopentyl)methyl)-3-((2-(2,2,2-trifluoroethoxy)pyridin-4-yl)methyl) urea

The title compound was prepared using an analogous reaction protocol to that described for Compound 1: 1-((2-(Benzyloxy)pyridin-4-yl)methyl)-3-(3-fluorophenyl)urea), from the appropriate commercial starting materials (2-(2,2,2-trifluoroethoxy)pyridin-4-yl)methanamine (CAS: 561297-93-6) and (3,3-difluorocyclopentyl)methanamine hydrochloride (CAS: 1439900-13-6). The title compound was purified by reverse phase HPLC (Table 2, Method 7) to afford a white solid (54 mg, 52%).

¹H NMR (400 MHz, DMSO-d₆) δ 8.10 (d, J=5.4 Hz, 1H), 6.96 (d, J=5.2 Hz, 1H), 6.76 (s, 1H), 6.44 (dd, J=6.1, 6.1 Hz, 1H), 6.24 (dd, J=5.9, 5.9 Hz, 1H), 4.97 (q, J=9.1 Hz, 2H), 4.21 (d, J=6.1 Hz, 2H), 3.03 (dd, J=6.3, 6.3 Hz, 2H), 2.27-1.96 (m, 4H), 1.87-1.69 (m, 2H), 1.49-1.38 (m, 1H).

LC/MS (Table 1, Method F) Rₜ=4.20 min; MS m/z: 368 [M+H]⁺.

### Example 2

Assessment of In Vitro Potency Against K_{V}7.2/K_{V}7.3 Channels

An automated patch-clamp assay on the Sophion Qube 384 was developed for potency testing to identify small molecule activators of heteromeric K_{V}7.2/K_{V}7.3 channels (KCNQ2, Uniprot ID 043526; KCNQ3, Uniprot 043525).

The cell line used was a stably transfected CHO-K1 cell line with constitutive K_{V}7.2/K_{V}7.3 expression.

CHO-K1/Kv7.2/Kv7.3 cells were maintained in the following culture media:
DMEM/F-12 with GlutaMAX™ (Gibco 31331-028),
10% Fetal clone 2 serum (Perbio Science SH30066.03),
1 mg/ml Geneticin™ selective antibiotic (G418, Invitrogen 1013027), and
5 μg/ml BlasticidinS HCl (Invivogen Ant-bl-5).

On the day of the experiment, cells were resuspended in serum free media, counted and diluted to a final concentration of 3.5×10⁶ cells per ml of media.

Cells were then placed onto the Sophion Qube 384 and rested for a minimum of 1 hour.

The following solutions were used for recording:
extracellular solution (in mM): 145 NaCl, 4 KCl, 1 MgCl2, 2 CaCl2), 10 HEPES, 10 glucose, pH 7.4, 315-320 mOsm.
intracellular solution (in mM): 120 KCl, 5.74 CaCl2), 1.75 MgCl2, 10 EGTA, 10 HEPES, 5 Na2ATP, pH 7.2, adjusted to 315 mOsm with sucrose.

After establishing the whole-cell configuration, cells were held at −80 mV throughout the experiment. A current-voltage (I-V) protocol stepping from −100 to +20 mV for 1 second was applied to measure Kv7.2/Kv7.3 currents, each step was followed by a 200 ms pulse to 0 mV to measure the tail currents. From the I-V protocol a Boltzmann fit was applied to generate activation curves. Data were sampled at 25 kHz and filtered at 5 kHz (Bessel). Data were produced using multihole QChips. The I-V protocol was applied several times to establish the response in control conditions (typically 0.3% DMSO) and in the presence of the test compound.

Data were reviewed in Sophion Analyser version 6.5.2 (Sophion Bioscience) for recording quality and filters were applied to remove any failed wells. Leak subtraction was applied to all recordings. Data filters for multihole QChips were typically: seal resistance>4 MΩ, capacitance >20 pF, baseline VHalf between 0 to −40 mV, baseline holding current between −2 to 2 nA, baseline steady state current at 20 mV >4 nA unless otherwise stated.

### Assessment of In Vitro Potency Against K_{V}7.4 Channels

A counter-screening assay was also developed on the Qube against the homomeric K_{V}7.4 channel (KCNQ4, Uniprot ID P56696). The cell line used was a stably transfected CHO-K1 with constitutive K_{V}7.4 expression supplied by Charles River Laboratories.

CHO-K1 cells stably expressing K_{V}7.4 channels were maintained in the following culture media:
Hams F-12 with GlutaMAX™ (Life Technologies 31765027),
10% Fetal clone 2 serum (Perbio Science SH30066.03), and
250 μg/ml Geneticin™ selective antibiotic (G418, Invitrogen 1013-027).

Cell maintenance, preparation, recording conditions and voltage protocols were all identical to those above.

Assessment of in vitro potency against K_{V}7.3/K_{V}7.5 channels

Counter-screening was also carried out against the heteromeric K_{V}7.3/K_{V}7.5 channel (KCNQ3, Uniprot ID 043525; KCNQ5, Uniprot ID Q9NR82). The cell line used was a stably transfected HEK293 cell line with constitutive Kv7.3 expression and tetracycline inducible K_{V}7.5 expression supplied by Charles River Laboratories.

Cells were maintained in the following culture media:
DMEM/F-12 with GlutaMAX™ (Life Technologies 31331-028),
10% Tet Systems FCS (Clonetech 631106),
500 μg/ml Geneticin™ selective antibiotic (G418, Invitrogen 1013-027),
5 μg/ml Blasticidin S HCl (InvivoGen ant-bl-5), and
100 μg/ml Zeocin (InvivoGen: ant-zn-5).

Cell maintenance, preparation, recording conditions and voltage protocols were all identical to those above.

### Analysis of Data for Assessment of Potency

10 concentrations of test compounds were applied to individual wells in quadruplicate to assess potency as a 1 in 3 dilution series from the maximal concentration of 30 μM. The average shift in the voltage for half activation (V_{half}) for each concentration was used to generate concentration-response curves fitted with a 4-parameter logistic model to estimate the EC₅₀ from the point of inflection of the curve and the maximal shift in the V_{half} from the top of the curve.

The results are summarized in Table 3 below. The lower the EC₅₀ value, the higher the potency of the analyzed compound. The selectivity criteria is assigned on the basis of the maximal shift in the Vhalf at 30 μM. Selective compounds showed Kv7.4 and/or Kv7.3/7.5 ΔV_{1/2} equal to or lower than 15 mV (letter A in table 3). Non-selective compounds showed Kv7.4 and/or Kv7.3/7.5 ΔV_{1/2} higher than 15 mV (letter B in table 3). In Table 3, ND means that selectivity was not determined.

TABLE 3

| | Kv7.2/7.3 | Kv7.2/7.3 | Kv7.4 | Kv7.3/7.5 |
|---|---|---|---|---|
| | EC50 | ΔV1/2 | selectivity | selectivity |
| Compound | (μM) | (mV) | criteria | criteria |
| 1 | 0.6 | 30.51 | B | B |
| 2 | 0.966 | 16.51 | A | A |
| 3 | 0.472 | 25.42 | A | A |
| 4 | 1.7 | 27.28 | A | B |
| 5 | 2.39 | 21.49 | A | A |
| 7 | 5.13 | 22.31 | A | A |
| 11 | 1.83 | 27.09 | A | B |
| 12 | 7.65 | 13.2 | A | A |
| 13 | 4.45 | 22.29 | A | A |
| 14 | 0.392 | 26.52 | B | A |
| 15 | 2.02 | 25.72 | A | A |
| 16 | 6.23 | 20.12 | A | A |
| 17 | 5.45 | 28.27 | A | A |
| 18 | 0.649 | 24.69 | A | A |
| 19 | 0.263 | 23.76 | A | A |
| 20 | 0.689 | 29.4 | A | A |
| 21 | 1.8 | 14.76 | B | B |
| 22 | 1.94 | 27.07 | A | A |
| 23 | 4.75 | 18.69 | A | A |
| 24 | 10.98 | 17.14 | A | A |
| 25 | 0.4 | 25.77 | A | A |
| 26 | 5.14 | 31.67 | B | A |
| 27 | 8.02 | 18.22 | B | B |
| 31 | 3.08 | 25.36 | A | B |
| 35 | 0.649 | 38.22 | B | B |
| 36 | 0.435 | 31.09 | A | A |
| 37 | 0.82 | 19.56 | A | A |
| 38 | 2.02 | 28.31 | A | A |
| 39 | 1.47 | 21.29 | A | A |
| 41 | 0.428 | 29.72 | A | A |
| 42 | 0.872 | 16.7 | A | A |
| 43 | 0.765 | 53.72 | B | B |
| 44 | 6.3 | 17.02 | A | A |
| 46 | 0.432 | 21.41 | B | B |
| 47 | 0.254 | 26.23 | A | A |
| 48 | 2.41 | 22.74 | A | A |
| 49 | 4.23 | 23.89 | A | A |
| 51 | 0.289 | 33.62 | A | A |
| 54 | 7.61 | 22.67 | A | A |
| 58 | 10.52 | 48.9 | B | B |
| 62 | 11.7 | 16.94 | A | B |
| 63 | 11.8 | 33.01 | B | B |
| 65 | 0.152 | 26.28 | A | A |
| 67 | 5 | 23.87 | A | A |
| 68 | 4.73 | 27.19 | A | A |
| 69 | 4.28 | 23.15 | A | A |
| 70 | 0.975 | 26.04 | A | A |
| 71 | 1.58 | 55.93 | B | B |
| 73 | 2.47 | 26.35 | A | A |
| 74 | 1.78 | 26.68 | A | A |
| 75 | 0.508 | 25.35 | B | A |
| 77 | 0.549 | 32.37 | B | A |
| 78 | 11.66 | 19.21 | A | A |
| 79 | 0.862 | 26.55 | A | A |
| 80 | 3.9 | 28.15 | B | A |
| 81 | 0.31 | 14.75 | A | A |
| 82 | 2.15 | 18.38 | B | A |
| 85 | 0.206 | 20.49 | A | A |
| 86 | 1.35 | 38.89 | A | A |
| 87 | 1.85 | 27.66 | A | A |
| 88 | 0.96 | 30.26 | A | B |
| 89 | 2.18 | 18.85 | B | A |
| 90 | 0.237 | 20.86 | A | A |
| 91 | 2.29 | 37.24 | A | B |
| 92 | 3.95 | 16.77 | A | A |
| 93 | 4.46 | 34.16 | B | A |
| 94 | 4.17 | 13.23 | A | A |
| 95 | 2.29 | 21.55 | A | A |
| 96 | 6.61 | 25.66 | A | A |
| 97 | 2.3 | 26.87 | B | B |
| 98 | 5.14 | 22.14 | A | A |
| 99 | 12.84 | 29.92 | B | B |
| 100 | 3.94 | 24.71 | A | B |
| 101 | 2.64 | 38.71 | B | B |
| 102 | 3.83 | 35.23 | B | B |
| 103 | 1.34 | 42.63 | A | B |
| 104 | 3.09 | 21.96 | A | A |
| 105 | 1.5 | 35.29 | B | A |
| 106 | 2.84 | 28.47 | A | A |
| 107 | 3.06 | 20.76 | A | A |
| 108 | 3.18 | 26.46 | A | A |
| 109 | 4.79 | 16.81 | A | A |
| 110 | 1.92 | 28.73 | A | B |
| 111 | 2.44 | 35.96 | A | B |
| 112 | 0.653 | 26.53 | A | A |
| 113 | 7.64 | 27.22 | A | B |
| 114 | 0.672 | 32.56 | A | B |
| 115 | 0.479 | 21.89 | A | A |
| 116 | 1.59 | 31.15 | A | B |
| 117 | 1.9 | 20.5 | A | A |
| 118 | 1.55 | 41.97 | B | B |
| 119 | 1.08 | 36.41 | A | B |
| 120 | 0.276 | 16.57 | B | A |
| 121 | 1.56 | 38.54 | B | B |
| 122 | 2.71 | 26.22 | A | A |
| 123 | 6.67 | 25.23 | A | A |
| 124 | 11.74 | 18.78 | A | A |
| 125 | 0.683 | 18.25 | A | A |
| 126 | 3.08 | 42.02 | B | B |
| 127 | 1.6 | 30.45 | B | B |
| 128 | 0.397 | 15.68 | A | A |
| 129 | 0.931 | 14.07 | A | A |
| 130 | 1.61 | 40.52 | B | B |
| 131 | 0.756 | 22.47 | B | A |
| 132 | 3.24 | 19.47 | A | A |
| 133 | 2.8 | 34.47 | A | B |
| 134 | 3.09 | 43.25 | A | B |
| 135 | 0.351 | 22.58 | A | A |
| 136 | 0.565 | 16.11 | A | A |
| 137 | 0.849 | 22.07 | B | B |
| 138 | 0.195 | 17.98 | A | A |
| 139 | 2.24 | 34.89 | A | A |
| 140 | 3.1 | 25.85 | A | A |
| 141 | 0.285 | 17.59 | A | A |
| 142 | 0.657 | 16.4 | A | A |
| 143 | 3.97 | 22.37 | A | B |
| 144 | 2.68 | 23.26 | A | A |
| 145 | 1.61 | 23.71 | A | A |
| 146 | 6.54 | 30.86 | B | B |
| 147 | 1.89 | 15.93 | A | A |
| 148 | 1.82 | 15.43 | A | A |
| 149 | 0.618 | 30.8 | A | A |
| 150 | 1.31 | 30.15 | B | A |
| 151 | 11.2 | 23.51 | A | A |
| 152 | 1.35 | 36.08 | B | B |
| 153 | 1.6 | 47.43 | B | B |
| 154 | 0.617 | 24.51 | A | A |
| 155 | 9.46 | 30.26 | B | B |
| 156 | 1.85 | 55.46 | B | B |
| 157 | 4.01 | 18.8 | A | A |
| 158 | 2.55 | 31.18 | B | B |
| 159 | 1.55 | 15.05 | A | A |
| 160 | 3.18 | 25.41 | A | A |
| 161 | 7 | 26.66 | A | A |
| 162 | 0.926 | 18.2 | B | A |
| 163 | 1.07 | 27.41 | A | A |
| 164 | 1.24 | 25.3 | A | A |
| 165 | 2.66 | 28.4 | A | A |
| 166 | 8.43 | 52.09 | A | A |
| 167 | 10.24 | 20.68 | ND | ND |
| 168 | 2.24 | 37.25 | ND | ND |
| 169 | 1.40 | 24.08 | ND | ND |
| 170 | 1.54 | 42.82 | ND | ND |
| 171 | 1.24 | 48.06 | ND | ND |
| 172 | 1.98 | 55.13 | ND | ND |
| 173 | 0.33 | 31.15 | ND | ND |
| 174 | 1.24 | 47.65 | ND | ND |
| 175 | 1.22 | 20.52 | ND | ND |
| 176 | 1.09 | 40.89 | ND | ND |
| 177 | 2.28 | 54.8 | ND | ND |
| 178 | 8.11 | 27.73 | ND | ND |
| 179 | 2.00 | 33.61 | ND | ND |
| 180 | 5.83 | 24.71 | ND | ND |
| 181 | 0.70 | 24.16 | ND | ND |
| 182 | 1.55 | 37.46 | ND | ND |
| 183 | 2.49 | 43.65 | ND | ND |
| 184 | 1.23 | 42.17 | ND | ND |
| 185 | 5.39 | 22.68 | ND | ND |
| 186 | 0.29 | 34.07 | ND | ND |
| 187 | 2.33 | 21.99 | ND | ND |
| 188 | 0.47 | 28.3 | ND | ND |
| 189 | 2.72 | 32.95 | ND | ND |
| 190 | 0.82 | 20.58 | ND | ND |
| 191 | 1.17 | 46.54 | ND | ND |
| 192 | 1.69 | 47.09 | ND | ND |
| 193 | 0.52 | 23.15 | ND | ND |
| 194 | 2.58 | 29.55 | ND | ND |
| 195 | 6.67 | 39.68 | ND | ND |
| 196 | 1.54 | 56.75 | ND | ND |
| 197 | 7.62 | 39.86 | ND | ND |
| 198 | 1.37 | 76.04 | ND | ND |
| 199 | 1.82 | 36.36 | ND | ND |
| 200 | 2.51 | 25.04 | ND | ND |
| 201 | 2.22 | 27.95 | ND | ND |
| 202 | 4.34 | 26.02 | ND | ND |
| 203 | 2.87 | 20.92 | ND | ND |
| 204 | 0.98 | 38.78 | ND | ND |
| 205 | 7.09 | 40.29 | ND | ND |
| 206 | 6.78 | 33.98 | ND | ND |
| 207 | 10.16 | 23.90 | ND | ND |
| 208 | 8.15 | 23.96 | ND | ND |
| 209 | 9.70 | 37.49 | ND | ND |
| 210 | 4.54 | 27.31 | ND | ND |
| 211 | 3.13 | 22.62 | ND | ND |
| 212 | 1.49 | 41.94 | ND | ND |
| 213 | 0.68 | 31.5 | ND | ND |
| 214 | 8.95 | 30.24 | ND | ND |
| 215 | 4.17 | 24.17 | ND | ND |
| 216 | 1.40 | 49.32 | ND | ND |
| 217 | 1.76 | 29.44 | ND | ND |
| 218 | 8.00 | 21.41 | ND | ND |
| 219 | 2.26 | 35.95 | ND | ND |
| 220 | 11.84 | 29.83 | ND | ND |
| 221 | 3.20 | 35.15 | ND | ND |
| 222 | 4.16 | 28.1 | ND | ND |
| 223 | 7.40 | 41.12 | ND | ND |
| 224 | 3.43 | 37.09 | ND | ND |
| 225 | 6.41 | 42.13 | ND | ND |
| 226 | 1.83 | 22.4 | ND | ND |
| 227 | 1.86 | 31.71 | ND | ND |
| 228 | 0.97 | 20.27 | ND | ND |
| 229 | 12.65 | 76.14 | ND | ND |
| 230 | 1.77 | 20.02 | ND | ND |
| 231 | >30 | 49.96 | ND | ND |
| 232 | 1.43 | 23.95 | ND | ND |
| 233 | 2.52 | 28.00 | ND | ND |
| 234 | 5.26 | 27.37 | ND | ND |
| 235 | 6.22 | 21.79 | ND | ND |
| 236 | 11.88 | 31.52 | ND | ND |
| 237 | 11.71 | 29.76 | ND | ND |
| 238 | 2.91 | 24.31 | ND | ND |
| 239 | 5.10 | 20.11 | ND | ND |
| 240 | 11.70 | 46.27 | ND | ND |
| 241 | 3.32 | 36.71 | ND | ND |
| 242 | 2.98 | 34.51 | ND | ND |
| 243 | 3.73 | 26.56 | ND | ND |
| 244 | 7.59 | 26.21 | ND | ND |
| 245 | 2.19 | 46.39 | ND | ND |
| 246 | 1.76 | 22.48 | ND | ND |
| 247 | 5.05 | 24.56 | ND | ND |
| 248 | 2.61 | 23.91 | ND | ND |
| 249 | 3.42 | 26.44 | ND | ND |
| 250 | 2.58 | 21.04 | ND | ND |

The obtained EC50 values showed that all the tested compounds have an ability to activate Kv7.2/7.3 potassium channels. Most of them are also selective versus the Kv7.4 and/or Kv7.3/7.5 potassium channels.

1: A method of treating a disorder modulated by a KV7.2/KV7.3 potassium channel, comprising administering to a subject in need thereof a Kv7.2/7.3 potassium channel activator compound, or a pharmaceutically acceptable salt thereof having the following formula (I): 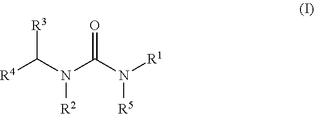
R1 is represented by -L1-A1, wherein
L1 is a bond or a linear or branched C1-C6 alkyl chain, optionally substituted by one or more halogen atom or hydroxyl group, and optionally comprising an oxy (—O—) group within or at any end of the alkyl chain, and
A1 is
(i) an aromatic or aliphatic ring having three to six members, optionally containing one or more heteroatoms selected from the group consisting of N, O and S, unsubstituted or substituted with one or more substituents selected from the group consisting of halogen, hydroxyl, methoxy, CN, CF₃, C1-C3 alkyl chain optionally substituted by one or more halogen atom, and AR, wherein AR is an aromatic or aliphatic ring, optionally containing one or more heteroatoms selected from the group consisting of N, O and S, unsubstituted or substituted with one or more substituents selected from the group consisting of halogen, hydroxyl, methoxy, CN, CF₃, and C1-C3 alkyl chain optionally substituted by one or more halogen atom, or
(ii) a fused or bridged bicyclic ring having five to twelve members, optionally containing one or more heteroatoms selected from the group consisting of O and S, unsubstituted or substituted with one or more substituents selected from the group consisting of halogen, hydroxyl, methoxy, CN, CF₃, O-Gr, and C1-C3 alkyl optionally substituted by one or more halogen atom, wherein Gr is an aliphatic ring having three to six members, optionally containing one or more heteroatoms selected from the group consisting of N, O and S, unsubstituted or substituted with one or more substituents selected from the group consisting of halogen, hydroxyl, methoxy, CN, CF₃, C1-C3 alkyl chain, optionally substituted by one or more halogen atom;
R2 is hydrogen, or a C1-C3 alkyl chain;
R3 is hydrogen or a C1-C3 alkyl chain optionally substituted by hydroxyl group, or an aliphatic ring, optionally containing one or more heteroatoms selected from the group consisting of N, O and S, unsubstituted or substituted with one or more substituents selected from the group consisting of halogen, hydroxyl, methoxy, CN, and CF₃;
R4 is represented by -L2-A2, wherein
L2 is a bond or a C1-C3 alkyl chain, and
A2 is an aromatic ring having 6 members and comprising one or two nitrogen atoms, optionally condensed with an aliphatic ring having 5 or 6 carbon atoms, said aromatic ring being substituted by one or more substituent selected from
(i) a halogen atom,
(ii) a C1-C3 alkyl chain, optionally substituted with one or more halogen atoms,
(iii) an aliphatic ring having 4 to 6 members comprising one or more heteroatoms selected from O and N, optionally substituted with one or more halogen atom or C1-C3 alkyl chain optionally substituted with one or more halogen or hydroxyl, and
(iv) a group represented by the following formula —S6-L3-A3, wherein S6 is an oxygen or nitrogen atom, L3 is a bond or a C1-C4 alkyl chain, and A3 is
(a) an aliphatic or aromatic ring having 3 to 6 members, optionally comprising one or more heteroatoms selected from O and N, and optionally substituted with a halogen atom or a C1-C3 alkyl chain, optionally substituted with one or more halogen, hydroxyl, or CN, or
(b) a C1-C3 alkyl chain, optionally substituted with one or more halogen atoms,
R5 is hydrogen, or a C1-C3 alkyl chain;
or R4 together with R2 or R3, and/or R5 together with R1, and/or R3 together with R2, form a five to six member aliphatic ring optionally substituted by an alkyl chain having 1-3 carbon atoms, a halogen atom, or a hydroxyl group, said aliphatic ring optionally comprising an oxygen atom and being optionally condensed with or substituted by a benzene or pyridine ring, said benzene or pyridine ring being optionally substituted by a halogen, or an alkyl or alkoxy chain having 1-3 carbon atoms, said chain being optionally substituted by one or more halogen atom;
provided that when R2, R3 and R5 are hydrogen atoms and R4 is 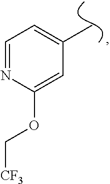
then R1 is different from 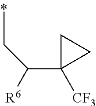
where R6 is H or OH, and
provided that when L1 is a bond, A1 is not a substituted or unsubstituted cyclobutane ring.

2: The method according to claim 1, wherein A1 is hydrogen, or
(i) an aromatic or aliphatic ring selected from the group consisting of phenyl, cyclopropyl, cyclobutyl, cyclopentyl, and cyclohexyl, optionally substituted with one or more substituents selected from the group consisting of halogen, hydroxyl, methoxy, CN, CF₃, C1-C3 alkyl chain, optionally substituted by one or more halogen atom, or
(ii) a bicyclic ring selected from the group consisting of bicycle[1,1,1]pentane, indane (2,3-dihydro-1H-indene), 2,3-dihydro-1H-indole, 6,7-dihydro-5H-cyclopenta[c]pyridine, chromane (3,4-dihydro-2H-1-benzopyran), coumaran (2,3-dihydro-1-benzofuran), tetralin (1,2,3,4-tetrahydronaphthalene), thiochroman (3,4-dihydro-2H-1-benzothiopyran), 5,6-dihydro-4H-cyclopenta[b]thiophene, 6,7-dihydro-5H-cyclopenta[b]pyridine, 5,6,7,8-tetrahydroisoquinoline, 5,6,7,8-tetrahydroquinoxaline, 2,3,4,5-tetrahydro-1-benzoxepine, and 6,7,8,9-tetrahydro-5H-benzo[7]annulene, optionally substituted with one or more substituents selected from the group consisting of halogen, hydroxyl, methoxy, CN, CF₃, C1-C3 alkyl chain, optionally substituted by one or more halogen atom.

3: The method according to claim 1, wherein A2 is pyridine, pyridazine, pyrimidine, or pyrazine, optionally condensed with an aliphatic ring having 5 or 6 carbon atoms.

4: The method according to claim 1, wherein A2 is substituted by an aliphatic ring selected from the group consisting of azetidine, pyrrolidine, piperidine, oxetane, tetrahydrofuran, tetrahydropyran oxazetidine, oxazolidine, or morpholine, optionally substituted with one or more halogen atom or C1-C3 alkyl chain optionally substituted with one or more halogen atoms.

5: The method according to claim 1, wherein A3 is an aliphatic ring having 3 to 6 members or an aromatic ring having 5 or 6 member, optionally containing one or more heteroatoms selected from O and N, and optionally substituted with a halogen atom or a C1-C3 alkyl chain, optionally substituted with one or more halogen, hydroxyl, CN.

6: The method according to claim 1, wherein A3 is cyclopropane, cyclobutane, azetidine, oxetane or tetrahydropyran.

7: The method according to claim 1, wherein A3 is phenyl, pyrrole, furan, oxazole, pyrimidine, or pyridine.

8: The method according to claim 1, wherein said pharmaceutically acceptable salt is selected from the group consisting of a salt with an organic acid, a salt with an organic base, a salt with an inorganic acid, and a salt with an inorganic base.

9: The method according to claim 1, wherein, said disorder which is modulated by a Kv7.2/7.3 potassium channel is a central nervous system (CNS) or peripheral nervous system (PNS) disorder.

10: The method according to claim 9, wherein said central nervous system (CNS) disorder is selected from the group consisting of epilepsy, epileptic syndrome, epileptic symptom, epilepsy resistant or refractory to treatment, a seizure, bipolar disorder, bipolar depression, schizophrenia, psychosis, mania, a stress-related disorder, an acute stress reaction, major depressive disorder, anxiety, a panic attack, social phobia, a sleep disorder, attention deficit hyperactivity disorder, post-traumatic stress disorder, obsessive-compulsive disorder, an impulsivity disorder, a personality disorder, Huntington's disease, Alzheimer's disease, Parkinson's disease, multiple sclerosis, amyotrophic lateral sclerosis, and tinnitus.

11: The method according to claim 9, wherein said peripheral nervous system (PNS) disorder is selected from the group consisting of migraine, chronic pain, acute pain, neuropathic pain, visceral pain, inflammatory pain, and muscle pain.

12: A Kv7.2/7.3 potassium channel activator compound or a pharmaceutically acceptable salt thereof, wherein said compound is selected from the group consisting of the compounds of the following Table A and Table B,
TABLE A

| No. | Structure |
|---|---|
| 1 | 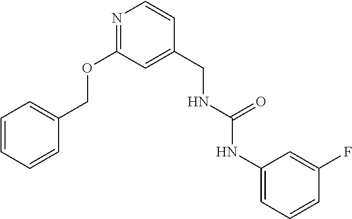 [SMILES: O=C(NCc1ccnc(OCc2ccccc2)c1)Nc1cccc(F)c1] |
| 2 | 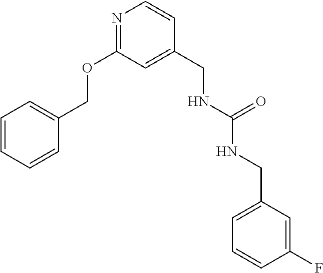 [SMILES: O=C(NCc1cccc(F)c1)NCc1ccnc(OCc2ccccc2)c1] |
| 3 | 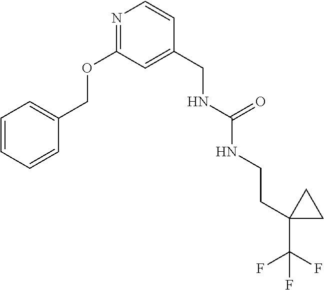 [SMILES: O=C(NCCC1(C(F)(F)F)CC1)NCc1ccnc(OCc2ccccc2)c1] |
| 4 | 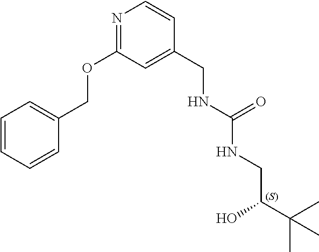 |
| 5 | 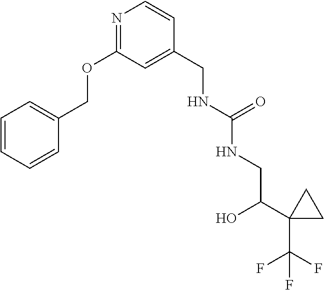 [SMILES: O=C(NCc1ccnc(OCc2ccccc2)c1)NCC(O)C1(C(F)(F)F)CC1] |
| 6 | 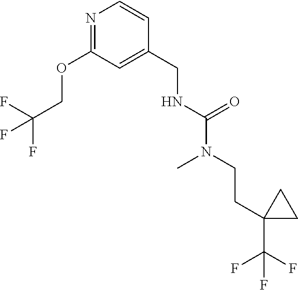 [SMILES: CN(CCC1(C(F)(F)F)CC1)C(=O)NCc1ccnc(OCC(F)(F)F)c1] |
| 7 | 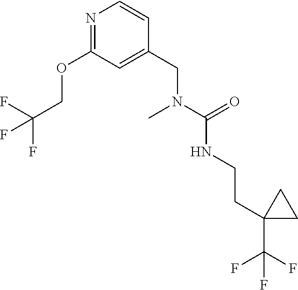 [SMILES: CN(Cc1ccnc(OCC(F)(F)F)c1)C(=O)NCCC1(C(F)(F)F)CC1] |
| 8 | 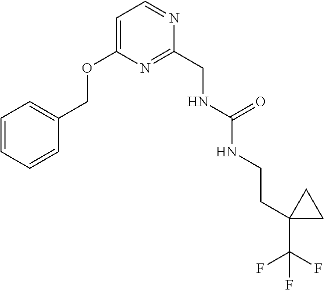 [SMILES: O=C(NCCC1(C(F)(F)F)CC1)NCc1nccc(OCc2ccccc2)n1] |
| 9 | 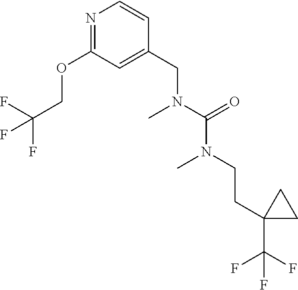 [SMILES: CN(CCC1(C(F)(F)F)CC1)C(=O)N(C)Cc1ccnc(OCC(F)(F)F)c1] |
| 10 | 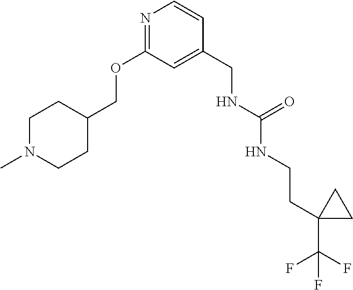 [SMILES: CN1CCC(COc2cc(CNC(=O)NCCC3(C(F)(F)F)CC3)ccn2)CC1] |
| 11 | 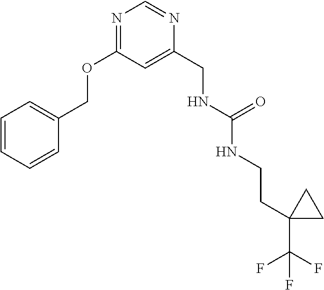 [SMILES: O=C(NCCC1(C(F)(F)F)CC1)NCc1cc(OCc2ccccc2)ncn1] |
| 12 | 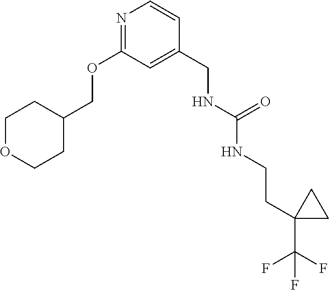 [SMILES: O=C(NCCC1(C(F)(F)F)CC1)NCc1ccnc(OCC2CCOCC2)c1] |
| 13 | 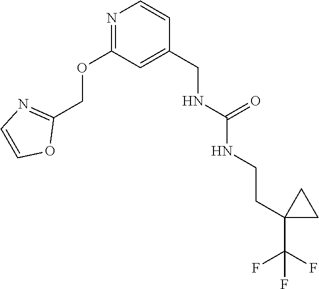 [SMILES: O=C(NCCC1(C(F)(F)F)CC1)NCc1ccnc(OCc2ncco2)c1] |
| 14 | 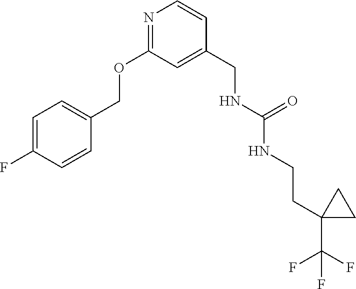 [SMILES: O=C(NCCC1(C(F)(F)F)CC1)NCc1ccnc(OCc2ccc(F)cc2)c1] |
| 15 | 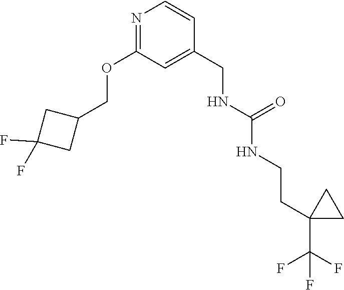 [SMILES: O=C(NCCC1(C(F)(F)F)CC1)NCc1ccnc(OCC2CC(F)(F)C2)c1] |
| 16 | 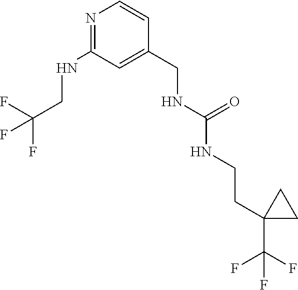 [SMILES: O=C(NCCC1(C(F)(F)F)CC1)NCc1ccnc(NCC(F)(F)F)c1] |
| 17 | 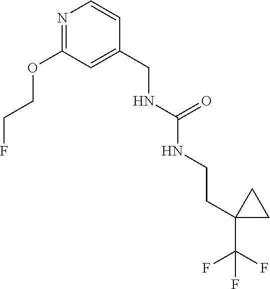 [SMILES: O=C(NCCC1(C(F)(F)F)CC1)NCc1ccnc(OCCF)c1] |
| 18 | 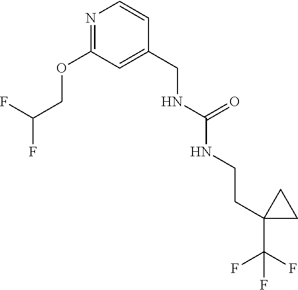 [SMILES: O=C(NCCC1(C(F)(F)F)CC1)NCc1ccnc(OCC(F)F)c1] |
| 19 | 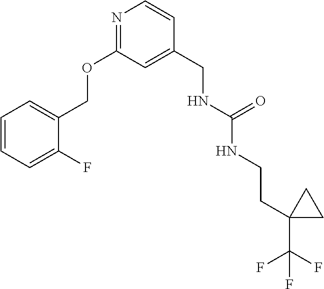 [SMILES: O=C(NCCC1(C(F)(F)F)CC1)NCc1ccnc(OCc2ccccc2F)c1] |
| 20 | 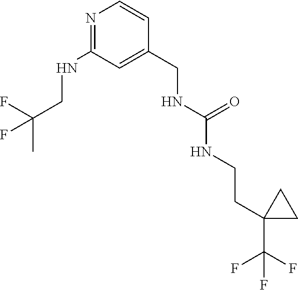 [SMILES: CC(F)(F)CNc1cc(CNC(=O)NCCC2(C(F)(F)F)CC2)ccn1] |
| 21 | 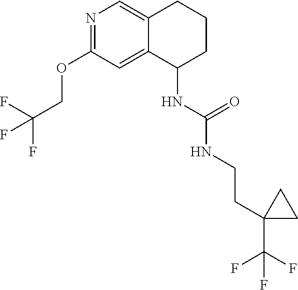 [SMILES: O=C(NCCC1(C(F)(F)F)CC1)NC1CCCc2cnc(OCC(F)(F)F)cc21] |
| 22 | 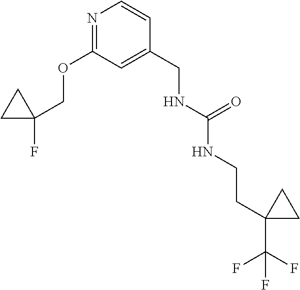 [SMILES: O=C(NCCC1(C(F)(F)F)CC1)NCc1ccnc(OCC2(F)CC2)c1] |
| 23 | 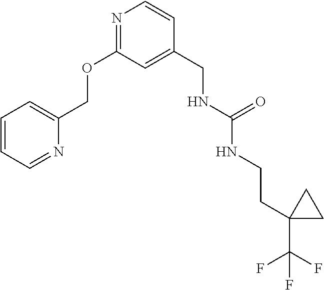 [SMILES: O=C(NCCC1(C(F)(F)F)CC1)NCc1ccnc(OCc2ccccn2)c1] |
| 24 | 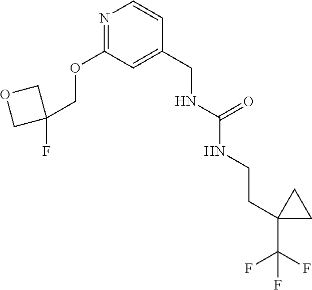 [SMILES: O=C(NCCC1(C(F)(F)F)CC1)NCc1ccnc(OCC2(F)COC2)c1] |
| 25 | 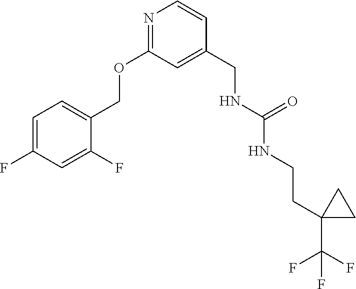 [SMILES: O=C(NCCC1(C(F)(F)F)CC1)NCc1ccnc(OCc2ccc(F)cc2F)c1] |
| 26 | 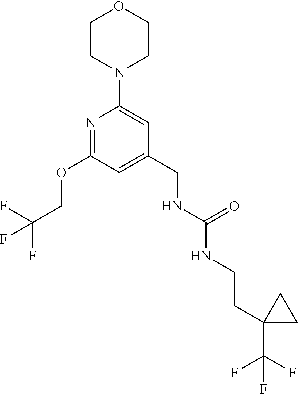 [SMILES: O=C(NCCC1(C(F)(F)F)CC1)NCc1cc(OCC(F)(F)F)nc(N2CCOCC2)c1] |
| 27 | 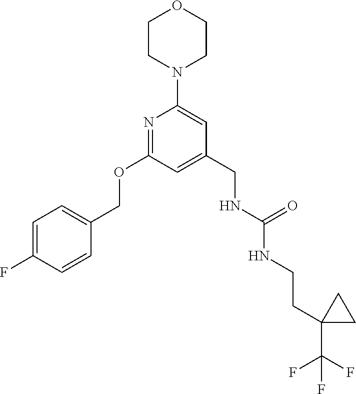 [SMILES: O=C(NCCC1(C(F)(F)F)CC1)NCc1cc(OCc2ccc(F)cc2)nc(N2CCOCC2)c1] |
| 28 | 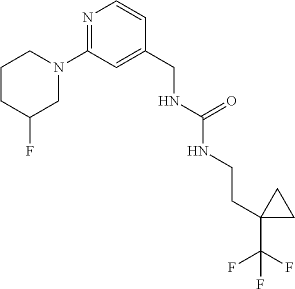 [SMILES: O=C(NCCC1(C(F)(F)F)CC1)NCc1ccnc(N2CCCC(F)C2)c1] |
| 29 | 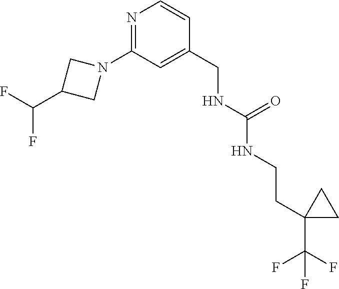 [SMILES: O=C(NCCC1(C(F)(F)F)CC1)NCc1ccnc(N2CC(C(F)F)C2)c1] |
| 30 | 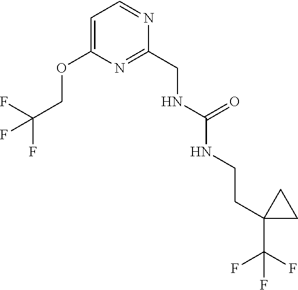 [SMILES: O=C(NCCC1(C(F)(F)F)CC1)NCc1nccc(OCC(F)(F)F)n1] |
| 31 | 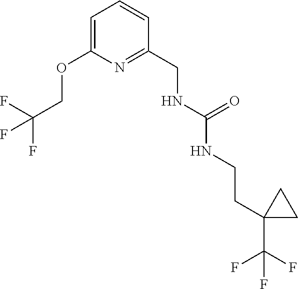 [SMILES: O=C(NCCC1(C(F)(F)F)CC1)NCc1cccc(OCC(F)(F)F)n1] |
| 32 | 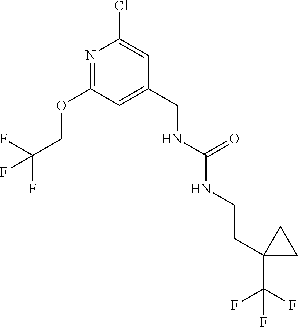 [SMILES: O=C(NCCC1(C(F)(F)F)CC1)NCc1cc(Cl)nc(OCC(F)(F)F)c1] |
| 33 | 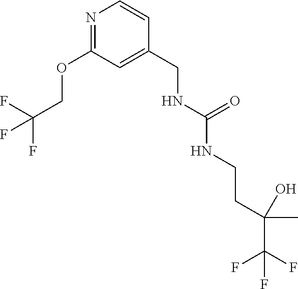 [SMILES: CC(O)(CCNC(=O)NCc1ccnc(OCC(F)(F)F)c1)C(F)(F)F] |
| 34 | 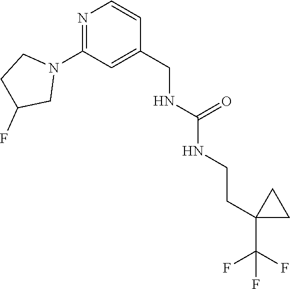 [SMILES: O=C(NCCC1(C(F)(F)F)CC1)NCc1ccnc(N2CCC(F)C2)c1] |
| 35 | 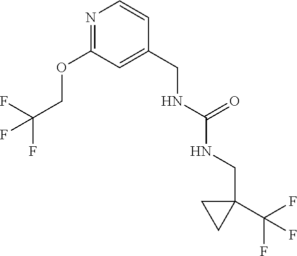 [SMILES: O=C(NCc1ccnc(OCC(F)(F)F)c1)NCC1(C(F)(F)F)CC1] |
| 36 | 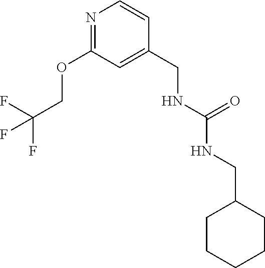 [SMILES: O=C(NCc1ccnc(OCC(F)(F)F)c1)NCC1CCCCC1] |
| 38 | 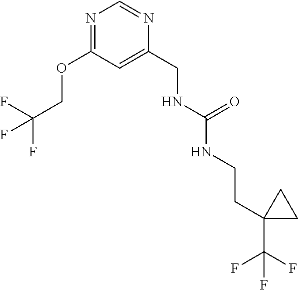 [SMILES: O=C(NCCC1(C(F)(F)F)CC1)NCc1cc(OCC(F)(F)F)ncn1] |
| 39 | 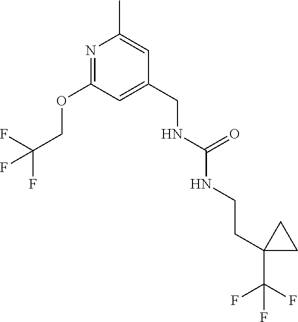 [SMILES: Cc1cc(CNC(=O)NCCC2(C(F)(F)F)CC2)cc(OCC(F)(F)F)n1] |
| 40 | 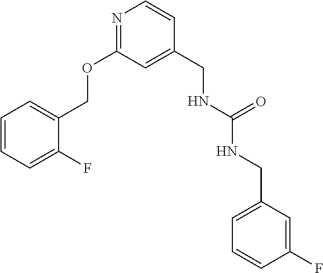 [SMILES: O=C(NCc1cccc(F)c1)NCc1ccnc(OCc2ccccc2F)c1] |
| 41 | 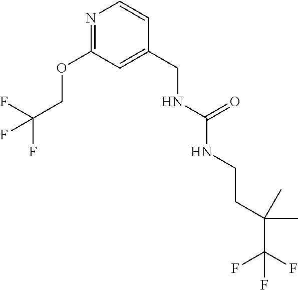 [SMILES: CC(C)(CCNC(=O)NCc1ccnc(OCC(F)(F)F)c1)C(F)(F)F] |
| 44 | 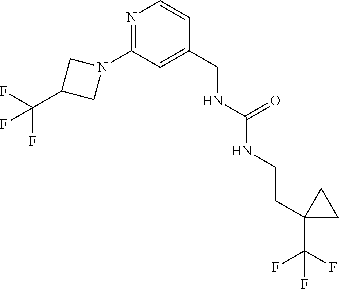 [SMILES: O=C(NCCC1(C(F)(F)F)CC1)NCc1ccnc(N2CC(C(F)(F)F)C2)c1] |
| 45 | 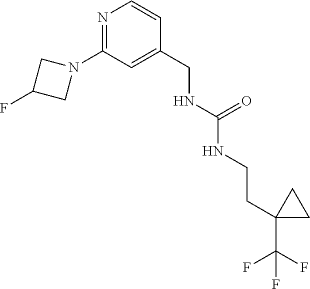 [SMILES: O=C(NCCC1(C(F)(F)F)CC1)NCc1ccnc(N2CC(F)C2)c1] |
| 46 | 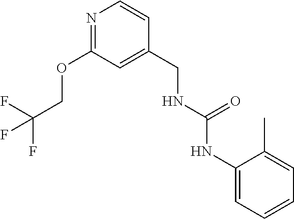 [SMILES: Cc1ccccc1NC(=O)NCc1ccnc(OCC(F)(F)F)c1] |
| 47 | 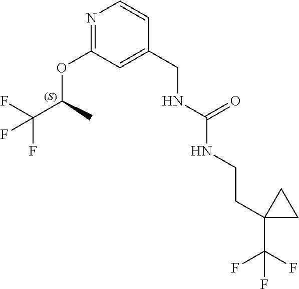 |
| 48 | 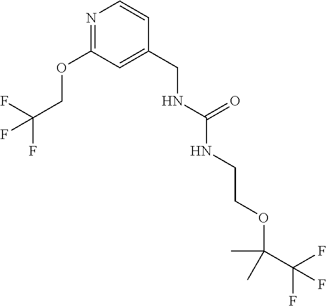 [SMILES: CC(C)(OCCNC(=O)NCc1ccnc(OCC(F)(F)F)c1)C(F)(F)F] |
| 49 | 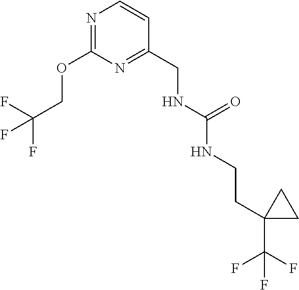 [SMILES: O=C(NCCC1(C(F)(F)F)CC1)NCc1ccnc(OCC(F)(F)F)n1] |
| 50 | 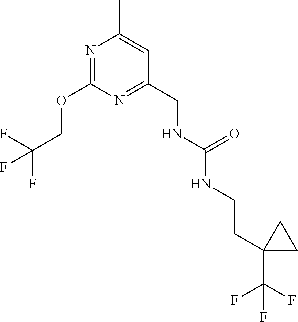 [SMILES: Cc1cc(CNC(=O)NCCC2(C(F)(F)F)CC2)nc(OCC(F)(F)F)n1] |
| 51 | 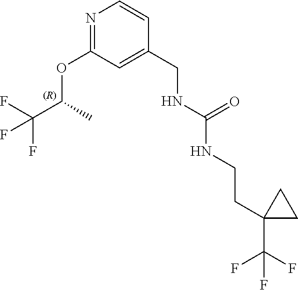 |
| 52 | 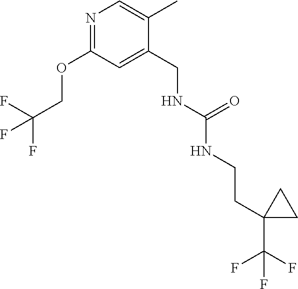 [SMILES: Cc1cnc(OCC(F)(F)F)cc1CNC(=O)NCCC1(C(F)(F)F)CC1] |
| 53 | 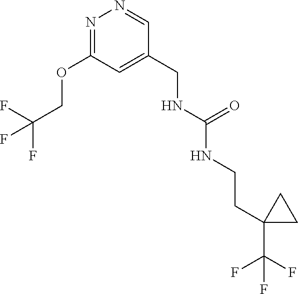 [SMILES: O=C(NCCC1(C(F)(F)F)CC1)NCc1cnnc(OCC(F)(F)F)c1] |
| 54 | 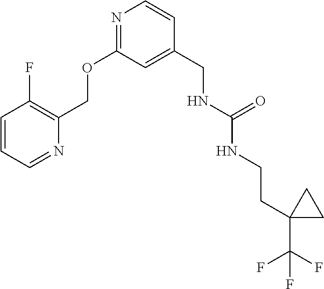 [SMILES: O=C(NCCC1(C(F)(F)F)CC1)NCc1ccnc(OCc2ncccc2F)c1] |
| 55 | 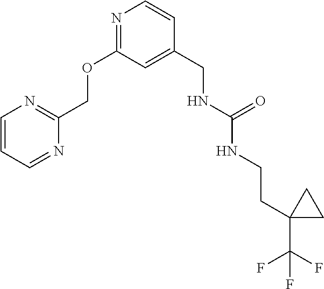 [SMILES: O=C(NCCC1(C(F)(F)F)CC1)NCc1ccnc(OCc2ncccn2)c1] |
| 56 | 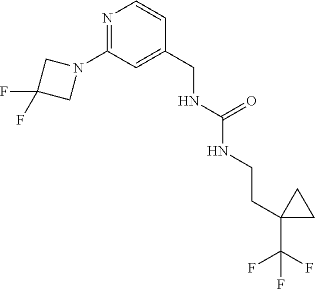 [SMILES: O=C(NCCC1(C(F)(F)F)CC1)NCc1ccnc(N2CC(F)(F)C2)c1] |
| 57 | 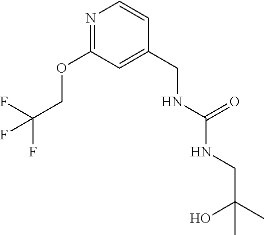 [SMILES: CC(C)(O)CNC(=O)NCc1ccnc(OCC(F)(F)F)c1] |
| 58 | 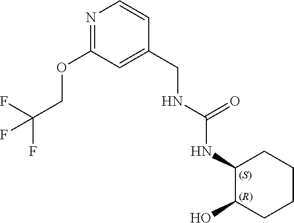 |
| 59 | 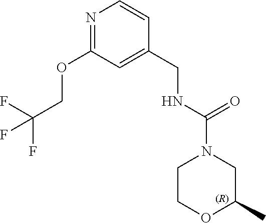 |
| 60 | 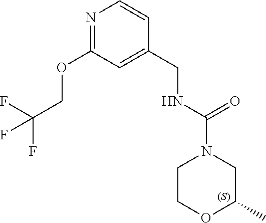 |
| 61 | 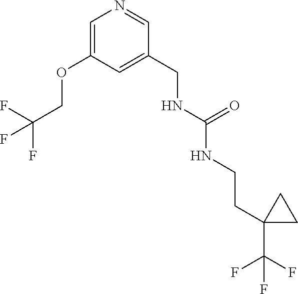 [SMILES: O=C(NCCC1(C(F)(F)F)CC1)NCc1cncc(OCC(F)(F)F)c1] |
| 62 | 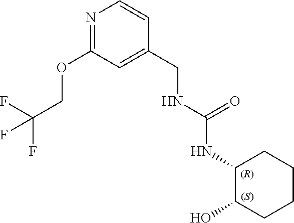 |
| 63 | 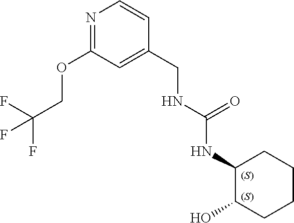 |
| 64 | 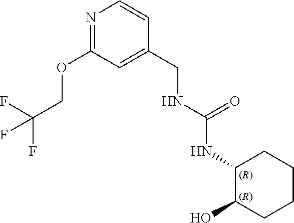 |
| 65 | 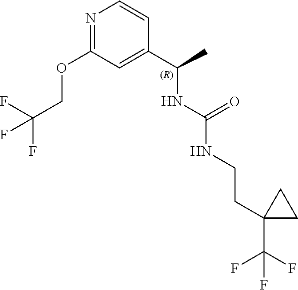 |
| 66 | 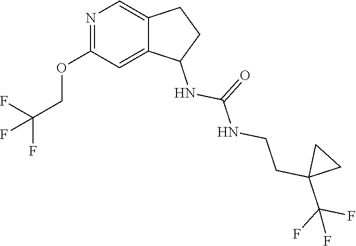 [SMILES: O=C(NCCC1(C(F)(F)F)CC1)NC1CCc2cnc(OCC(F)(F)F)cc21] |
| 67 | 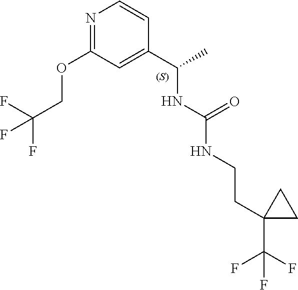 |
| 68 | 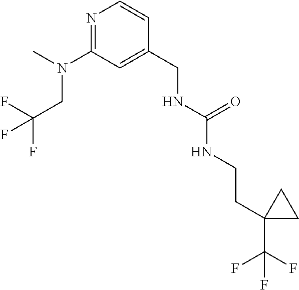 [SMILES: CN(CC(F)(F)F)c1cc(CNC(=O)NCCC2(C(F)(F)F)CC2)ccn1] |
| 69 | 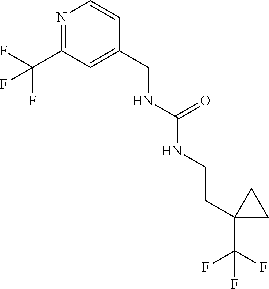 [SMILES: O=C(NCCC1(C(F)(F)F)CC1)NCc1ccnc(C(F)(F)F)c1] |
| 70 | 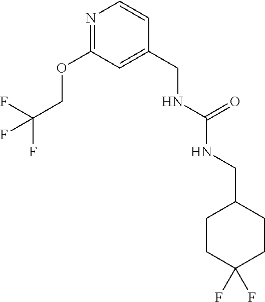 [SMILES: O=C(NCc1ccnc(OCC(F)(F)F)c1)NCC1CCC(F)(F)CC1] |
| 71 | 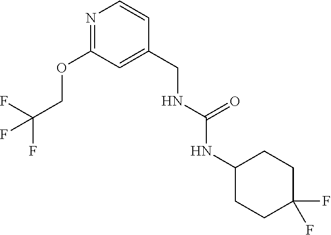 [SMILES: O=C(NCc1ccnc(OCC(F)(F)F)c1)NC1CCC(F)(F)CC1] |
| 72 | 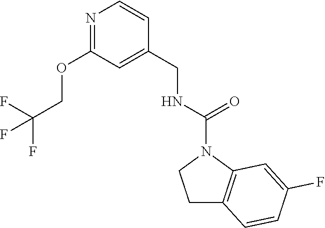 [SMILES: O=C(NCc1ccnc(OCC(F)(F)F)c1)N1CCc2ccc(F)cc21] |
| 73 | 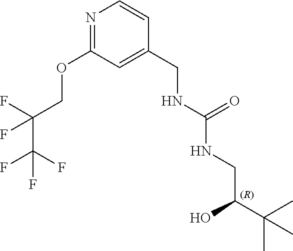 |
| 74 | 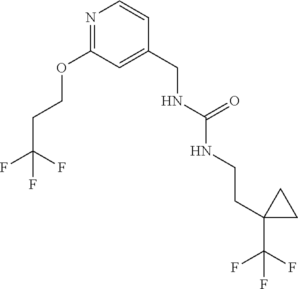 [SMILES: O=C(NCCC1(C(F)(F)F)CC1)NCc1ccnc(OCCC(F)(F)F)c1] |
| 75 | 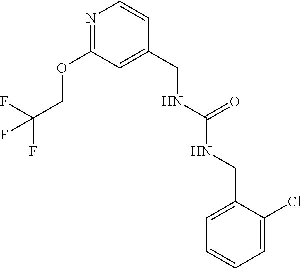 [SMILES: O=C(NCc1ccnc(OCC(F)(F)F)c1)NCc1ccccc1Cl] |
| 76 | 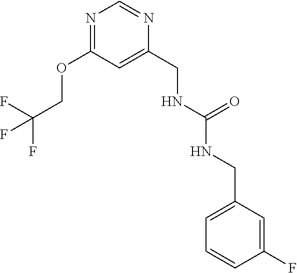 [SMILES: O=C(NCc1cccc(F)c1)NCc1cc(OCC(F)(F)F)ncn1] |
| 77 | 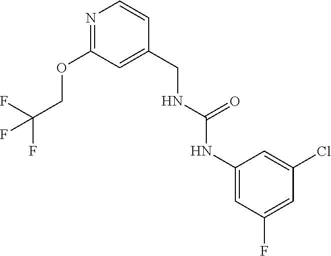 [SMILES: O=C(NCc1ccnc(OCC(F)(F)F)c1)Nc1cc(F)cc(Cl)c1] |
| 78 | 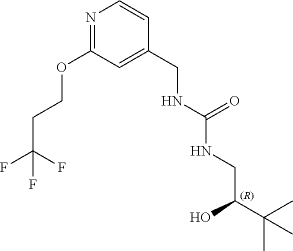 |
| 79 | 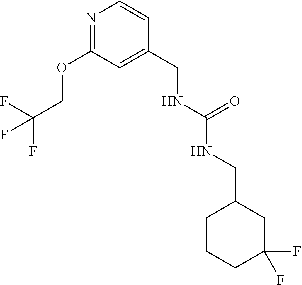 [SMILES: O=C(NCc1ccnc(OCC(F)(F)F)c1)NCC1CCCC(F)(F)C1] |
| 80 | 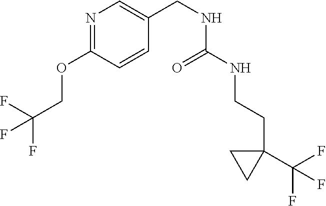 [SMILES: O=C(NCCC1(C(F)(F)F)CC1)NCc1ccc(OCC(F)(F)F)nc1] |
| 81 | 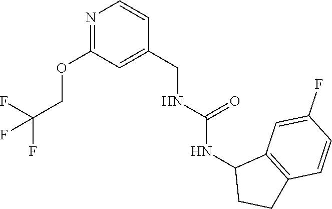 [SMILES: O=C(NCc1ccnc(OCC(F)(F)F)c1)NC1CCc2ccc(F)cc21] |
| 82 | 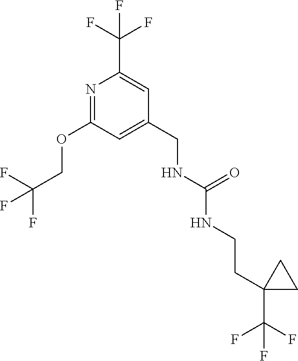 [SMILES: O=C(NCCC1(C(F)(F)F)CC1)NCc1cc(OCC(F)(F)F)nc(C(F)(F)F)c1] |
| 83 | 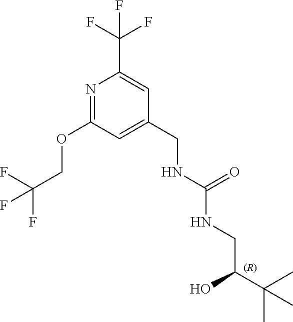 |
| 84 | 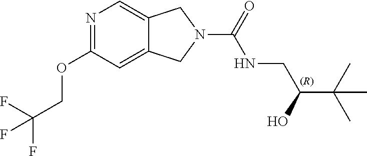 |
| 85 | 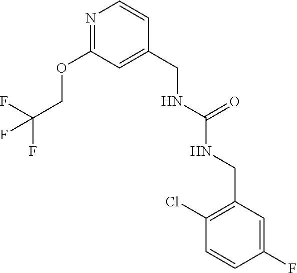 [SMILES: O=C(NCc1ccnc(OCC(F)(F)F)c1)NCc1cc(F)ccc1Cl] |
| 86 | 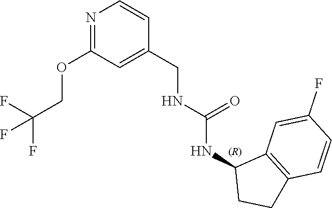 |
| 87 | 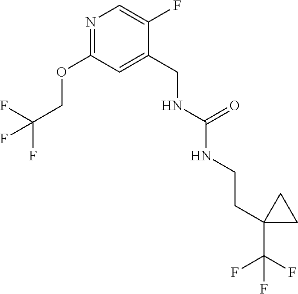 [SMILES: O=C(NCCC1(C(F)(F)F)CC1)NCc1cc(OCC(F)(F)F)ncc1F] |
| 88 | 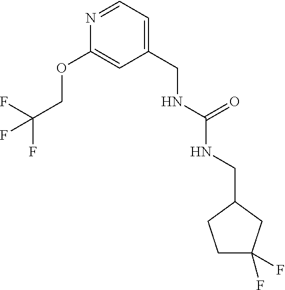 [SMILES: O=C(NCc1ccnc(OCC(F)(F)F)c1)NCC1CCC(F)(F)C1] |
| 89 | 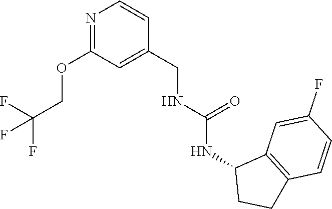 [SMILES: O=C(NCc1ccnc(OCC(F)(F)F)c1)N[C@H]1CCc2ccc(F)cc21] |
| 90 | 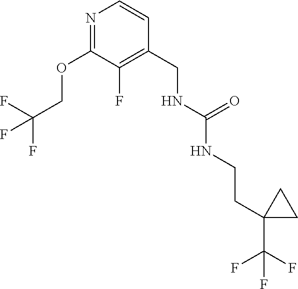 [SMILES: O=C(NCCC1(C(F)(F)F)CC1)NCc1ccnc(OCC(F)(F)F)c1F] |
| 91 | 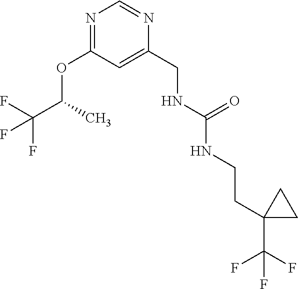 [SMILES: C[C@@H](Oc1cc(CNC(=O)NCCC2(C(F)(F)F)CC2)ncn1)C(F)(F)F] |
| 92 | 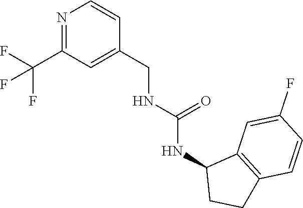 [SMILES: O=C(NCc1ccnc(C(F)(F)F)c1)N[C@@H]1CCc2ccc(F)cc21] |
| 93 | 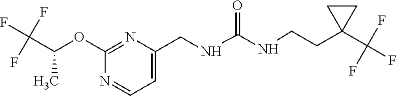 [SMILES: C[C@@H](Oc1nccc(CNC(=O)NCCC2(C(F)(F)F)CC2)n1)C(F)(F)F] |
| 94 | 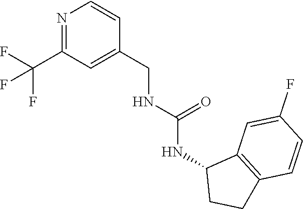 [SMILES: O=C(NCc1ccnc(C(F)(F)F)c1)N[C@H]1CCc2ccc(F)cc21] |
| 95 | 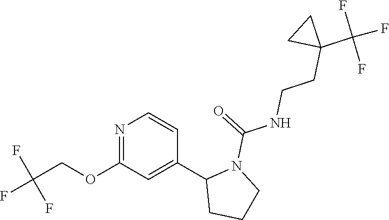 [SMILES: O=C(NCCC1(C(F)(F)F)CC1)N1CCCC1c1ccnc(OCC(F)(F)F)c1] |
| 96 | 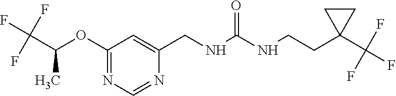 [SMILES: C[C@H](Oc1cc(CNC(=O)NCCC2(C(F)(F)F)CC2)ncn1)C(F)(F)F] |
| 97 | 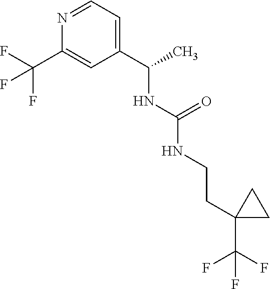 [SMILES: C[C@H](NC(=O)NCCC1(C(F)(F)F)CC1)c1ccnc(C(F)(F)F)c1] |
| 98 | 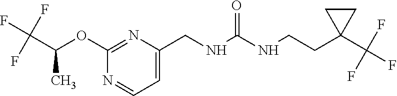 [SMILES: C[C@H](Oc1nccc(CNC(=O)NCCC2(C(F)(F)F)CC2)n1)C(F)(F)F] |
| 99 | 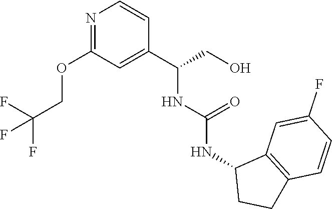 [SMILES: O=C(N[C@@H](CO)c1ccnc(OCC(F)(F)F)c1)N[C@H]1CCc2ccc(F)cc21] |
| 100 | 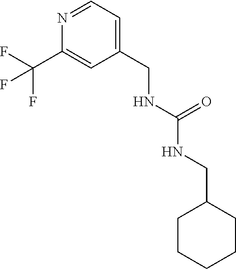 [SMILES: O=C(NCc1ccnc(C(F)(F)F)c1)NCC1CCCCC1] |
| 101 | 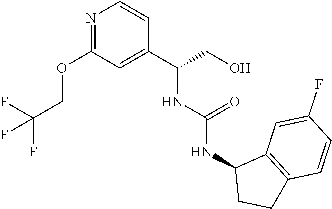 [SMILES: O=C(N[C@@H](CO)c1ccnc(OCC(F)(F)F)c1)N[C@@H]1CCc2ccc(F)cc21] |
| 102 | 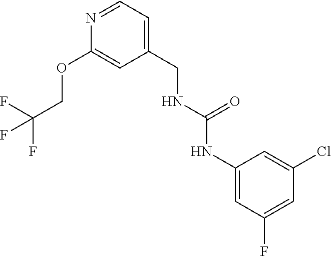 [SMILES: O=C(NCc1ccnc(OCC(F)(F)F)c1)Nc1cc(F)cc(Cl)c1] |
| 103 | 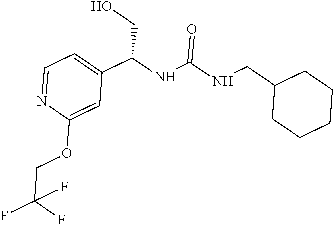 [SMILES: O=C(NCC1CCCCC1)N[C@@H](CO)c1ccnc(OCC(F)(F)F)c1] |
| 104 | 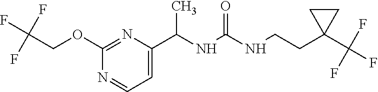 [SMILES: CC(NC(=O)NCCC1(C(F)(F)F)CC1)c1ccnc(OCC(F)(F)F)n1] |
| 105 | 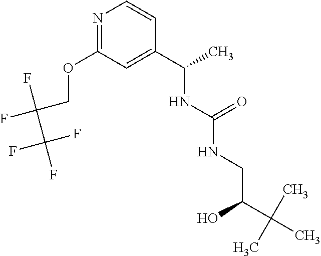 [SMILES: C[C@H](NC(=O)NC[C@H](O)C(C)(C)C)c1ccnc(OCC(F)(F)C(F)(F)F)c1] |
| 106 | 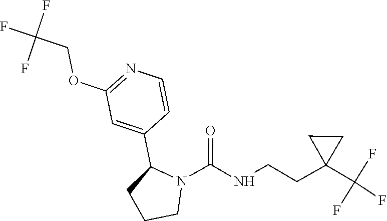 [SMILES: O=C(NCCC1(C(F)(F)F)CC1)N1CCC[C@H]1c1ccnc(OCC(F)(F)F)c1] |
| 107 | 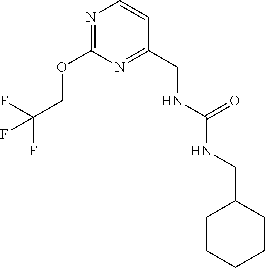 [SMILES: O=C(NCc1ccnc(OCC(F)(F)F)n1)NCC1CCCCC1] |
| 108 | 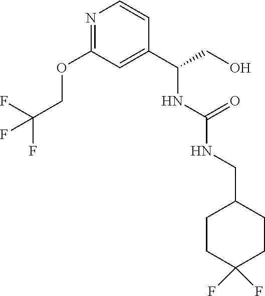 [SMILES: O=C(NCC1CCC(F)(F)CC1)N[C@@H](CO)c1ccnc(OCC(F)(F)F)c1] |
| 109 | 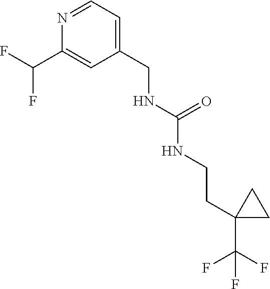 [SMILES: O=C(NCCC1(C(F)(F)F)CC1)NCc1ccnc(C(F)F)c1] |
| 110 | 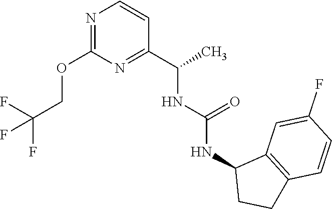 [SMILES: C[C@H](NC(=O)N[C@@H]1CCc2ccc(F)cc21)c1ccnc(OCC(F)(F)F)n1] |
| 111 | 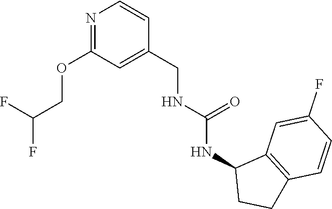 [SMILES: O=C(NCc1ccnc(OCC(F)F)c1)N[C@@H]1CCc2ccc(F)cc21] |
| 112 | 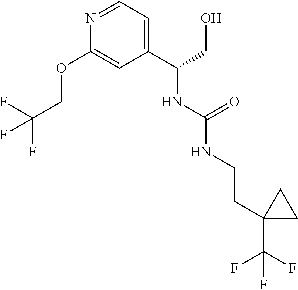 [SMILES: O=C(NCCC1(C(F)(F)F)CC1)N[C@@H](CO)c1ccnc(OCC(F)(F)F)c1] |
| 113 | 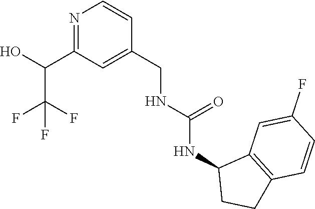 [SMILES: O=C(NCc1ccnc(C(O)C(F)(F)F)c1)N[C@@H]1CCc2ccc(F)cc21] |
| 114 | 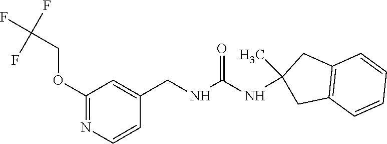 [SMILES: CC1(NC(=O)NCc2ccnc(OCC(F)(F)F)c2)Cc2ccccc2C1] |
| 115 | 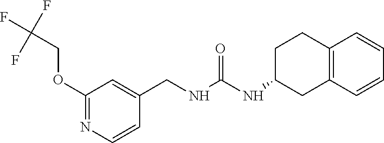 [SMILES: O=C(NCc1ccnc(OCC(F)(F)F)c1)N[C@@H]1CCc2ccccc2C1] |
| 116 | 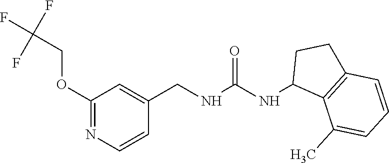 [SMILES: Cc1cccc2c1C(NC(=O)NCc1ccnc(OCC(F)(F)F)c1)CC2] |
| 117 | 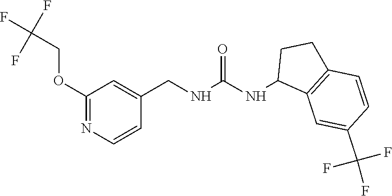 [SMILES: O=C(NCc1ccnc(OCC(F)(F)F)c1)NC1CCc2ccc(C(F)(F)F)cc21] |
| 118 | 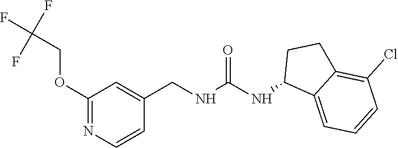 [SMILES: O=C(NCc1ccnc(OCC(F)(F)F)c1)N[C@@H]1CCc2c(Cl)cccc21] |
| 119 | 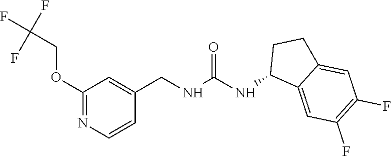 [SMILES: O=C(NCc1ccnc(OCC(F)(F)F)c1)N[C@@H]1CCc2cc(F)c(F)cc21] |
| 120 | 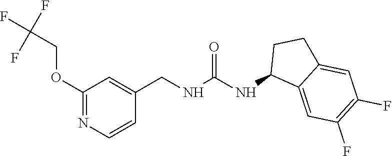 [SMILES: O=C(NCc1ccnc(OCC(F)(F)F)c1)N[C@H]1CCc2cc(F)c(F)cc21] |
| 121 | 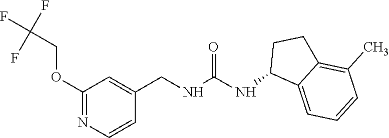 [SMILES: Cc1cccc2c1CC[C@H]2NC(=O)NCc1ccnc(OCC(F)(F)F)c1] |
| 122 | 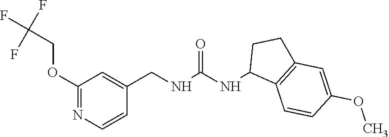 [SMILES: COc1ccc2c(c1)CCC2NC(=O)NCc1ccnc(OCC(F)(F)F)c1] |
| 123 | 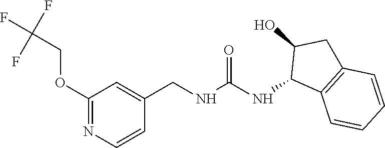 [SMILES: O=C(NCc1ccnc(OCC(F)(F)F)c1)N[C@H]1c2ccccc2C[C@@H]1O] |
| 124 | 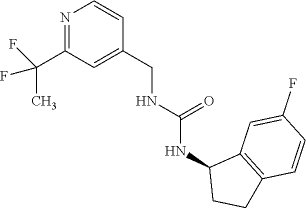 [SMILES: CC(F)(F)c1cc(CNC(=O)N[C@@H]2CCc3ccc(F)cc32)ccn1] |
| 125 | 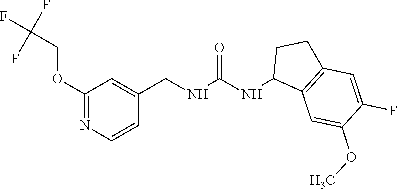 [SMILES: COc1cc2c(cc1F)CCC2NC(=O)NCc1ccnc(OCC(F)(F)F)c1] |
| 126 | 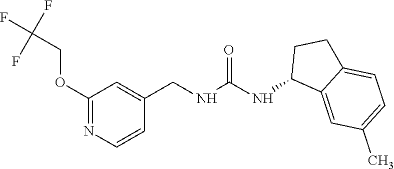 [SMILES: Cc1ccc2c(c1)[C@H](NC(=O)NCc1ccnc(OCC(F)(F)F)c1)CC2] |
| 127 | 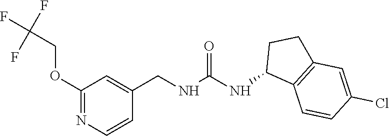 [SMILES: O=C(NCc1ccnc(OCC(F)(F)F)c1)N[C@@H]1CCc2cc(Cl)ccc21] |
| 128 | 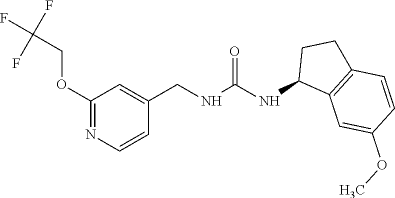 [SMILES: COc1ccc2c(c1)[C@@H](NC(=O)NCc1ccnc(OCC(F)(F)F)c1)CC2] |
| 129 | 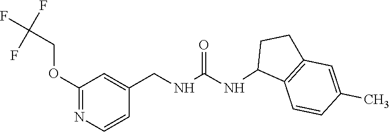 [SMILES: Cc1ccc2c(c1)CCC2NC(=O)NCc1ccnc(OCC(F)(F)F)c1] |
| 130 | 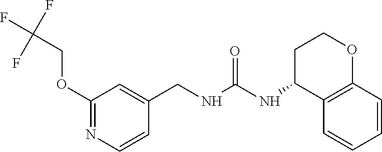 [SMILES: O=C(NCc1ccnc(OCC(F)(F)F)c1)N[C@@H]1CCOc2ccccc21] |
| 131 | 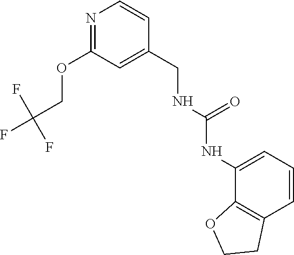 [SMILES: O=C(NCc1ccnc(OCC(F)(F)F)c1)Nc1cccc2c1OCC2] |
| 132 | 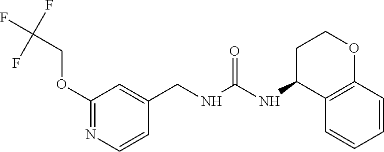 [SMILES: O=C(NCc1ccnc(OCC(F)(F)F)c1)N[C@H]1CCOc2ccccc21] |
| 133 | 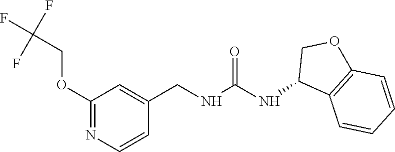 [SMILES: O=C(NCc1ccnc(OCC(F)(F)F)c1)N[C@@H]1COc2ccccc21] |
| 134 | 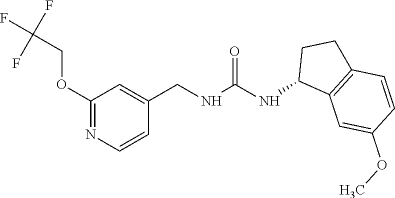 [SMILES: COc1ccc2c(c1)[C@H](NC(=O)NCc1ccnc(OCC(F)(F)F)c1)CC2] |
| 135 | 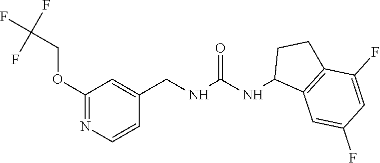 [SMILES: O=C(NCc1ccnc(OCC(F)(F)F)c1)NC1CCc2c(F)cc(F)cc21] |
| 136 | 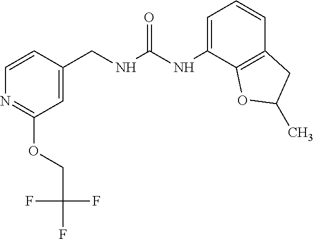 [SMILES: CC1Cc2cccc(NC(=O)NCc3ccnc(OCC(F)(F)F)c3)c2O1] |
| 137 | 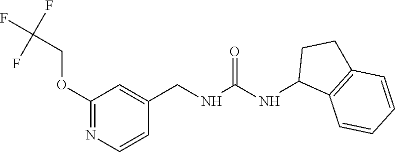 [SMILES: O=C(NCc1ccnc(OCC(F)(F)F)c1)NC1CCc2ccccc21] |
| 138 | 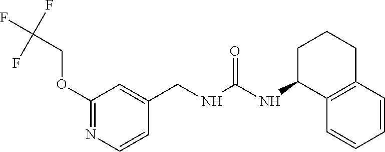 [SMILES: O=C(NCc1ccnc(OCC(F)(F)F)c1)N[C@H]1CCCc2ccccc21] |
| 139 | 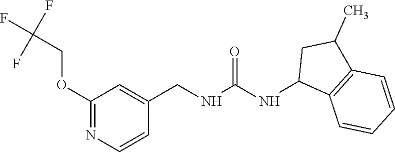 [SMILES: CC1CC(NC(=O)NCc2ccnc(OCC(F)(F)F)c2)c2ccccc21] |
| 140 | 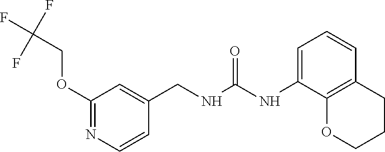 [SMILES: O=C(NCc1ccnc(OCC(F)(F)F)c1)Nc1cccc2c1OCCC2] |
| 141 | 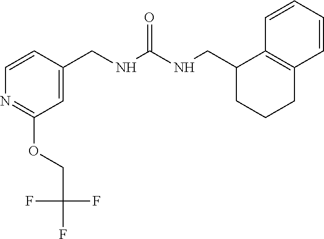 [SMILES: O=C(NCc1ccnc(OCC(F)(F)F)c1)NCC1CCCc2ccccc21] |
| 142 | 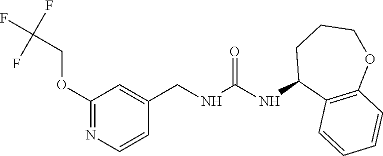 [SMILES: O=C(NCc1ccnc(OCC(F)(F)F)c1)N[C@H]1CCCOc2ccccc21] |
| 143 | 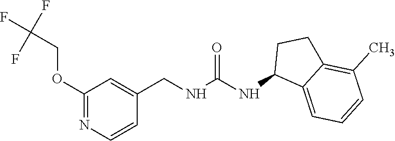 [SMILES: Cc1cccc2c1CC[C@@H]2NC(=O)NCc1ccnc(OCC(F)(F)F)c1] |
| 144 | 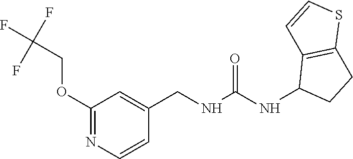 [SMILES: O=C(NCc1ccnc(OCC(F)(F)F)c1)NC1CCc2sccc21] |
| 145 | 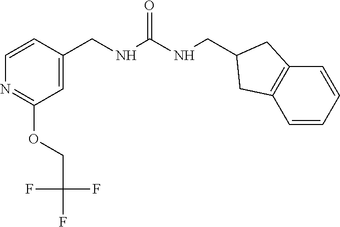 [SMILES: O=C(NCc1ccnc(OCC(F)(F)F)c1)NCC1Cc2ccccc2C1] |
| 146 | 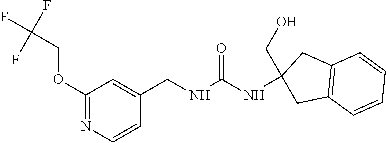 [SMILES: O=C(NCc1ccnc(OCC(F)(F)F)c1)NC1(CO)Cc2ccccc2C1] |
| 147 | 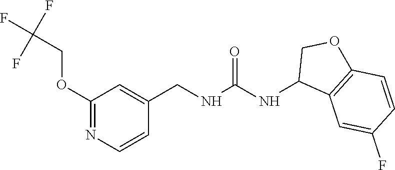 [SMILES: O=C(NCc1ccnc(OCC(F)(F)F)c1)NC1COc2ccc(F)cc21] |
| 148 | 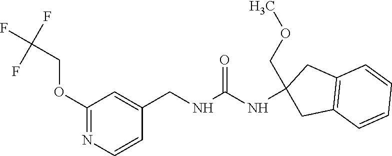 [SMILES: COCC1(NC(=O)NCc2ccnc(OCC(F)(F)F)c2)Cc2ccccc2C1] |
| 149 | 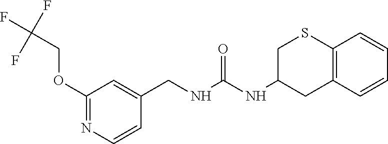 [SMILES: O=C(NCc1ccnc(OCC(F)(F)F)c1)NC1CSc2ccccc2C1] |
| 150 | 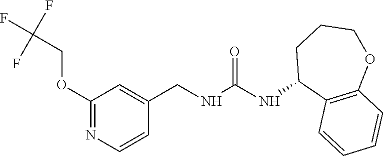 [SMILES: O=C(NCc1ccnc(OCC(F)(F)F)c1)N[C@@H]1CCCOc2ccccc21] |
| 151 | 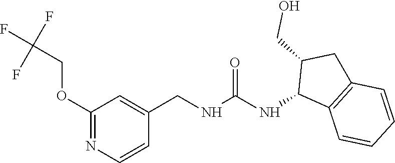 [SMILES: O=C(NCc1ccnc(OCC(F)(F)F)c1)N[C@H]1c2ccccc2C[C@H]1CO] |
| 152 | 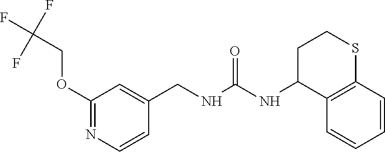 [SMILES: O=C(NCc1ccnc(OCC(F)(F)F)c1)NC1CCSc2ccccc21] |
| 153 | 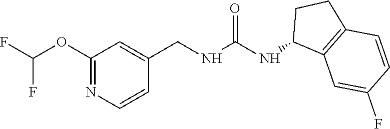 [SMILES: O=C(NCc1ccnc(OC(F)F)c1)N[C@@H]1CCc2ccc(F)cc21] |
| 154 | 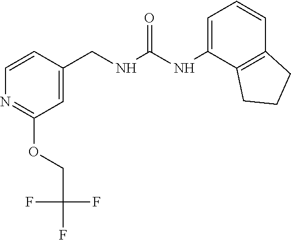 [SMILES: O=C(NCc1ccnc(OCC(F)(F)F)c1)Nc1cccc2c1CCC2] |
| 155 | 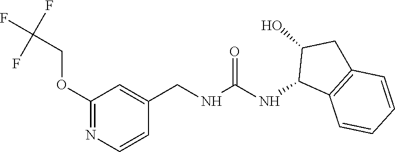 [SMILES: O=C(NCc1ccnc(OCC(F)(F)F)c1)N[C@H]1c2ccccc2C[C@H]1O] |
| 156 | 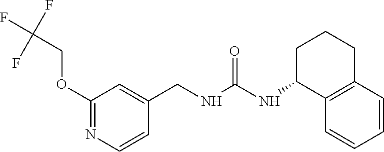 [SMILES: O=C(NCc1ccnc(OCC(F)(F)F)c1)N[C@@H]1CCCc2ccccc21] |
| 157 | 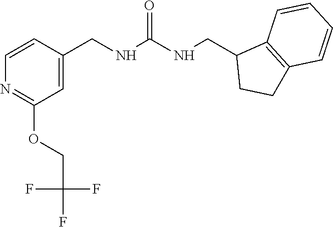 [SMILES: O=C(NCc1ccnc(OCC(F)(F)F)c1)NCC1CCc2ccccc21] |
| 158 | 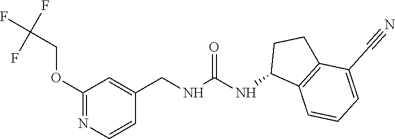 [SMILES: N#Cc1cccc2c1CC[C@H]2NC(=O)NCc1ccnc(OCC(F)(F)F)c1] |
| 159 | 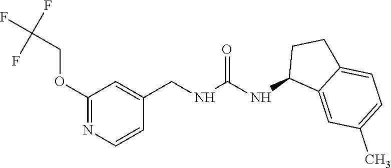 [SMILES: Cc1ccc2c(c1)[C@@H](NC(=O)NCc1ccnc(OCC(F)(F)F)c1)CC2] |
| 160 | 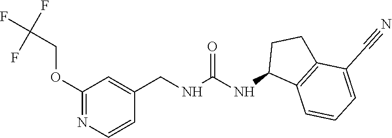 [SMILES: N#Cc1cccc2c1CC[C@@H]2NC(=O)NCc1ccnc(OCC(F)(F)F)c1] |
| 161 | 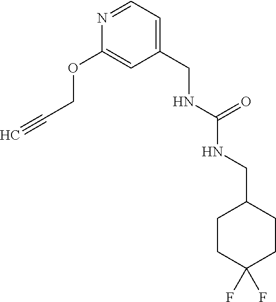 [SMILES: C#CCOc1cc(CNC(=O)NCC2CCC(F)(F)CC2)ccn1] |
| 162 | 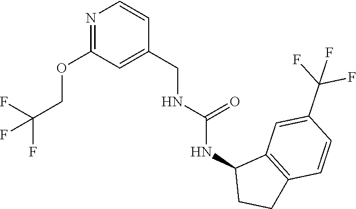 [SMILES: O=C(NCc1ccnc(OCC(F)(F)F)c1)N[C@@H]1CCc2ccc(C(F)(F)F)cc21] |
| 163 | 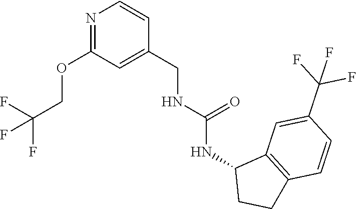 [SMILES: O=C(NCc1ccnc(OCC(F)(F)F)c1)N[C@H]1CCc2ccc(C(F)(F)F)cc21] |
| 164 | 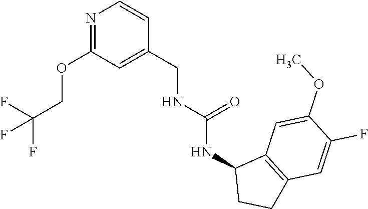 [SMILES: COc1cc2c(cc1F)CC[C@H]2NC(=O)NCc1ccnc(OCC(F)(F)F)c1] |
| 165 | 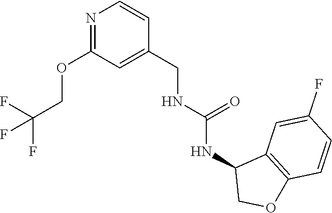 [SMILES: O=C(NCc1ccnc(OCC(F)(F)F)c1)N[C@@H]1COc2ccc(F)cc21] |
| 166 | 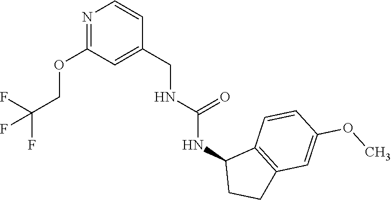 [SMILES: COc1ccc2c(c1)CC[C@H]2NC(=O)NCc1ccnc(OCC(F)(F)F)c1] |

TABLE B

| No. | Structure |
|---|---|
| 167 | 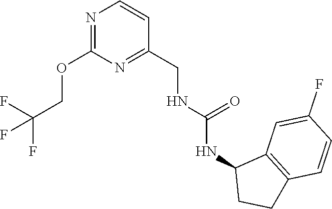 [SMILES: O=C(NCc1ccnc(OCC(F)(F)F)n1)N[C@@H]1CCc2ccc(F)cc21] |
| 168 | 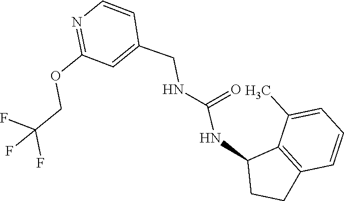 [SMILES: Cc1cccc2c1[C@H](NC(=O)NCc1ccnc(OCC(F)(F)F)c1)CC2] |
| 169 | 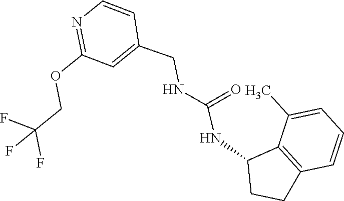 [SMILES: Cc1cccc2c1[C@@H](NC(=O)NCc1ccnc(OCC(F)(F)F)c1)CC2] |
| 170 | 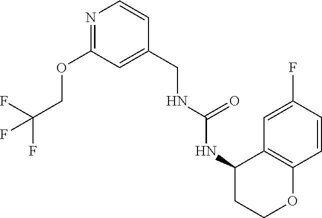 [SMILES: O=C(NCc1ccnc(OCC(F)(F)F)c1)N[C@@H]1CCOc2ccc(F)cc21] |
| 171 | 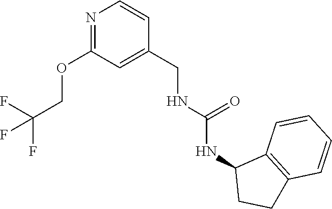 [SMILES: O=C(NCc1ccnc(OCC(F)(F)F)c1)N[C@@H]1CCc2ccccc21] |
| 172 | 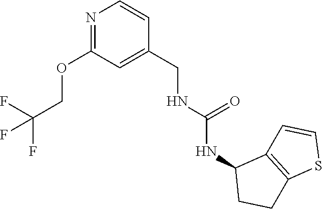 [SMILES: O=C(NCc1ccnc(OCC(F)(F)F)c1)N[C@@H]1CCc2sccc21] |
| 173 | 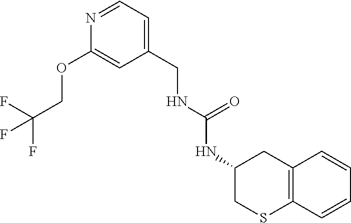 [SMILES: O=C(NCc1ccnc(OCC(F)(F)F)c1)N[C@H]1CSc2ccccc2C1] |
| 174 | 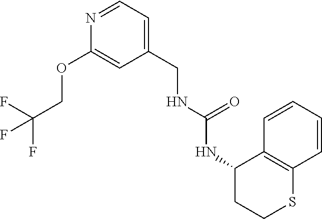 [SMILES: O=C(NCc1ccnc(OCC(F)(F)F)c1)N[C@H]1CCSc2ccccc21] |
| 175 | 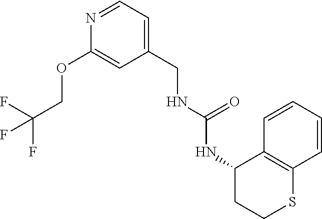 [SMILES: O=C(NCc1ccnc(OCC(F)(F)F)c1)N[C@H]1CCSc2ccccc21] |
| 176 | 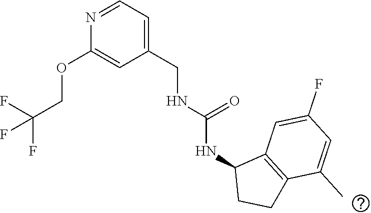 [SMILES: Cc1cc(F)cc2c1CC[C@H]2NC(=O)NCc1ccnc(OCC(F)(F)F)c1] |
| 177 | 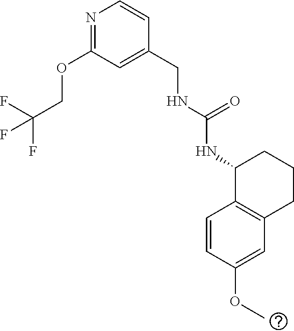 [SMILES: COc1ccc2c(c1)CCC[C@H]2NC(=O)NCc1ccnc(OCC(F)(F)F)c1] |
| 178 | 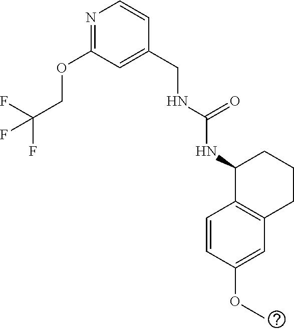 [SMILES: COc1ccc2c(c1)CCC[C@@H]2NC(=O)NCc1ccnc(OCC(F)(F)F)c1] |
| 179 | 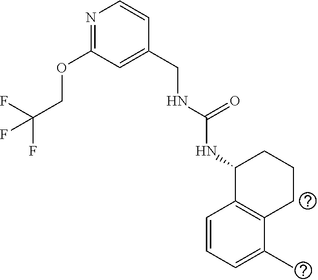 [SMILES: Cc1cccc2c1CCC[C@H]2NC(=O)NCc1ccnc(OCC(F)(F)F)c1] |
| 180 | 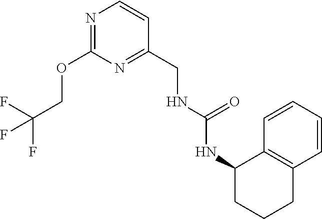 [SMILES: O=C(NCc1ccnc(OCC(F)(F)F)n1)N[C@@H]1CCCc2ccccc21] |
| 181 | 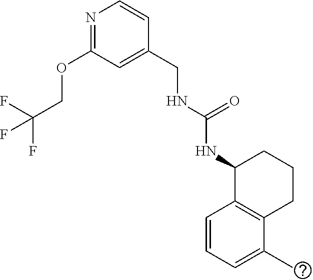 [SMILES: Cc1cccc2c1CCC[C@@H]2NC(=O)NCc1ccnc(OCC(F)(F)F)c1] |
| 182 | 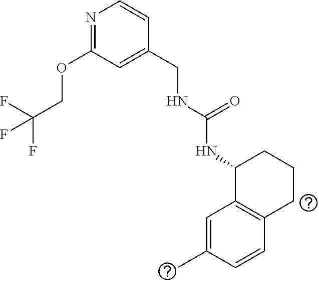 [SMILES: Cc1ccc2c(c1)[C@H](NC(=O)NCc1ccnc(OCC(F)(F)F)c1)CCC2] |
| 183 | 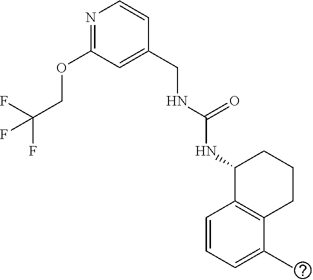 [SMILES: Cc1cccc2c1CCC[C@H]2NC(=O)NCc1ccnc(OCC(F)(F)F)c1] |
| 184 | 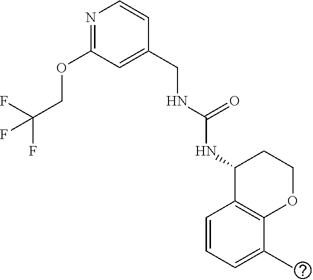 [SMILES: Cc1cccc2c1OCC[C@H]2NC(=O)NCc1ccnc(OCC(F)(F)F)c1] |
| 185 | 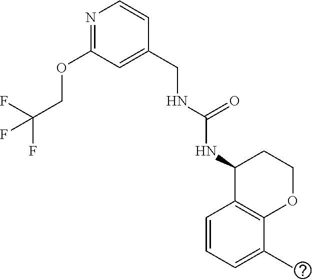 [SMILES: Cc1cccc2c1OCC[C@@H]2NC(=O)NCc1ccnc(OCC(F)(F)F)c1] |
| 186 | 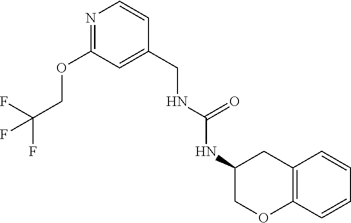 [SMILES: O=C(NCc1ccnc(OCC(F)(F)F)c1)N[C@@H]1COc2ccccc2C1] |
| 187 | 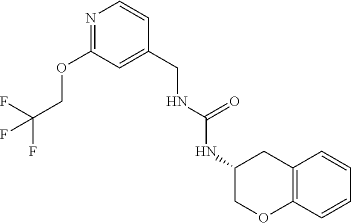 [SMILES: O=C(NCc1ccnc(OCC(F)(F)F)c1)N[C@H]1COc2ccccc2C1] |
| 188 | 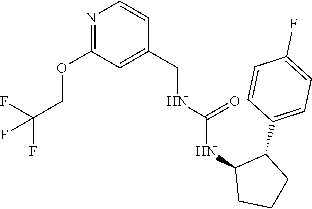 [SMILES: O=C(NCc1ccnc(OCC(F)(F)F)c1)N[C@@H]1CCC[C@H]1c1ccc(F)cc1] |
| 189 | 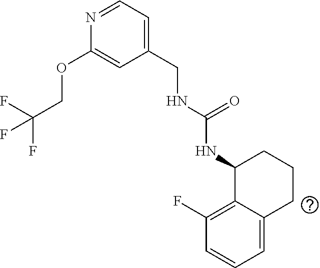 [SMILES: O=C(NCc1ccnc(OCC(F)(F)F)c1)N[C@H]1CCCc2cccc(F)c21] |
| 190 | 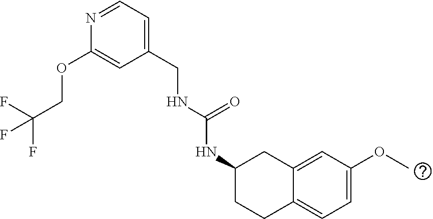 [SMILES: COc1ccc2c(c1)C[C@H](NC(=O)NCc1ccnc(OCC(F)(F)F)c1)CC2] |
| 191 | 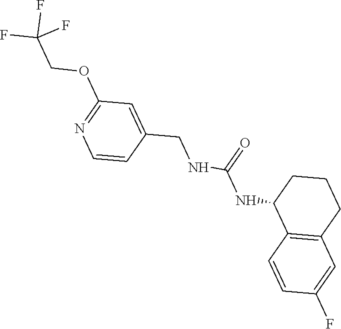 [SMILES: O=C(NCc1ccnc(OCC(F)(F)F)c1)N[C@@H]1CCCc2cc(F)ccc21] |
| 192 | 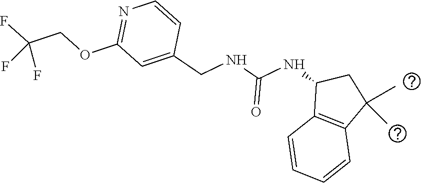 [SMILES: CC1(C)C[C@@H](NC(=O)NCc2ccnc(OCC(F)(F)F)c2)c2ccccc21] |
| 193 | 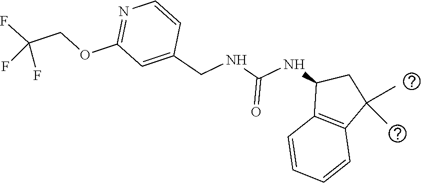 [SMILES: CC1(C)C[C@H](NC(=O)NCc2ccnc(OCC(F)(F)F)c2)c2ccccc21] |
| 194 | 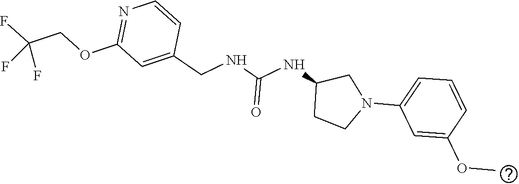 [SMILES: COc1cccc(N2CC[C@@H](NC(=O)NCc3ccnc(OCC(F)(F)F)c3)C2)c1] |
| 195 | 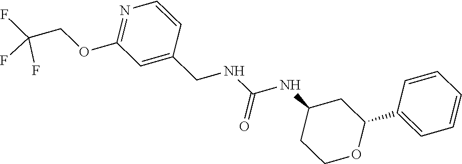 [SMILES: O=C(NCc1ccnc(OCC(F)(F)F)c1)N[C@@H]1CCO[C@@H](c2ccccc2)C1] |
| 196 | 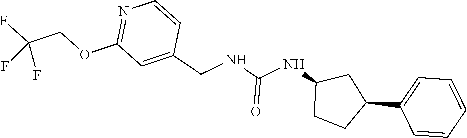 [SMILES: O=C(NCc1ccnc(OCC(F)(F)F)c1)N[C@@H]1CC[C@H](c2ccccc2)C1] |
| 197 | 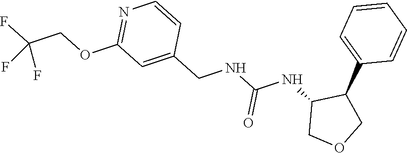 [SMILES: O=C(NCc1ccnc(OCC(F)(F)F)c1)N[C@H]1COC[C@@H]1c1ccccc1] |
| 198 | 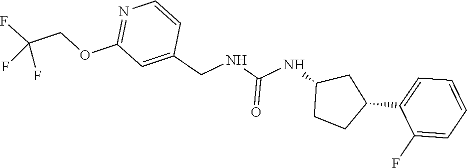 [SMILES: O=C(NCc1ccnc(OCC(F)(F)F)c1)N[C@H]1CC[C@@H](c2ccccc2F)C1] |
| 199 | 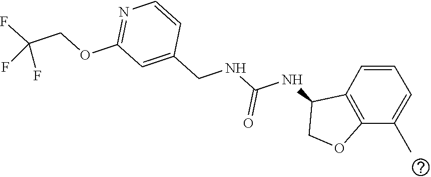 [SMILES: Cc1cccc2c1OC[C@H]2NC(=O)NCc1ccnc(OCC(F)(F)F)c1] |
| 200 | 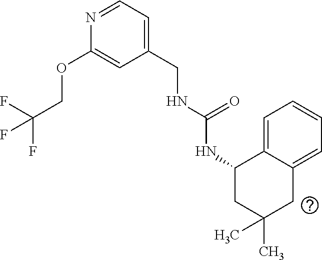 [SMILES: CC1(C)Cc2ccccc2[C@@H](NC(=O)NCc2ccnc(OCC(F)(F)F)c2)C1] |
| 201 | 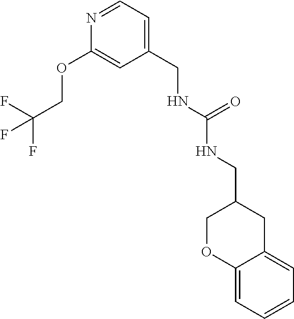 [SMILES: O=C(NCc1ccnc(OCC(F)(F)F)c1)NCC1COc2ccccc2C1] |
| 202 | 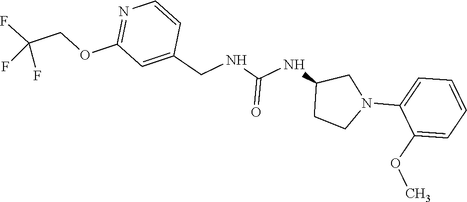 [SMILES: COc1ccccc1N1CC[C@@H](NC(=O)NCc2ccnc(OCC(F)(F)F)c2)C1] |
| 203 | 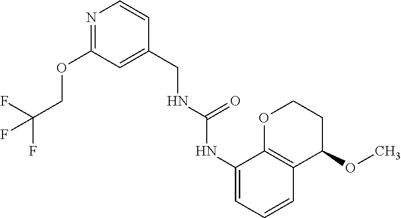 [SMILES: CO[C@@H]1CCOc2c(NC(=O)NCc3ccnc(OCC(F)(F)F)c3)cccc21] |
| 204 | 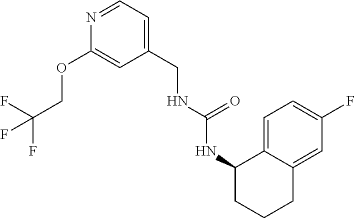 [SMILES: O=C(NCc1ccnc(OCC(F)(F)F)c1)N[C@@H]1CCCc2cc(F)ccc21] |
| 205 | 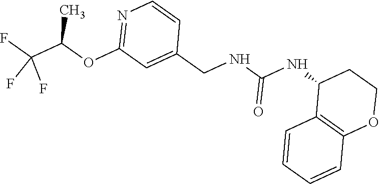 [SMILES: C[C@@H](Oc1cc(CNC(=O)N[C@@H]2CCOc3ccccc32)ccn1)C(F)(F)F] |
| 206 | 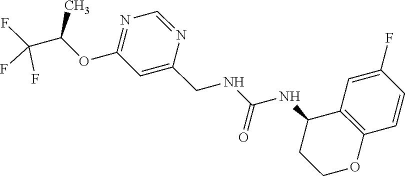 [SMILES: C[C@@H](Oc1cc(CNC(=O)N[C@@H]2CCOc3ccc(F)cc32)ncn1)C(F)(F)F] |
| 207 | 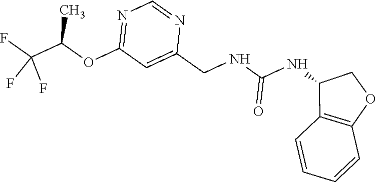 [SMILES: C[C@@H](Oc1cc(CNC(=O)N[C@@H]2COc3ccccc32)ncn1)C(F)(F)F] |
| 208 | 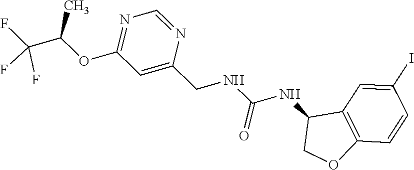 [SMILES: C[C@@H](Oc1cc(CNC(=O)N[C@@H]2COc3ccc(I)cc32)ncn1)C(F)(F)F] |
| 209 | 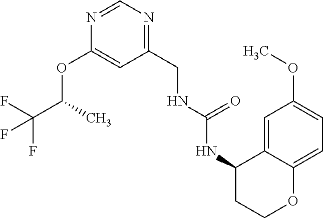 [SMILES: COc1ccc2c(c1)[C@H](NC(=O)NCc1cc(O[C@H](C)C(F)(F)F)ncn1)CCO2] |
| 210 | 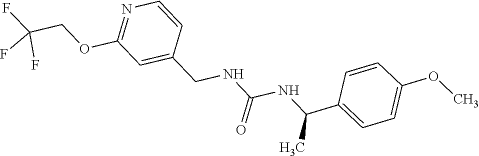 [SMILES: COc1ccc([C@@H](C)NC(=O)NCc2ccnc(OCC(F)(F)F)c2)cc1] |
| 211 | 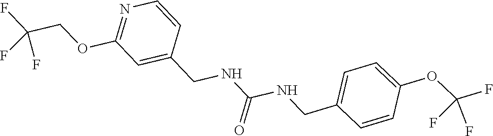 [SMILES: O=C(NCc1ccc(OC(F)(F)F)cc1)NCc1ccnc(OCC(F)(F)F)c1] |
| 212 | 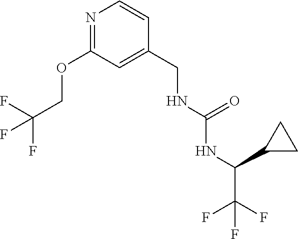 [SMILES: O=C(NCc1ccnc(OCC(F)(F)F)c1)N[C@@H](C1CC1)C(F)(F)F] |
| 213 | 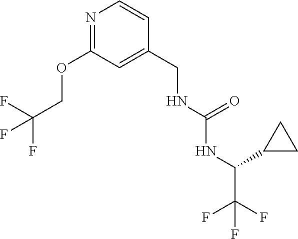 [SMILES: O=C(NCc1ccnc(OCC(F)(F)F)c1)N[C@H](C1CC1)C(F)(F)F] |
| 214 | 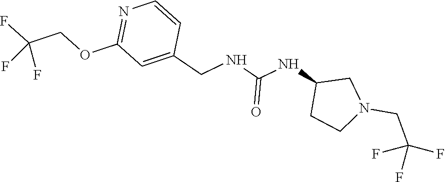 [SMILES: O=C(NCc1ccnc(OCC(F)(F)F)c1)N[C@@H]1CCN(CC(F)(F)F)C1] |
| 215 | 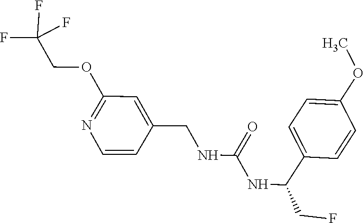 [SMILES: COc1ccc([C@H](CF)NC(=O)NCc2ccnc(OCC(F)(F)F)c2)cc1] |
| 216 | 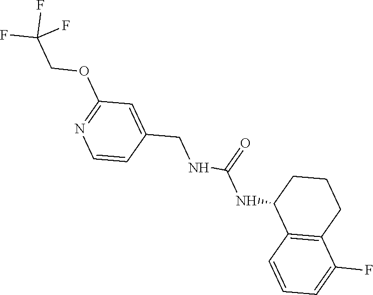 [SMILES: O=C(NCc1ccnc(OCC(F)(F)F)c1)N[C@@H]1CCCc2c(F)cccc21] |
| 217 | 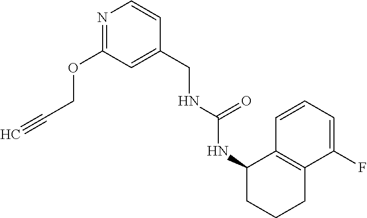 [SMILES: C#CCOc1cc(CNC(=O)N[C@@H]2CCCc3c(F)cccc32)ccn1] |
| 218 | 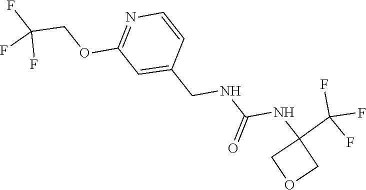 [SMILES: O=C(NCc1ccnc(OCC(F)(F)F)c1)NC1(C(F)(F)F)COC1] |
| 219 | 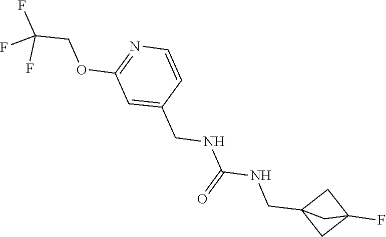 [SMILES: O=C(NCc1ccnc(OCC(F)(F)F)c1)NCC12CC(F)(C1)C2] |
| 220 | 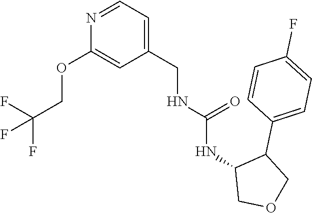 [SMILES: O=C(NCc1ccnc(OCC(F)(F)F)c1)N[C@H]1COCC1c1ccc(F)cc1] |
| 221 | 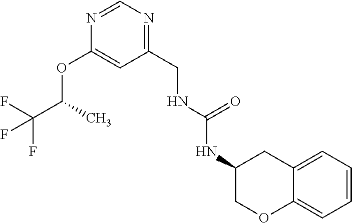 [SMILES: C[C@@H](Oc1cc(CNC(=O)N[C@@H]2COc3ccccc3C2)ncn1)C(F)(F)F] |
| 222 | 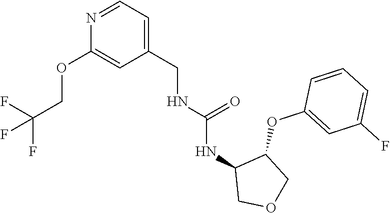 [SMILES: O=C(NCc1ccnc(OCC(F)(F)F)c1)N[C@@H]1COC[C@H]1Oc1cccc(F)c1] |
| 223 | 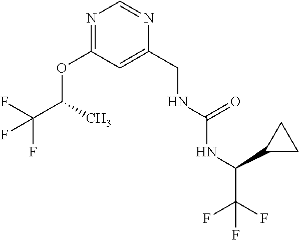 [SMILES: C[C@@H](Oc1cc(CNC(=O)N[C@@H](C2CC2)C(F)(F)F)ncn1)C(F)(F)F] |
| 224 | 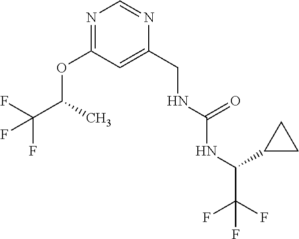 [SMILES: C[C@@H](Oc1cc(CNC(=O)N[C@H](C2CC2)C(F)(F)F)ncn1)C(F)(F)F] |
| 225 | 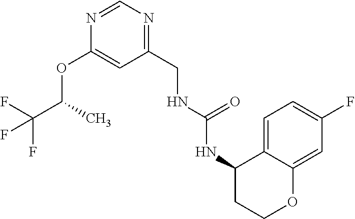 [SMILES: C[C@@H](Oc1cc(CNC(=O)N[C@@H]2CCOc3cc(F)ccc32)ncn1)C(F)(F)F] |
| 226 | 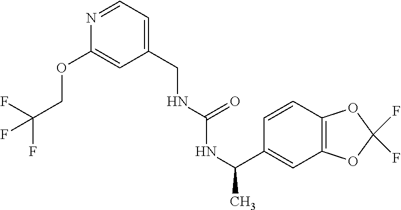 [SMILES: C[C@@H](NC(=O)NCc1ccnc(OCC(F)(F)F)c1)c1ccc2c(c1)OC(F)(F)O2] |
| 227 | 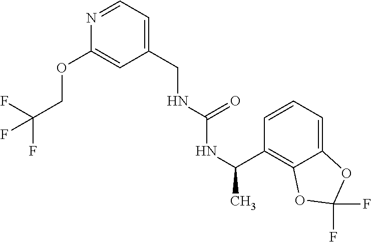 [SMILES: C[C@@H](NC(=O)NCc1ccnc(OCC(F)(F)F)c1)c1cccc2c1OC(F)(F)O2] |
| 228 | 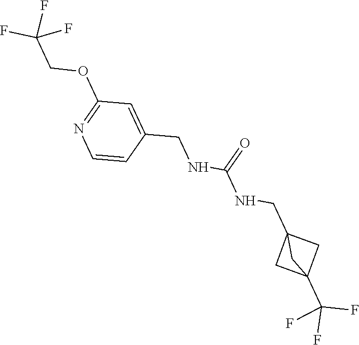 [SMILES: O=C(NCc1ccnc(OCC(F)(F)F)c1)NCC12CC(C(F)(F)F)(C1)C2] |
| 229 | 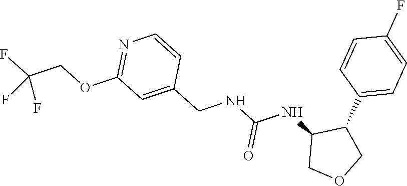 [SMILES: O=C(NCc1ccnc(OCC(F)(F)F)c1)N[C@@H]1COC[C@H]1c1ccc(F)cc1] |
| 230 | 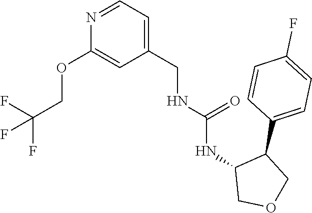 [SMILES: O=C(NCc1ccnc(OCC(F)(F)F)c1)N[C@H]1COC[C@@H]1c1ccc(F)cc1] |
| 231 | 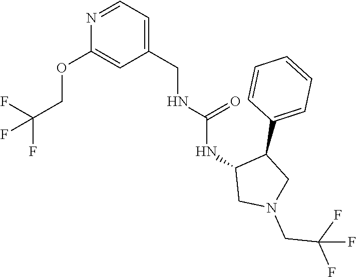 [SMILES: O=C(NCc1ccnc(OCC(F)(F)F)c1)N[C@H]1CN(CC(F)(F)F)C[C@@H]1c1ccccc1] |
| 232 | 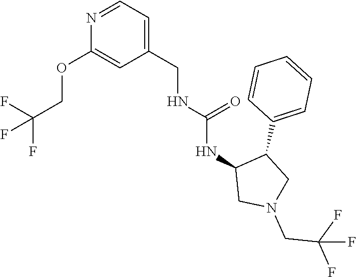 [SMILES: O=C(NCc1ccnc(OCC(F)(F)F)c1)N[C@@H]1CN(CC(F)(F)F)C[C@H]1c1ccccc1] |
| 233 | 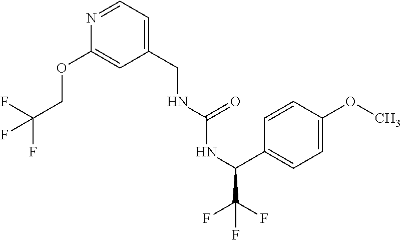 [SMILES: COc1ccc([C@H](NC(=O)NCc2ccnc(OCC(F)(F)F)c2)C(F)(F)F)cc1] |
| 234 | 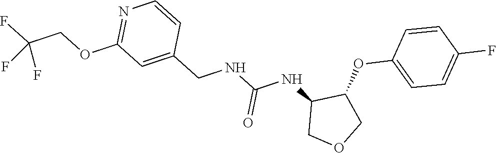 [SMILES: O=C(NCc1ccnc(OCC(F)(F)F)c1)N[C@@H]1COC[C@H]1Oc1ccc(F)cc1] |
| 235 | 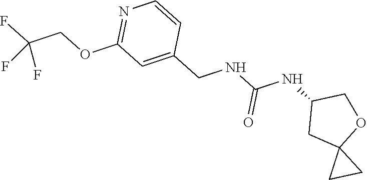 [SMILES: O=C(NCc1ccnc(OCC(F)(F)F)c1)N[C@@H]1COC2(CC2)C1] |
| 236 | 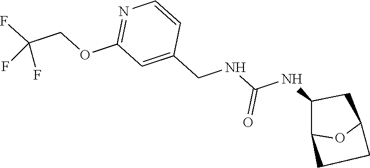 [SMILES: O=C(NCc1ccnc(OCC(F)(F)F)c1)N[C@H]1C[C@H]2CC[C@@H]1O2] |
| 237 | 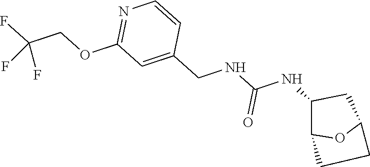 [SMILES: O=C(NCc1ccnc(OCC(F)(F)F)c1)N[C@@H]1C[C@@H]2CC[C@H]1O2] |
| 238 | 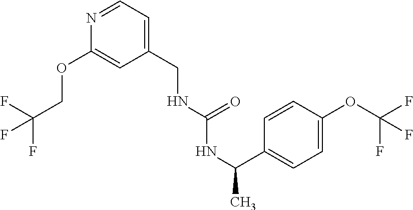 [SMILES: C[C@@H](NC(=O)NCc1ccnc(OCC(F)(F)F)c1)c1ccc(OC(F)(F)F)cc1] |
| 239 | 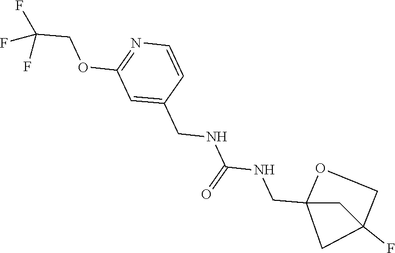 [SMILES: O=C(NCc1ccnc(OCC(F)(F)F)c1)NCC12CC(F)(CO1)C2] |
| 240 | 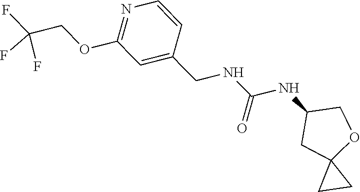 [SMILES: O=C(NCc1ccnc(OCC(F)(F)F)c1)N[C@H]1COC2(CC2)C1] |
| 241 | 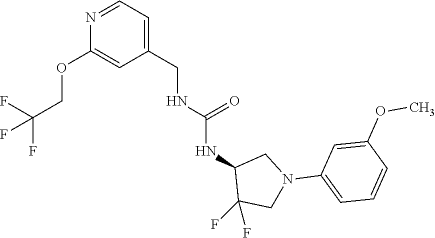 [SMILES: COc1cccc(N2C[C@H](NC(=O)NCc3ccnc(OCC(F)(F)F)c3)C(F)(F)C2)c1] |
| 242 | 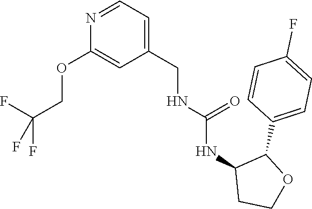 [SMILES: O=C(NCc1ccnc(OCC(F)(F)F)c1)N[C@@H]1CCO[C@H]1c1ccc(F)cc1] |
| 243 | 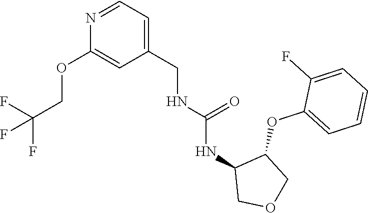 [SMILES: O=C(NCc1ccnc(OCC(F)(F)F)c1)N[C@@H]1COC[C@H]1Oc1ccccc1F] |
| 244 | 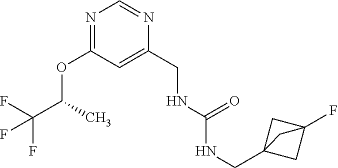 [SMILES: C[C@@H](Oc1cc(CNC(=O)NCC23CC(F)(C2)C3)ncn1)C(F)(F)F] |
| 245 | 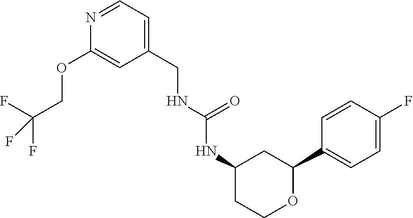 [SMILES: O=C(NCc1ccnc(OCC(F)(F)F)c1)N[C@@H]1CCO[C@H](c2ccc(F)cc2)C1] |
| 246 | 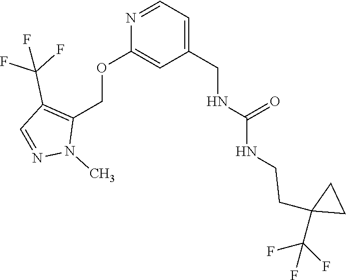 [SMILES: Cn1ncc(C(F)(F)F)c1COc1cc(CNC(=O)NCCC2(C(F)(F)F)CC2)ccn1] |
| 247 | 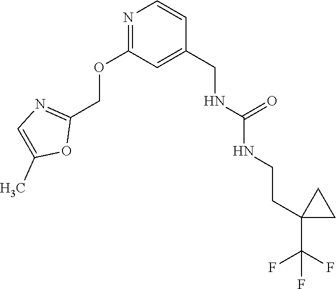 [SMILES: Cc1cnc(COc2cc(CNC(=O)NCCC3(C(F)(F)F)CC3)ccn2)o1] |
| 248 | 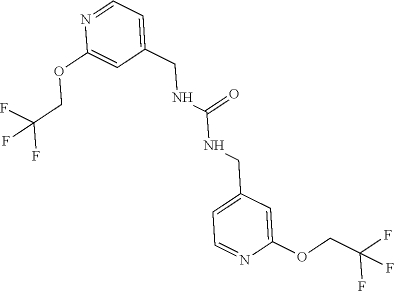 [SMILES: O=C(NCc1ccnc(OCC(F)(F)F)c1)NCc1ccnc(OCC(F)(F)F)c1] |
| 249 | 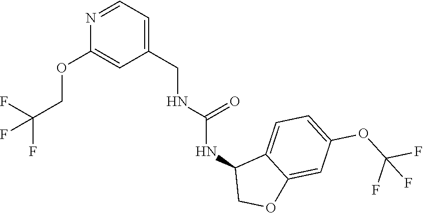 [SMILES: O=C(NCc1ccnc(OCC(F)(F)F)c1)N[C@@H]1COc2cc(OC(F)(F)F)ccc21] |
| 250 | 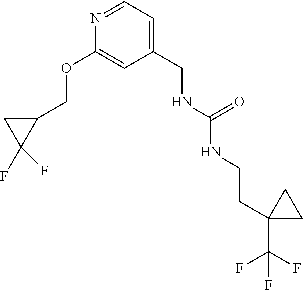 [SMILES: O=C(NCCC1(C(F)(F)F)CC1)NCc1ccnc(OCC2CC2(F)F)c1] |

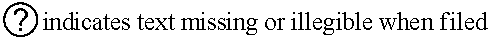

13: The Kv7.2/7.3 potassium channel activator compound according to claim 12, wherein said pharmaceutically acceptable salt is selected from the group consisting of a salts with an organic acid, a salt with an organic base, a salt with an inorganic acid, and a salt with an inorganic base.

14. (canceled)

15: A method for treating a disorder modulated by a KV7.2/KV7.3 potassium channel, comprising administering to a subject in need thereof an effective amount of a pharmaceutical composition comprising (i) at least one Kv7.2/7.3 potassium channel activating compound according to claim 1, and (ii) at least one pharmaceutically acceptable excipient.

16: A pharmaceutical composition comprising (i) at least one Kv7.2/7.3 potassium channel activating compound according to claim 12, or a pharmaceutically acceptable salt thereof, and (ii) at least one pharmaceutically acceptable excipient.

17: A method of treating a disorder modulated by a KV7.2/KV7.3 potassium channel comprising administering to a subject in need thereof an effective amount of a pharmaceutical composition according to claim 16.

18: The method according to claim 15, wherein said disorder which is modulated by a Kv7.2/7.3 potassium channel is a central nervous system (CNS) or a peripheral nervous system (PNS) disorder.

19: The method according to claim 18, wherein said central nervous system (CNS) disorder is selected from the group consisting of epilepsy, epileptic syndrome, epileptic symptom, epilepsy resistant or refractory to treatment, a seizure, bipolar disorder, bipolar depression, schizophrenia, psychosis, mania, a stress-related disorder, an acute stress reaction, major depressive disorder, anxiety, a panic attack, social phobia, a sleep disorder, attention deficit hyperactivity disorder, post-traumatic stress disorder, obsessive-compulsive disorder, an impulsivity disorder, a personality disorders, Huntington's disease, Alzheimer's disease, Parkinson's disease, multiple sclerosis, amyotrophic lateral sclerosis, and tinnitus.

20: The method according to claim 18, wherein said peripheral nervous system (PNS) disorder is selected from the group consisting of migraine, chronic pain, acute pain, neuropathic pain, visceral pain, inflammatory pain, and muscle pain.

21: A method of treatment of disorder which is modulated by a Kv7.2/7.3 potassium channel, selected from the group consisting of a central nervous system (CNS) and a peripheral nervous system (PNS) disorder, comprising administering to a human being in need thereof of an effective amount of a compound according to claim 12.

22: The method according to claim 1, wherein said pharmaceutically acceptable salt is selected from the group consisting of a salt with an organic acid selected from the group consisting of oxalic, maleic, methanesulfonic, paratoluenesulfonic, succinic, citric, malic, tartaric and lactic acid, a salt with an organic base selected from the group consisting of thromethamine, lysine, arginine, glycine, alanine and ethanolamine, a salt with an inorganic acid selected from the group consisting of hydrochloric, hydrobromic, phosphoric and sulfuric acid, and a salt with an inorganic base selected from the group consisting of a hydroxide and carbonate of an alkaline or alkaline-earth metal.

23: The Kv7.2/7.3 potassium channel activator compound according to claim 12, wherein said pharmaceutically acceptable salt is selected from the group consisting of a salt with an organic acid selected from the group consisting of oxalic, maleic, methanesulfonic, paratoluenesulfonic, succinic, citric, malic, tartaric and lactic acid, a salt with an organic base selected from the group consisting of thromethamine, lysine, arginine, glycine, alanine and ethanolamine, a salt with an inorganic acid selected from the group consisting of hydrochloric, hydrobromic, phosphoric and sulfuric acid, and a salt with an inorganic base selected from the group consisting of a hydroxide and carbonate of an alkaline or alkaline-earth metal.
